(19) United States
(12) Patent Application Publication
(10) Pub. No.: US 2014/0288113 A1
(43) Pub. Date: Sep. 25, 2014

(54) Extended Release Compositions Comprising Hydrocodone And Acetaminophen For Rapid Onset And Prolonged Analgesia That May Be Administered Without Regard To Food

(71) Applicant: MALLINCKRODT LLC, Hazelwood, MO (US)

(72) Inventors: Krishna R. Devarakonda, Saint Louis, MO (US); Michael J. Giuliani, Creve Coeur, MO (US); Vishal K. Gupta, Hillsborough, NJ (US); Ralph A. Heasley, Webster Groves, MO (US); Susan Shelby, Town and Country, MO (US)

(73) Assignee: MALLINCKRODT LLC, Hazelwood, MO (US)

(21) Appl. No.: 14/187,939

(22) Filed: Feb. 24, 2014

Related U.S. Application Data
(60) Provisional application No. 61/799,341, filed on Mar. 15, 2013.
(60) Provisional application No. 61/798,525, filed on Mar. 15, 2013.
(60) Provisional application No. 61/871,956, filed on Aug. 30, 2013.
(60) Provisional application No. 61/928,853, filed on Jan. 17, 2014.
(60) Provisional application No. 61/926,523, filed on Jan. 13, 2014.

(51) Int. Cl. A61K 47/08; A61K 31/485; A61K 31/167

(52) U.S. Cl. CPC A61K 47/08; A61K 31/167; A61K 31/485
USPC 514/282

(57) ABSTRACT

The present disclosure provides an extended release pharmaceutical composition comprising hydrocodone and acetaminophen that provides a rapid onset of analgesia, and reduced levels of acetaminophen near the end of the dosing interval. Also provided are methods for reducing the risk of acetaminophen-induced hepatic damage in a subject being treated with an acetaminophen containing composition, as well as methods for treating pain in a subject in need thereof.

### CLAIM OF PRIORITY

This application claims priority to U.S. Provisional Application No. 61/799,341, filed on Mar. 15, 2013, U.S. Provisional Application No. 61/798,525, filed on Mar. 15, 2013, U.S. Provisional Application No. 61/871,956, filed on Aug. 30, 2013, U.S. Provisional Application No. 61/928,853, filed on Jan. 17, 2014, and U.S. Provisional Application No. 61/926,523, filed on Jan. 13, 2014.

### FIELD OF THE INVENTION

The present disclosure relates to an extended release pharmaceutical composition comprising hydrocodone and acetaminophen that provides a rapid onset of analgesia, followed by an extended duration of analgesia of about 12 hours.

### BACKGROUND OF THE INVENTION

Oral drug administration remains the route of choice for the majority of clinical applications. Modified release (MR) dosage forms that are administered once or twice daily offer advantages over their immediate release (IR) counterparts because they reduce the magnitude of peaks and troughs of drug plasma concentration, provide longer dosing intervals, sustained analgesic effect, and increased patient compliance. These modified release formulations may be referred to as controlled release (CR), sustained release (SR) and/or extended release (ER) etc. For certain types of patients, such as those suffering from pain, these MR products may permit the patient to sleep through the night without having to wake up during the night to take the next dose. Thus, it can significantly increase the quality of life for such patients.

Both IR and MR products for pain are widely available in the market. Examples of IR products include those containing NSAIDs, opioids, profens, COX II inhibitors and aspirin (Tylenol, Advil, Celebrex, Vioxx, Aleve, Voltaren). Examples of MR products include those containing NSAIDs and opioids (Tylenol SR, Oxycontin).

Researchers have also combined various classes of pain drugs to provide better analgesia to patients. For example, a combination of acetaminophen-hydrocodone bitartrate is commercially available as Vicodin, and acetaminophen-oxycodone hydrochloride is commercially available as Percocet. In randomized controlled trials, it was shown that the combination product Percocet was statistically superior to MR oxycodone in various outcome measures of pain relief. Other combination products such as acetaminophen-tramadol are either available or described in the literature. It is postulated that the combination of two analgesic drugs with complementary mechanisms of action results in enhanced analgesia due to an additive effect, an “opioid-sparing” effect, and an improved side effect and safety profile. The improved safety profile results from the use of reduced doses of two analgesics with different side-effects rather than an equi-effective dose of a single agent.

Acetaminophen is absorbed from the small intestine and primarily metabolized by conjugation, like glucuronidation and sulfation, in the liver to nontoxic, water-soluble compounds that are eliminated in the urine. When the maximum daily dose is exceeded over a prolonged period, metabolism by conjugation becomes saturated, and excess acetaminophen is oxidatively metabolized by cytochrome P450 (CYP) enzymes (e.g., CYP2E1, 1A2, 2A6, 3A4) to a reactive metabolite, N-acetyl-p-benzoquinone-imine (NAPQI). NAPQI is a reactive free radical with an extremely short half-life that is rapidly inactivated by conjugation with glutathione, which is acting as a sulfhydryl donor. Once the pool of available glutathione is exhausted, the cysteines of cellular proteins become sulfhydryl donors to NAPQI, binding covalently and initiating a cascade of oxidative and cellular damage, resulting in necrosis and, ultimately, liver failure. Thus, avoiding excessive NAPQI formation is an important strategy when using acetaminophen, although to date acetaminophen-sparing has not been an approach any manufacturers have chosen to take. However, due to the prevalence of acetaminophen in many over-the-counter products, it is prudent to consider acetaminophen-sparing precautions when considering combination therapy lasting more than a few days to avoid an inadvertent reduction in glutathione stores.

Thus, various options for pain management are available that are both IR and MR, and contain either a single drug or a combination of analgesics. While these combination products provide the benefits associated with combining two analgesics as described above, both IR and MR, in itself, have a significant disadvantage. IR combination products lack the advantages of MR products described previously. MR combination products lack a significant benefit associated with IR products—rapid onset of analgesia—that is extremely desirable for pain management. Because MR products retard the rate of drug release to sustain the drug effect over prolonged period, release of drug is slow resulting in significant time before effective analgesic drug concentration is attained in the bloodstream. There exists a clinical need for pain management that combines the desirable features of IR and MR in combination pain products.

### SUMMARY OF THE INVENTION

Among the various aspects of the present disclosure is a pharmaceutical composition for extended release of hydrocodone and acetaminophen comprising at least one extended release portion comprising, hydrocodone, acetaminophen or a combination thereof, and at least one extended release component. The composition, when orally administered to a subject, maintains a therapeutic plasma concentration of hydrocodone of at least about 5 ng/mL from about 0.75 hour to about 10 hours after administration of the composition. Additionally, at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration.

A further aspect of the disclosure encompasses a pharmaceutical composition for extended release of hydrocodone and acetaminophen comprising (a) at least one immediate release portion comprising hydrocodone, acetaminophen or a combination thereof, and (b) at least one extended release portion comprising hydrocodone, acetaminophen or a combination thereof, and an extended release component, wherein about 30% of the hydrocodone in the pharmaceutical composition is released in about 15 minutes and at least about 90% of the acetaminophen in the pharmaceutical composition is released in about 8 hours when measured in 900 ml of 0.1N HCl using a USP type II apparatus at a paddle speed of about 150 rpm and a constant temperature of 37° C.

Yet another aspect of the disclosure is a pharmaceutical composition for extended release of hydrocodone and acetaminophen, comprising at least one extended release portion comprising hydrocodone or a pharmaceutically acceptable salt thereof, acetaminophen, and an extended release component; wherein upon administration to a subject in need thereof, the composition provides an AUC0-1.27 h for acetaminophen of about 3 ng·h/mL/mg to about 13 ng·h/mL/mg; an AUC1.27-36 h for acetaminophen of about 20 ng·h/mL/mg to about 75 ng·h/mL/mg; an AUC0-2.4 h for hydrocodone or salt of about 0.5 ng·h/mL/mg to about 5 ng·h/mL/mg; and AUC2.4-36 h for hydrocodone or salt of about 5 ng·h/mL/mg to about 25 ng·h/mL/mg.

A further aspect of the disclosure is a pharmaceutical composition for oral administration in the treatment of pain, comprising at least one extended release portion comprising hydrocodone or a pharmaceutically acceptable salt thereof, acetaminophen, and an extended release component wherein when the composition is administered to a subject in need thereof, the subject attains therapeutic blood levels of both the hydrocodone and the acetaminophen within about one hour after administration of the composition and maintains analgesia for about 12 hours after administration of the composition. Further, upon placement of the composition in an in vitro dissolution test comprising USP Paddle Method at a paddle speed of about 100 rpm in 900 ml of 0.1N HCl using a USP type II apparatus at a constant temperature of 37° C., no more than about 65%, by weight, of the total amount of the hydrocodone or salt is released and no more than about 80%, by weight, of the total amount of the acetaminophen is released by 2 hours; from about 65% to about 85%, by weight, of the total amount of the hydrocodone or salt is released and from about 65% to about 95%, by weight, of the total amount of the acetaminophen is released after 4 hours; from about 80% to about 100%, by weight, of the total amount of the hydrocodone or salt is released and from about 80% to about 100%, by weight, of the total amount of the acetaminophen is released after 8 hours; and about 85% to about 100%, by weight, of the total amount of the hydrocodone or salt is released and from about 85% to about 100%, by weight, of the total amount of the acetaminophen is released after 12 hours.

Still another aspect of the disclosure provides a dosage form comprising (a) an immediate release portion comprising acetaminophen and hydrocodone, wherein the immediate release portion comprises, by weight of the immediate release portion, from about 70% to about 80% of acetaminophen and from about 0.5% to about 1% of hydrocodone; and (b) an extended release portion comprising acetaminophen, hydrocodone, and an extended release polymer, wherein the extended release portion comprises, by weight of the extended release portion, from about 20% to about 40% of acetaminophen, from about 0.5% to about 2% of hydrocodone, and from about 30% to about 50% of the extended release polymer.

Another aspect provides a dosage form comprising from about 7.5 mg to about 30 mg of hydrocodone and from about 325 mg to about 650 mg of acetaminophen. The dosage form comprises (a) at least one immediate release portion comprising about 25% of the total amount of hydrocodone in the composition and about 50% of the total amount of acetaminophen in the composition; and (b) at least one extended release portion comprising about 75% of the total amount of hydrocodone in the composition, about 50% of the total amount of acetaminophen in the composition, and about 35% to about 45%, by weight of the at least one extended release portion, of an extended release polymer comprising a polyethylene oxide.

A further aspect of the disclosure provides a method for reducing the risk of acetaminophen-induced hepatic damage in a subject being treated for pain with a dosage regimen that comprises administering to the subject at least two consecutive doses of a pharmaceutical composition comprising hydrocodone and acetaminophen. The method comprises (a) administering a first dose of the pharmaceutical composition comprising at least one extended release portion comprising acetaminophen, hydrocodone or a combination thereof, and an extended release component to the subject, wherein the composition maintains a therapeutic blood plasma concentration of hydrocodone of at least 5 ng/mL from about 0.75 hours to about 10 hours after administration of the composition, and wherein at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration; and (b) administering a second dose of the pharmaceutical composition to the subject at about 12 hours after administration of the first dose.

Yet another aspect of the disclosure encompasses a method for treating pain in a subject in need thereof with a pharmaceutical composition that comprises hydrocodone and acetaminophen. The method comprises orally administering to the subject an effective amount of the pharmaceutical composition comprising at least one extended release portion comprising hydrocodone, acetaminophen or a combination thereof, and an extended release component, wherein the composition maintains a therapeutic plasma concentration of hydrocodone of at least about 5 ng/mL from about 0.75 hour to about 10 hours after administration of the composition, and wherein at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration.

Other features and aspects of the disclosure are described in detail below.

### REFERENCE TO COLOR FIGURES

This application file contains at least one drawing executed in color. Copies of this patent application publication with color drawings will be provided by the Office upon request and payment of the necessary fee.

### BRIEF DESCRIPTION OF THE DRAWINGS

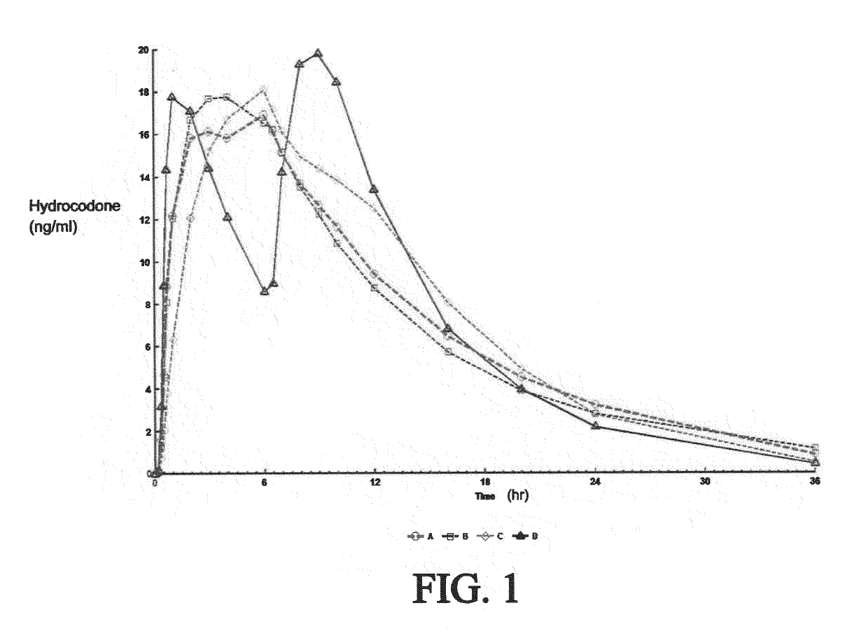

FIG. 1 presents the mean plasma concentrations of hydrocodone versus time by treatment for 0 to 36 hours. Treatment A (formulation A) was a single, two-tablet dose containing a total of 15 mg hydrocodone and 650 mg acetaminophen having slow release properties as compared to formulation B, administered orally under fasted conditions. Treatment B (formulation B) was a single, two-tablet dose containing a total of 15 mg hydrocodone and 650 mg acetaminophen having faster release properties as compared to formulation A, administered orally under fasted conditions. Treatment C (formulation B) was a single, two-tablet dose containing a total of 15 mg hydrocodone and 650 mg acetaminophen administered orally under fed conditions. Treatment D was one tablet of an immediate release 7.5 hydrocodone/325 acetaminophen tablet administered orally every 6 hours for 2 doses under fasted conditions.

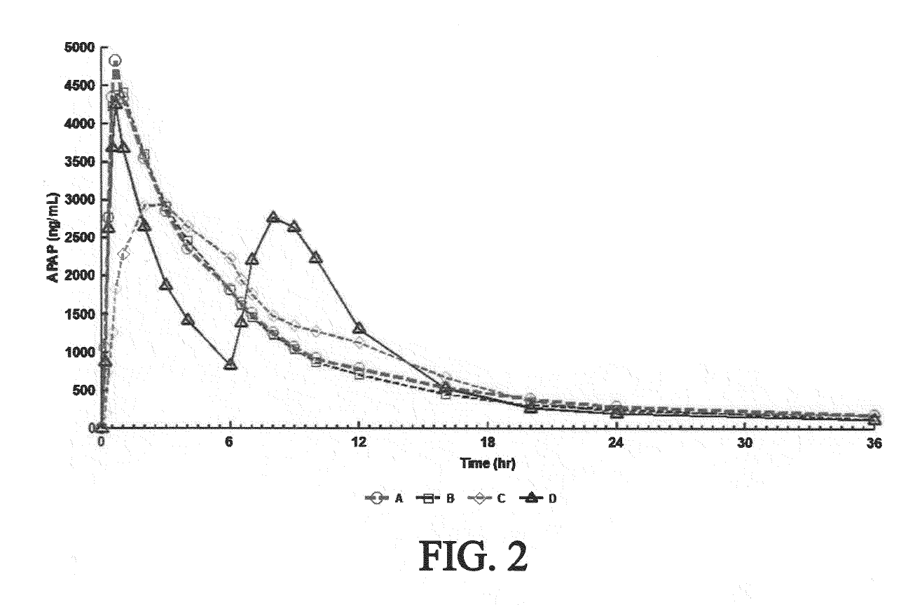

FIG. 2 presents the mean plasma concentrations of acetaminophen versus time by treatment for 0 to 36 hours. Treatment A (formulation A) was a single, two-tablet dose containing a total of 15 mg hydrocodone and 650 mg acetaminophen having slow release properties as compared to formulation B, administered orally under fasted conditions. Treatment B (formulation B) was a single, two-tablet dose containing a total of 15 mg hydrocodone and 650 mg acetaminophen having faster release properties as compared to formulation A, administered orally under fasted conditions. Treatment C (formulation B) was a single, two-tablet dose containing a total of 15 mg hydrocodone and 650 mg acetaminophen administered orally under fed conditions. Treatment D was one tablet of an immediate release 7.5 hydrocodone/325 acetaminophen tablet administered orally every 6 hours for 2 doses under fasted conditions.

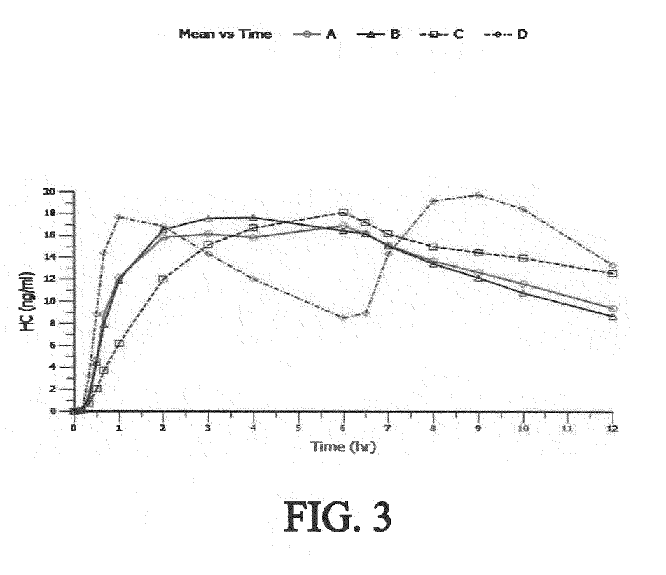

FIG. 3 presents the mean plasma concentrations of hydrocodone versus time by treatment as indicated in FIG. 1, but represented for 0 to 12 hours.

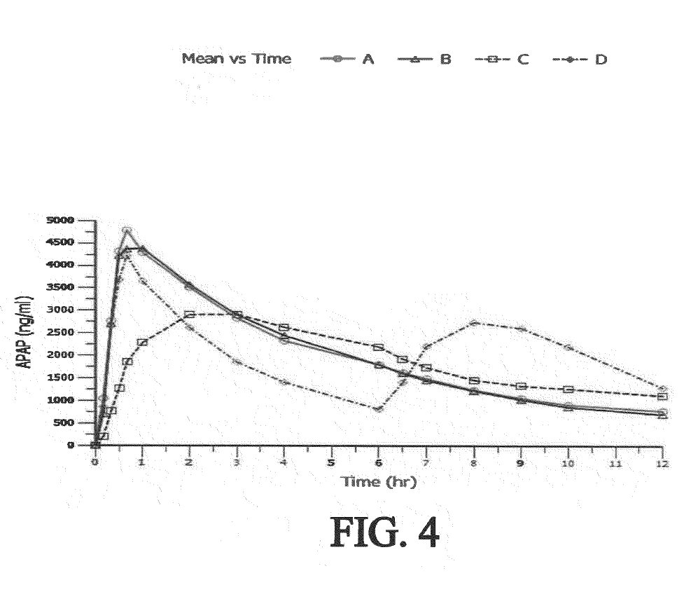

FIG. 4 presents the mean plasma concentrations of acetaminophen versus time by treatment as indicated in FIG. 2, but represented for 0 to 12 hours.

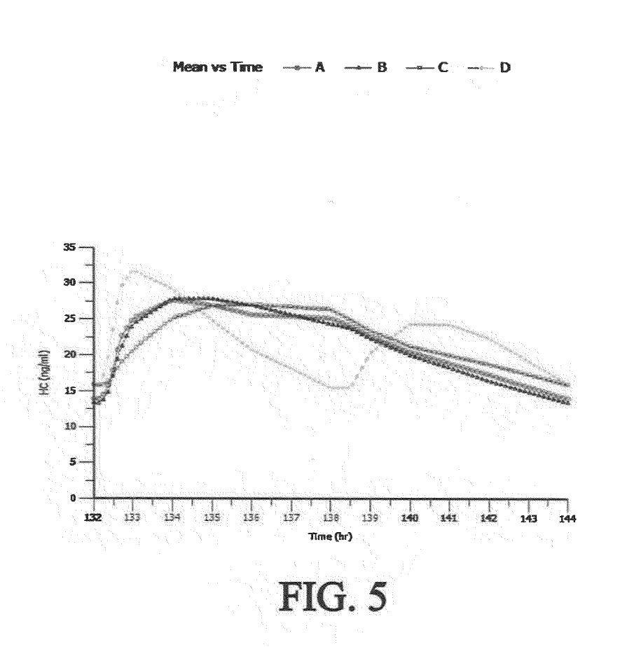

FIG. 5 presents simulated hydrocodone pharmacokinetic profiles at steady state versus time by treatment for 0 to 144 hours for Treatments A, B, C, and D of Example 1.

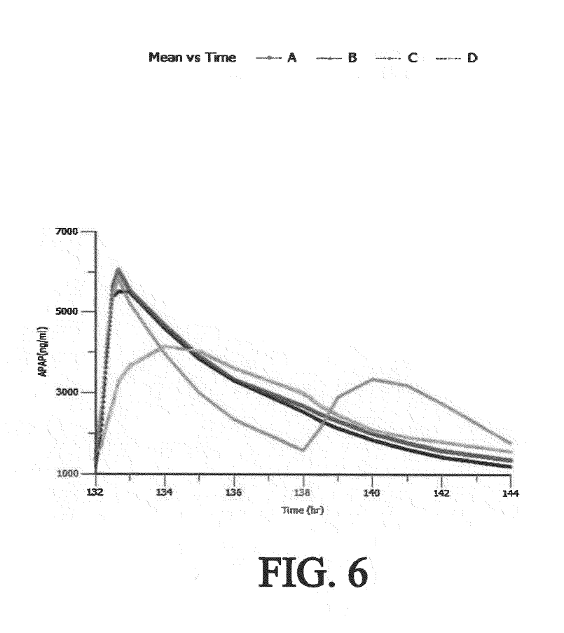

FIG. 6 presents simulated acetaminophen pharmacokinetic profiles at steady state versus time by treatment for 0 to 144 hours for Treatments A, B, C, and D of Example 1.

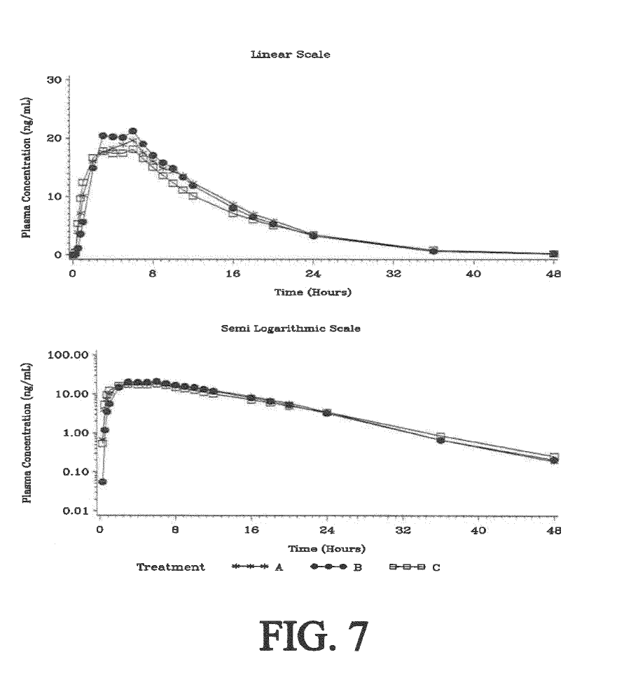

FIG. 7 presents mean plasma concentrations of hydrocodone as a function of time by treatment following oral administration of two tablets of 7.5 mg of hydrocodone and 325 mg of acetaminophen. Treatment A was under fed (high fat) conditions. Treatment B was under fed (low fat) conditions. Treatment C was under fasted conditions.

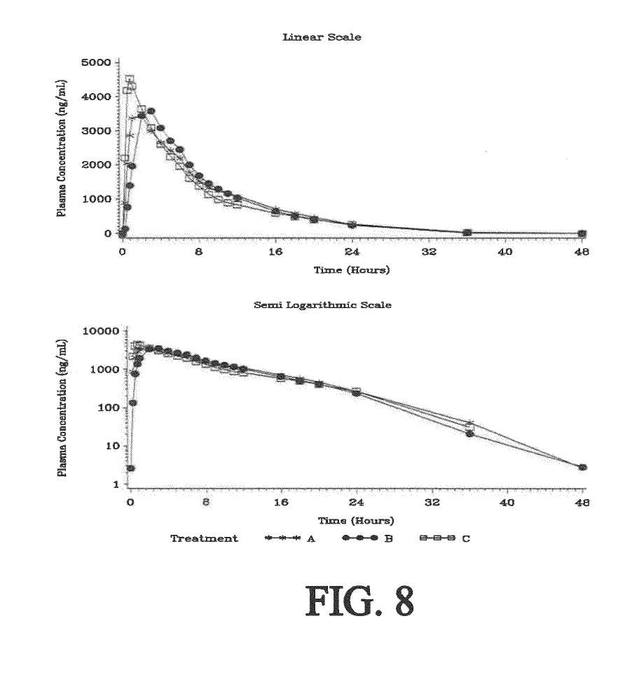

FIG. 8 presents mean plasma concentrations of acetaminophen as a function of time by treatment following oral administration of two tablets of 7.5 mg of hydrocodone and 325 mg of acetaminophen. Treatment A was under fed (high fat) conditions. Treatment B was under fed (low fat) conditions. Treatment C was under fasted conditions.

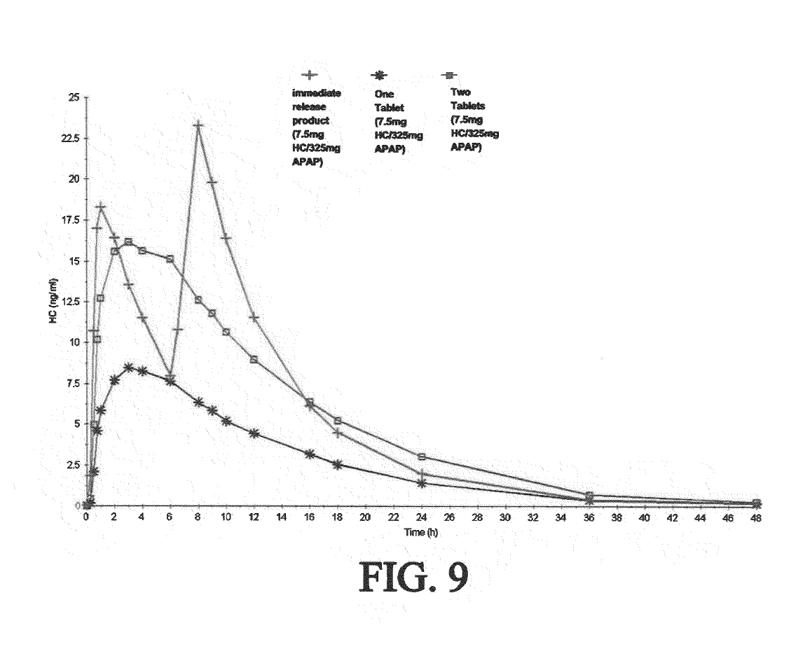

FIG. 9 presents mean plasma concentrations of hydrocodone as a function of time by treatment following oral administration of a single dose of Treatments A, B, and C of Example 3.

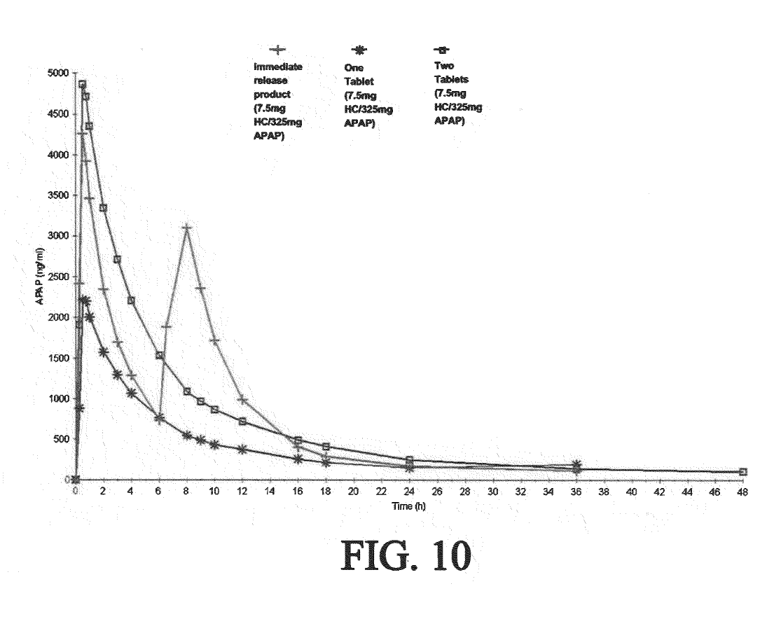

FIG. 10 presents mean plasma concentrations of acetaminophen as a function of time by treatment following oral administration of a single dose of Treatments A, B, and C of Example 3.

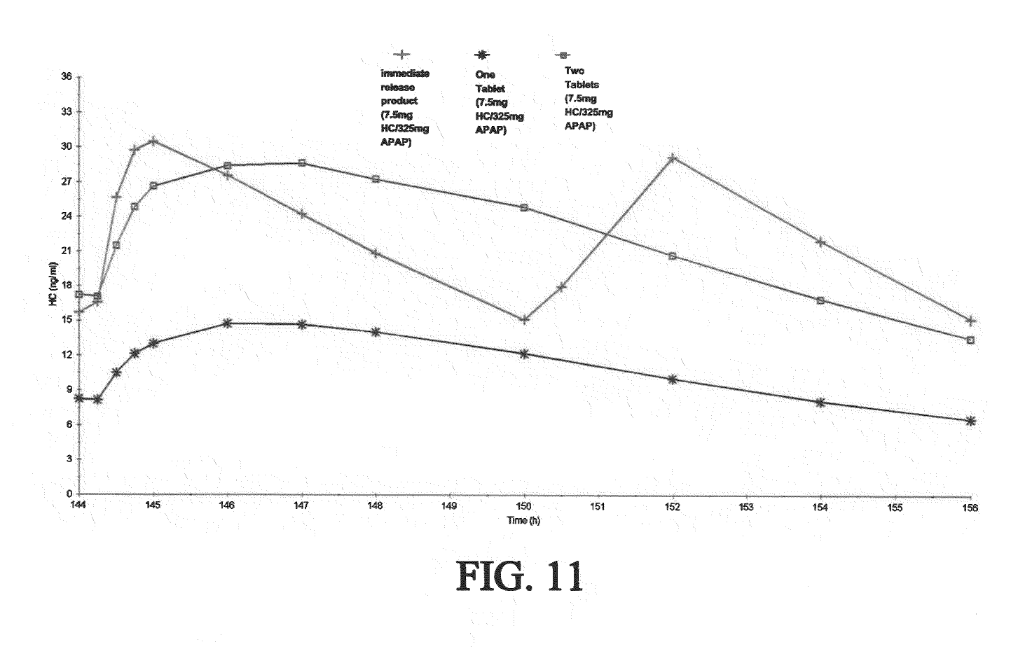

FIG. 11 presents mean plasma concentrations of hydrocodone as a function of time by treatment following oral administration of multiple doses of Treatments A, B, and C of Example 3.

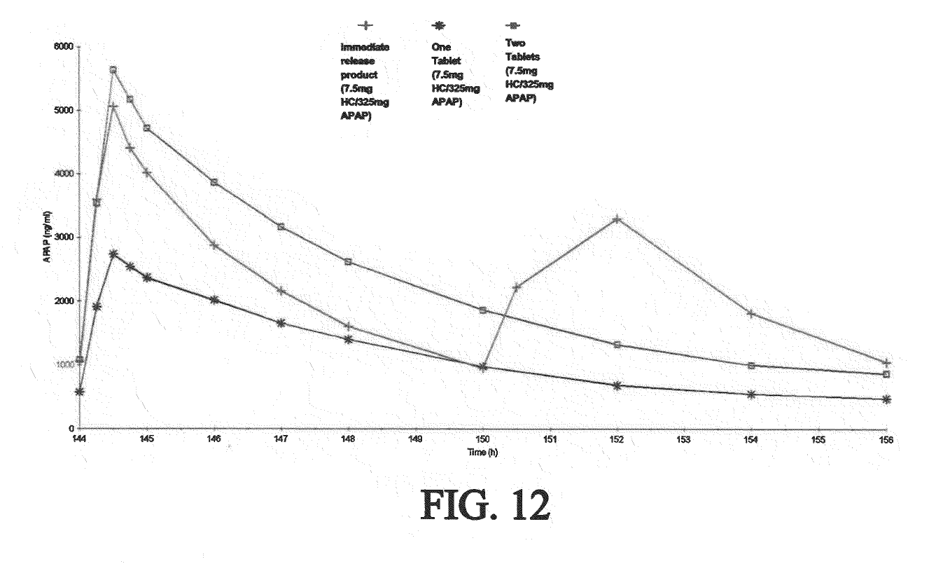

FIG. 12 presents mean plasma concentrations of acetaminophen as a function of time by treatment following oral administration of multiple doses of Treatments A, B, and C of Example 3.

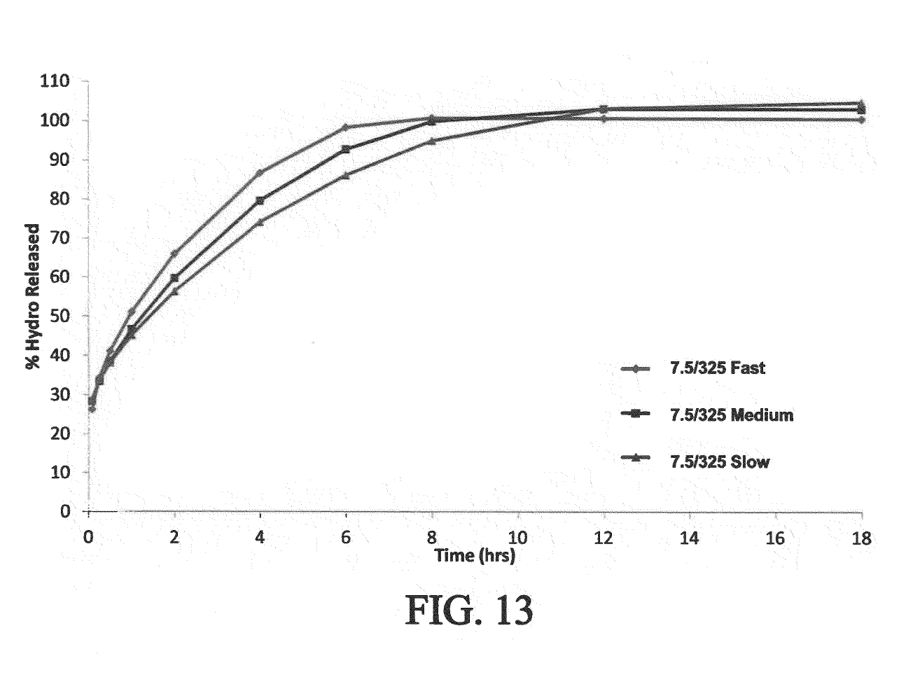

FIG. 13 presents dissolution data for the release of hydrocodone from fast-release, medium-release, and slow-release pharmaceutical compositions containing 7.5 mg hydrocodone and 325 acetaminophen.

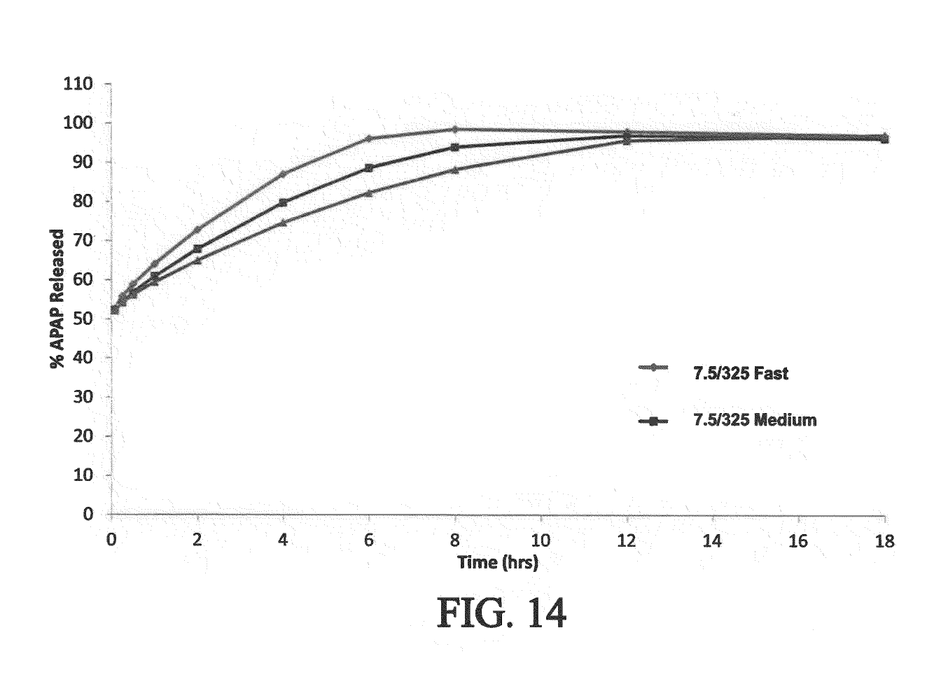

FIG. 14 presents dissolution data for the release of acetaminophen from fast-release, medium-release, and slow-release pharmaceutical compositions containing 7.5 mg hydrocodone and 325 acetaminophen.

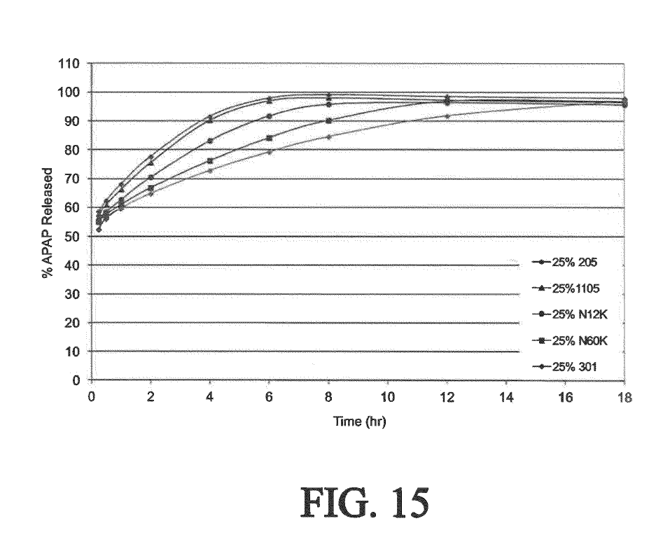

FIG. 15 presents acetaminophen dissolution data for five pharmaceutical formulations described herein. Each formulation tablet contained a total of 15 mg hydrocodone bitartrate HCl and a total of 500 mg acetaminophen. The ER portions of the five pharmaceutical formulations contained 25% by weight POLYOX® 205, 1105, N-12K, N-60K, and 301 respectively.

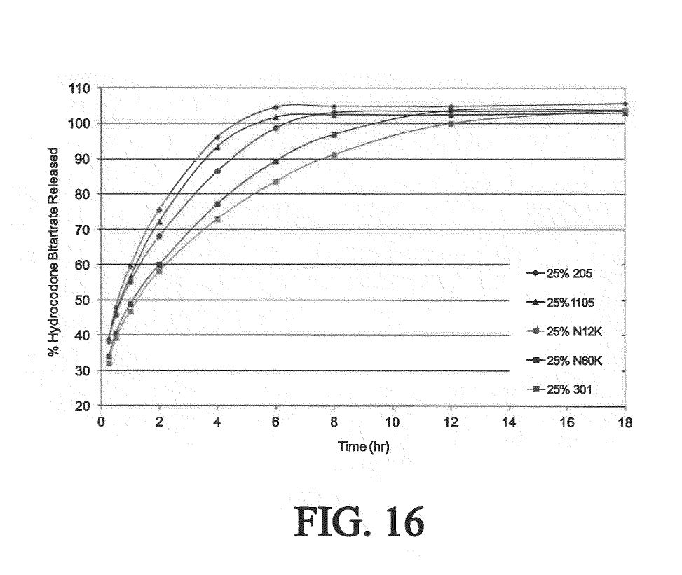

FIG. 16 presents hydrocodone bitartrate dissolution data for the five pharmaceutical formulations described in FIG. 15.

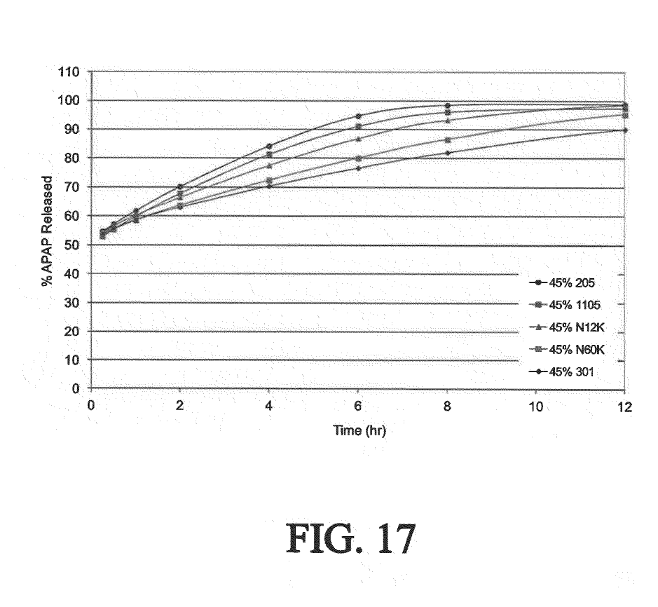

FIG. 17 presents acetaminophen dissolution data for five pharmaceutical formulations described herein. Each formulation tablet contained a total of 15 mg hydrocodone bitartrate and a total of 500 mg acetaminophen. The ER portions of the five pharmaceutical formulations contained 45% by weight POLYOX® 205, 1105, N-12K, N-60K, and 301 respectively.

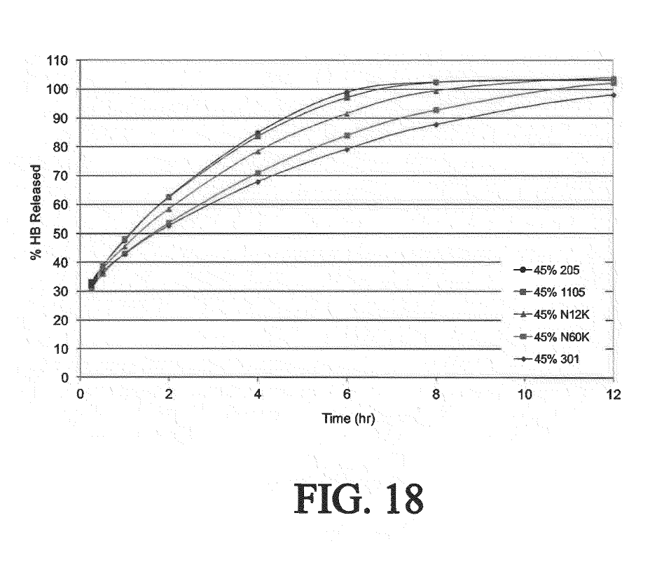

FIG. 18 presents hydrocodone bitartrate dissolution data for the five pharmaceutical formulations described in FIG. 17.

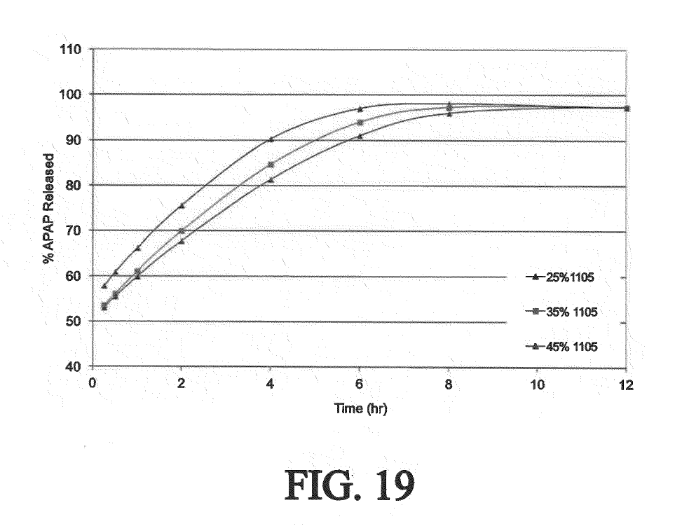

FIG. 19 presents acetaminophen dissolution data for three pharmaceutical formulations described herein. Each formulation tablet contained a total of 15 mg hydrocodone bitartrate and a total of 500 mg acetaminophen. The ER portions of the three pharmaceutical formulations contained 25% by weight, 35% by weight, and 45% by weight POLYOX® 1105, respectively.

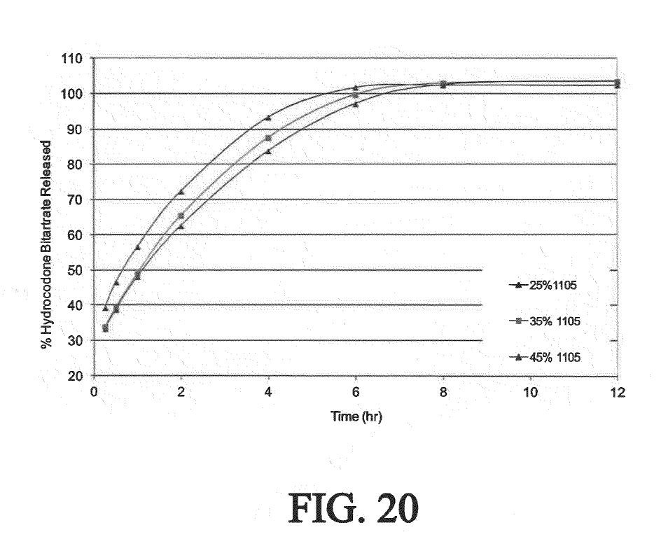

FIG. 20 presents hydrocodone bitartrate dissolution data for the three pharmaceutical formulations described in FIG. 19.

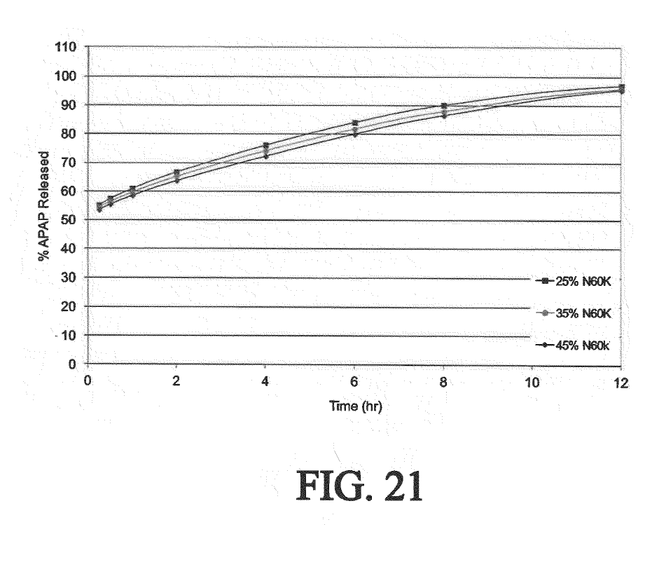

FIG. 21 presents acetaminophen dissolution data for three pharmaceutical formulations described herein. Each formulation tablet contained a total of 15 mg hydrocodone bitartrate and a total of 500 mg acetaminophen. The ER portions of the three pharmaceutical formulations contained 25% by weight, 35% by weight, and 45% by weight POLYOX® N-60K, respectively.

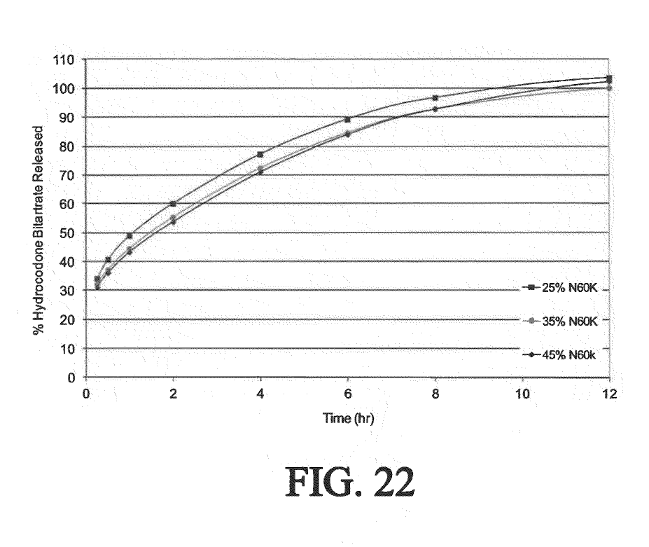

FIG. 22 presents hydrocodone bitartrate dissolution data for the three pharmaceutical formulations described in FIG. 21.

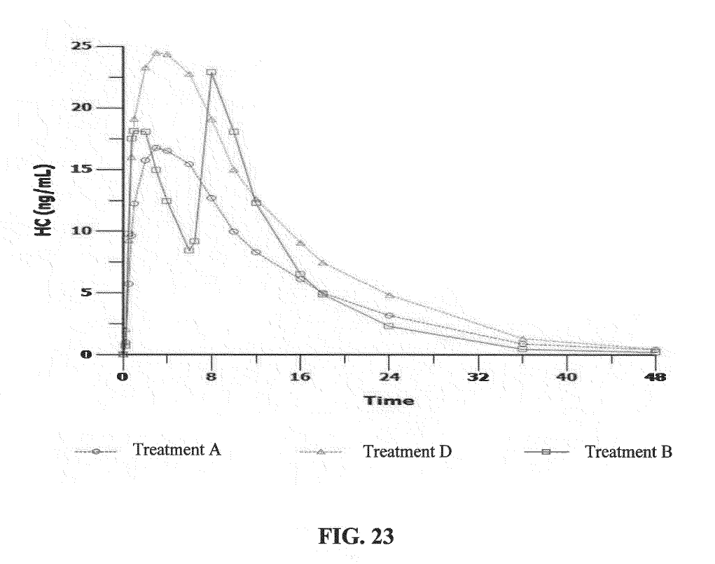

FIG. 23 presents mean plasma concentrations of hydrocodone as a function of time by treatment following oral administration of a single dose of Treatments A, D, and B of Example 12.

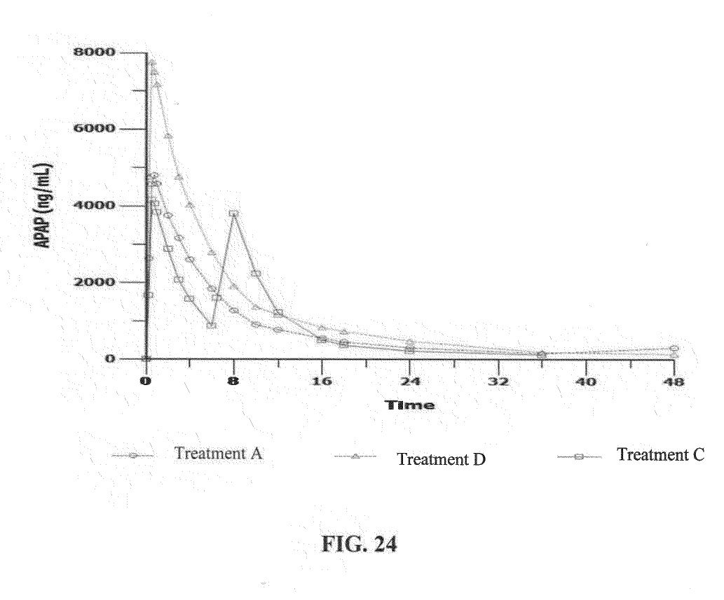

FIG. 24 presents mean plasma concentrations of acetaminophen as a function of time by treatment following oral administration of a single dose of Treatments A, D, and C of Example 12.

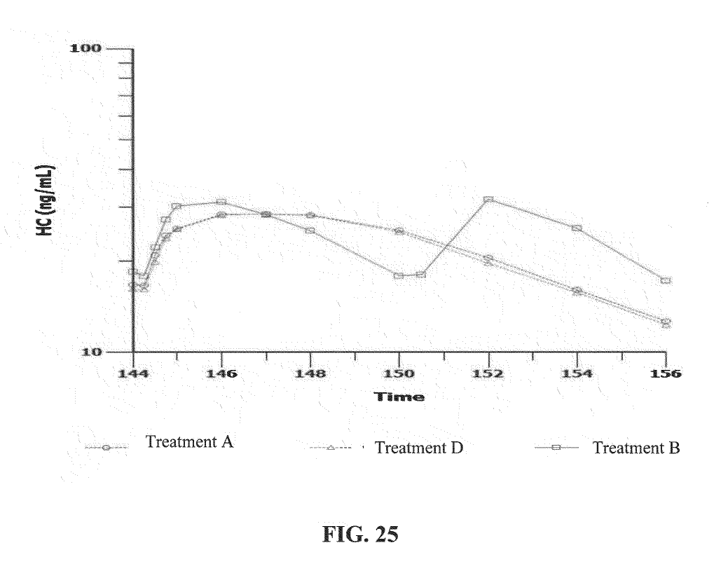

FIG. 25 presents mean plasma concentrations of hydrocodone as a function of time by treatment following oral administration of a loading dose and a subsequent dose of Treatments A, D, and B of Example 12.

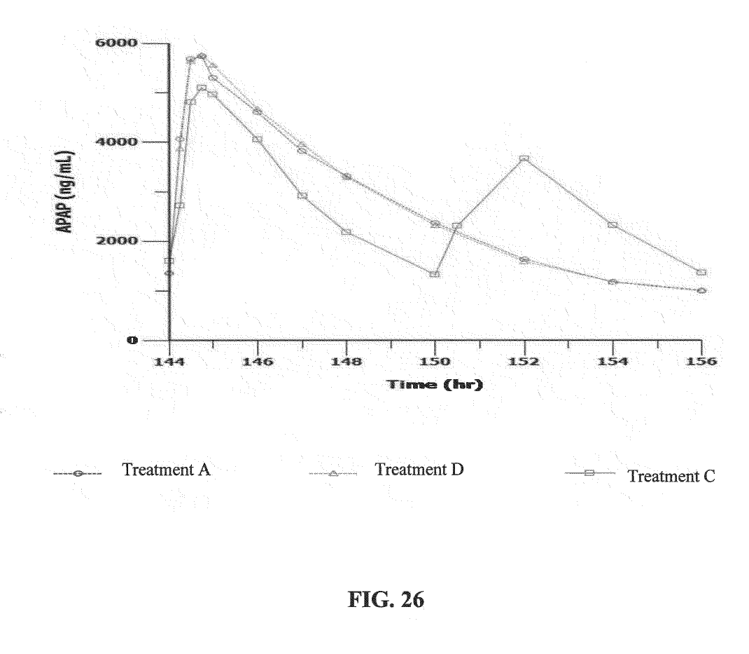

FIG. 26 presents mean plasma concentrations of acetaminophen as a function of time by treatment following oral administration of a loading dose and a subsequent dose of Treatments A, D, and C of Example 12.

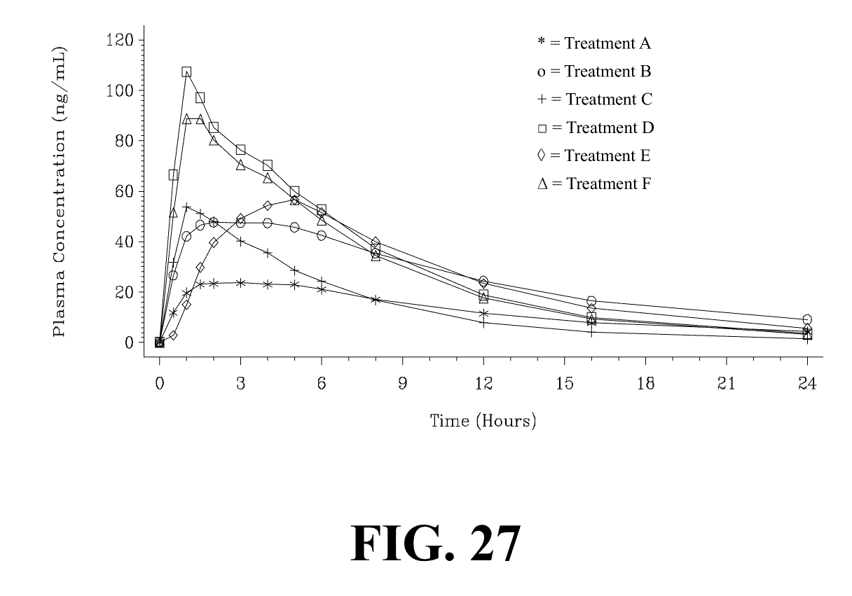

FIG. 27 presents mean plasma concentrations of hydrocodone as a function of time for Treatments A, B, C, D, E, and F of Example 16.

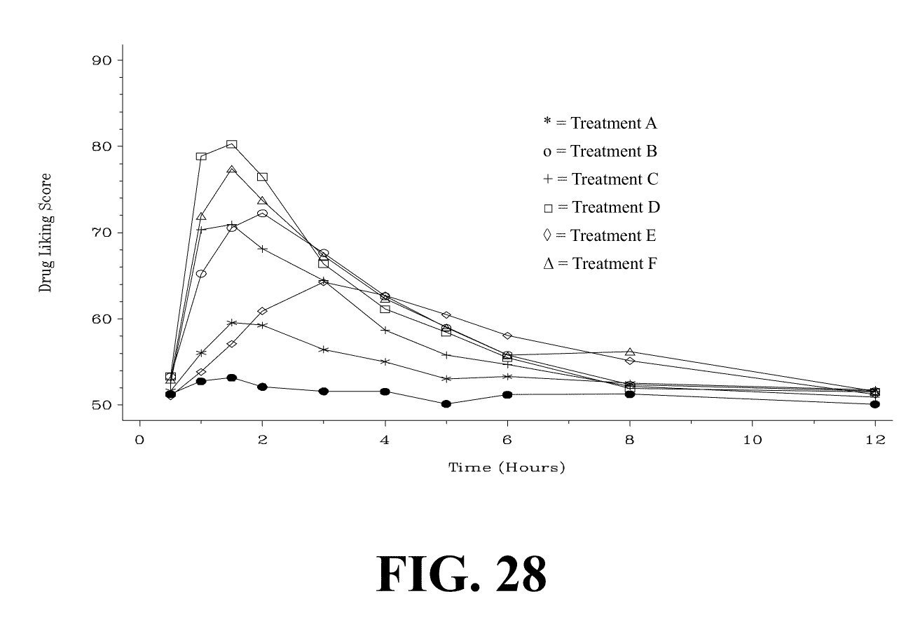

FIG. 28 presents mean drug liking scores over a period of 12 hours for Treatments A, B, C, D, E, F, and G of Example 16.

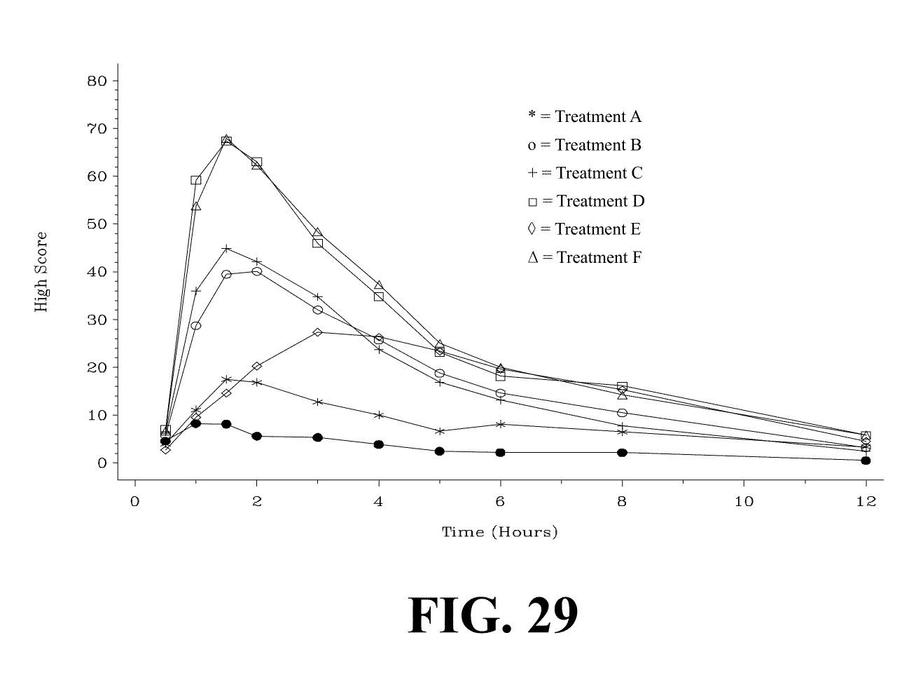

FIG. 29 presents mean high scores over a period of 12 hours for Treatments A, B, C, D, E, F, and G of Example 16.

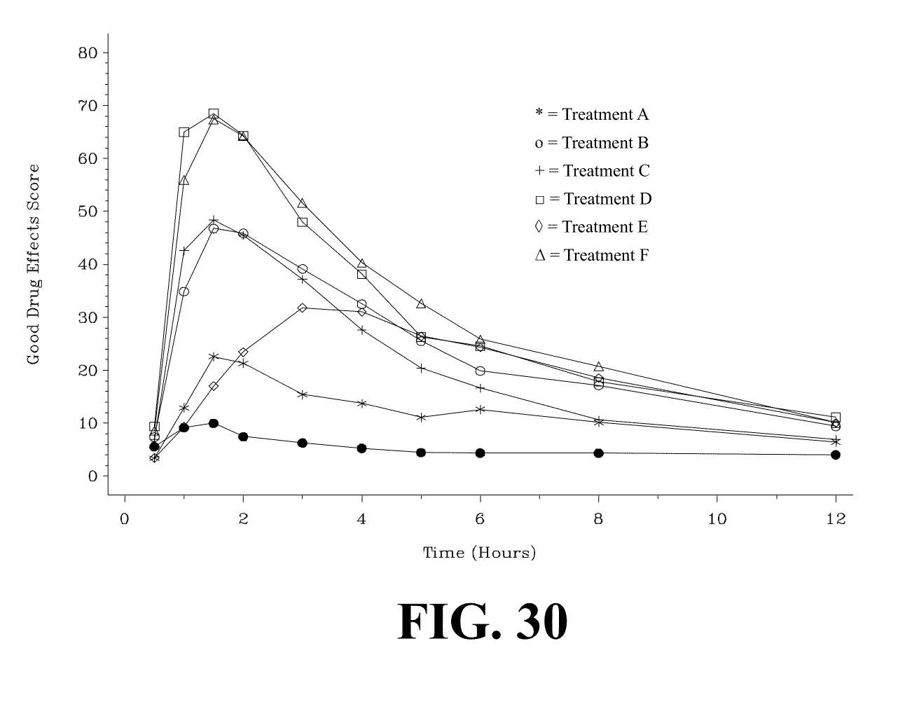

FIG. 30 presents mean good drug effects scores over a period of 12 hours for Treatments A, B, C, D, E, F, and G of Example 16.

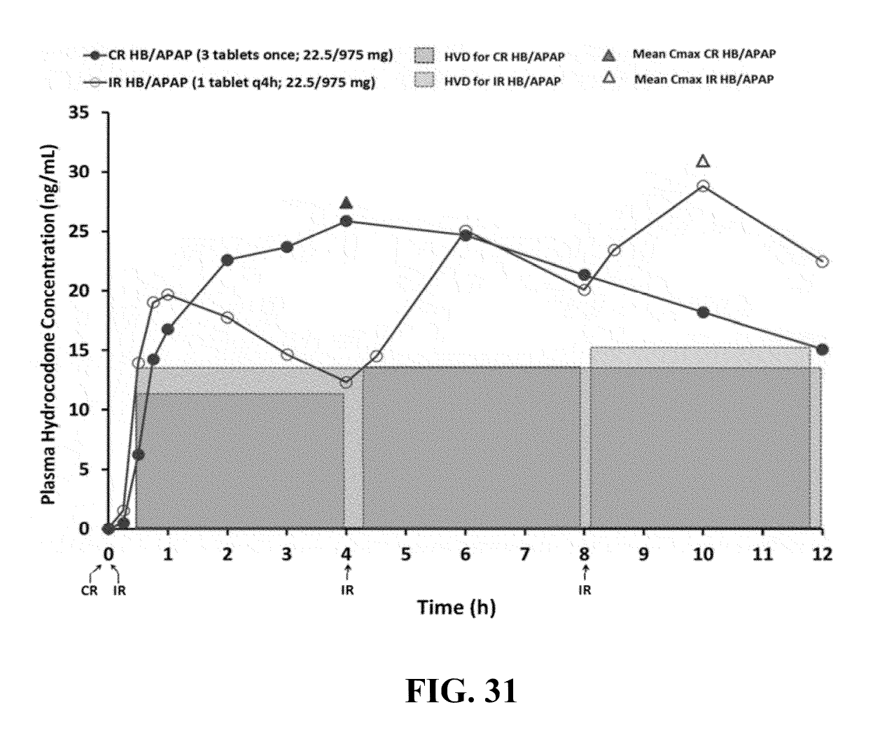

FIG. 31 presents plasma concentration over time and half-value duration for hydrocodone on Day 1 of the multiple-dose study of Example 17.

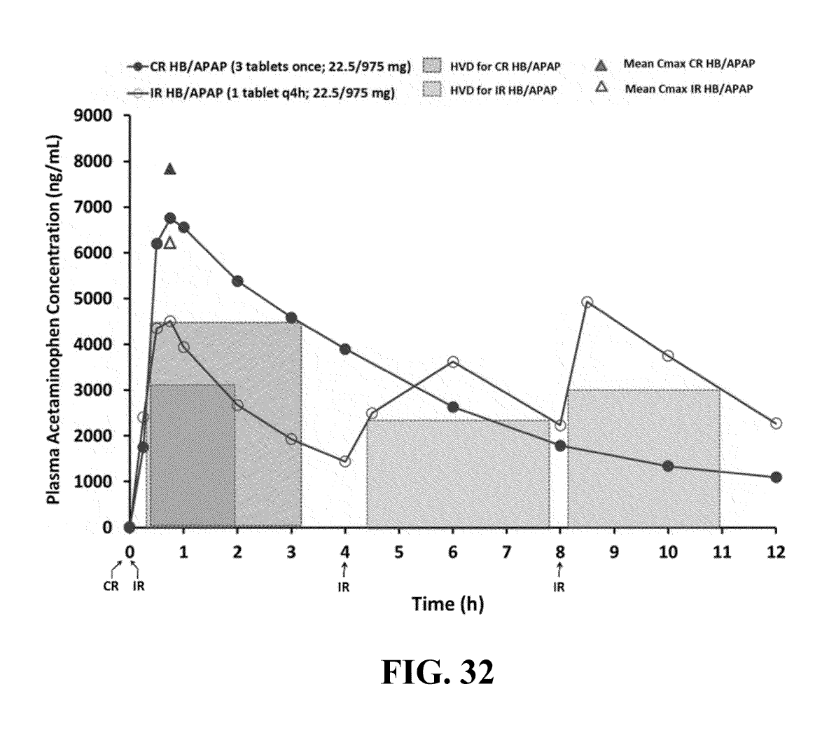

FIG. 32 presents plasma concentration over time and half-value duration for acetaminophen on Day 1 of the multiple-dose study of Example 17.

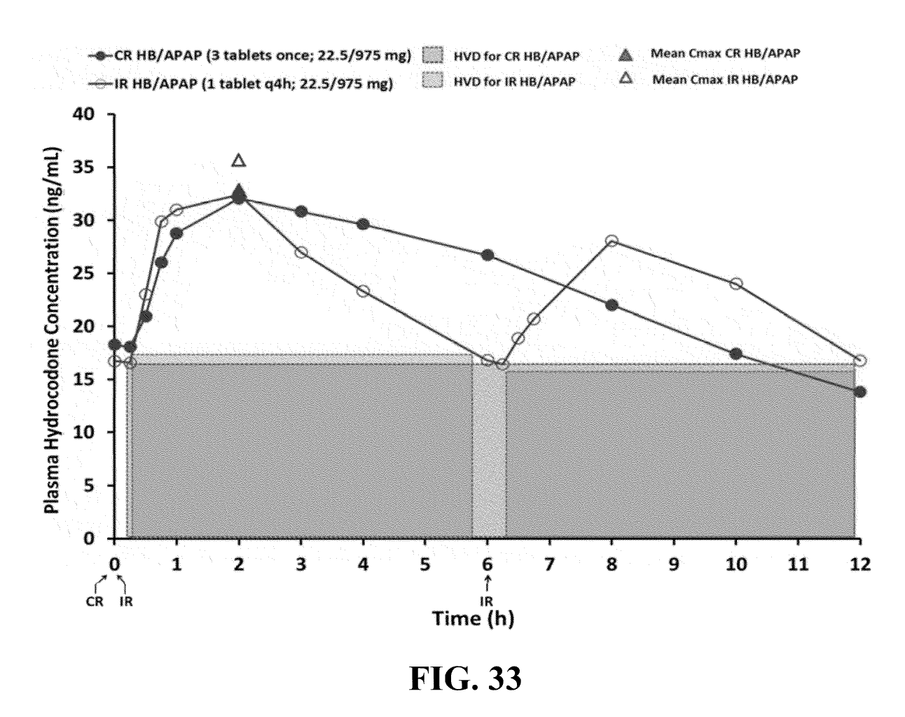

FIG. 33 presents plasma concentration over time and half-value duration for hydrocodone on Day 5 of the multiple-dose study of Example 17.

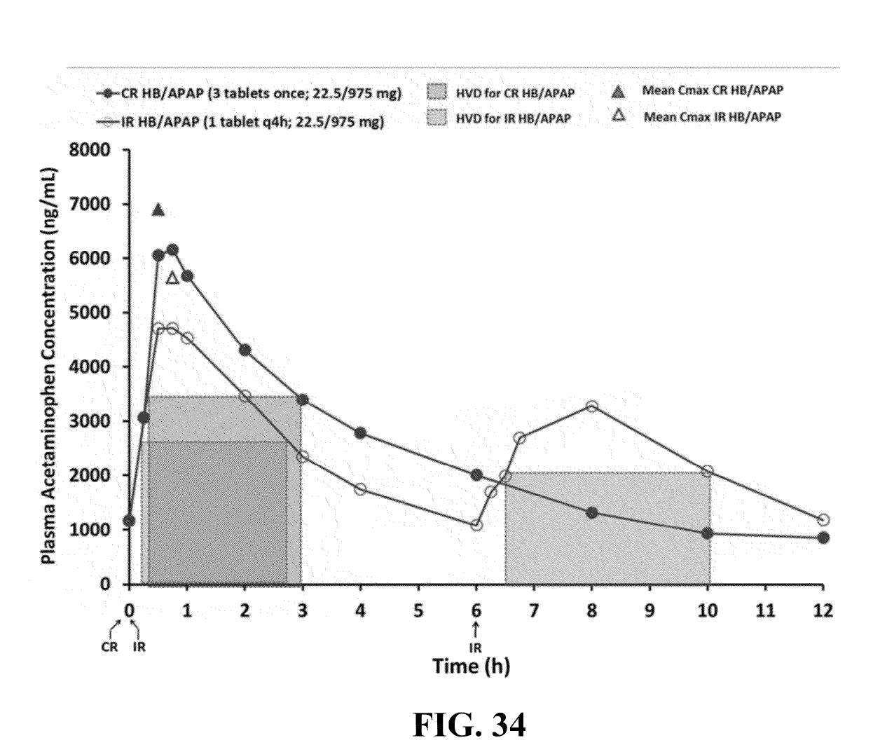

FIG. 34 presents plasma concentration over time and half-value duration for hydrocodone on Day 5 of the multiple-dose study of Example 17.

### DETAILED DESCRIPTION OF THE INVENTION

Disclosed herein is a, combination product of hydrocodone and acetaminophen that has the desirable attributes of both IR and MR products. The extended release pharmaceutical composition disclosed herein comprises at least one extended release portion and, optionally, at least one immediate release portion. The extended release and immediate release portions may comprise hydrocodone, acetaminophen, or combinations thereof. The at least one immediate release portion releases acetaminophen (APAP) and/or hydrocodone instantly in an immediate release fashion that provides rapid onset for the attainment of therapeutically effective plasma concentrations within about the first 5 minutes, 10 minutes, 15 minutes, 20 minutes, 25 minutes, 30 minutes, 35 minutes, 40 minutes, 45 minutes, 50 minutes, 55 minutes, or 60 minutes after administration of the composition. The at least one extended release portion releases acetaminophen and/or hydrocodone in an extended release fashion to maintain plasma concentrations above the minimum effective concentration for about 8-12 hours. In addition, two other important features of this composition are: 1) to allow the plasma concentrations of hydrocodone to fall as rapidly as an immediate release formulation to provide the same rate of termination of drug effects as the immediate release product, and 2) to allow the concentrations of APAP to fall even quicker towards the later part of the dosing interval and bring down the levels of APAP lower than those of the immediate release product. The concentrations of APAP in the last quarter of the dosing interval are comparable to the pre-dose concentrations in a multiple dose setting, allowing for the glutathione synthase enzyme cycle to replenish the body's levels of glutathione to avoid the formation of toxic intermediates with subsequent doses of APAP. Moreover, the concentrations of APAP in the later part of the dosing interval are lower than those present when administered a conventional extended release formulation. This feature has been deliberately introduced to reduce the hepatic injury due to APAP and is termed “APAP time-off”.

Abuse potential is a concern with any opioid product. The addition of APAP to the opioid, however, is likely to reduce the amount of abuse by illicit routes of administration, particularly intravenous or intranasal administration. This deterrence is likely due to the bulk (grams) that the APAP provides as well as the relative aqueous insolubility compared to freely soluble opioid salts. Further, APAP is known to be irritating to nasal passages and to make drug abusers sneeze violently when they are trying to snort it. In addition, embodiments disclosed herein may be tamper resistant in that the compositions are difficult to crush for administration intravenously or intranasally; difficult to extract with water or alcohol because the mixture becomes too viscous for injecting or snorting; and resistant to dose dumping in alcohol.

In one embodiment, the pharmaceutical composition disclosed herein, therefore, provides: 1) rapid onset of analgesia within about 15, 30, 45, or 60 minutes after administration of the composition mediated by both hydrocodone and APAP, with APAP providing maximal contribution during the early phase; 2) prolonged analgesia for the entire 12 hours period, mainly contributed by hydrocodone, with minimal fluctuations during this period; 3) relatively low levels of APAP toward end of dosing interval to allow for recovery of the depleted hepatic glutathione system; 4) low abuse quotient; and 5) abuse deterrence.

Headings included herein are simply for ease of reference, and are not intended to limit the disclosure in any way.

### I. DEFINITIONS

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

When introducing elements of the various embodiment(s) of the present disclosure thereof, the articles “a”, “an”, “the” and “said” are intended to mean that there are one or more of the elements. The terms “comprising”, “including” and “having” are intended to be inclusive and mean that there may be additional elements other than the listed elements.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

The term “abuse quotient” for a pharmaceutical composition as used herein is the numerical value obtained via dividing the Cmaxfor a drug by the Tmax for the same drug. Generally speaking, the abuse quotient provides a means for predicting the degree of addictiveness of a given pharmaceutical composition. Pharmaceutical compositions with lower abuse quotients typically are less addictive compared to pharmaceutical compositions with higher abuse quotients.

The term “active agent” or “drug,” is used herein to refer to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “bioequivalent,” as used herein, refers to two compositions, products or methods where the 90% Confidence Intervals (CI) for AUC, partial AUC and Cmax are between 0.80 to 1.25.

The term “bulk density,” as used herein, refers to a property of powders and is defined as the mass of many particles of the material divided by the total volume they occupy. The total volume includes particle volume, inter-particle void volume and internal pore volume.

The term “content uniformity,” as used herein refers to the testing of compressed tablets to provide an assessment of how uniformly the micronized or submicron active ingredient is dispersed in the powder mixture. Content uniformity is measured by use of USP Method (General Chapters, Uniformity of Dosage Forms), unless otherwise indicated. A plurality refers to five, ten or more tablet compositions.

The term “friability,” as used herein, refers to the ease with which a tablet will break or fracture. The test for friability is a standard test known to one skilled in the art. Friability is measured under standardized conditions by weighing out a certain number of tablets (generally 20 tablets or less), placing them in a rotating Plexiglas drum in which they are lifted during replicate revolutions by a radial lever, and then dropped approximately 8 inches. After replicate revolutions (typically 100 revolutions at 25 rpm), the tablets are reweighed and the percentage of composition abraded or chipped is calculated.

The term “ER” as used herein refers to extended release. The phrases “extended release layer,” “ER layer,” “ER portion,” and “extended release portion” are used interchangeable in this document. Further, as used herein the “extended release layer,” “ER layer,” “ER portion,” and “extended release portion” can be either (i) a discrete part(s) of the pharmaceutical composition, (ii) integrated within the pharmaceutical composition, or (iii) a combination thereof.

The term “IR” as used herein refers to immediate release. The phrases “immediate release layer,” “IR layer,” “IR portion” and “immediate release portion” are used interchangeable in this document. In addition, as used herein the “immediate release layer,” “IR layer,” “IR portion” and “immediate release portion” can be either (i) a discrete part(s) of the pharmaceutical composition, (ii) integrated within the pharmaceutical composition, or (iii) a combination thereof.

The term “half life” as used herein refers to the time required for a drug's blood or plasma concentration to decrease by one half. This decrease in drug concentration is a reflection of its metabolism plus excretion or elimination after absorption is complete and distribution has reached an equilibrium or quasi equilibrium state. The half life of a drug in the blood may be determined graphically off of a pharmacokinetic plot of a drug's blood-concentration time plot, typically after intravenous administration to a sample population. The half life can also be determined using mathematical calculations that are well known in the art. Further, as used herein the term “half life” also includes the “apparent half-life” of a drug. The apparent half life may be a composite number that accounts for contributions from other processes besides elimination, such as absorption, reuptake, or enterohepatic recycling.

“Optional” or “optionally” means that the subsequently described element, component or circumstance may or may not occur, so that the description includes instances where the element, component, or circumstance occurs and instances where it does not.

“Partial AUC” means an area under the drug concentration-time curve (AUC) calculated using linear trapezoidal summation for a specified interval of time, for example, AUC₍₀₋₁ₕᵣ₎ AUC₍₀₋₂ₕᵣ₎, AUC₍₀₋₄ₕᵣ₎, AUC₍₀₋₆ₕᵣ₎, AUC₍₀₋₈ₕᵣ₎, AUC_{(0-(Tmax of IR product+2SD))}, AUC₍₀₋₍ₓ₎ₕᵣ₎, AUC_{(x-yhr)}, AUC_{(Tmax-t)}, AUC(0-(t)hr), AUC_{(Tmax of IR product+2SD)-t)}, or AUC_{(0-∞)}.

A drug “release rate,” as used herein, refers to the quantity of drug released from a dosage form or pharmaceutical composition per unit time, e.g., milligrams of drug released per hour (mg/hr). Drug release rates for drug dosage forms are typically measured as an in vitro rate of dissolution, i.e., a quantity of drug released from the dosage form or pharmaceutical composition per unit time measured under appropriate conditions and in a suitable fluid. The specific results of dissolution tests claimed herein are performed on dosage forms or pharmaceutical compositions immersed in 900 mL of 0.1 N HCl using a USP Type II apparatus at a paddle speed of either about 100 rpm or about 150 rpm and a constant temperature of about 37° C. Suitable aliquots of the release rate solutions are tested to determine the amount of drug released from the dosage form or pharmaceutical composition. For example, the drug can be assayed or injected into a chromatographic system to quantify the amounts of drug released during the testing intervals.

The terms “subject” or “patient” are used interchangeably herein and refer to a vertebrate, preferably a mammal. Mammals include, but are not limited to, humans.

The term “tap density” or “tapped density,” as used herein, refers to a measure of the density of a powder. The tapped density of a pharmaceutical powder is determined using a tapped density tester, which is set to tap the powder at a fixed impact force and frequency. Tapped density by the USP method is determined by a linear progression of the number of taps.

### II. PHARMACEUTICAL COMPOSITIONS COMPRISING EXTENDED AND IMMEDIATE RELEASE PORTIONS COMPRISING HYDROCODONE AND ACETAMINOPHEN

The present disclosure provides pharmaceutical compositions comprising an opioid (e.g., hydrocodone) and its pharmaceutical salts and acetaminophen. It would be understood that when present in a pharmaceutical composition, the opioid would be in its salt form. The pharmaceutical composition comprises at least one extended release portion comprising hydrocodone, acetaminophen or a combination thereof, and an extended release component. The pharmaceutical composition may also comprise at least one immediate release portion comprising hydrocodone, acetaminophen, or a combination thereof. The compositions disclosed herein are formulated to deliver therapeutic concentrations of hydrocodone and acetaminophen within about the first hour after oral administration and to maintain therapeutic concentrations of hydrocodone and acetaminophen for an extended period of time (e.g., 10-12 hours).

The present disclosure further provides for gastric retentive, extended release compositions comprising at least one opioid (e.g., hydrocodone) and at least one other (API) (e.g., acetaminophen) that is preferably absorbed in the upper gastrointestinal tract. In general, the gastric retentive, extended release composition comprises at least one extended release portion. The extended release portion(s) may comprise at least one opioid, at least one API, or combinations thereof. The gastric retentive, extended release composition disclosed herein may further comprise at least one immediate release portion. The immediate release portion(s) may comprise at least one opioid (e.g., hydrocodone), at least one other API (e.g., acetaminophen), or combinations thereof.

### (a) Active Agents

The composition disclosed herein comprises at least one opioid and at least one additional API, each of which is discussed in more detail below. In one embodiment, the same opioid or combination of opioids is present in both the at least one immediate release portion and the at least one extended release portion of the composition; and the same API or combination of APIs is present in both the at least one immediate release portion and the at least one extended release portion of the composition.

(i) Opioids

The opioid(s) useful in the present invention include adulmine, alfentanil, allocryptopine, allylprodine, alphaprodine, anileridine, aporphine, benzylmorphine, berberine, bicuculine, bicucine, bezitramide, buprenorphine, bulbocaprine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propoxyphene, sufentanil, tapentadol, tilidine, tramadol, and pharmaceutical salts of any of the foregoing.

In various embodiments, the extended release dosage form may comprise one, two, three, four, or more than four opioids. In another embodiment, the opioid is selected from the group consisting of oxycodone, hydrocodone, tramadol, codeine, and pharmaceutical salts of any of the foregoing. In yet another embodiment, opioid is selected from the group consisting of adulmine, alfentanil, allocryptopine, allylprodine, alphaprodine, anileridine, aporphine, benzylmorphine, berberine, bicuculine, bicucine, bezitramide, buprenorphine, bulbocaprine, butorphanol, clonitazene, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxymorphone, papavereturn, pentazocine, phenadoxone, phenomorphan, phenazocine, phenoperidine, piminodine, piritramide, propheptazine, promedol, properidine, propoxyphene, sufentanil, tapentadol, tilidine, and pharmaceutical salts of any of the foregoing. In one embodiment, the extended release dosage form comprises one opioid. In a further embodiment, the dosage form comprises hydrocodone.

In one embodiment, the composition may comprise from about 1.0 mg to about 500 mg of the opioid. In another embodiment, the composition may comprise from about 1.4 mg to about 400 mg of the opioid. In yet another embodiment, the amount of opioid in the composition may range from about 5 mg to about 300 mg. In still another embodiment, the amount of opioid in the composition may range from about 4 mg to about 30 mg. In another embodiment, the amount of opioid in the composition may range from about 30 mg to about 60 mg. In yet another embodiment, the amount of opioid in the composition may range from about 60 mg to about 120 mg. In an alternate embodiment, the amount of opioid in the composition may range from about 120 mg to about 300 mg. In various embodiments, the amount of opioid in the composition may be about 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 22 mg, 24 mg, 26 mg, 28 mg, 30 mg, 32 mg, 34 mg, 36 mg, 38 mg, 40 mg, 42 mg, 44 mg, 46 mg, 48 mg, 50 mg, 52 mg, 54 mg, 56 mg, 58 mg, 60 mg, 62 mg, 64 mg, 66 mg, 68 mg, 70 mg, 80 mg, 90 mg, 100 mg, 110 mg, 120 mg, 130 mg, 140 mg, 150 mg, 160 mg, 170 mg, 180 mg, 190 mg, 200 mg, 220 mg, 240 mg, 260 mg, 280 mg, 300 mg, 320 mg, 340 mg, 360 mg, 380 mg, or 400 mg. In one embodiment, the amount of opioid in the composition may range from about 7.5 mg to about 30 mg. In another embodiment, the amount of opioid in the composition may range from about 7.5 mg to about 15 mg. In still another embodiment, the amount of opioid in the composition may range from about 15 mg to about 30 mg.

The total amount of hydrocodone present in the pharmaceutical composition can and will vary. In some embodiments, the total amount of hydrocodone present in the pharmaceutical composition may range from about 2 mg to about 160 mg, about 5 mg to about 75 mg, about 5 mg to about 40 mg, or about 10 mg to about 30 mg. In another embodiment, the total amount of hydrocodone in the pharmaceutical composition may range from about 5 mg to about 30 mg. In various embodiments, the total amount of hydrocodone present in the pharmaceutical composition may be about 5 mg, 5.5 mg, 6.0 mg, 6.5 mg, 7.0 mg, 7.5 mg, 8.0 mg, 8.5 mg, 9.0 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 22.5 mg, 25 mg, 27.5 mg, 30 mg, 32.5 mg, 35 mg, 37.5 mg, 40 mg, 45 mg, 50 mg, 60 mg, 70 mg, 80 mg, 100 mg, 110 mg, 120 mg, 130 mg, 140 mg, 150 mg, or 160 mg. In one embodiment, the total amount of hydrocodone in the pharmaceutical composition may be about 30 mg. In another embodiment, the total amount of hydrocodone in the pharmaceutical composition may be about 15 mg. In still another embodiment, the total amount of hydrocodone in the pharmaceutical composition may be about 7.5 mg.

(ii) Other API

The composition disclosed herein may also comprise at least one other API. In general, the other API is preferentially absorbed in the upper gastrointestinal tract (GIT). Accordingly, optimal absorption of the API may occur in the upper GIT (i.e., duodenum, jejunum, and ileum of the small intestine), with little or no absorption in the lower GIT (i.e., cecum and colon of the large intestine).

In some embodiments, the other API may be a non-opioid analgesic. Suitable non-opioid analgesics include acetaminophen (also known as paracetamol), acetylsalicylic acid, diclofenac, diflunisol, ibuprofen, indomethacin, ketoprofen, ketorolac, naproxen, mefamanic acid, phenacetin, piroxicam, sulindac, and tolmetin. In other embodiments, the other API may be a steroidal anti-inflammatory agent such as celecoxib, deracoxib, ketoprofen, lumiracoxib, meloxicam, parecoxib, rofecoxib, or valdecoxib. In a further embodiment, the other API may be a steroidal anti-inflammatory agent such as alclometasone, dexamethasone, fluocinonide, hydrocortisone, methylprednisolone, prednisone, prednisolone, or triamcinolone. In further embodiments, the other API may be a norepinephrine transporter modulator such as tapentadol, a tricyclic antidepressant such as amitriptyline, an alpha-2 adrenergic agonist such as clonidine, a calcium channel blocker such as nimodipine, a GABA B agonist such as baclofen, a cannabinoid, a NMDA receptor antagonist, a CCK receptor antagonist, a beta blocker, or a serotonin receptor antagonist. Any of the aforementioned APIs may be in the form of a pharmaceutically acceptable salt. In various embodiments, the at least one extended release portion may comprise one, two, three, four, or more APIs. In one embodiment, one extended release portion may comprise one of the other APIs.

The amount of the other API in the gastric retentive, extended release composition can and will vary. In one embodiment, the composition may comprise from about 1.0 mg to about 1500 mg of the API. In another embodiment, the amount of API in the composition may range from about 100 mg to about 1000 mg. In still another embodiment, the amount of API in the composition may range from about 50 mg to about 500 mg. In another embodiment, the amount of API in the composition may range from about 10 mg to about 100 mg. In yet another embodiment, the amount of API in the composition may range from about 1.0 mg to about 10 mg. In one embodiment, the amount of API in the composition may range from about 250 mg to about 1300 mg. In another embodiment, the amount of API in the composition may range from about 325 mg to about 650 mg. In still another embodiment, the amount of API in the composition may range from about 650 mg to about 1300 mg.

The total amount of acetaminophen present in the pharmaceutical composition also may vary. In one embodiment, the total amount of acetaminophen present in the pharmaceutical composition may range from about 80 mg to about 1600 mg. In another embodiment, the total amount of acetaminophen present in the pharmaceutical composition may be about 250 mg to about 1300 mg. In a further embodiment, the total amount of acetaminophen present in the pharmaceutical composition may be about 300 mg to about 600 mg. In yet another embodiment, the total amount of acetaminophen present in the pharmaceutical composition may be about 325 mg to about 650 mg. In another embodiment, the total amount of acetaminophen present in the pharmaceutical composition may be about 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 600 mg, 650 mg, 700 mg, 750 mg, 1000 mg, or 1300 mg. In one embodiment, the total amount of acetaminophen in the pharmaceutical composition may be about 650 mg. In another embodiment, the total amount of acetaminophen in the pharmaceutical composition may be about 500 mg. In yet another embodiment, the total amount of acetaminophen in the pharmaceutical composition may be about 325 mg.

### (b) Immediate Release Portion

The pharmaceutical composition disclosed herein may comprise at least one immediate release portion. In one embodiment, the at least one immediate release portion may comprise hydrocodone. In another embodiment, the at least one immediate release portion may comprise acetaminophen. In a further embodiment, the at least one immediate release portion may comprise hydrocodone and acetaminophen.

The at least one immediate release portion of the pharmaceutical composition is designed to release more than 80%, more than 90%, or essentially all of the opioid(s) and/or the other API(s) in the at least one immediate release portion(s) within about one hour. In one embodiment, more than 80%, more than 90%, or essentially all of the opioid(s) and/or the other API(s) in the at least one immediate release portion(s) may be released in less than about 45 minutes. In another embodiment, more than 80%, more than 90%, or essentially all of the opioid(s) and/or the other API(s) in the at least one immediate release portion(s) may be released in less that about 30 minutes. In a further embodiment, more than 80%, more than 90%, or essentially all of the opioid(s) and/or the other API(s) in the at least one immediate release portion(s) may be released in less than about 20 minutes. In yet another embodiment, more than 80%, more than 90%, or essentially all of the opioid(s) and/or the other API(s) in the at least one immediate release portion(s) may be released in less that about 15 minutes. In an alternate embodiment, more than 80%, more than 90%, or essentially all of the opioid(s) and/or the other API(s) in the at least one immediate release portion(s) may be released in less that about 10 minutes. In yet another embodiment, more than 80%, more than 90%, or essentially all of the opioid(s) and/or the other API(s) in the at least one immediate release portion may be released in less that about 5 minutes.

In some embodiments, the immediate release portion may be part of or homogeneously mixed with the extended release portion.

(i) Opioid(s)

At least one immediate release portion of the composition may comprise at least one opioid. Suitable opioids are detailed above in Section (II)(a)(i). In one embodiment, the opioid may be codeine or a salt thereof. In another embodiment, the opioid may be hydrocodone or a salt thereof. In yet another embodiment, the opioid may be hydromorphone or a salt thereof. In still another embodiment, the opioid may be morphine or a salt thereof. In a further embodiment, the opioid may be oxymorphone or a salt thereof. In an alternate embodiment, the opioid may be tramadol or a salt thereof. In another embodiment, the opioid may be hydrocodone or a salt thereof.

The amount of opioid present in the at least one immediate release portion of the pharmaceutical composition can and will vary. In one embodiment, the amount of opioid in the at least one immediate release portion may range from about 0.4 mg to about 100 mg. In another embodiment, the amount of opioid in the at least one immediate release portion may range from about 1.25 mg to about 75 mg. In another embodiment, the amount of opioid in the at least one immediate release portion may range from about 1 mg to about 20 mg. In still another embodiment, the amount of opioid in the at least one immediate release portion may range from about 0.5 mg to about 10 mg. In another embodiment, the amount of opioid in the at least one immediate release portion may range from about 7.5 mg to about 15 mg. In yet another embodiment, the amount of opioid in the at least one immediate release portion may range from about 15 mg to about 30 mg. In an alternate embodiment, the amount of opioid in the at least one immediate release portion may range from about 30 mg to about 75 mg. In various embodiments, the amount of opioid in the at least one immediate release portion may be about 1.25 mg, 1.3 mg, 1.325 mg, 1.35 mg, 1.375 mg, 1.4 mg, 1.425 mg, 1.45 mg, 1.475 mg, 1.5 mg, 1.525 mg, 1.55 mg, 1.575 mg, 1.6 mg, 1.625 mg, 1.65 mg, 1.675 mg, 1.7 mg, 1.725 mg, 1.75 mg, 1.775 mg, 1.8 mg, 1.825 mg, 1.85 mg, 1.875 mg, 1.9 mg, 1.925 mg, 1.95 mg, 1.975 mg, 2.0 mg, 2.25 mg, 2.5 mg, 2.75 mg, 3.0 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6.0 mg, 6.25 mg, 6.5 mg, 6.75 mg, 7.0 mg, 7.25 mg, 7.5 mg, 7.75 mg, 8.0 mg, 8.25 mg, 8.5 mg, 8.75 mg, 9.0 mg, 9.25 mg, 9.5 mg, 9.75 mg, 10.0 mg, 0 mg, 12.0 mg, 13.0 mg, 14.0 mg, 15.0 mg, 20.0 mg, 25 mg, 30 mg, 35 mg or 40.0 mg. In one embodiment, the amount of opioid in the at least one immediate release portion may range from about 1.0 mg and about 2.0 mg, for example, about 1.875 mg. In yet another embodiment, the amount of opioid in the at least one immediate release portion may range from about 2.0 mg and about 3.0 mg, for example, about 2.25 mg. In another embodiment, the amount of opioid in the at least one immediate release portion may range from 7.0 mg and about 8.0 mg, for example, about 7.5 mg. In still another embodiment, the amount of opioid in the at least one immediate release portion may range from between about 7.0 mg and about 8.0 mg, for example, about 7.5 mg. In a further embodiment, the amount of opioid in the at least one immediate release portion may range from about 1.0 mg and about 5.0 mg. In yet another embodiment, the amount of opioid in the at least one immediate release portion may range from about 1.0 mg and about 4.5 mg. In another embodiment, the amount of opioid in the at least one immediate release portion may range from about 1.0 mg and about 4.0 mg. In still another embodiment, the amount of opioid in the at least one immediate release portion may range from about 1.0 mg and about 3.5 mg. In yet another embodiment, the amount of opioid in the at least one immediate release portion may range from about 1.0 mg and about 3.0 mg.

The amount of opioid present in the at least one immediate release portion(s) may be expressed as a percentage (w/w) of the total amount of opioid in the pharmaceutical composition. In one embodiment, the at least one immediate release portion may comprise from about 20% to about 40% (w/w) of the total amount of opioid present in the pharmaceutical composition. In certain embodiments, the percentage of opioid present in the at least one immediate release portion of the pharmaceutical composition may be about 20%, 21%, 22%, 23%, 24%, 25%, 26%, 27%, 28%, 29%, 30%, 31%, 32%, 33%, 34%, 35%, 36%, 37%, 38%, 39%, or 40% (w/w) of the total amount of opioid present in the composition. In one embodiment, the percentage of opioid present in the at least one immediate release portion may range from about 20% to about 30% (w/w) of the total amount of opioid present in the composition. In another embodiment, the percentage of opioid present in the at least one immediate release portion of the pharmaceutical composition may be about 25% (w/w) of the total amount of opioid present in the pharmaceutical composition.

The amount of opioid in the at least one immediate release portion also may be expressed as a percentage (w/w) of the total weight of the immediate release portion(s) of the pharmaceutical composition. In one embodiment, the amount of opioid in an immediate release portion may range from about 0.2% (w/w) to about 20% (w/w) of the total weight of such immediate release portion of the pharmaceutical composition. In another embodiment, the amount of opioid in an immediate release portion may range from about 0.5% (w/w) to about 5% (w/w) of the total weight of such immediate release portion. In various embodiments, an immediate release portion may comprise an amount of opioid that is approximately 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, 10%, 11%, 12%, 13%, 14%, 15%, 16%, 17%, 18%, 19%, or 20% (w/w) of the total weight of such immediate release portion of the pharmaceutical composition. In yet another embodiment, the amount of opioid in an immediate release portion may be about 0.5% (w/w) to about 1.0% (w/w) of the total weight of such immediate release portion of the pharmaceutical composition.

In some embodiments, the opioid in the at least one immediate release portion(s) of the pharmaceutical composition may be in the form of particles comprising opioid and at least one excipient. The at least one immediate release portion, therefore, may comprise particles of opioid(s) that are admixed with other API(s) and optional excipient(s). Suitable hydrocodone particles are described in co-pending application U.S. application Ser. No. 13/166,770, filed Jun. 22, 2011, which is incorporated herein by reference in its entirety. The opioid particles may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

In one embodiment, the opioid found in the at least one immediate release portion of the pharmaceutical composition may comprise hydrocodone. The amount of hydrocodone in the at least one immediate release portion of the pharmaceutical composition can and will vary. In one embodiment, the amount of hydrocodone in the at least one immediate release portion may range from about 0.4 mg to about 100 mg. In one embodiment, the amount of hydrocodone in the at least one immediate release portion may range from about 1 mg to about 40 mg. In a further embodiment, the amount of hydrocodone in the at least one immediate release portion of the pharmaceutical composition may range from about 1 mg to about 7.5 mg. In another embodiment, the amount of hydrocodone in the at least one immediate release portion may range from about 7.5 mg to about 15 mg. In yet another embodiment, the amount of hydrocodone in the at least one immediate release portion may range from about 15 mg to about 40 mg. In various embodiments, the amount of in the at least one immediate release portion may be about 1.25 mg, 1.3 mg, 1.325 mg, 1.35 mg, 1.375 mg, 1.4 mg, 1.425 mg, 1.45 mg, 1.475 mg, 1.5 mg, 1.525 mg, 1.55 mg, 1.575 mg, 1.6 mg, 1.625 mg, 1.65 mg, 1.675 mg, 1.7 mg, 1.725 mg, 1.75 mg, 1.775 mg, 1.8 mg, 1.825 mg, 1.85 mg, 1.875 mg, 1.9 mg, 1.925 mg, 1.95 mg, 1.975 mg, 2.0 mg, 2.25 mg, 2.5 mg, 2.75 mg, 3.0 mg, 3.25 mg, 3.5 mg, 3.75 mg, 4.0 mg, 4.25 mg, 4.5 mg, 4.75 mg, 5.0 mg, 5.25 mg, 5.5 mg, 5.75 mg, 6.0 mg, 6.25 mg, 6.5 mg, 6.75 mg, 7.0 mg, 7.25 mg, 7.5 mg, 7.75 mg, 8.0 mg, 8.25 mg, 8.5 mg, 8.75 mg, 9.0 mg, 9.25 mg, 9.5 mg, 9.75 mg, 10.0 mg, 11.0 mg, 12.0 mg, 12.5 mg, 13.0 mg, 14.0 mg, 15.0 mg, 17.5 mg, 20.0 mg, 22.5 mg, 25 mg, 27.5 mg, 30 mg, 40.0 mg, 75 mg, or 100 mg. In one embodiment, the amount of hydrocodone in the at least one immediate release portion may range from about 7.0 mg and about 8.0 mg, for example, about 7.5 mg. In another embodiment, the amount of hydrocodone in the at least one immediate release portion may be between about 3.0 mg and about 4.0 mg, for example, about 3.75 mg. In still another embodiment, the amount of hydrocodone in the at least one immediate release portion may be between about 1.0 mg and about 2.0 mg, for example, about 1.875 mg. In a further embodiment, the amount of hydrocodone in the at least one immediate release portion may range from about 1.0 mg and about 5.0 mg. In yet another embodiment, the amount of hydrocodone in the at least one immediate release portion may range from about 1.0 mg and about 4.5 mg. In another embodiment, the amount of hydrocodone in the at least one immediate release portion may range from about 1.0 mg and about 4.0 mg. In still another embodiment, the amount of hydrocodone in the at least one immediate release portion may range from about 1.0 mg and about 3.5 mg. In yet another embodiment, the amount of hydrocodone in the at least one immediate release portion may range from about 1.0 mg and about 3.0 mg.

The amount of hydrocodone present in the at least one immediate release portion(s) may be expressed as a percentage (w/w) of the total amount of hydrocodone in the pharmaceutical composition. In one embodiment, the at least one immediate release portion may comprise from about 20% to about 40% (w/w) of the total amount of hydrocodone present in the pharmaceutical composition. In still another embodiment, the at least one immediate release portion may comprise from about 20% to about 30% (w/w) of the total amount of hydrocodone present in the pharmaceutical composition. In certain embodiments, the percentage of hydrocodone present in the at least one immediate release portion of the pharmaceutical composition may be about 20%, 21%, 22%, 23%, 24%, 25%, 26%, 27%, 28%, 29%, 30%, 31%, 32%, 33%, 34%, 35%, 36%, 37%, 38%, 39%, or 40% (w/w) of the total amount of hydrocodone. In another embodiment, the percentage of hydrocodone present in the at least one immediate release portion of the pharmaceutical composition may be about 25% (w/w) of the total amount of hydrocodone present in the pharmaceutical composition.

The amount of hydrocodone in the at least one immediate release portion also may be expressed as a percentage (w/w) of the total weight of the immediate release portion(s) of the pharmaceutical composition. In one embodiment, the amount of hydrocodone in an immediate release portion may range from about 0.2% (w/w) to about 15.0% (w/w) of the total weight of such immediate release portion of the pharmaceutical composition. one embodiment, the amount of hydrocodone in an immediate release portion may range from about 0.2% (w/w) to about 20.0% (w/w) of the total weight of such immediate release portion of the pharmaceutical composition. In another embodiment, the amount of hydrocodone in an immediate release portion may range from about 0.5% (w/w) to about 5% (w/w) of the total weight of such immediate release portion. In yet another embodiment, the amount of hydrocodone in an immediate release portion may range from about 0.5% (w/w) to about 2% (w/w) of the total weight of such immediate release portion In various embodiments, an immediate release portion may comprise an amount of hydrocodone that is approximately 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, 10%, 11%, 12%, 13%, 14%, 15%, 16%, 17%, 18%, 19%, or 20% (w/w) of the total weight of such immediate release portion of the pharmaceutical composition. In yet another embodiment, the amount of hydrocodone in an immediate release portion may be about 0.5% (w/w) to about 1.0% (w/w) of the total weight of such immediate release portion of the pharmaceutical composition.

In some embodiments, the hydrocodone of the at least one immediate release portion(s) of the pharmaceutical composition may be in the form of particles comprising hydrocodone and at least one excipient. The at least one immediate release portion, therefore, may comprise particles of hydrocodone that are admixed with other API(s), such as acetaminophen and optional excipient(s). Suitable hydrocodone particles are described in co-pending application U.S. application Ser. No. 13/166,770, filed Jun. 22, 2011, which is incorporated herein by reference in its entirety. The opioid particles, such as hydrocodone particles, may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

(ii) Other API(s)

At least one immediate release portion of the composition may comprise at least one other API. Examples of suitable APIs that may be included in the at least one immediate release portion are presented above in Section (II)(a)(ii). In one embodiment, the other API may be acetylsalicylic acid or a salt thereof. In another embodiment, the other API may be diclofenac or a salt thereof. In yet another embodiment, the other API may be ibuprofen or a salt thereof. In still another embodiment, the other API may be indomethacin or a salt thereof. In a further embodiment, the other API may be ketoprofen or a salt thereof. In an alternate embodiment, the other API may be naproxen or a salt thereof. In another embodiment, the other API may be piroxicam or a salt thereof. In still another embodiment, the other API may be prednisolone or a salt thereof. In one embodiment, the other API may be acetaminophen or salt thereof.

The amount of the other API in the at least one immediate release portion can and will vary. In one embodiment, the immediate release portion may comprise from about 0.5 mg to about 750 mg of the API. In another embodiment, the amount of API in the at least one immediate release portion may range from about 50 mg to about 500 mg. In another embodiment, the amount of API in the at least one immediate release portion may range from about 25 mg to about 250 mg. In another embodiment, the amount of API in the at least one immediate release portion may range from about 150 mg to about 500 mg. In yet another embodiment, the amount of API in the at least one immediate release portion may range from about 0.5 mg to about 5 mg. In one embodiment, the amount of API in the at least one immediate release portion may range from about 125 mg to about 650 mg. In another embodiment, the amount of API in the at least one immediate release portion may range from about 162.5 mg to about 325 mg. In still another embodiment, the amount of API in the at least one immediate release portion may range from about 325 mg to about 650 mg. In an additional embodiment, the amount of API in the at least one immediate release portion may range from about 100 mg to about 400 mg. In still another embodiment, the amount of API in the at least one immediate release portion may range from about 125 mg to about 325 mg.

The amount of other API in the at least one immediate release portion of the pharmaceutical composition can and will vary. In general, the amount of other API present in the at least one immediate release portion may range from about 30% to about 70% (w/w) of the total amount of other API in the composition. In one embodiment, the amount of other API present in the at least one immediate release portion ranges from about 40% to about 60% (w/w) of the total amount of API in the composition. In various embodiments, the at least one immediate release portion of the composition may comprise about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% (w/w) of the total amount of API in the composition.

The amount of other API in an immediate release portion of the composition may range from about 15% to about 95% (w/w) of the total weight of such immediate release portion of the composition. In various embodiments, the amount of other API(s) in an immediate release portion may be about 15%, 20%, 25%, 30%, 35%, 40%, 45%, 50%, 55%, 60%, 65%, 68%, 69%, 70%, 71%, 72%, 73%, 74%, 75%, 76%, 77%, 78%, 79%, 80%, 82%, 84%, 86%, 88%, 90%, 92%, or 95% (w/w) of the total weight of such immediate release portion.

In embodiments in which the other API is acetaminophen, the amount of acetaminophen in the at least one immediate release may range from about 40 mg to about 800 mg. In still another embodiment, the at least one immediate release portion of the pharmaceutical composition may comprise from about 100 mg to about 600 mg of acetaminophen. In another embodiment, the at least one immediate release portion may comprise from about 150 mg to about 400 mg of acetaminophen. In a further embodiment, the amount of acetaminophen in the at least one immediate release portion may range from about 160 mg to about 325 mg. In an additional embodiment, the amount of acetaminophen in the at least one immediate release portion may range from about 100 mg to about 400 mg. In yet another embodiment, the amount of acetaminophen in the at least one immediate release portion may range from about 125 mg to about 400 mg In still another embodiment, the amount of acetaminophen in the at least one immediate release portion may range from about 125 mg to about 325 mg. In yet another embodiment, the amount of acetaminophen in the at least one immediate release portion may range from about 125 mg to about 400 mg.

In still another embodiment, the amount of acetaminophen in the at least one immediate release portion may be about 120 mg, 125 mg, 130 mg, 135 mg, 140 mg, 145 mg, 150 mg, 155 mg, 160 mg, 162.5 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 500 mg, 520 mg, 600 mg, 650 mg, 700 mg, 750 mg, or 780 mg. In one embodiment, the at least one immediate release portion may comprise about 325 mg of acetaminophen. In another embodiment, the amount of acetaminophen in the at least one immediate release portion may be about 250 mg. In yet another embodiment, the amount of acetaminophen in the at least one immediate release portion may be about 162.5 mg. In still another embodiment, the amount of acetaminophen in the at least one immediate release portion may be about 125 mg.

The at least one immediate release portion(s) of the pharmaceutical composition may comprise from about 40% to about 60% (w/w) of the total amount of acetaminophen present in the pharmaceutical composition. The amount of acetaminophen in the at least one immediate release portion may be about 40%, 41%, 42%, 43%, 44%, 45%, 46%, 47%, 48%, 49%, 50%, 51%, 52%, 53%, 54%, 55%, 56%, 57%, 58%, 59%, or 60% (w/w) of the total amount of acetaminophen present in the pharmaceutical composition. In one embodiment, the percentage of acetaminophen present in the at least one immediate release portion may be about 50% (w/w) of the total amount of acetaminophen present in the pharmaceutical composition.

The amount of acetaminophen in an immediate release portion(s) of the pharmaceutical composition may range from about 20% (w/w) to about 95% (w/w) of the total weight of such immediate release portion of the composition. In various embodiments, an immediate release portion may comprise an amount of acetaminophen that is approximately about 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% (w/w) of the total weight of such immediate release portion. In one embodiment, the amount of acetaminophen in an immediate release portion may range from about 70% to about 80% (w/w) of the total weight of such immediate release portion of the pharmaceutical composition.

(iii) Excipients

The at least one immediate release portion(s) of the pharmaceutical composition may further comprise at least one excipient. Suitable excipients include binders, fillers, disintegrants, lubricants, antioxidants, chelating agents, and color agents.

In one embodiment, the at least one immediate release portion(s) of the pharmaceutical composition may comprise at least one binder. Suitable binders include, without limit, starches (including corn starch and pregelatinized starch), gelatin, sugars (including sucrose, glucose, dextrose and lactose), polyethylene glycol, polyols, polyvinylalcohols, C12-C18 fatty acid alcohols, waxes, gums (e.g., guar gum, arabic gum, acacia gum, xantham gum, etc.), gelatin, pectin, sodium alginate, polyvinylpyrrolidone, cellulosic polymers (including hydroxypropyl cellulose, hydroxypropyl methylcellulose, hydroxylcellulose, methylcellulose, microcrystalline cellulose, ethylcellulose, hydroxyethyl cellulose, and the like), polyacrylamides, and polyvinyloxoazolidone. In one embodiment, the amount of binder or binders in an immediate release portion of the pharmaceutical composition may range from about 5% to about 10% (w/w) of the total weight of such immediate release portion. In various embodiments, an immediate release portion of the pharmaceutical composition may comprise at least one binder that is present in an amount that is about 5.0%, 5.25%, 5.5%, 5.75%, 6.0%, 6.25%, 6.5%, 6.75%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, or 9.0% (w/w) of such immediate release portion of the composition.

In another embodiment, the at least one immediate release portion(s) of the pharmaceutical composition may comprise at least one filler. Suitable fillers include but are not limited to microcrystalline cellulose (MCC), dibasic calcium phosphate, tribasic calcium phosphate, magnesium carbonate, magnesium oxide, calcium silicate, magnesium aluminum silicate, silicon dioxide, titanium dioxide, alumina, talc, kaolin, polyvinylpyrrolidone, dibasic calcium sulfate, tribasic calcium sulfate, starch, calcium carbonate, magnesium carbonate, carbohydrates, modified starches, lactose, sucrose, dextrose, mannitol, sorbitol, and inorganic compounds. In one embodiment, the amount of filler or fillers in an immediate release portion may range from about 1.0% to about 10.0% (w/w) of the total weight of such immediate release portion. In various embodiments, an immediate release portion of the pharmaceutical composition may comprise at least one filler that is present in an amount that is about 1.0%, 1.5%, 2.0%, 2.5%, 3.0%, 3.5%, 4.0%, 4.5%, 5.0%, 5.5%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0%, (w/w), of such immediate release portion of the pharmaceutical composition.

In still another embodiment, the at least one immediate release portion(s) of the pharmaceutical composition may further comprise a disintegrant. The disintegrant may be selected from the group consisting of croscarmellose sodium, crospovidone, alginic acid, carboxymethylcellulose calcium, carboxymethylcellulose sodium, low substituted hydroxypropylcellulose, microcrystalline cellulose, and sodium starch glycolate. In one embodiment, the amount of disintegrant in an immediate release portion may range from about 2.0% to about 15.0% (w/w) of the total weight of such immediate release portion. In some embodiments, the amount of disintegrant in an immediate release portion may be about 4.0%, 4.2%, 4.4%, 4.6%, 4.8%, 5.0%, 5.2%, 5.4%, 5.6%. 5.8%, 6.0%, 6.2%, 6.4%. 6.6%, 6.8%, or 7.0% (w/w) of such immediate release portion of the pharmaceutical composition.

In a further embodiment, the at least one immediate release portion(s) of the pharmaceutical composition may further comprise a lubricant. Useful lubricants include magnesium stearate, calcium stearate, stearic acid, and hydrogenated vegetable oil (preferably comprised of hydrogenated and refined triglycerides of stearic and palmitic acids). The lubricant may be present in an amount ranging from about 0.1% to about 3.0% (w/w) of the total weight of an immediate release portion. In certain embodiments, the amount of lubricant in at least one immediate release portion may be about 0.25%, 0.5%, 0.75%, 1.0%, 1.5%, 1.55%, 1.6%, 1.65%, 1.7%, 1.75%, 1.80%, 1.85%, 1.90%, 1.95%, or 2.0% (w/w) of the total weight of such immediate release portion

In yet another embodiment, the at least one immediate release portion(s) of the pharmaceutical composition may comprise at least one antioxidant. Suitable antioxidants include, without limitation, ascorbic acid, citric acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate. The amount of antioxidant present in an immediate release portion of the pharmaceutical composition may range from about 0.01% to about 4.0% (w/w), or from about 0.02% to about 0.10% (w/w) of the total weight of such immediate release portion. In various embodiments, the amount of antioxidant present in an immediate release portion of the pharmaceutical composition may be about 0.01%, 0.02%, 0.03%, 0.04%, 0.05%, 0.06%, 0.07%, 0.08%, 0.09%, 0.10%, 0.12%, 0.14%, 0.16%, 0.18%. 0.20%, 0.25%, 0.50%, 0.75%, 1.00%, 1.50%, or 2.00% (w/w) of the total weight of such immediate release portion.

In still another embodiment, the at least one immediate release portion(s) of the pharmaceutical composition may comprise at least one chelating agent. Suitable chelating agents include ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. In one embodiment, the chelating agent may be the sodium salt of EDTA. The amount of chelating agent present in an immediate release portion of the pharmaceutical composition may range from about 0.001% to about 0.20% (w/w) of such immediate release portion. In some embodiments, the amount of chelating agent present in an immediate release portion of the pharmaceutical composition may be about 0.001%, 0.002%, 0.003%, 0.004%, 0.005%, 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% (w/w) of the total weight of such immediate release portion.

In an alternate embodiment, the at least one immediate release portion of the pharmaceutical composition may comprise a color agent. Suitable color additives include, but are not limited to, food, drug and cosmetic colors (FD&C), drug and cosmetic colors (D&C), and external drug and cosmetic colors (Ext. D&C). In various embodiments, the amount of color agent present in an immediate release portion may range from about 2.0% to about 5.0% (w/w) of the total weight of such immediate release portion of the composition. In other embodiments, the amount of color agent present in an immediate release portion may be about 1.0%, 1.5%, 2.0%, 2.5%, 3.0%, 3.5%, 4.0%, 4.5%, or 5.0% (w/w) of the total weight of such immediate release portion.

### (c) Extended Release Portion

The pharmaceutical composition disclosed herein comprises at least one extended release portion. The at least one extended release portion may comprise at least one opioid, such as hydrocodone, at least one other API, such as acetaminophen, or combinations thereof. The at least one extended release portion(s) further comprises at least one extended release component. The extended release component may comprise at least one extended release polymer.

The at least one extended release portion of the pharmaceutical composition is designed to release the active agents over an extended period of time. In general, the at least one extended release portion(s) provides release of the opioid(s), such as hydrocodone, and/or the API(s), such as acetaminophen, for a period of time ranging from at least about 3 hours (hrs) to at least about 12 hrs. In one embodiment, the opioid(s) and/or the other API(s) may be released from the at least one extended release portion(s) over a period of at least about 5 hours, or over a period of at least about 6 hours. In another embodiment, the at least one extended release portion may release the opioid(s) and/or the other API(s) over a period of at least about 7 hours, or over a period of at least about 8 hours. In still another embodiment, the opioid(s) and/or the other API(s) may be released from the at least one extended release portion over a period of at least about 9 hours, or over a period of at least about 10 hours. In a further embodiment, the at least one extended release portion may release the opioid(s) and/or the other API(s) over a period of at least about 11 hours, or over a period of at least about 12 hours.

(i) Opioids

At least one extended release portion of the pharmaceutical composition comprises at least one opioid. Suitable opioids are detailed above in Section (II)(a)(i). In one embodiment, the opioid may be codeine or a salt thereof. In another embodiment, the opioid may be hydrocodone or a salt thereof. In yet another embodiment, the opioid may be hydromorphone or a salt thereof. In still another embodiment, the opioid may be morphine or a salt thereof. In a further embodiment, the opioid may be oxymorphone or a salt thereof. In an alternate embodiment, the opioid may be tramadol or a salt thereof. In another embodiment, the opioid may be oxycodone or a salt thereof.

The amount of opioid present in the at least one extended release portion(s) can and will vary. In one embodiment, the amount of opioid in the at least one extended release portion may range from about 1 mg to about 300 mg. In another embodiment, the amount of opioid in the at least one extended release portion may range from about 3.75 mg to about 225 mg. In yet another embodiment, the amount of opioid in the at least one extended release portion may range from about 3.75 mg to about 120 mg. In a further embodiment, the at least one extended release portion of the pharmaceutical composition may comprise from about 1 mg to about 22.5 mg of opioid. In another embodiment, the amount of opioid in the at least one extended release portion may be from about 22.5 mg to about 45 mg. In yet another embodiment, the amount opioid in the at least one extended release portion may be from about 45 mg to about 90 mg. In still another embodiment, the amount of opioid in the at least one extended release portion may be from about 90 mg to about 225 mg. In yet another embodiment, the amount of opioid in the at least one extended release portion may be about 10 mg to about 30 mg. In yet another embodiment, the amount of opioid in the at least one extended release portion may be about 30 mg to about 60 mg.

In one embodiment, the amount of opioid in the at least one extended release portion may be from about 22 mg to about 23 mg, for example, about 22.5 mg. In another embodiment, the amount of opioid in the at least one extended release portion may be about 10 mg to about 12 mg, for example, about 11.25 mg.

In a further embodiment, the amount of opioid may be about 5.625 mg. In an additional embodiment, the amount of opioid may be about 10 mg to about 12.5 mg. In a further embodiment, the amount of opioid may be about 12 mg to about 18 mg. In another embodiment, the amount of opioid in the at least one extended release portion may be about 20 mg to about 25 mg. In another embodiment, the at least one extended release portion comprises about 5 mg to about 7 mg of opioid. In a further embodiment, the amount of opioid may be about 5.625 mg to about 11.25 mg. In still another embodiment, the amount of opioid may be about 3.75 mg. In a yet another embodiment, the amount of opioid may be about 5.625 mg. In still another embodiment, the amount of opioid may be about 7.5 mg. In a yet an additional embodiment, the amount of opioid may be about 11.25 mg. In an additional embodiment, the amount of opioid may be about 2.0 mg to about 7.0 mg. In a further embodiment, the amount of opioid may be about 3.0 mg to about 7.0 mg. In still a further embodiment, the amount of opioid may be about 4.0 mg to about 7.0 mg. In a another embodiment, the amount of opioid may be about 4.0 mg to about 6.5 mg. In yet another embodiment, the amount of opioid may be about 4.5 mg to about 6.5 mg.

In yet another embodiment, the amount of opioid may be about 1.0 mg, 1.5 mg, 2.0 mg, 2.5 mg, 3.0 mg, 3.5 mg, 3.75, 4.0 mg, 4.5 mg, 5.0 mg, 5.5 mg, 5.625 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.25 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 22.5 mg, or 25 mg, 27.5 mg, 30 mg, 35 mg, 40 mg, 45 mg, or 50 mg.

The amount of opioid present in the at least one extended release portion(s) may be expressed as a percentage of the total amount of opioid in the pharmaceutical composition. In one embodiment, the at least one extended release portion of the pharmaceutical composition comprises from about 60% to about 80% (w/w) of the total amount of opioid present in the pharmaceutical composition. In certain embodiments, the percentage of opioid present in the at least one extended release portion of the pharmaceutical composition may be about 60%, 61%, 62%, 63%, 64%, 65%, 66%, 67%, 68%, 69% 70%, 71%, 72%, 73%, 74%, 75%, 76%, 77%, 78%, 79%, or 80% (w/w) of the total amount of opioid present in the composition. In one embodiment, the percentage of opioid present in the at least one extended release portion of the pharmaceutical composition may be about 75% of the total amount of opioid present in the pharmaceutical composition.

The amount of opioid in the extended release portion(s) also may be expressed as a percentage of the total weight of the extended release portion(s) of the pharmaceutical composition. In one embodiment, the amount of opioid in an extended release portion may range from about 0.3% to about 8.0% (w/w) of the total weight of the extended release portion of the pharmaceutical composition. In various embodiments, an extended release portion may comprise an amount of opioid that is approximately 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or 4.0%, 4.5%, 5%, 5.5%, 6%, 6.5%, 7%, 7.5%, or 8% (w/w) of the total weight of such extended release portion of the pharmaceutical composition. In one embodiment, the amount of opioid in an extended release portion comprises about 0.5% to about 2% (w/w) of the total weight of such extended release portion of the pharmaceutical composition.

In some embodiments, the opioid of the at least one extended release portion of the composition(s) may be in the form of particles comprising the opioid and at least one excipient. Thus, the at least one extended release portion may comprise particles of opioid(s) which are admixed with the additional API(s), such as acetaminophen, and the extended release component, both of which are detailed below, as well as optional excipient(s). Suitable hydrocodone particles are described in co-pending application U.S. application Ser. No. 13/166,770, filed Jun. 22, 2011, which is incorporated herein by reference in its entirety. The opioid particles may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

In embodiments in which the opioid is hydrocodone, the amount of hydrocodone in the at least one extended release portion(s) can and will vary. In one embodiment, the amount of hydrocodone in the at least one extended release portion may range from about 1 mg to about 300 mg. In another embodiment, the amount of opioid in the at least one extended release portion may range from about 3.75 mg to about 225 mg. In another embodiment, the amount of opioid in the at least one extended release portion may range from about 3.75 mg to about 120 mg. In still another embodiment, the amount of opioid in the at least one extended release portion may range from about 45 mg to about 90 mg.

In yet another embodiment, the amount of hydrocodone present in the at least one extended release portion(s) can and will vary. In one embodiment, the amount of hydrocodone in the at least one extended release portion may range from about 1 mg to about 120 mg. In a further embodiment, the at least one extended release portion of the pharmaceutical composition may comprise from about 1 mg to about 22.5 mg of hydrocodone. In another embodiment, the amount of in the at least one extended release portion may be about 10 mg to about 30 mg. In yet another embodiment, the amount of hydrocodone e in the at least one extended release portion may be about 30 mg to about 60 mg. In still another embodiment, the amount of hydrocodone in the at least one extended release portion may be about 22.5 mg to about 45 mg. In another embodiment, the at least one extended release portion comprises about 5 mg to about 7 mg of hydrocodone. In a further embodiment, the amount of hydrocodone may be about 5.625 mg to about 11.25 mg. In an additional embodiment, the amount of hydrocodone may be about 10 mg to about 12.5 mg. In a further embodiment, the amount of hydrocodone may be about 12 mg to about 18 mg. In another embodiment, the amount of hydrocodone in the at least one extended release portion may be about 20 mg to about 25 mg. In an additional embodiment, the amount of hydrocodone may be about 2.0 mg to about 7.0 mg. In a further embodiment, the amount of hydrocodone may be about 3.0 mg to about 7.0 mg. In still a further embodiment, the amount of hydrocodone may be about 4.0 mg to about 7.0 mg. In a another embodiment, the amount of hydrocodone may be about 4.0 mg to about 6.5 mg. In yet another embodiment, the amount of hydrocodone may be about 4.5 mg to about 6.5 mg.

In yet another embodiment, the amount of hydrocodone may be about 1.0 mg, 1.5 mg, 2.0 mg, 2.5 mg, 3.0 mg, 3.5 mg, 3.75 mg, 4.0 mg, 4.5 mg, 5.0 mg, 5.5 mg, 5.625 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.25 mg, 11.5 mg, 12.0 mg, 12.5 mg, 13.0 mg, 13.5 mg, 14.0 mg, 14.5 mg, 15.0 mg, 15.5 mg, 16.0 mg, 16.5 mg, 17.0 mg, 17.5 mg, 18.0 mg, 18.5 mg, 19.0 mg, 19.5 mg, 20.0 mg, 22.5 mg, 25 mg, 27.5 mg, 30 mg, 35 mg, 40 mg, 45 mg, or 50 mg. In one embodiment, the amount of hydrocodone in the at least one extended release portion may be from about 22 mg to about 23 mg, for example, about 22.5 mg. In another embodiment, the amount of hydrocodone in the at least one extended release portion may be about 10 mg to about 12 mg, for example, about 11.25 mg. In still another embodiment, the amount of hydrocodone in the at least one extended release portion may be from about 5 mg to about 6 mg, for example, about 5.625 mg. In yet embodiment, the amount of hydrocodone in the at least one extended release portion may be about 3 mg to about 4 mg, for example, about 3.75 mg. In still a further embodiment, the amount of hydrocodone in the at least one extended release portion may be from about 7 mg to about 8 mg, for example, about 7.5 mg.

The amount of hydrocodone present in the at least one extended release portion(s) may be expressed as a percentage of the total amount of hydrocodone in the pharmaceutical composition. In one embodiment, the at least one extended release portion of the pharmaceutical composition comprises from about 60% to about 80% (w/w) of the total amount of hydrocodone present in the pharmaceutical composition. In another embodiment, the at least one extended release portion of the pharmaceutical composition comprises from about 70% to about 80% (w/w) of the total amount of hydrocodone present in the pharmaceutical composition. In certain embodiments, the percentage of hydrocodone present in the at least one extended release portion of the pharmaceutical composition may be about 60%, 61%, 62%, 63%, 64%, 65%, 66%, 67%, 68%, 69% 70%, 71%, 72%, 73%, 74%, 75%, 76%, 77%, 78%, 79%, or 80% (w/w) of the total amount of hydrocodone. In one embodiment, the percentage of hydrocodone present in the at least one extended release portion of the pharmaceutical composition may be about 75% of the total amount of hydrocodone present in the pharmaceutical composition.

The amount of hydrocodone in the extended release portion(s) also may be expressed as a percentage of the total weight of the extended release portion(s) of the pharmaceutical composition. In one embodiment, the amount of hydrocodone in an extended release portion may range from about 0.3% to about 8.0% (w/w) of the total weight of the such extended release portion of the pharmaceutical composition. In another embodiment, the amount of hydrocodone in an extended release portion may range from about 0.5% to about 5.0% (w/w) of the total weight of the such extended release portion of the pharmaceutical composition. In various embodiments, an extended release portion may comprise an amount of hydrocodone that is approximately 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or 4.0%, 4.5%, 5%, 5.5%, 6%, 6.5%, 7%, 7.5%, or 8% (w/w) of the total weight of such extended release portion of the pharmaceutical composition. In one embodiment, the amount of hydrocodone in an extended release portion comprises about 0.5% to about 2% (w/w) of the total weight of such extended release portion of the pharmaceutical composition.

In some embodiments, the hydrocodone of the at least one extended release portion of the composition(s) may be in the form of particles comprising hydrocodone and at least one excipient. Thus, the at least one extended release portion may comprise particles of hydrocodone which are admixed with the additional API(s), such as acetaminophen and the extended release component, both of which are detailed below, as well as optional excipients. Suitable hydrocodone particles are described in co-pending application U.S. application Ser. No. 13/166,770, filed Jun. 22, 2011, which is incorporated herein by reference in its entirety. The hydrocodone particles may be coated or uncoated. The average size or average diameter of the particles may vary. In general, the average diameter of the particles may range from about 50 microns to about 2000 microns, from about 100 microns to about 1000 microns, or from about 150 microns to about 200 microns. In one embodiment, the maximum diameter of about 50% of the particles (d50) may be about 40 microns, 50 microns, 100 microns, 150 microns, 200 microns, 250 microns, 300 microns, 400 microns, or 500 microns. In another embodiment, the maximum diameter of about 90% of the particles (d90) may be about 100 microns, 150 microns, 200 microns, 250 microns, 300 microns, 400 microns, or 500 microns.

(ii) Other API(s)

The at least one extended release portion of the pharmaceutical composition may comprise at least one other API. Examples of suitable APIs that may be included in the at least one extended release portion are presented above in Section (I)(a)(ii). In one embodiment, the other API may be acetylsalicylic acid or a salt thereof. In another embodiment, the API may be diclofenac or a salt thereof. In yet another embodiment, the API may be ibuprofen or a salt thereof. In still another embodiment, the API may be indomethacin or a salt thereof. In a further embodiment, the API may be ketoprofen or a salt thereof. In an alternate embodiment, the API may be naproxen or a salt thereof. In another embodiment, the API may be piroxicam or a salt thereof. In still another embodiment, the API may be prednisolone or a salt thereof. In one embodiment, the API may be acetaminophen or salt thereof.

The amount of the other API in the at least one extended release portion can and will vary. In one embodiment, the at least one extended release portion may comprise from about 0.5 mg to about 750 mg of the API. In another embodiment, the amount of API in the at least one extended release portion may range from about 50 mg to about 500 mg. In another embodiment, the amount of API in the at least one extended release portion may range from about 25 mg to about 250 mg. In another embodiment, the amount of API in the at least one extended release portion may range from about 150 mg to about 500 mg. In yet another embodiment, the amount of API in the at least one extended release portion may range from about 0.5 mg to about 5 mg. In one embodiment, the amount of API in the at least one extended release portion may range from about 125 mg to about 650 mg. In another embodiment, the amount of API in the at least one extended release portion may range from about 162.5 mg to about 325 mg. In still another embodiment, the amount of API in the at least one extended release portion may range from about 325 mg to about 650 mg. In yet another embodiment, the amount of API in the at least one extended release portion may range from about 100 mg to about 400 mg. In an additional embodiment, the amount of API in the at least one extended release portion may range from about 125 mg to about 325 mg.

The amount of other API(s) in the at least one extended release portion of the pharmaceutical composition can and will vary, depending upon the identity of the API(s). In general, the amount of other API present in the at least one extended release portion may range from about 30% to about 70% (w/w) of the total amount of other API in the composition. In one embodiment, the amount of other API present in the at least one extended release portion may range from about 40% to about 60% (w/w) of the total amount of other API in the composition. In various embodiments, the at least one extended release portion of the pharmaceutical composition may comprise about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% (w/w) of the total amount of other API in the composition.

The amount of other API in an extended release portion also may be expressed as a percentage of the total weight of such extended release portion of the pharmaceutical composition. In various embodiments, the amount of other API in an extended release portion may range from about 10% to about 70% (w/w) of the total weight of such extended release portion of the composition. In various embodiments, the amount of other API in an extended release portion may be about 10%, 11%, 12%, 13%, 14%, 15%, 16%, 17%, 18%, 19%, 20%, 21%, 22%, 23%, 24%, 25%, 26%, 27%, 28%, 29%, 30%, 32%, 34%, 36%, 38%, 40%, 42%, 44%, 46%, 48%, 50%, 52%, 54%, 56%, 58%, 60%, 62%, 64%, 66%, 68%, or 70% (w/w) of the total weight of such extended release portion of the composition.

In embodiments in which the other API is acetaminophen, the amount of acetaminophen in the at least one extended release portion may range from about 40 mg to about 800 mg. In still another embodiment, the at least one extended release portion of the pharmaceutical composition may comprise from about 100 mg to about 600 mg of acetaminophen. In another embodiment, the at least one extended release portion may comprise from about 125 mg to about 400 mg of acetaminophen. In a further embodiment, the amount of acetaminophen in the at least one extended release portion may range from about 160 mg to about 325 mg. In yet another embodiment, the amount of acetaminophen in the at least one extended release portion may range from about 100 mg to about 400 mg. In an additional embodiment, the amount of acetaminophen in the at least one extended release portion may range from about 125 mg to about 325 mg.

In yet another embodiment, the amount of acetaminophen in the at least one extended release portion may be about 100 mg, 110 mg, 120 mg, 125 mg, 130 mg, 135 mg, 140 mg, 145 mg, 150 mg, 155 mg, 160 mg, 162.5 mg, 165 mg, 170 mg, 175 mg, 180 mg, 185 mg, 190 mg, 195 mg, 200 mg, 210 mg, 220 mg, 230 mg, 240 mg, 250 mg, 260 mg, 270 mg, 280 mg, 290 mg, 300 mg, 310 mg, 320 mg, 325 mg, 330 mg, 340 mg, 350 mg, 360 mg, 370 mg, 380 mg, 390 mg, 400 mg, 450 mg, 500 mg, 520 mg, 550 mg, 600 mg, 625 mg, 650 mg, 700 mg, 750 mg, 775 mg, 780 mg, or 800 mg. In one embodiment, the at least one extended release portion comprises about 325 mg of acetaminophen. In another embodiment, the amount of acetaminophen in the at least one extended release portion may be about 250 mg. In yet another embodiment, the amount of acetaminophen in the at least one extended release portion may be about 162.5 mg. In still another embodiment, the amount of acetaminophen in the at least one extended release portion may be about 125 mg.

The amount of acetaminophen in the at least one extended release portion(s) of the pharmaceutical composition may comprise from about 40% to about 60% of the total amount of acetaminophen present in the pharmaceutical composition. The amount of acetaminophen in the at least one extended release portion may be about 40%, 41%, 42%, 43%, 44%, 45%, 46%, 47%, 48%, 49%, 50%, 51%, 52%, 53%, 54%, 55%, 56%, 57%, 58%, 59%, or 60% (w/w) of the total amount of acetaminophen present in the pharmaceutical composition. In one embodiment, the percentage of acetaminophen present in the at least one extended release portion(s) of the pharmaceutical composition may be about 50% (w/w) of the total amount of acetaminophen present in the composition.

The amount of acetaminophen in an extended release portion of the pharmaceutical composition may range from about 15% to about 60% (w/w) of the total weight of such extended release portion of the pharmaceutical composition. In various embodiments, the amount of acetaminophen in an extended release portion may comprise an amount of acetaminophen that is approximately about 15%, 16%, 17%, 18%, 19%, 20%, 21%, 22%, 23%, 24%, 25%, 26%, 27%, 28%, 29%, 30%, 32%, 35%, 37%, 40%, 42%, 45%, 47%, 50%, 52%, 55%, 57%, or 60% (w/w) of the total weight of such extended release portion. In one embodiment, the amount of acetaminophen in an extended release portion may range from about 20% to about 40% (w/w) of the total weight of such extended release portion of the pharmaceutical composition.

(iii) Extended Release Component

The extended release portion(s) of the pharmaceutical composition also comprise(s) an extended release component. Suitable extended release components include polymers, resins, hydrocolloids, hydrogels, and the like.

In one embodiment, the extended release component may comprise at least one extended release polymer. Suitable polymers for inclusion in the at least one extended release portion of the pharmaceutical composition may be linear, branched, dendrimeric, or star polymers, and include synthetic hydrophilic polymers as well as semi-synthetic and naturally occurring hydrophilic polymers. The polymers may be homopolymers or copolymers, such as random copolymers, block copolymers, and graft copolymers. Suitable hydrophilic polymers include, but are not limited to: polyalkylene oxides, particularly poly(ethylene oxide), polyethylene glycol and poly(ethylene oxide)-poly(propylene oxide) copolymers; cellulosic polymers, such as methylcellulose, hydroxymethylcellulose, hydroxyethylcellulose, hydroxypropylcellulose, hydroxypropylmethylcellulose, and carboxymethylcellulose, microcrystalline cellulose, and polysaccharides and their derivatives; acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate; maleic anhydride copolymers; polymaleic acid; poly(acrylamides) such as polyacrylamide per se, poly(methacrylamide), poly(dimethylacrylamide), and poly(N-isopropyl-acrylamide); polyalkylene oxides; poly(olefinic alcohol)s such as poly(vinyl alcohol); poly(N-vinyl lactams) such as poly(vinyl pyrrolidone), poly(N-vinyl caprolactam), and copolymers thereof; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol and polyoxyethylated glucose; polyoxazolines, including poly(methyloxazoline) and poly(ethyloxazoline); polyvinylamines; polyvinylacetates, including polyvinylacetate per se as well as ethylene-vinyl acetate copolymers, polyvinyl acetate phthalate, and the like, polyimines, such as polyethyleneimine; starch and starch-based polymers; polyurethane hydrogels; chitosan; polysaccharide gums; xanthan gum; zein; and shellac, ammoniated shellac, shellac-acetyl alcohol, and shellac n-butyl stearate. The polymers may be used individually or in combination. Certain combinations will often provide a more controlled release of opioid(s), such as hydrocodone, and API(s), such as acetaminophen, than their components when used individually. Suitable combinations include cellulose-based polymers combined with gums, such as hydroxyethyl cellulose or hydroxypropyl cellulose combined with xanthan gum, and poly(ethylene oxide) combined with xanthan gum.

In one embodiment, the extended release polymer(s) may be a cellulosic polymer, such as an alkyl substituted cellulose derivative as detailed above. In terms of their viscosities, one class of exemplary alkyl substituted celluloses includes those whose viscosity is within the range of about 100 to about 110,000 centipoise as a 2% aqueous solution at 20° C. Another class includes those whose viscosity is within the range of about 1,000 to about 4,000 centipoise as a 1% aqueous solution at 20° C.

In one embodiment, the extended release polymer(s) may be a polyalkylene oxide. In another aspect, the polyalkylene oxide may be poly(ethylene) oxide. In a further embodiment, the poly(ethylene) oxide may have an approximate molecular weight between 500,000 Daltons (Da) to about 10,000,000 Da or about 900,000 Da to about 7,000,000 Da. In yet a further embodiment, the poly(ethylene) oxide may have a molecular weight of approximately about 600,000 Da, about 700,000 Da, about 800,000 Da, about 900,000 Da, about 1,000,000 Da, about 2,000,000 Da, about 3,000,000 Da, about 4,000,000 Da, about 5,000,000 Da, about 6,000,000 Da, about 7,000,000 Da, about 8,000,000 Da, 9,000,000 Da, or 10,000,000 Da.

In another embodiment, the polyethylene oxide may be any desirable grade of POLYOX™ or any combination thereof. By way of example and without limitation, the POLYOX™ grade may be WSR N-10, WSR N-80, WSR N-750, WSR 205, WSR 1105, WSR N-12K, WSR N-60K, WSR-301, WSR Coagulant, WSR-303, WSR-308, WSR N-3000, UCARFLOC Polymer 300, UCARFLOC Polymer 302, UCARFLOC Polymer 304, and UCARFLOC Polymer 309. In one embodiment, the polyethylene oxide may have an average molecular weight of from about 100,000 Da to about 8,000,000 Da. In another embodiment, the polyethylene oxide may have an average molecular weight of about 100,000 Da, about 200,000 Da, about 300,000 Da, about 400,000 Da, about 500,000 Da, about 600,000 Da, about 700,000 Da, about 800,000 Da, about 900,000 Da, about 1,000,000 Da, about 2,000,000 Da, about 3,000,000 Da, about 4,000,000 Da, about 5,000,000 Da, about 6,000,000 Da, about 7,000,000 Da, or about 8,000,000 Da. In still another embodiment, the polyethylene oxide may have an average number of repeating ethylene oxide units (—CH₂CH₂O—) of about 2,000 to about 160,000. In yet another embodiment, the polyethylene oxide may have an average number of repeating ethylene oxide units of about 2,275, about 4,500, about 6,800, about 9,100, about 14,000, about 20,000, about 23,000, about 45,000, about 90,000, about 114,000, or about 159,000.

The release profile of the extended release compositions disclosed herein will depend partially upon the molecular weight of the extended release polymer(s). In certain embodiments, the polymers are of a moderate to high molecular weight (900,000 Da to 4,000,000 Da) to control release of the opioid, such as hydrocodone and/or the API(s), such as acetaminophen from the composition via diffusion of the opioid(s) and/or other active agent(s) out of the polymer and/or erosion of the polymer. An example of suitable polyethylene oxide polymers are those having molecular weights (viscosity average) on the order of about 900,000 Da to about 2,000,000 Da. Using a lower molecular weight (“MW”) polyethylene oxide, such as POLYOX® 1105 (900,000 MW), the release rates for both drugs are higher. Using a higher molecular weight polyethylene oxide (such as POLYOX® N-60K (2,000,000 MW) or POLYOX® WSR-301 (4,000,000 MW) reduces the rate of release for both drugs. In another embodiment of the invention, a hydroxypropylmethylcellulose polymer of such molecular weight is utilized so that the viscosity of a 2% aqueous solution is about 4000 cps to greater than about 100,000 cps.

The release profile of the extended release pharmaceutical composition disclosed herein may also depend upon the amount of the extended release polymer(s) in the pharmaceutical composition. In general, the release rates for all active agents may be decreased by increasing the amount of the extended release polymer(s) in the pharmaceutical composition. Stated another way, the release rates for the opioid, such as hydrocodone, and/or the additional API, such as acetaminophen, may be decreased by increasing the amount of the extended release polymer(s) in the pharmaceutical composition. By way of example and without limitation, the release profile of all active agents (e.g., acetaminophen and hydrocodone) may be decreased by increasing the amount of POLYOX® 1105 from about 25% by weight of the ER portion to about 35% by weight of the ER portion.

The amount of extended release polymer or polymers present in the extended release portion(s) of the pharmaceutical composition can and will vary. In one embodiment, the polymer present in an extended release portion of the pharmaceutical composition may range from about 15% to about 70% (w/w), or about 20% to about 60% (w/w), or about 25% to about 55% (w/w) of the total weight of such extended release portion of the dosage form. In another embodiment, the amount of polymer present in an extended release portion of the pharmaceutical composition may range from about 30% to about 50% (w/w) of the total weight of such extended release portion. In still another embodiment, the amount of polymer present in an extended release portion of the pharmaceutical composition may range from about 35% to about 45% (w/w) of the total weight of such extended release portion. In yet another embodiment, the amount of polymer present in an extended release portion of the pharmaceutical composition may be about 30%, 35%, 40%, 45%, 50%, 55%, or 60% (w/w) of the total weight of such extended release portion. In one embodiment, the amount of polymer present in an extended release portion of the pharmaceutical composition may be about 35% (w/w) of the total weight of such extended release portion. In another embodiment, the amount of polymer present in an extended release portion of the pharmaceutical composition may be about 45% (w/w) of the total weight of such extended release portion. In one embodiment, the ER layer swells upon imbibition of fluid to a size which is about 15%, 20%, 25%, 30%, 35% 40%, 45%, 50%, 55%, 60%, 65%, 70%, 75%, 80%, 85%, 90%, 95% or 100% larger than the size of the ER layer prior to imbibition of fluid. In another embodiment, the ER layer swells upon imbibition of fluid to a size at least about 25% larger than the size of the ER layer prior to imbibition of fluid within about 15 minutes of the start of fluid imbibition. In still another embodiment, the ER layer swells upon imbibition of fluid to a size at least about 100% larger than the size of the ER layer prior to imbibition of fluid within about 45 min, 50 min, 60 min, 75 min, or 90 min of the start of fluid imbibitions.

(iv) Excipients

The extended release portion(s) of the pharmaceutical composition may further comprise at least one excipient. Suitable excipients include binders, fillers, lubricants, antioxidants, chelating agents, and color agents.

In one embodiment, the extended release portion(s) of the pharmaceutical composition may comprise at least one binder. Suitable binders include, without limit, starches (including corn starch and pregelatinized starch), gelatin, sugars (including sucrose, glucose, dextrose and lactose), polyethylene glycol, polyols, polyvinylalcohols, C12-C18 fatty acid alcohols, waxes, gums (e.g., guar gum, arabic gum, acacia gum, xanthan gum, etc.), gelatin, pectin, sodium alginate, polyvinylpyrrolidone, cellulosic polymers (including hydroxypropyl cellulose, hydroxypropyl methylcellulose, hydroxylcellulose, methylcellulose, microcrystalline cellulose, ethylcellulose, hydroxyethyl cellulose, and the like), polyacrylamides, and polyvinyloxoazolidone. In one embodiment, the amount of binder or binders in an extended release portion of the pharmaceutical composition may range from about 0.5% to about 8.0% (w/w) of such extended release portion. In various embodiments, an extended release portion of the pharmaceutical composition may comprise at least one binder that is present in an amount that is about 0.5%, 1.0%, 1.1%, 1.2%, 1.3%, 1.4%, 1.5%, 1.6%, 1.7%, 1.8%, 1.9%, 2.0%, 2.5%, 3.0%, 3.5%, 4.0%, 4.5%, 5.0%, 5.5%, 6.0%, 6.5%, 7.0%, 7.5%, or 8.0% (w/w) of such extended release portion of the composition.

In another embodiment, the at least one extended release portion(s) of the pharmaceutical composition may comprise at least one filler. Suitable fillers include but are not limited to microcrystalline cellulose (MCC), dibasic calcium phosphate, tribasic calcium phosphate, magnesium carbonate, magnesium oxide, calcium silicate, magnesium aluminum silicate, silicon dioxide, titanium dioxide, alumina, talc, kaolin, polyvinylpyrrolidone, dibasic calcium sulfate, tribasic calcium sulfate, starch, calcium carbonate, magnesium carbonate, carbohydrates, modified starches, lactose, sucrose, dextrose, mannitol, sorbitol, and inorganic compounds. In one embodiment, the amount of filler or fillers in an extended release portion may range from about 2% to about 50% (w/w) of the total weight of such extended release portion. In various embodiments, an extended release portion of the pharmaceutical composition may comprise at least one filler that is present in an amount that is about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% (w/w) of such extended release portion of the composition.

In a further embodiment, the extended release portion(s) of the pharmaceutical composition may further comprise a lubricant. Useful lubricants include magnesium stearate, calcium stearate, stearic acid, and hydrogenated vegetable oil (preferably comprised of hydrogenated and refined triglycerides of stearic and palmitic acids). The lubricant may be present in an amount ranging from about 0.1% to about 3.0% (w/w) of the total weight of the extended release portion. In certain embodiments, the amount of lubricant in an extended release portion may be about 0.25%, 0.5%, 0.75%, 1.0%, 1.5%, 1.75%, 1.80%, 1.85%, 1.90%, or 2.0% (w/w) of the total weight of such extended release portion of the composition.

In yet another embodiment, the extended release portion(s) of the pharmaceutical composition may comprise at least one antioxidant. Suitable antioxidants include, without limit, ascorbic acid, citric acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, and propylgallate. The amount of antioxidant present in an extended release portion of the pharmaceutical composition may range from about 0.01% to about 4.0% (w/w), or from about 0.02% to about 0.10% (w/w). In various embodiments, the amount of antioxidant present in an extended release portion of the pharmaceutical composition may be about 0.01%, 0.02%, 0.03%, 0.04%, 0.05%, 0.06%, 0.07%, 0.08%, 0.09%, 0.10%, 0.12%, 0.14%, 0.16%, 0.18%. 0.20%, 0.25%, 0.50%, 0.75%, 1.00%, 1.50%, or 2.00% (w/w) of the total weight of such extended release portion.

In still another embodiment, the extended release portion(s) of the pharmaceutical composition may comprise at least one chelating agent. Suitable chelating agents include ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. In one embodiment, the chelating agent may be the sodium salt of EDTA. The amount of chelating agent present in an extended release portion of the pharmaceutical composition may range from about 0.001% to about 0.20% (w/w) of such extended release portion. In some embodiments, the amount of chelating agent present in an extended release portion of the pharmaceutical composition may be about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% (w/w) of the total weight of such extended release portion.

In an alternate embodiment, the extended release portion(s) of the pharmaceutical composition may comprise a color agent. Suitable color additives include, but are not limited to, food, drug and cosmetic colors (FD&C), drug and cosmetic colors (D&C), and external drug and cosmetic colors (Ext. D&C). In various embodiments, the amount of color agent present in an extended release portion may range from about 2.0% to about 5.0% (w/w) of such extended release portion of the composition. In other embodiments, the amount of color agent present in an extended release portion may be about 1.0%, 1.5%, 2.0%, 2.5%, 3.0%, 3.5%, 4.0%, 4.5%, or 5.0% (w/w) of such extended release portion.

### (d) Dosage Forms of the Pharmaceutical Composition

(i) Physical Properties

The physical form of the pharmaceutical composition disclosed herein can and will vary. In general, the pharmaceutical composition is a solid dosage form comprising at least one extended release portion and, optionally, at least one immediate release portion. Suitable solid dosage forms include tablets, caplets, capsules, encapsulated beads, and gelcaps. Non-limiting types of tablets include coated tablets, uncoated tablets, bilayer tablets, multiparticle tablets, monolithic tablets, matrix tablets, compressed tablets, and molded tablets. Non-limiting types of capsules include hard capsules and multi-layer capsules.

In one embodiment, the dosage form may be a capsule. Non-limiting examples of suitable hard capsules include hard starch capsules, hard gelatin capsules, hard cellulose capsules, and hydrogel capsules. In one example, the core of the capsule may comprise the at least one extended release portion and the shell of the capsule may comprise the at least one immediate release portion of the composition. In another example, the core of the capsule may comprises one extended release portion, comprising hydrocodone, acetaminophen and an extended release component, and the shell of the capsule may comprise one immediate release portion of the composition comprising hydrocodone and acetaminophen. In yet another example, the core of the capsule may comprise two extended release portions, each comprising an extended release component and one of hydrocodone or acetaminophen, and the shell of the capsule may comprise two immediate release portions of the composition, each comprising one of the hydrocodone and the acetaminophen. In still another embodiment, the dosage form may be a sustained release capsule comprising the hydrocodone or acetaminophen and exhibiting immediate release and/or extended release properties. In yet another embodiment, the dosage form may be a delayed release capsule comprising the hydrocodone and/or acetaminophen and exhibiting immediate release and/or extended release properties. The capsule may comprise a coating. In one embodiment, the capsule may comprise an enteric coating.

In another embodiment, the dosage form may be a tablet comprising at least one extended release portion and at least one immediate release portion. The at least one immediate release portion may be adjacent to, abutting, or surrounding the at least one extended release portion. In one embodiment, the dosage form may be a bilayer tablet comprising one extended release layer comprising the hydrocodone and the acetaminophen and one immediate release layer comprising the hydrocodone and the acetaminophen. In still another embodiment, the dosage form may be a sustained release tablet comprising the hydrocodone and/or acetaminophen and exhibiting immediate release and/or extended release properties. In yet another embodiment, the dosage form may be a delayed release tablet comprising the hydrocodone and/or acetaminophen and exhibiting immediate release and/or extended release properties. The bilayer tablet may comprise a coating. In one embodiment, the bilayer tablet may comprise an enteric coating.

In another embodiment, the dosage form may be a multilayer tablet comprising two extended release portions, each comprising one of the hydrocodone and the acetaminophen, and one immediate release portion comprising both the hydrocodone and the acetaminophen. In yet another embodiment, the dosage form may be a multilayer tablet comprising two extended release portions, each comprising one of the hydrocodone and the acetaminophen, and two immediate release portions, each comprising one of the hydrocodone and the acetaminophen. In still another embodiment, the dosage form may be a sustained release tablet comprising hydrocodone or acetaminophen and exhibiting immediate release and/or extended release properties.

In certain embodiments, the tablet may have a friability of no greater than about 0.1%, 0.2%, 0.3%, 0.4%, 0.5%, 0.7% or 1.0%. In another embodiment, the tablet may have a friability of greater than 0 but less that about 1.0%, greater than 0 but less than about 0.5%, greater than 0 but less than about 0.3%, or greater than 0 but less than about 0.2%. In still another embodiment, the tablet may have a friability of zero.

In another embodiment, the tablet may have a hardness of at least about 10 Kilopond (also known as kilopons) (kp). In some embodiments, the tablet may have a hardness of about 9 kp to about 25 kp, or about 12 kp to about 20 kp. In further embodiments, the tablet may have a hardness of about 11 kp, 12 kp, 13 kp, 14 kp, 15 kp, 16 kp, 17 kp, 18 kp, 19 kp, or 20 kp.

In additional embodiments, the tablet may have a content uniformity of from about 85 to about 115 percent by weight or from about 90 to about 110 percent by weight, or from about 95 to about 105 percent by weight. In other embodiments, the content uniformity may have a relative standard deviation (RSD) equal to or less than about 3.5%, 3.0%, 2.5%, 2.0%, 1.5%, 1.0%, or 0.5%.

The pharmaceutical composition disclosed herein includes one or more dosage forms that are designed to achieve the therapeutic concentrations of the active ingredients. In some embodiments, therefore, a therapeutically effective dose of the pharmaceutical composition may comprise one dosage form. In other embodiments, a therapeutically effective dose of the pharmaceutical composition may comprise two dosage forms. In additional embodiments, a therapeutically effective dose of the pharmaceutical composition may comprise three or more dosage forms.

In still other embodiments, prior to administration to a patient or immersion in fluid, the pharmaceutical composition may have (i) a length of approximately 18 mm, 18.01 mm, 18.02 mm, 18.03 mm, 18.04 mm, 18.05 mm, 18.06 mm, 18.07 mm, 18.08 mm, 18.09 mm, 18.1 mm, 18.11 mm, 18.12 mm, 18.13 mm, 18.14 mm, 18.15 mm, 18.16 mm, 18.17 mm, 18.18 mm, 18.19 mm, 18.2 mm, 18.21 mm, 18.22 mm, 18.23 mm, 18.24 mm, 18.25 mm, 18.26 mm, 18.27 mm, 18.28 mm, 18.29 mm, 18.3 mm, 18.31 mm, 18.32 mm, 18.33 mm, 18.34 mm, 18.35 mm, 18.36 mm, 18.37 mm, 18.38 mm, 18.39 mm, 18.4 mm, 18.41 mm, 18.42 mm, 18.43 mm, 18.44 mm, 18.45 mm, 18.46 mm, 18.47 mm, 18.48 mm, 18.49 mm, 18.5 mm, 18.51 mm, 18.52 mm, 18.53 mm, 18.54 mm, 18.55 mm, 18.56 mm, 18.57 mm, 18.58 mm, 18.59 mm, 18.6 mm, 18.61 mm, 18.62 mm, 18.63 mm, 18.64 mm, 18.65 mm, 18.66 mm, 18.67 mm, 18.68 mm, 18.69 mm, 18.7 mm, 18.71 mm, 18.72 mm, 18.73 mm, 18.74 mm, 18.75 mm, 18.76 mm, 18.77 mm, 18.78 mm, 18.79 mm, 18.8 mm, 18.81 mm, 18.82 mm, 18.83 mm, 18.84 mm, 18.85 mm, 18.86 mm, 18.87 mm, 18.88 mm, 18.89 mm, 18.9 mm, 18.91 mm, 18.92 mm, 18.93 mm, 18.94 mm, 18.95 mm, 18.96 mm, 18.97 mm, 18.98 mm, 18.99 mm, 19 mm, 19.01 mm, 19.02 mm, 19.03 mm, 19.04 mm, 19.05 mm, 19.06 mm, 19.07 mm, 19.08 mm, 19.09 mm, 19.1 mm, 19.11 mm, 19.12 mm, 19.13 mm, 19.14 mm, 19.15 mm, 19.16 mm, 19.17 mm, 19.18 mm, 19.19 mm, 19.2 mm, 19.21 mm, 19.22 mm, 19.23 mm, 19.24 mm, 19.25 mm, 19.26 mm, 19.27 mm, 19.28 mm, 19.29 mm, 19.3 mm, 19.31 mm, 19.32 mm, 19.33 mm, 19.34 mm, 19.35 mm, 19.36 mm, 19.37 mm, 19.38 mm, 19.39 mm, 19.4 mm, 19.41 mm, 19.42 mm, 19.43 mm, 19.44 mm, 19.45 mm, 19.46 mm, 19.47 mm, 19.48 mm, 19.49 mm, 19.5 mm, 19.51 mm, 19.52 mm, 19.53 mm, 19.54 mm, 19.55 mm, 19.56 mm, 19.57 mm, 19.58 mm, 19.59 mm 19.6 mm, 19.61 mm, 19.62 mm, 19.63 mm, 19.64 mm, 19.65 mm, 19.66 mm, 19.67 mm, 19.68 mm, 19.69 mm, 19.7 mm, 19.71 mm, 19.72 mm, 19.73 mm, 19.74 mm, 19.75 mm, 19.76 mm, 19.77 mm, 19.78 mm, 19.79 mm, 19.8 mm, 19.81 mm, 19.82 mm, 19.83 mm, 19.84 mm, 19.85 mm, 19.86 mm, 19.87 mm, 19.88 mm, 19.89 mm, 19.9 mm, 19.91 mm, 19.92 mm, 19.93 mm, 19.94 mm, 19.95 mm, 19.96 mm, 19.97 mm, 19.98 mm, 19.99 mm, or 20 mm as measured on the major axis, (ii) a width of approximately 11 mm, 11.01 mm, 11.02 mm, 11.03 mm, 11.04 mm, 11.05 mm, 11.06 mm, 11.07 mm, 11.08 mm, 11.09 mm, 11.1 mm, 11.11 mm, 11.12 mm, 11.13 mm, 11.14 mm, 11.15 mm, 11.16 mm, 11.17 mm, 11.18 mm, 11.19 mm, 11.2 mm, 11.21 mm, 11.22 mm, 11.23 mm, 11.24 mm, 11.25 mm, 11.26 mm, 11.27 mm, 11.28 mm, 11.29 mm, 11.3 mm, 11.31 mm, 11.32 mm, 11.33 mm, 11.34 mm, 11.35 mm, 11.36 mm, 11.37 mm, 11.38 mm, 11.39 mm, 11.4 mm, 11.41 mm, 11.42 mm, 11.43 mm, 11.44 mm, 11.45 mm, 11.46 mm, 11.47 mm, 11.48 mm, 11.49 mm, 11.5 mm, 11.51 mm, 11.52 mm, 11.53 mm, 11.54 mm, 11.55 mm, 11.56 mm, 11.57 mm, 11.58 mm, 11.59 mm, 11.6 mm, 11.61 mm, 11.62 mm, 11.63 mm, 11.64 mm, 11.65 mm, 11.66 mm, 11.67 mm, 11.68 mm, 11.69 mm, 11.7 mm, 11.71 mm, 11.72 mm, 11.73 mm, 11.74 mm, 11.75 mm, 11.76 mm, 11.77 mm, 11.78 mm, 11.79 mm, 11.8 mm, 11.81 mm, 11.82 mm, 11.83 mm, 11.84 mm, 11.85 mm, 11.86 mm, 11.87 mm, 11.88 mm, 11.89 mm, 11.9 mm, 11.91 mm, 11.92 mm, 11.93 mm, 11.94 mm, 11.95 mm, 11.96 mm, 11.97 mm, 11.98 mm, 11.99 mm, 12 mm, 12.01 mm, 12.02 mm, 12.03 mm, 12.04 mm, 12.05 mm, 12.06 mm, 12.07 mm, 12.08 mm, 12.09 mm, 12.1 mm, 12.11 mm, 12.12 mm, 12.13 mm, 12.14 mm, 12.15 mm, 12.16 mm, 12.17 mm, 12.18 mm, 12.19 mm, 12.2 mm, 12.21 mm, 12.22 mm, 12.23 mm, 12.24 mm, 12.25 mm, 12.26 mm, 12.27 mm, 12.28 mm, 12.29 mm, 12.3 mm, 12.31 mm, 12.32 mm, 12.33 mm, 12.34 mm, 12.35 mm, 12.36 mm, 12.37 mm, 12.38 mm, 12.39 mm, 12.4 mm, 12.41 mm, 12.42 mm, 12.43 mm, 12.44 mm, 12.45 mm, 12.46 mm, 12.47 mm, 12.48 mm, 12.49 mm, 12.5 mm, 12.51 mm, 12.52 mm, 12.53 mm, 12.54 mm, 12.55 mm, 12.56 mm, 12.57 mm, 12.58 mm, 12.59 mm, 12.6 mm, 12.61 mm, 12.62 mm, 12.63 mm, 12.64 mm, 12.65 mm, 12.66 mm, 12.67 mm, 12.68 mm, 12.69 mm, 12.7 mm, 12.71 mm, 12.72 mm, 12.73 mm, 12.74 mm, 12.75 mm, 12.76 mm, 12.77 mm, 12.78 mm, 12.79 mm, 12.8 mm, 12.81 mm, 12.82 mm, 12.83 mm, 12.84 mm, 12.85 mm, 12.86 mm, 12.87 mm, 12.88 mm, 12.89 mm, 12.9 mm, 12.91 mm, 12.92 mm, 12.93 mm, 12.94 mm, 12.95 mm, 12.96 mm, 12.97 mm, 12.98 mm, 12.99 mm, or 13 mm, and (iii) a height or thickness of approximately 5 mm, 5.01 mm, 5.02 mm, 5.03 mm, 5.04 mm, 5.05 mm, 5.06 mm, 5.07 mm, 5.08 mm, 5.09 mm, 5.1 mm, 5.11 mm, 5.12 mm, 5.13 mm, 5.14 mm, 5.15 mm, 5.16 mm, 5.17 mm, 5.18 mm, 5.19 mm, 5.2 mm, 5.21 mm, 5.22 mm, 5.23 mm, 5.24 mm, 5.25 mm, 5.26 mm, 5.27 mm, 5.28 mm, 5.29 mm, 5.3 mm, 5.31 mm, 5.32 mm, 5.33 mm, 5.34 mm, 5.35 mm, 5.36 mm, 5.37 mm, 5.38 mm, 5.39 mm, 5.4 mm, 5.41 mm, 5.42 mm, 5.43 mm, 5.44 mm, 5.45 mm, 5.46 mm, 5.47 mm, 5.48 mm, 5.49 mm, 5.5 mm, 5.51 mm, 5.52 mm, 5.53 mm, 5.54 mm, 5.55 mm, 5.56 mm, 5.57 mm, 5.58 mm, 5.59 mm, 5.6 mm, 5.61 mm, 5.62 mm, 5.63 mm, 5.64 mm, 5.65 mm, 5.66 mm, 5.67 mm, 5.68 mm, 5.69 mm, 5.7 mm, 5.71 mm, 5.72 mm, 5.73 mm, 5.74 mm, 5.75 mm, 5.76 mm, 5.77 mm, 5.78 mm, 5.79 mm, 5.8 mm, 5.81 mm, 5.82 mm, 5.83 mm, 5.84 mm, 5.85 mm, 5.86 mm, 5.87 mm, 5.88 mm, 5.89 mm, 5.9 mm, 5.91 mm, 5.92 mm, 5.93 mm, 5.94 mm, 5.95 mm, 5.96 mm, 5.97 mm, 5.98 mm, 5.99 mm, or 6 mm. In yet another embodiment, the pharmaceutical composition may have (i) a length of approximately 19.1 mm, 19.11 mm, 19.12 mm, 19.13 mm, 19.14 mm, 19.15 mm, 19.16 mm, 19.17 mm, 19.18 mm, 19.19 mm, 19.2 mm, 19.21 mm, 19.22 mm, 19.23 mm, 19.24 mm, 19.25 mm, 19.26 mm, 19.27 mm, 19.28 mm, 19.29 mm, or 19.3 mm as measured on the major axis, (ii) a width of approximately 12.4 mm, 12.41 mm, 12.42 mm, 12.43 mm, 12.44 mm, 12.45 mm, 12.46 mm, 12.47 mm, 12.48 mm, 12.49 mm, or 12.5 mm, and (iii) a height or thickness of approximately 5.6 mm, 5.61 mm, 5.62 mm, 5.63 mm, 5.64 mm, 5.65 mm, 5.66 mm, 5.67 mm, 5.68 mm, 5.69 mm, 5.7 mm, 5.71 mm, 5.72 mm, 5.73 mm, 5.74 mm, 5.75 mm, 5.76 mm, 5.77 mm, 5.78 mm, 5.79 mm, or 5.8 mm.

In additional embodiments, the pharmaceutical composition may expand upon immersion in fluid to have (i) a length of about 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, or 21 mm; and (ii) a width of about 11 mm, 11.1 mm, 11.2 mm, 11.3 mm, 11.4 mm, 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, or 14 mm within about 5 minutes of immersion in fluid. In other embodiments, the pharmaceutical composition may expand upon immersion in fluid to (i) a length of about 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, or 22 mm; and (ii) a width of about 11 mm, 11.1 mm, 11.2 mm, 11.3 mm, 11.4 mm, 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, or 15 mm within about 10 minutes to about 15 minutes of immersion in fluid. In still other embodiments, the pharmaceutical composition may expand upon immersion in fluid to (i) a length of about 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, or 22.5 mm; and (ii) a width of about 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, or 15 mm within about 20 minutes to about 25 minutes of immersion in fluid. In additional embodiments, the pharmaceutical composition may expand upon immersion in fluid to (i) a length of about 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, 22.5 mm, 22.6 mm, 22.7 mm, 22.8 mm, 22.9 mm, or 23 mm; and (ii) a width of about 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, or 15 mm within about 30 minutes to about 35 minutes of immersion in fluid. In still other embodiments, the pharmaceutical composition may expand upon immersion in fluid to (i) a length of about 18 mm, 18.1 mm, 18.2 mm, 18.3 mm, 18.4 mm, 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, 22.5 mm, 22.6 mm, 22.7 mm, 22.8 mm, 22.9 mm, 23 mm, 23.1 mm, 23.2 mm, 23.3 mm, 23.4 mm, or 23.5; (ii) a width of about 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, or 16 mm; and (iii) a height or thickness of about 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, or 7 mm within about 50 minutes to about 55 minutes of immersion in fluid. In yet another embodiment, the pharmaceutical composition may expand upon immersion in fluid to (i) a length of about 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, 22.5 mm, 22.6 mm, 22.7 mm, 22.8 mm, 22.9 mm, 23 mm, 23.1 mm, 23.2 mm, 23.3 mm, 23.4 mm, or 23.5; (ii) a width of about 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, or 16 mm; and (iii) a height or thickness of about 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, or 7 mm within about 60 minutes of immersion in fluid.

In yet another embodiment, the length of the pharmaceutical composition increases by about 4%, 4.25%, 4.5% 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, or 13% within about 10 minutes of immersion in fluid. In still another embodiment, the length of the pharmaceutical composition increases by about 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, or 15% within about 15 minutes of immersion in fluid. In yet another embodiment, the length of the pharmaceutical composition increases by about 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, or 15% within about 20 minutes of immersion in fluid. In a further embodiment, the length of the pharmaceutical composition increases by about 7%, 7.25%, 7.5%, 7.75% 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, or 18% within about 30 minutes of immersion in fluid. In another embodiment, the length of the pharmaceutical composition increases by about 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, or 19% within about 45 minutes of immersion in fluid. In yet another embodiment, the length of the pharmaceutical composition increases by about 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, or 19% within about 55 minutes of immersion in fluid. In still another embodiment, the length of the pharmaceutical composition increases by about 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, or 20% within about 60 minutes of immersion in fluid.

In a further embodiment, the width of the pharmaceutical composition increases by about 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, or 15% within about 10 minutes of immersion in fluid. In still another embodiment, the width of the pharmaceutical composition increases by about 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, or 18%, within about 15 minutes of immersion in fluid. In yet another embodiment, the width of the pharmaceutical composition increases by about 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, or 18%, within about 20 minutes of immersion in fluid. In a further embodiment, the width of the pharmaceutical composition increases by about 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20%, 20.25%, 20.5%, 20.75%, 21%, 21.25%, 21.5%, 21.75%, 22%, 22.25%, 22.5%, 22.75%, 23%, 23.25%, 23.5%, 23.75%, or 24% within about 30 minutes of immersion in fluid. In another embodiment, the width of the pharmaceutical composition increases by about 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20.0%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, or 25% within about 45 minutes of immersion in fluid. In yet another embodiment, the width of the pharmaceutical composition increases by about 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, or 25% within about 55 minutes of immersion in fluid. In still another embodiment, the width of the pharmaceutical composition increases by about 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, 25%, 25.25%, 25.5%, 25.75%, or 26% within about 60 minutes of immersion in fluid.

In some embodiments, the composition disclosed herein may have gastric retentive properties. These gastric retentive properties of the composition may be due to a combination of a physical property of the composition and/or the release of the opioid. In one embodiment, the gastric retentive properties of the opioid-containing extended release composition is provided by the use of a polymer. In one embodiment, the opioid-containing extended release composition comprises a gastric retentive polymer in an amount of about 1% to about 99%. In another embodiment, the opioid-containing extended release composition comprises a gastric retentive polymer in an amount of about 10% to about 80%. In yet another embodiment, the opioid-containing extended release composition comprises a gastric retentive polymer in an amount of about 20% to about 60%. In other embodiments, the opioid-containing extended release composition comprises a gastric retentive polymer in an amount of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In another embodiment, the composition may be expandable. That is, the composition has size that is small enough for oral intake, but the composition absorbs water from the gastric fluid and swells to a size that prevents its passage through the pylorus. Such a composition comprises at least one swellable, expandable material, such as a polymer, resin, hydrocolloid, hydrogel, or the like. In various embodiments, the composition may swell to a size that is about 110% to about 200% of the original volume within about 30 minutes of administration. For example, the composition may swell to approximately 115% of it original volume within 30 minutes of administration, and at a later time may swell to a volume that is 130% or more of the original volume. In other embodiments, the composition may exhibit a volume increase of two-fold or more. Additionally, the composition may become slippery upon absorption of water, which provides resistance to peristalsis and further promotes gastric retention. The swellable material degrades or erodes over a specified period of time (e.g., the dosing interval) such that the composition is no longer retained in the stomach. In one embodiment, the ER layer swells upon imbibition of fluid to a size which is about 15%, 20%, 25%, 30%, 35% 40%, 45%, 50%, 55%, 60%, 65%, 70%, 75%, 80%, 85%, 90%, 95% or 100% larger than the size of the ER layer prior to imbibition of fluid. In another embodiment, the ER layer swells upon imbibition of fluid to a size at least about 25% larger than the size of the ER layer prior to imbibition of fluid within about 15 minutes of the start of fluid imbibition. In still another embodiment, the ER layer swells upon imbibition of fluid to a size at least about 100% larger than the size of the ER layer prior to imbibition of fluid within about 45 min, 50 min, 60 min, 75 min, or 90 min of the start of fluid imbibitions.

In a further embodiment, the composition contains at least one swellable polymer. For example, the composition may include chitosan, methylcellulose, polyvinyl acetate, purified shellac, polyethylene oxide, polypropylene oxide, or an expansive polymeric film, such as one composed of polyvinyl acetate and shellac. In another embodiment, the composition may contain a combination of polymers in a matrix that is swellable. Exemplary swellable matrices are described in U.S. Pat. Nos. 6,723,340, 6,340,475, and 6,635,280, the disclosures of which are herein incorporated by reference in their entirety.

In still another embodiment, the physical property of the composition that imparts gastric retention may be the shape of the composition. For example, the composition may have a ring, tetrahedron, spiral, coil, planar disc, planar multilobe, continuous stick, sheet, oval, parallelogram, or string geometic configuration, wherein the composition is unable to pass through the pyloric sphincter. In some iterations, the composition may be folded into a pharmaceutical carrier (e.g., a gelatin capsule) or secured by readily dissolvable (e.g., gelatin) strips such that, upon dissolution of the carrier or strips, the composition unfolds in the stomach. In general, unfoldable compositions comprise biodegradable polymers such that the composition is degraded and/or reduced in size over a specified period of time (e.g., the dosing interval). In another embodiment, the composition has a diameter of greater than or equal to 7.5 mm. Exemplary shaped dosage forms are described in U.S. Pat. No. 6,488,962, the entirety of which is herein incorporated by reference.

In yet another embodiment, the physical property of the composition that imparts gastric retention may be the adhesivity of the composition. Bio-mucoadhesive compositions bind to the gastric epithelial cell surface, or mucin, and increase gastric retention time by increasing the intimacy and duration of contact between the composition and the biological membrane. Bio-mucoadhesive compositions generally comprise polycarbophil, carbopol, cholestyramine, chitosan, polymeric acids, or a natural or synthetic polymer that is capable of adhering to a biological membrane (e.g., a bioadhesive polymer) or the mucus lining of the stomach or intestinal tract (e.g., a mucoadhesive polymer). Exemplary adhesive polymers include anionic (e.g., carboxymethylcellulose, chondroitin sulfate, polyacrylic acid, pectin, carageenan, chitosan, and alginic acid), cationic (e.g., polylysine and polybrene), and neutral (e.g., polyethylene glycol, polyvinyl pyrrolidone, and dextran) polymers. Certain hydrophilic polymers tend to imbibe large amounts of water and become sticky, thereby acquiring bioadhesive properties. The adhesion of polymers to a mucus or epithelial cell surface may involve various bonding mechanisms, including physical-mechanical bonding and chemical bonding. Physical-mechanical bonding may result from the insertion of the adhesive material into the crevices or folds of the mucosa. Chemical bonds may be either covalent or non-covalent (e.g., ionic bonds, hydrogen bonds, van der Waals interactions, etc). Moreover, certain polymers may bind to specific receptor sites on the surface of cells, thereby enhancing the gastric retention. For example, certain plant lectins interact specifically with the sugar groups present in mucus or on the glycocalyx.

In still another embodiment, the physical property of the composition that imparts gastric retention may be the density of the composition. In one iteration, the composition may have a low density with sufficient buoyancy such that the composition floats over the gastric contents and remains in the stomach for a prolonged period. Floating compositions may be effervescent or noneffervescent. Effervescent compositions generally comprise matrices prepared with swellable polymers and an effervescent component. For example, the effervescent component can be either a carbonate or bicarbonate salt (e.g., sodium bicarbonate, calcium bicarbonate), an organic acid (e.g., citric acid, tartaric acid), or any combination thereof. The effervescent component can also be a floating chamber filled with vacuum, air, an inert gas, or a liquid that gasifies at body temperature. Floatability is generally achieved by generation of gas bubbles. Gas may be introduced into the floating chamber by the volatilization of an organic solvent, or by an effervescent reaction between a carbonate-bicarbonate salt and an organic acid. The matrices may be fabricated so that upon arrival in the stomach, carbon dioxide is liberated by the acidity of the gastric contents and is entrapped in the gellified matrix. This maintains the buoyancy of the composition, causing it to float. In another embodiment, the composition may also contain a polymer which exhibits floating characteristics, such as hydroxypropyl cellulose, hydroxypropyl methylcellulose, crospovidone, sodium carboxymethyl cellulose, or ethyl cellulose. In a further embodiment, the composition may comprise a device having a hollow deformable unit that converts from a collapsed to expanded form and vise versa. The unit is supported by a housing that is internally divided into two chambers separated by a pressure-sensitive movable bladder. The first chamber contains the therapeutic agent and the second contains a volatile liquid (e.g., cyclopentane, ether) that vaporizes at body temperature and imparts buoyancy to the system. The system also contains a bioerodible plug to aid in the exit from the body. Further embodiments of this two chamber system are disclosed in U.S. Pat. Nos. 3,901,232 and 3,786,813, which are hereby incorporated by reference. In still a further embodiment, the composition may contain hollow microspheres or microballoons, which cause the composition to float. The composition may also comprise floating microparticles such as polypropylene foam, Eudragit, ethyl cellulose, or polymethyl metha acylate (PMMA).

Noneffervescent compositions incorporate a high level of one or more gel-forming, highly swellable, cellulosic hydrocolloids. Upon contact with the gastric contents, these hydrocolloids hydrate and forms a colloidal gel barrier, wherein air trapped by the swollen hydrocolloid confers buoyancy to this composition. In another iteration, the composition may have a density that exceeds the density of normal gastric contents such the composition sinks to the bottom of the stomach (i.e., the antrum) where it is entrapped in the folds of the antrum and withstands the peristaltic waves of the gastric wall. In yet another iteration, the composition has a density that is greater than or equal to 1.3 g/mL.

In one embodiment, the composition is retained in the stomach due to the presence of an extended release polymer that absorbs water from the gastric contents and swells or expands to a size that cannot pass through the pyloric sphincter. As a consequence, the opioid and the other API are slowly released from the composition in the stomach and absorbed in the upper gastrointestinal tract.

In still another embodiment, the physical property of the composition that results in gastric retention may be the physical size of the composition. That is, the composition may have a size that is small enough to be orally ingested and enter the stomach, but large enough to prevent passage through the pyloric sphincter into the small intestine. In some embodiments in which the composition is designed for humans, the composition may have a length (or diameter) of more than about 7 mm, 8 mm, 9 mm, or mm. In other embodiments in which the composition is designed for humans, the composition may have a length (or diameter) of more than about 11 mm, 12 mm, or 13 mm, 14 mm, 15 mm, 16 mm, 17 mm, 18 mm, 19 mm, 20 mm or longer. In still other embodiments, the composition may have (i) a length of approximately 19 mm, 19.1 mm, 19.2 mm, 19.3 mm, 19.4 mm, 19.5 mm, 19.6 mm, 19.7 mm, 19.8 mm, 19.9 mm, or 20 mm as measured on the major axis, (ii) a width of approximately 12 mm, 12.1 mm, 12.2 mm, 12.3 mm, 12.4 mm, 12.5 mm, 12.6 mm, 12.7 mm, 12.8 mm, 12.9 mm, or 13 mm as measured on the minor axis, and (iii) a height or thickness of approximately 5 mm, 5.1 mm, 5.2 mm, 5.3 mm, 5.4 mm, 5.5 mm, 5.6 mm, 5.7 mm, 5.8 mm, 5.9 mm, or 6 mm. In yet another embodiment, the composition may have (i) a length of approximately 19.1 mm, 19.11 mm, 19.12 mm, 19.13 mm, 19.14 mm, 19.15 mm, 19.16 mm, 19.17 mm, 19.18 mm, 19.19 mm, 19.2 mm, 19.21 mm, 19.22 mm, 19.23 mm, 19.24 mm, 19.25 mm, 19.26 mm, 19.27 mm, 19.28 mm, 19.29 mm, or 19.3 mm as measured on the major axis, (ii) a width of approximately 12.4 mm, 12.41 mm, 12.42 mm, 12.43 mm, 12.44 mm, 12.45 mm, 12.46 mm, 12.47 mm, 12.48 mm, 12.49 mm, or 12.5 mm as measured on the minor axis, and (iii) a height or thickness of approximately 5.6 mm, 5.61 mm, 5.62 mm, 5.63 mm, 5.64 mm, 5.65 mm, 5.66 mm, 5.67 mm, 5.68 mm, 5.69 mm, 5.7 mm, 5.71 mm, 5.72 mm, 5.73 mm, 5.74 mm, 5.75 mm, 5.76 mm, 5.77 mm, 5.78 mm, 5.79 mm, or 5.8 mm. In general, such compositions are designed to degrade, disintegrate, decrease in size, or collapse in a specified time interval (e.g., dosing interval) such that they may pass through the pyloric valve or be evacuated from the stomach by a housekeeper wave of gastric contractions

In still another embodiment, the composition may contain an agent which delays the passage of the composition through the pyloric sphincter. For example, the composition may include triethanol amine myristate or propantheline.

(ii) opioid Release

Because opioids reduce gastric motility, the erosion time of the dosage form can be increased (thus, hindering drug release) if the opioid is not properly dosed. The gastric retentive extended release composition disclosed herein is engineered to release the opioid(s) at a rate that is sufficient to delay gastric emptying such that the composition is retained in the stomach for a longer period of time than a comparable composition that is not gastric retentive. For example, the composition may be designed to release the opioid(s) at a rate that delays gastric emptying by about 15 minutes, 30 minutes, 60 minutes, 90 minutes, 2.0 hours, 2.5 hours, 3.0 hours, 3.5 hours, 4.0 hours, 4.5 hours, or 5.0 hours. The rate of release of the opioid(s) may be manipulated by selecting a suitable extended release component for inclusion in an extended release portion of the composition. For example, in embodiments in which the extended release component is an extended release polymer, the extended release polymer generally is selected such that the composition releases the opioid(s) at a rate that delays gastric emptying by the desired amount. Additionally, the rate of release of the opioid(s) from the composition may be adjusted by selecting the proper ratio of opioid present in the at least one immediate release and the at least one extended release portions of the composition. For instance, the proportion of the opioid(s) in the at least one immediate release portion and the at least one extended release portion may be about 20:80, 21:79, 22:78, 23:77; 24:76, 25:75, 26:74, 27:73, 28;72, 29:71, 30:70, 31:69, 32:68, 33:67, 34:66, 35:65, 34:66, 35:65, 36:64, 37:63, 38:62, 39:61, or 40:60.

Additionally, the gastric retentive extended release composition is engineered to release the opioid(s) at a rate that is insufficient to cause any serious adverse gastrointestinal effects. Adverse gastrointestinal effects include, but are not limited to, intestinal hypomotility, intestinal blockage, intestinal pseudo-obstruction, abdominal distention, bloating, constipation, intestinal distress, severe intestinal contractions, colon spasms, hypoactive bowel, and increased anal sphincter tone.

(iii) Overall Composition

With the knowledge of the preferred dissolution and pharmacokinetic profiles for the opioid and the additional API, and the pharmacodynamics effects of the opioid and the additional API, as discovered by the applicants and first described herein, a composition exhibiting the same or similar dissolution and pharmacokinetic profiles and pharmacodynamics effects can be developed using any of the dosage forms discussed above. Moreover, a composition under the present invention can be developed using another dosage form that achieves the same or similar dissolution, pharmacokinetic, and pharmacodynamic profiles as the compositions disclosed herein. For example, in one embodiment, a controlled=release dosage form can be developed that exhibits pharmacokinetic and pharmacodynamic parameters (e.g., Cmax, AUC) which are within 80% to 125% at a confidence interval of 90% of those parameters for the compositions described herein. In another embodiment, a sustained release dosage form can be developed that exhibits pharmacokinetic and pharmacodynamic parameters which are within 80% to 125% at a confidence interval of 90% of those parameters for the compositions described herein. A composition could also be developed that lacks one of the specific gastric retentive dosage forms discussed above, yet, achieves the same dissolution and pharmacokinetic profiles, and exhibits the pharmacodynamic effects.

For example, a gastric retentive extended release composition as described herein may comprise an opioid, such as hydrocodone, an additional API, such as acetaminophen, an immediate release portion, and a gastric retentive portion, wherein the immediate release and gastric retentive portions comprise a filler and a lubricant. In one embodiment, the immediate release and gastric retentive portions may each comprise a filler in an amount of about 5 mg to about 500 mg. In another embodiment, the immediate release and gastric retentive portions may each comprise a filler in an amount of about 20 mg to about 400 mg. In yet another embodiment, the immediate release and gastric retentive portions may each comprise a filler in an amount of about 40 mg to about 300 mg.

In one embodiment, the immediate release and gastric retentive portions may each comprise a lubricant in an amount of about 0.1 mg to about 25 mg. In another embodiment, the immediate release and gastric retentive portions may each comprise a lubricant in an amount of about 0.4 mg to about 15 mg. In still another embodiment, the immediate release and gastric retentive portions may each comprise a lubricant in an amount of about 0.7 mg to about 5 mg. In another aspect, the gastric retentive portion may further comprise between about 0 mg to about 50 mg of an effervescent agent, such as a bicarbonate salt.

As discussed above, an extended release composition without gastric retention is also described herein. In one embodiment, an extended release composition as described herein may comprise an opioid, such as hydrocodone, an additional API, such as acetaminophen, an immediate release portion, and an extended release portion, wherein the immediate release and extended release portions comprise a filler in an amount of about 5 mg to about 500 mg and a lubricant in an amount of about 0.1 mg to about 25 mg. The extended release portion may comprise any suitable extended release polymer. In one embodiment, the extended release polymer is present in an amount of about 5 mg to about 500 mg. In another embodiment, the extended release polymer is present in an amount of about 20 mg to about 400 mg. In a further embodiment, the extended release polymer is present in an amount of about 40 mg to about 300 mg

For example, some exemplary formulations of the gastric retentive (Examples A-I) or sustained release (Examples J-R) dosage forms described above are as follows:

CHART A
Exemplary Gastric Retentive and Sustained Release Dosage Forms.

| | A | B | C |
|---|---|---|---|
| Immediate Release | Hydrocodone | Hydrocodone | Hydrocodone |
| Portion | Bitartrate (0-15 mg) | Bitartrate (0-7.5 mg) | Bitartrate (0-15 mg) |
| | APAP (0-325 mg) | APAP (0-175 mg) | APAP (200-325 mg) |
| | Filler (5-100 mg) | Filler (5-250 mg) | Filler (50-75 mg) |
| | Lubricant (0.1-5 mg) | Stearate salt | Lubricant (2-3 mg) |
| | | (0.1-10 mg) | |
| Gastric Retentive | Hydrocodone | Hydrocodone | Hydrocodone |
| Portion | Bitartrate (0-15 mg) | Bitartrate (0-7.5 mg) | Bitartrate (0-15 mg) |
| | APAP (0-325 mg) | APAP (0-175 mg) | APAP (100-325 mg) |
| | Polymer (5-500 mg) | Polymer (50-750 mg) | Polymer (100-250 mg) |
| | Filler (5-100 mg) | Filler (5-250 mg) | Filler (25-50 mg) |
| | Lubricant (0.1-5 mg) | Stearate Salt | Lubricant (1-3 mg) |
| | | (0.1-10 mg) | |
| | D | E | F |
| Immediate Release | Hydrocodone | Hydrocodone | Hydrocodone |
| Portion | Bitartrate (5-10 mg) | Bitartrate (5-10 mg) | Bitartrate (2-5 mg) |
| | APAP (100-400 mg) | APAP (100-400 mg) | APAP (300-450 mg) |
| | Filler (25-50 mg) | Filler (25-50 mg) | Filler (25-75 mg) |
| | Lubricant (3-5 mg) | Lubricant (3-5 mg) | Lubricant (2-5 mg) |
| Gastric Retentive | Hydrocodone | Hydrocodone | Hydrocodone |
| Portion | Bitartrate (5-10 mg) | Bitartrate (5-10 mg) | Bitartrate (3-10 mg) |
| | APAP (50-250 mg) | APAP (50-250 mg) | APAP (50-300 mg) |
| | Filler (50-75 mg) | Filler (50-75 mg) | Filler (50-75 mg) |
| | Polycarbophil | Polymer (5-500 mg) | Polymer (100-450 mg) |
| | (5-500 mg) | Bicarbonate salt | Bicarbonate salt |
| | Lubricant (3-5 mg) | (0-10 mg) | (0-10 mg) |
| | | Lubricant (3-5 mg) | Lubricant (3-5 mg) |
| | G | H | I |
| Immediate Release | Hydrocodone | Hydrocodone | Hydrocodone |
| Portion | Bitartrate (1-10 mg) | Bitartrate (0-15 mg) | Bitartrate (0-15 mg) |
| | APAP (100-325 mg) | APAP (0-325 mg) | APAP (0-325 mg) |
| | Filler (25-75 mg) | Filler (5-100 mg) | Filler (5-100 mg) |
| | Lubricant (2-5 mg) | Lubricant (0.1-5 mg) | Lubricant (0.1-5 mg) |
| Gastric Retentive | Hydrocodone | Hydrocodone | Hydrocodone |
| Portion | Bitartrate (3-10 mg) | Bitartrate (0-15 mg) | Bitartrate (0-15 mg) |
| | APAP (100-450 mg) | APAP (0-325 mg) | APAP (0-325 mg) |
| | Filler (5-100 mg) | Polyacrylate | Cholestyramine |
| | Carbopol (5-500 mg) | (100-300 mg) | (100-300 mg) |
| | Lubricant (3-5 mg) | Filler (5-100 mg) | Filler (5-100 mg) |
| | J | K | L |
| Immediate Release | Hydrocodone | Hydrocodone | Hydrocodone |
| Portion | Bitartrate (0-15 mg) | Bitartrate (0-7.5 mg) | Bitartrate (0-15 mg) |
| | APAP (0-325 mg) | APAP (0-175 mg) | APAP (200-325 mg) |
| | Filler (5-100 mg) | Filler (5-250 mg) | Filler (50-75 mg) |
| | Lubricant (0.1-5 mg) | Stearate salt | Lubricant (2-3 mg) |
| | | (0.1-10 mg) | |
| Extended Release | Hydrocodone | Hydrocodone | Hydrocodone |
| Portion | Bitartrate (0-15 mg) | Bitartrate (0-7.5 mg) | Bitartrate (0-15 mg) |
| | APAP (0-325 mg) | APAP (0-175 mg) | APAP (100-325 mg) |
| | Polymer (5-500 mg) | Polymer (50-750 mg) | Polymer (100-250 mg) |
| | Filler (5-100 mg) | Filler (5-250 mg) | Filler (25-50 mg) |
| | Lubricant (0.1-5 mg) | Stearate Salt | Lubricant (1-3 mg) |
| | | (0.1-10 mg) | |
| | M | N | O |
| Immediate Release | Hydrocodone | Hydrocodone | Hydrocodone |
| Portion | Bitartrate (5-10 mg) | Bitartrate (5-10 mg) | Bitartrate (2-5 mg) |
| | APAP (100-400 mg) | APAP (100-400 mg) | APAP (300-450 mg) |
| | Filler (25-50 mg) | Filler (25-50 mg) | Filler (25-75 mg) |
| | Lubricant (3-5 mg) | Lubricant (3-5 mg) | Lubricant (2-5 mg) |
| Extended Release | Hydrocodone | Hydrocodone | Hydrocodone |
| Portion | Bitartrate (5-10 mg) | Bitartrate (5-10 mg) | Bitartrate (3-10 mg) |
| | APAP (50-250 mg) | APAP (50-250 mg) | APAP (50-300 mg) |
| | Filler (50-75 mg) | Filler (50-75 mg) | Filler (50-75 mg) |
| | Methacrylate | Hydroxy | Propylmethyl |
| | copolymer (5-500 mg) | propylmethyl | cellulose (100-450 mg) |
| | Lubricant (0.1-10 mg) | cellulose (5-500 mg) | Lubricant (0.1-10 mg) |
| | | Lubricant (0.1-10 mg) | |
| | P | Q | R |
| Immediate Release | Hydrocodone | Hydrocodone | Hydrocodone |
| Portion | Bitartrate (1-10 mg) | Bitartrate (0-15 mg) | Bitartrate (0-15 mg) |
| | APAP (100-325 mg) | APAP (0-325 mg) | APAP (0-325 mg) |
| | Filler (25-75 mg) | Filler (5-100 mg) | Filler (5-100 mg) |
| | Lubricant (2-5 mg) | Lubricant (0.1-5 mg) | Lubricant (0.1-5 mg) |
| Extended Release | Hydrocodone | Hydrocodone | Hydrocodone |
| Portion | Bitartrate (3-10 mg) | Bitartrate (0-15 mg) | Bitartrate (0-15 mg) |
| | APAP (100-450 mg) | APAP (0-325 mg) | APAP (0-325 mg) |
| | Filler (5-100 mg) | Ethylcellulose | Polyacrylate (5-500 mg) |
| | Alginate (5-500 mg) | (5-500 mg) | Filler (5-100 mg) |
| | Lubricant (3-5 mg) | Filler (5-100 mg) | |
| | S | T | U |
| Sustained Release | Hydrocodone | Hydrocodone | Hydrocodone |
| Formulation | Bitartrate (1-15 mg) | Bitartrate (1-15 mg) | Bitartrate (1-15 mg) |
| | APAP (50-650 mg) | APAP (50-650 mg) | APAP (50-650 mg) |
| | Filler (0-100 mg) | Filler (0-100 mg) | Filler (0-100 mg) |
| | Methacrylate | Hydroxy | Propylmethyl |
| | copolymer (5-500 mg) | propylmethyl | cellulose (5-550 mg) |
| | Lubricant (0.1-10 mg) | cellulose (5-500 mg) | Lubricant (0.1-10 mg) |
| | | Lubricant (0.1-10 mg) | |
| | V | W | X |
| Sustained Release | Hydrocodone | Hydrocodone | Hydrocodone |
| Formulation | Bitartrate (1-15 mg) | Bitartrate (1-15 mg) | Bitartrate (1-15 mg) |
| | APAP (50-650 mg) | APAP (50-650 mg) | APAP (50-650 mg) |
| | Filler (0-100 mg) | Filler (0-100 mg) | Filler (0-100 mg) |
| | Alginate (5-500 mg) | Polysorbate (5-500 mg) | Polyacrylate (5-550 mg) |
| | Lubricant (0.1-10 mg) | Lubricant (0.1-10 mg) | Lubricant (0.1-10 mg) |

The pharmaceutical composition disclosed herein includes one or more dosage forms that are designed to achieve the therapeutic concentrations of the active ingredients. In some embodiments, therefore, a therapeutically effective dose of the pharmaceutical composition may comprise one dosage form. In other embodiments, a therapeutically effective dose of the pharmaceutical composition may comprise two dosage forms. In additional embodiments, a therapeutically effective dose of the pharmaceutical composition may comprise three or more dosage forms.

### (e) Abuse and Tamper Resistant Properties of the Composition

Extended release pain medications have provided many benefits to patients in the management of their chronic pain by providing a sustained release over time of a larger quantity of drug than is typically contained in an immediate release formulation. Consequently, these dosage forms (especially if they contain opioids) are attractive targets for drug abusers looking to defeat the extended release formulation to allow immediate bolus administration or “dose-dumping” of the entire drug contents of the dosage form.

Dosage forms of the pharmaceutical composition disclosed herein may be more resistant to crushing, grinding, pulverizing, or other common means used to produce a powder than an immediate release product. Accordingly, some embodiment forms are tamper resistant and less prone to abuse or misuse. For example, certain embodiments may not be crushed into a powder and snorted. Additionally, some embodiments comprising an extended release polymer may not be crushed, mixed with an aqueous solution, and injected (e.g., the resultant mixture becomes extremely viscous and cannot be effectively drawn into a syringe.)

For example, dosage forms of the pharmaceutical composition disclosed herein form a pasty semi-solid mixture when dissolved. Thus, the pharmaceutical composition is difficult to draw into a syringe and inject intravenously. The yield of active pharmaceutical ingredient(s) obtained from the pharmaceutical composition is also low (less than 20%).

Further, dosage forms of the pharmaceutical composition disclosed herein cannot easily be snorted. In order for a drug abuser to successfully snort a drug obtained from a dosage form, he must prepare a crushed, finely divided powder form of the dosage form for insufflating the powder into the nasal cavity. However, the pharmaceutical compositions disclosed herein form a clumpy, solid mass and do not allow acceptable absorption through the nasal tissue.

Dosage forms of the pharmaceutical composition disclosed herein also do not allow “dose dumping” caused by the deliberate introduction of alcohol into a drug abuser's stomach which accelerates the release of active ingredient(s) from the time-release formulation. The pharmaceutical compositions disclosed herein are resistant to the accelerated release of active ingredient(s).

In addition, dosage forms of the pharmaceutical composition disclosed herein do not allow for “free basing.” Successful free basing by a drug abuser requires the generation of a salt free form of the active pharmaceutical ingredient(s). This requires physical and chemical manipulation to release the active pharmaceutical ingredient(s) from its salt(s) and selective extraction from other matrix excipients. The pharmaceutical composition disclosed herein cannot be easily manipulated to generate a free base preparation.

Moreover, the tamper resistance properties of the pharmaceutical compositions disclosed herein may be increased by increasing the average molecular weight of the extended release polymer used in the pharmaceutical composition. In another embodiment, the tamper resistance properties of the pharmaceutical compositions disclosed herein may be increased by increasing the amount of the extended release polymer used in the pharmaceutical composition.

In further embodiments, the solid oral dosage forms of the pharmaceutical compositions disclosed herein comprising hydrocodone and acetaminophen will exhibit substantial differences in the release profiles when the dosage forms are crushed or ground versus when the dosage forms are intact. For example, the intact solid oral dosage forms comprising hydrocodone and acetaminophen will exhibit a higher release rate of both active ingredients than one that is crushed or ground. This suggests that upon grinding or crushing the solid oral dosage forms disclosed herein, the immediate release portion and extended release portion of the dosage form combine, and the hydration and swelling of the polymer(s) in the extended release portion of the dosage form retards the release of the hydrocodone and acetaminophen in the immediate release portion, and will also retard the release of the hydrocodone and acetaminophen in the extended release portion. Hence the incorporation of the ground or crushed components from the immediate release portion into a mixture with the ground or crushed components of the extended release portion will cause the pharmaceutical composition to lose its immediate release characteristics. This feature will effectively negate a drug abuser's purpose for crushing the solid oral dosage form in the first place—to obtain an early onset of analgesia. Thus, when the dosage forms disclosed herein comprising hydrocodone and acetaminophen are crushed or ground, the absorption of the hydrocodone and acetaminophen will be delayed, thereby delaying the onset of euphoria as compared to when the dosage forms are ingested in an intact state.

In one embodiment, an extended release dosage form disclosed herein (such as a bilayer tablet comprising an immediate release layer and an extended release layer), containing hydrocodone and acetaminophen will provide a subject with AUC measurements for hydrocodone and acetaminophen that are higher when the tablets are administered in an intact state versus when the tablet is administered in a crushed or ground state. For example, in one embodiment, the AUC measurements for either hydrocodone and/or acetaminophen will be about 5% to about 60% higher when a subject ingests the tablet in an intact state versus a crushed or ground state. In another embodiment, the AUC measurements for either hydrocodone and/or acetaminophen will be about 10% to about 50% higher when a subject ingests the tablet in an intact state versus a crushed or ground. In yet another embodiment, the AUC measurements for either hydrocodone and/or acetaminophen will be about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% higher when a subject ingests the tablet in an intact state versus in a crushed or ground state.

In a further embodiment, the AUC(0-1 hr) for either hydrocodone and/or acetaminophen will be about 50%, 100%, 150%, 200%, 250%, 300%, 350%, 400%, 450%, 500%, 550%, 600%, 650%, 700%, 750%, 800%, 850%, 900%, 950% or 1000% higher when a subject ingested the tablet in an intact state versus in a crushed or ground state. In another embodiment, the AUC(0-1 hr) for either hydrocodone and/or acetaminophen will be about 50% to about 1000% higher when a subject ingested the tablet in an intact state versus in a crushed or ground state. In a further embodiment, the AUC(0-1 hr) for either hydrocodone and/or acetaminophen will be about 100% to about 900% higher when a subject inigested the tablet in an intact state versus in a crushed or ground state. In still a further embodiment, the AUC(0-1 hr) for either hydrocodone and/or acetaminophen will be about 200% to about 800% higher when a subject ingested the tablet in an intact state versus in a crushed or ground state. In yet another embodiment, the AUC(0-1 hr) for either hydrocodone and/or acetaminophen will be about 300% to about 700% higher when a subject ingested the tablet in an intact state versus in a crushed or ground state.

In another embodiment, the AUC(0-2 hr) for either hydrocodone and/or acetaminophen will be about 50%, 100%, 150%, 200%, 250%, 300%, 350%, 400%, 450%, or 500% higher when a subject ingested the tablet in an intact state versus in a crushed or ground state. In another embodiment, the AUC(0-2 hr) for either hydrocodone and/or acetaminophen will be about 50% to about 500% higher when a subject ingested the tablet in an intact state versus in a crushed or ground state. In a further embodiment, the AUC(0-2 hr) for either hydrocodone and/or acetaminophen will be about 100% to about 400% higher when a subject inigested the tablet in an intact state versus in a crushed or ground state. In still a further embodiment, the AUC(0-2 hr) for either hydrocodone and/or acetaminophen will be about 150% to about 300% higher when a subject ingested the tablet in an intact state versus in a crushed or ground state. In an additional embodiment, the AUC(0-2 hr) for either hydrocodone and/or acetaminophen will be about 50% to about 250% higher when a subject ingested the tablet in an intact state versus in a crushed or ground state.

In another embodiment, the AUC(0-4 hr) for either hydrocodone and/or acetaminophen will be about 5%, 10%, 15%, 20%, 25%, 30%, 35%, 40%, 45%, 50%, 55%, 60%, 65%, 70%, 75%, 80%, 85%, 90%, 95%, or 100% higher when a subject ingested the tablet in an intact state versus in a crushed or ground state. In a further embodiment, the AUC(0-4 hr) for either hydrocodone and/or acetaminophen will be about 25% to about 75% higher when a subject ingested the tablet in an intact state versus in a crushed or ground state. In still another embodiment, the AUC(0-8 hr) for either hydrocodone and/or acetaminophen will be about 10%, 20%, 30%, 40%, 50%, 60%, 70% or 80% higher when a subject ingested the tablet in an intact state versus in a crushed or ground state. In an additional embodiment, the AUC(0-8 hr) for either hydrocodone and/or acetaminophen will be about 10% to about 45% higher when a subject ingested the tablet in an intact state versus in a crushed or ground state.

In another embodiment, the AUC(0-inf) for either hydrocodone and/or acetaminophen will be about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% higher when a subject ingested the tablet in an intact state versus in a crushed or ground state. In still another embodiment, the AUC(0-inf) for either hydrocodone and/or acetaminophen will be from about 5% to about 40% higher when a subject ingested the tablet in an intact state versus in a crushed or ground state. In still another embodiment, the AUC(0-inf) for either hydrocodone and/or acetaminophen will be from about 7% to about 30% higher when a subject ingested the tablet in an intact state versus in a crushed or ground state. In a further embodiment, the AUC(0-inf) for either hydrocodone and/or acetaminophen will be from about 10% to about 30% higher when a subject ingested the tablet in an intact state versus in a crushed or ground state.

In another embodiment, the AUC(0-t) for either hydrocodone and/or acetaminophen will be about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% higher when a subject ingested the tablet in an intact state versus in a crushed or ground state. In another embodiment, the AUC(0-t) for either hydrocodone and/or acetaminophen will be from about 2% to about 40% higher when a subject ingested the tablet in an intact state versus in a crushed or ground state. In still another embodiment, the AUC(0-t) for either hydrocodone and/or acetaminophen will be from about 3% to about 30% higher when a subject ingested the tablet in an intact state versus in a crushed or ground state. In a further embodiment, the AUC(0-t) for either hydrocodone and/or acetaminophen will be from about 4% to about 30% higher when a subject ingested the tablet in an intact state versus in a crushed or ground state. In another embodiment, the AUC(0-t) for either hydrocodone and/or acetaminophen will be from about 5% to about 20% higher when a subject ingested the tablet in an intact state versus in a crushed or ground state.

In another embodiment, the Tmax for both hydrocodone and/or acetaminophen will be lower when the tablet was administered in an intact state versus when the tablet was administered in a crushed or ground state. For instance, in one embodiment, the Tmax for either hydrocodone and/or will be about 5% to about 70% lower when the tablet is administered in an intact state versus when the tablet is administered in a crushed or ground state. In an additional embodiment, the Tmax for either hydrocodone and/or acetaminophen will be 10% to about 40% lower when the tablet is administered in an intact state versus when the tablet is administered in a crushed or ground state. In another embodiment, the Tₘₐₓ for either hydrocodone and/or acetaminophen will be about 20% to about 60%. In still another embodiment, the Tmax for either hydrocodone and/or acetaminophen will be about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 46%, 48%, 49% or 50% higher when a subject ingests the tablet in a crushed or ground state versus in an intact state. In yet another embodiment, the Tmax for either hydrocodone and/or acetaminophen will be about 25%, 50%, 75%, 100%, 125%, 150%, 175%, 200%, 225%, 250%, 300% or 325% higher when a subject ingests the tablet in a crushed or ground state versus in an intact state. In an additional embodiment, administration of the tablet to a subject produces a mean Tmax for either hydrocodone or acetaminophen that will be at least about 30 minutes greater when the tablet is administered in a crushed or ground state as compared to an intact state. In a further embodiment, administration of the tablet to a subject produces a mean Tmax for either hydrocodone or acetaminophen that will be at least about 30 minutes, 45 minutes, 60 minutes, 75 minutes, 90 minutes, 105 minutes, 120 minutes, 135 minutes, or 150 minutes greater when the tablet is administered in a crushed or ground state as compared to an intact state.

The Cmax for acetaminophen will be higher when the tablet was administered in an intact state versus when the tablet was administered in a crushed or ground state. For example, in one embodiment, the Cmax for acetaminophen will be about 5% to about 50% higher when the tablet was administered in an intact state versus when the tablet was administered in a crushed or ground state. In yet another embodiment, the Cmax for acetaminophen will be about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 46%, 48%, 49% or 50% higher when the tablet was administered in an intact state versus when the tablet is administered in a crushed or ground state.

In one embodiment, the abuse quotient of the tablet will be higher when the tablet is administered in an intact state versus when the tablet is administered in a crushed or ground state. For example, in another embodiment, the abuse quotient may decrease in an amount of from about 5% to about 90% when the tablet is administered in a crushed or ground state versus in an intact state. In a further embodiment, the abuse quotient may decrease in an amount from about 10% to about 80% when the tablet is administered in a crushed or ground state versus in an intact state. In yet another embodiment, the abuse quotient may decrease in an amount from about 15% to about 80% when the tablet is administered in a crushed or ground state versus in an intact state. In still another embodiment, the abuse quotient may decrease in an amount of from about 20% to about 70% when the tablet is administered in a crushed or ground state versus in an intact state. In another embodiment, the abuse quotient may decrease in an amount of about 5%, 10%, 15%, 20%, 25%, 30%, 35%, 40%, 45%, 50%, 55%, 60%, 65%, 70%, 75%, 80%, 85%, or 90% when the tablet is administered in a crushed or ground state versus in an intact state.

As a result of these pharmacokinetic parameters, a drug abuser will be more likely to take the extended release dosage forms disclosed herein in an intact form rather than in a crushed form. Moreover, drug abusers “like” the dosage forms disclosed herein better when the dosage forms are taken in an intact state rather than in a crushed or ground state. See FDA's Guidance for Industry Document titled, “Assessment of Abuse Potential of Drug,” dated January 2010. Both overall and “at the moment” drug liking will be assessed on a bipolar visual analog scale (VAS) anchored by “strong disliking” (0), “neutral” (50), and “strong liking” (100).

In another embodiment, as the amount of hydrocodone in the pharmaceutical composition increases, so does the duration of gastric retention after administration to a subject. Consequently, if a subject either intentionally or accidentally ingests a larger dose of the pharmaceutical composition than prescribed, the pharmaceutical composition will be retained in the stomach for a longer time period than an IR or traditional ER pharmaceutical composition, thereby giving a medical provider additional time to perform gastric lavage, induce vomiting, or administer activated charcoal to prevent the body from absorbing the hydrocodone. In a further embodiment, the pharmaceutical composition provides a medical provider with about an additional 15 minutes, 30 minutes, 45 minutes, 60 minutes, 75 minutes, 90 minutes, 105 minutes, 2.0 hours, 2.25 hours, 2.5 hours, 2.75 hours, 3.0 hours, 3.25 hours, 3.5 hours, 3.75 hours, or 4 hours in which to prevent the absorption of hydrocodone in the subject. In another embodiment, the pharmaceutical composition provides a medical provider with sufficient time to treat a subject who has overdosed on hydrocodone so that death, difficulty breathing, cardiac arrest, and limp muscles do not occur in the subject.

In yet another embodiment, if vomiting is induced or naturally occurs as a result of an increased dose of hydrocodone, the entire pharmaceutical composition is expelled from the subject. Thus, toxic concentrations of the hydrocodone due to absorption into the subject's blood are prevented by removing the further release of hydrocodone. In still another embodiment, if vomiting is induced or naturally occurs as a result of the increased dose of hydrocodone about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the pharmaceutical composition is expelled from the subject. In yet another embodiment, if vomiting is induced or naturally occurs within about 30 minutes to about 60 minutes after ingestion of the increased dose of hydrocodone about 50% to about 65% of the hydrocodone dose is expelled from the subject.

### (f) In Vitro Release Properties of the Composition

The in vitro release rates of hydrocodone and acetaminophen from the pharmaceutical compositions disclosed herein may be measured in 900 mL of 0.1 N HCl using a USP type II paddle apparatus and at a paddle speed of either about 100 rpm or 150 rpm and a constant temperature of 37° C.

In one embodiment, the at least one immediate release portion of the composition may have in vitro release rates of hydrocodone and acetaminophen as follows: more than about 90% of the hydrocodone and/or the acetaminophen present in the at least one immediate release portion is released in about 15 minutes, or essentially 100% of the hydrocodone and/or the acetaminophen present in the at least one immediate release portion may be released within about 15 minutes. In another embodiment, more than about 90% of the hydrocodone and/or the acetaminophen present in the at least one immediate release portion may be released within about 5 min. In yet another embodiment, essentially 100% of the hydrocodone and/or the acetaminophen present in the at least one immediate release portion may be released within about 5 min.

In one embodiment, the at least one extended release portion of the composition may have in vitro release rate of hydrocodone as follows: from about 1% to about 20% of the hydrocodone present in the at least one extended release portion may be released within about 15 min, from about 30% to about 50% of the hydrocodone present in the at least one extended release portion is released within about 2 hours, from about 50% to about 75% of the hydrocodone present in the at least one extended release portion is released in about 4 hours, at least about 80% of the hydrocodone present in the at least one extended release portion is released within about 8 hours, and at least about 90% of the hydrocodone present in the at least one extended release portion is released within about 12 hours.

In yet another embodiment, the at least one extended release portion may have in vitro release rates of hydrocodone as follows: from about 1% to about 20% of the hydrocodone present in the extended release portion may be released within about 15 min, from about 30% to about 50% of the hydrocodone present in the extended release portion may be released within about 2 hours, from about 50% to about 75% of the hydrocodone present in the extended release portion may be released within about 4 hours, and from about 80% to about 100% of the hydrocodone present in the extended release portion may be released within about 8 hours.

In one embodiment, the in vitro release rates of hydrocodone from the composition may be as follows: about 20% to about 50% of hydrocodone may be released from the composition within about 15 minutes, from about 25% to about 55% of hydrocodone may be released from the composition within about 30 minutes, from about 35% to about 65% of hydrocodone may be released within about 1 hour, from about 40% to about 80% of hydrocodone may be released from the composition in about 2 hours, from about 60% to about 100% of hydrocodone may be released from the composition within about 4 hours, from about 70% to about 100% of hydrocodone may be released from the composition within about 6 hours, from about 80% to about 100% of hydrocodone may be released from the composition within about 8 hours, from about 90% to about 100% of hydrocodone may be released from the composition within about 12 hours, and from about 90% to about 100% of hydrocodone may be released from the composition within about 18 hours.

In another embodiment, the in vitro release rates of hydrocodone from the composition may be as follows: about 20% to about 40% of hydrocodone may be released from the composition within about 15 minutes, from about 25% to about 45% of hydrocodone may be released from the composition within about 30 minutes, from about 35% to about 55% of hydrocodone may be released within about 1 hour, from about 45% to about 65% of hydrocodone may be released from the composition in about 2 hours, from about 60% to about 85% of hydrocodone may be released from the composition within about 4 hours, from about 70% to about 100% of hydrocodone may be released from the composition within about 6 hours, from about 80% to about 100% of hydrocodone may be released from the composition within about 8 hours, from about 85% to about 100% of hydrocodone may be released from the composition within about 12 hours, and from about 90% to about 100% of hydrocodone may be released from the composition within about 18 hours.

In another embodiment, the in vitro release rates of hydrocodone from the composition may be as follows: about 30% to about 35% of hydrocodone may be released from the composition within about 15 minutes, from about 35% to about 40% of hydrocodone may be released from the composition within about 30 minutes, from about 40% to about 50% of hydrocodone may be released within about 1 hour, from about 50% to about 60% of hydrocodone may be released from the composition in about 2 hours, from about 65% to about 75% of hydrocodone may be released from the composition within about 4 hours, from about 80% to about 90% of hydrocodone may be released from the composition within about 6 hours, from about 90% to about 100% of hydrocodone may be released from the composition within about 8 hours, and from about 95% to about 100% of hydrocodone may be released from the composition within about 12 hours.

In one embodiment, the in vitro release rates of acetaminophen from the composition may be as follows: about 40% to about 65% of acetaminophen may be released from the composition within about 15 minutes, from about 45% to about 65% of acetaminophen may be released from the composition with about 30 minutes, from about 50% to about 70% of acetaminophen may be released from the composition within about 1 hour, from about 55% to about 80% of acetaminophen may be released from the composition within about 2 hours, from about 65% to about 95% of acetaminophen may be released from the composition within about 4 hours, from about 75% to about 100% of acetaminophen may be released from the composition within about 6 hours, from about 80% to about 100% of acetaminophen may be released from the composition within about 8 hours, from about 85% to about 100% of acetaminophen may be released from the composition within about 12 hours, and from about 90% to about 100% of acetaminophen may be released from the composition within about 18 hours.

In a further embodiment, the in vitro release rates of acetaminophen from the composition may be as follows: about 50% to about 55% of acetaminophen may be released from the composition within about 15 minutes, from about 52% to about 58% of acetaminophen may be released from the composition within about 30 minutes, from about 55% to about 60% of acetaminophen may be released from the composition within about 1 hour, from about 60% to about 65% of acetaminophen may be released from the composition within about 2 hours, from about 70% to about 75% of acetaminophen may be released from the composition within about 4 hours, from about 80% to about 85% of acetaminophen may be released from the composition within about 6 hours, from about 90% to about 95% of acetaminophen may be released from the composition with about 8 hours, and from about 95% to about 100% of acetaminophen may be released from the composition within about 12 hours.

In one embodiment, the in vitro release rates of hydrocodone and acetaminophen from the pharmaceutical composition generally are not affected by low concentrations of ethanol (e.g., from about 5% v/v to about 20% v/v) when measured in 900 mL of 0.1 N HCl containing the desired percentage of ethanol using a USP type II paddle apparatus and at a paddle speed of about 150 rpm and a constant temperature of 37° C. For example, from about 25% to about 35% of hydrocodone and about 50% to about 55% of acetaminophen may be released from the pharmaceutical composition within about 15 minutes when measured in the presence of 5% to 20% ethanol, and from about 50% to about 65% of hydrocodone and from about 60% to about 70% of acetaminophen may be released from the pharmaceutical composition within about 2 hours when measured in the presence of 5% to 20% ethanol.

The in vitro release rates of hydrocodone and acetaminophen from the pharmaceutical compositions disclosed herein generally are reduced, however, in the presence of 40% ethanol. For example, from about 5% to about 15% of the hydrocodone and from about 15% to about 30% of the acetaminophen may be released from the pharmaceutical composition within about 15 minutes when measured in the presence of 40% ethanol, and from about 30% to about 45% of hydrocodone and from about 45% to about 55% of acetaminophen may be released from the pharmaceutical composition within about 2 hours when measured in the presence of 40% ethanol.

Stated another way, less hydrocodone is extracted from the pharmaceutical composition by a solution of 0.1 N HCl and 40% ethanol than is extracted by a solution of 0.1 N HCl. In some embodiments, less than about 75% of the hydrocodone that is released in the presence of 0.1N HCl may be released in the presence of 0.1N HCl containing 40% ethanol. In additional embodiments, less than about 70%, 65%, 60%, 55%, 50%, 45%, or 40% of the hydrocodone that may be released in the presence of 0.1N HCl may be released in the presence of 0.1N HCl and 40% ethanol. For example, less than about 40% of the hydrocodone that may be released in the presence of 0.1N HCl in about 15 minutes may be released in the presence of 0.1N HCl and 40% ethanol within about 15 minutes. In other embodiments, less than about 60% of the hydrocodone that may be released in the presence of 0.1N HCl in about 30 minutes may be released in the presence of 0.1N HCl and 40% ethanol within about 30 minutes. In additional embodiments, less than about 75% of the hydrocodone that may be released in the presence of 0.1N HCl in about 2 hours may be released in the presence of 0.1N HCl and 40% ethanol within about 2 hours.

### (g) Stability Data for the Pharmaceutical Composition

In one embodiment, p-aminophenol may be present in the pharmaceutical composition as a degradation product of acetaminophen in any amount up to and including, but no more than, about 100 ppm. In other embodiments, p-aminophenol may be present in the pharmaceutical composition as a degradation product of acetaminophen in an amount of about 0.2 ppm to about 6.0 ppm after storage for about 1, 2, or 3 months at a temperature of about 25° C. to about 40° C. and at about 60% to about 75% relative humidity. In yet another embodiment, p-aminophenol may be present in the pharmaceutical composition as a degradation product of acetaminophen in an amount of about 0.6 ppm to about 6.0 ppm after storage for about 1, 2, or 3 months at a temperature of about 25° C. to about 40° C. and at about 60% to about 75% relative humidity. In still another embodiment, p-aminophenol may be present in the pharmaceutical composition as a degradation product of acetaminophen in an amount of about 0.2 ppm, 0.3 ppm, 0.4 ppm, 0.5 ppm, 0.6 ppm, 0.7 ppm, 0.8 ppm, 0.9 ppm, 1.0 ppm, 1.1 ppm, 1.2 ppm, 1.3 ppm, 1.4 ppm, 1.5 ppm, 1.6 ppm, 1.7 ppm, 1.8 ppm, 1.9 ppm, 2.0 ppm, 2.1 ppm, 2.2 ppm, 2.3 ppm, 2.4 ppm, 2.5 ppm, 2.6 ppm, 2.7 ppm, 2.8 ppm, 2.9 ppm, 3.0 ppm, 3.1 ppm, 3.2 ppm, 3.3 ppm, 3.4 ppm, 3.5 ppm, 3.6 ppm, 3.7 ppm, 3.8 ppm, 3.9 ppm, 4.0 ppm, 4.1 ppm, 4.2 ppm, 4.3 ppm, 4.4 ppm, 4.5 ppm, 4.6 ppm, 4.7 ppm, 4.8 ppm, 4.9 ppm, 5.0 ppm, 5.1 ppm, 5.2 ppm, 5.3 ppm, 5.4 ppm, 5.5 ppm, 5.6 ppm, 5.7 ppm, 5.8 ppm, 5.9 ppm, and 6.0 ppm after storage for about 1, 2, or 3 months at a temperature of 25° C. to about 40° C. and at about 60% to about 75% relative humidity

In one embodiment, each unspecified acetaminophen degradation product may be present in the pharmaceutical composition in any amount up to about 0.15% by weight of the acetaminophen. In another embodiment, each unspecified acetaminophen degradation product may be present in the pharmaceutical composition as a degradation product of acetaminophen in an amount of about 0.01% and about 0.15% by weight of the acetaminophen after storage for about 1, 2, or 3 months at a temperature of about 25° C. to about 40° C. and at about 60% to about 75% relative humidity. In still another embodiment, each unspecified acetaminophen degradation product may be present in the pharmaceutical composition as a degradation product of acetaminophen in an amount of about 0.05% and about 0.15% by weight of the acetaminophen after storage for about 1, 2, or 3 months at a temperature of about 25° C. to about 40° C. and at about 60% to about 75% relative humidity. In other embodiments, each unspecified acetaminophen degradation product may be present in the pharmaceutical composition as a degradation product of acetaminophen in an amount of about 0.01%, 0.02%, 0.03%, 0.04%, 0.05%, 0.06%, 0.07%, 0.08%, 0.09%, 0.1%, 0.11%, 0.12%, 0.13%, 0.14%, and 0.15% by weight of the acetaminophen after storage for about 1, 2, or 3 months at a temperature of about 25° C. to about 40° C. and at about 60% to about 75% relative humidity.

In one embodiment, the total acetaminophen degradation products may be present in the pharmaceutical composition in a maximum amount of about 1.0% by weight of the acetaminophen. In other embodiments, the total acetaminophen degradation products may be present in the pharmaceutical composition in an amount of about 0.05% to about 1.0% by weight of the acetaminophen after storage for about 1, 2, or 3 months at a temperature of about 25° C. to about 40° C. and at about 60% to about 75% relative humidity. In further embodiments, the total acetaminophen degradation products may be present in the pharmaceutical composition in an amount of about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, and 1.0% by weight of the acetaminophen after storage for about 1, 2, or 3 months at a temperature of about 25° C. to about 40° C. and at about 60% to about 75% relative humidity.

In one embodiment, the total hydrocodone degradation products may be present in the pharmaceutical composition in a maximum amount of about 1.0% by weight of the hydrocodone. In further embodiments, the total hydrocodone degradation products may be present in the pharmaceutical composition in an amount of about 0.05% to about 1.0% by weight of the hydrocodone after storage for about 1, 2, or 3 months at a temperature of about 25° C. to about 40° C. and at about 60% to about 75% relative humidity. In yet other embodiments, the total hydrocodone degradation products may be present in the pharmaceutical composition in an amount of about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, and 1.0% by weight of the hydrocodone after storage for about 1, 2, or 3 months at a temperature of about 25° C. to about 40° C. and at about 60% to about 75% relative humidity.

### (h) In Vivo and Pharmacokinetic Properties of the Pharmaceutical Composition

The pharmaceutical composition disclosed herein comprises at least one immediate release portion for immediate release of hydrocodone and acetaminophen such that therapeutic plasma concentrations are quickly attained (e.g., within one hour) and the initial onset of action is achieved within about 5 minutes, 10 minutes, 15 minutes, 20 minutes, 25 minutes, 30 minutes, 35 minutes, 40 minutes, 45 minutes, 50 minutes, 55 minutes, or 60 minutes after administration of the composition upon oral administration to a subject. The pharmaceutical composition disclosed herein also comprises at least one extended release portion for sustained release of hydrocodone and acetaminophen over an extended period of time, e.g., about 3 to about 12 hours, or about 4 to about 9 hours, or at least about 6 hours, or at least about 8 hours, to the upper gastrointestinal tract where acetaminophen, and potentially hydrocodone, is best absorbed.

The pharmaceutical composition may be orally administered to a subject once in a 24 hour period (q.d. or once-daily), two times in a 24 hour period (b.i.d. or twice-daily), or three times in a 24 hour period (t.i.d. or three times daily). In one embodiment, the pharmaceutical composition may be orally administered to the subject twice a day (i.e., every 12 hours). The subject may be a mammal, and in certain embodiments, the subject may be a human.

In another embodiment, the subject may be administered a first or loading dose of the pharmaceutical composition. This first or loading dose may assist the subject in more quickly attaining steady state blood levels of the active drugs. In a further embodiment, the subject may be administered a first or loading dose of the pharmaceutical composition comprising about 22.5 mg of hydrocodone and about 975 mg of acetaminophen. In yet another embodiment, the subject may be administered a first or loading dose of the pharmaceutical composition comprising 2 tablets, each tablet comprising about 11.25 mg of hydrocodone and about 462.5 mg of acetaminophen. In yet another embodiment, the subject may be administered a first or loading dose of the pharmaceutical composition comprising 3 tablets, each tablet comprising about 7.5 mg of hydrocodone and about 325 mg of acetaminophen. In still another embodiment, the subject may be administered a first or loading dose of the pharmaceutical composition comprising 4 tablets, each tablet comprising about 5.625 mg of hydrocodone and about 231.25 mg of acetaminophen. In yet another embodiment, the subject may be administered a first or loading dose of the pharmaceutical composition comprising 2 capsules, each capsule comprising about 11.25 mg of hydrocodone and about 462.5 mg of acetaminophen. In yet another embodiment, the subject may be administered a first or loading dose of the pharmaceutical composition comprising 3 capsules, each capsules comprising about 7.5 mg of hydrocodone and about 325 mg of acetaminophen. In still another embodiment, the subject may be administered a first or loading dose of the pharmaceutical composition comprising 4 capsules, each capsules comprising about 5.625 mg of hydrocodone and about 231.25 mg of acetaminophen.

Further, upon oral administration to a subject, the pharmaceutical composition disclosed herein may maintain a therapeutic blood plasma concentration of hydrocodone of at least about 5 ng/mL from about 0.75 hours to about 12 hours after administration of the composition. In another embodiment, the plasma concentration of hydrocodone may be maintained at a concentration of at least about 7.5 ng/mL from about 0.5 hour to about 10 hours after administration of the composition. In yet another embodiment, the plasma concentration of hydrocodone may be maintained at a concentration of at least about 7.5 ng/mL from about 1 hour to about 12 hours after administration of the composition. In a further embodiment, the plasma concentration of hydrocodone may be maintained at a concentration of at least about 10 ng/mL from about 2 hours to about 10 hours after administration of the composition. In yet another embodiment, the plasma concentration of hydrocodone may be maintained at a concentration of at least about 10 ng/mL from about 1 hour to about 10 hours after administration of the composition. In still another embodiment, the plasma concentration of hydrocodone may be maintained at a concentration of at least about 10 ng/mL from about 0.75 hour to about 10 hours after administration of the composition.

In another embodiment, the pharmaceutical composition, when orally administered to a subject, may produce a plasma profile characterized by a mean Cmax (peak plasma concentration) for hydrocodone from about 0.9 ng/mL/mg to about 2.0 ng/mL/mg. In still another embodiment, the mean Cmax for hydrocodone may range from about 1.0 ng/mL/mg to about 1.6 ng/mL/mg. In yet another embodiment, the pharmaceutical composition, when orally administered to a subject, may produce a plasma profile characterized by a mean Cmax (peak plasma concentration) for hydrocodone from about 0.9 ng/mL/mg to about 1.6 ng/mL/mg. In another embodiment, the mean Cmax for hydrocodone may range from about 1.0 ng/mL/mg to about 1.5 ng/mL/mg. In an additional embodiment, the mean Cmax for hydrocodone may be about 0.9, 1.0, 1.1, 1.2, 1.3, 1.4, 1.5, 1.6, 1.7, 1.8, 1.9, or 2.0 ng/mL/mg. Moreover, the mean Cmax for hydrocodone at steady state may range from about 1.5 ng/mL/mg to about 2.0 ng/mL/mg, from about 1.6 ng/mL/mg to about 1.95 ng/mL/mg, or from about 1.7 ng/mL/mg to about 1.85 ng/mL/mg. In other embodiments, the mean Cmax for hydrocodone at steady state may range from about 1.3 ng/mL/mg to about 2.0 ng/mL/mg, from about 1.5 ng/mL/mg to about 1.95 ng/mL/mg, or from about 1.6 ng/mL/mg to about 1.85 ng/mL/mg.

In a further embodiment, the pharmaceutical composition, when orally administered to a subject, surprisingly may produce a plasma profile characterized by a biphasic absorption of hydrocodone. Deconvolution of the pharmaceutical composition and the target plasma profiles can be done in WinNonLin (version 5.2, Pharsight Corp., Mountain View, Calif.). The biphasic absorption of hydrocodone may be characterized by an initial rapid absorption resulting in a first peak in plasma concentrations between about 1 hour and 2 hours, which contributes to the early onset of action, and a second peak in plasma concentrations between about 3 hours and 7 hours as a result of slower absorption taking place from the at least one extended release portion after administration of the composition, which contributes to the duration or maintenance of analgesia. In some instances, the second peak may correspond to the overall Cmax of the composition. The biphasic of hydrocodone may be characterized by a plasma concentration-time profile for hydrocodone in which the slope of a line drawn between 0 hour and about 2 hours is greater than the slope of a line drawn between about 2 hours and about 5 hours.

This biphasic increase in hydrocodone levels resulting from the composition has several benefits. For example, providing rapid but not too high concentrations of hydrocodone for quick onset of analgesia followed by maintenance of hydrocodone levels over an extended time period could prevent a human subject from developing liking or dependence (abuse) for hydrocodone. Further fluctuations in the hydrocodone plasma levels could also prevent development of tolerance at the active site. Thus, the biphasic increase in hydrocodone levels helps to prevent this acute tolerance.

In an additional embodiment, the pharmaceutical composition, when orally administered to a subject, may produce a plasma profile characterized by a mean AUC for hydrocodone from about 9.0 ng·hr/mL/mg to about 24.0 ng·hr/mL/mg. In yet another embodiment, the pharmaceutical composition, when orally administered to a subject, may produce a plasma profile characterized by a mean AUC for hydrocodone from about 9.0 ng·hr/mL/mg to about 18.5 ng·hr/mL/mg. In a further embodiment, the mean AUC for hydrocodone may be from about 10.0 ng·hr/mL/mg to about 22.0 ng·hr/mL/mg. In a further embodiment, the mean AUC for hydrocodone may be from about 12.0 ng·hr/mL/mg to about 16.0 ng·hr/mL/mg. In another embodiment, the mean AUC for hydrocodone may be about 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, or 24.0 ng·hr/mL/mg. Additionally, the mean AUC for hydrocodone at steady state may range from about 10.0 ng·hr/mL/mg to about 20.0 ng·hr/mL/mg, from about 12.0 ng·hr/mL/mg to about 19.0 ng·hr/mL/mg, or from about 13.0 ng·hr/mL/mg to about 18.0 ng·hr/mL/mg. in yet other embodiments, the mean AUC for hydrocodone at steady state may range from about 10.0 ng·hr/mL/mg to about 20.0 ng·hr/mL/mg, from about 12.0 ng·hr/mL/mg to about 16.0 ng·hr/mL/mg, or from about 13.0 ng·hr/mL/mg to about 15.0 ng·hr/mL/mg.

In a further embodiment, the pharmaceutical composition, when orally administered to a subject, may produce a plasma profile characterized by a median Tmax (time to peak plasma concentration) for hydrocodone from about 2.0 hours to about 8.0 hours. In an alternate embodiment, the median Tmax for hydrocodone may be from about 3.0 hours to about 6.0 hours. In another embodiment, the median Tmax for hydrocodone may be about 2.0, 2.5, 3.0, 3.5, 4.0, 4.5, 5.0, 5.5, 6.0, 6.5, 7.0, 7.5, or 8.0 hours. Moreover, the median Tmax for hydrocodone at steady state may range from about 1.5 hours to about 5.0 hours, or from about 2 hours to about 4 hours. Further, the median Tmax for hydrocodone at steady state may range from about 1.5 hours to about 3.5 hours, or from about 2 hours to about 3 hours.

In still another embodiment, the pharmaceutical composition, when orally administered to a subject, may produce a plasma profile characterized by a median tlag for hydrocodone from about 0 hours to about 0.5 hours. In an alternate embodiment, the median tlag for hydrocodone may be from about 0 hours to about 0.33 hours. In an alternate embodiment, the median tlag for hydrocodone may be from about 0 hours to about 0.25 hours.

Rates of absorption are often assessed by comparing standard pharmacokinetic parameters such as Tmax and Cmax. The extent of absorption is assessed by the AUC. A short Tmax has been used to indicate rapid absorption. The U.S. FDA, Guidance for Industry: Bioavailability and Bioequivalence Studies for Orally Administered Drug Products—General Considerations (March 2003) and related publications (Chen et al, Clin. Pharmacokinet. 40(8):565-72, 2001) also recommends the use of partial AUC for some modified-release drugs (“MR drugs”), such as the pharmaceutical compositions disclosed herein. A partial AUC calculation may be used to measure early exposure to a drug, which may signify an initial onset of pain relief and/or to measure prolonged exposure of a drug in achieving sustained relief. Partial AUC calculations can also demonstrate whether two MR drugs are truly bioequivalent by comparing, for example, an early partial AUC, which will be associated with a drug's response onset, and a late partial AUC, which will be associated with a drug's sustained response. The parameters for compositions vary greatly between subjects. The parameters also vary depending on aspects of the study protocol such as the sampling scheduling, subject posture and general subject health. Values quoted in this specification are given as mean±standard deviation unless otherwise noted.

For partial AUC calculations, the standard linear trapezoidal summation over each time interval is used. The partial AUCs are calculated from the mean pharmacokinetic profile. For time 0 to 1 hour the partial AUC is AUC₍₀₋₁ₕᵣ₎; for time 0 to 2 hours the partial AUC is AUC₍₀₋₂ₕᵣ₎; for time 0-4 hours the partial AUC is AUC₍₀₋₄ₕᵣ₎; for time 0 to 6 hour the partial AUC is AUC₍₀₋₆ₕᵣ₎; for time 0 to 8 hours the partial AUC is AUC₍₀₋₈ₕᵣ₎; and for time 0 to the last measurable time point (“x”) the partial AUC is AUC₍₀₋₍ₓ₎ₕᵣ₎ where each partial AUC is calculated according to standard pharmaceutical industry pharmacokinetic calculation methodologies as given by:

AUC₍₀₋₁ₕᵣ₎—Area under the drug concentration-time curve calculated using linear trapezoidal summation from time zero to time 1 hour.

AUC₍₀₋₂ₕᵣ₎—Area under the drug concentration-time curve calculated using linear trapezoidal summation from time zero to time 2 hours.

AUC₍₀₋₄ₕᵣ₎—Area under the drug concentration-time curve calculated using linear trapezoidal summation from time zero to time 4 hours.

AUC₍₀₋₆ₕᵣ₎—Area under the drug concentration-time curve calculated using linear trapezoidal summation from time zero to time 6 hours.

AUC₍₀₋₈ₕᵣ₎—Area under the drug concentration-time curve calculated using linear trapezoidal summation from time zero to time 8 hours.

AUC₍₀₋₍ₜ₎ₕᵣ₎—Area under the drug concentration-time curve calculated using linear trapezoidal summation from time zero to the last measurable time point.

AUC_{(0-(Tmax of IR product+2SD))}—Area under the drug concentration-time curve calculated using linear trapezoidal summation from time zero to the time of the mean peak (Tmax) for the immediate release version of the drug plus two standard deviations (“2SD”) for the immediate release drug. The FDA has identified this calculation in association with an early onset of response for certain modified-release dosage forms, which show complex pharmacokinetic characteristics. (See supra March 2003 Guidance; Draft Guidance on Dexmethylphenidate Hydrochloride (March 2012); Draft Guidance on Methylphenidate Hydrocholoride (November 2011)).

AUC_{((Tmax of IR product+2SD)-t)}—Area under the drug concentration-time curve calculated using linear trapezoidal summation from the time of the mean peak (Tmax) for the immediate release version of the drug plus two standard deviations (“2SD”) for the immediate release drug to the last measurable time point. The FDA has identified this parameter in association with sustaining the response for modified-release dosage forms, which shows complex pharmacokinetic characteristics. (See March 2003 Guidance supra; Draft Guidance on Dexmethylphenidate Hydrochloride (March 2012); Draft Guidance on Methylphenidate Hydrocholoride (November 2011)).

AUC_{(x-(y)hr)}—Area under the drug concentration-time curve calculated using linear trapezoidal summation from time “x” (e.g., any measurable time point, such as 8 hours) to time “y” (e.g., any other measurable time point later than “x”, such as 12 hours).

AUC_{(0-∞)}—Area under the drug concentration-time curve calculated using linear trapezoidal summation from time 0 to infinity.

Further, partial AUC may be calculated using trapezoidal summation from time Tmax to time t (the last measured time point of plasma concentration profile).

In one embodiment, the pharmaceutical composition, when orally administered to a subject, may produce a plasma profile characterized by an AUC_{(0-(Tmax of IR product+2SD))} for hydrocodone after a single dose from about 1.0 ng·hr/mL/mg to about 5.0 ng·hr/mL/mg, from about 1.50 ng·hr/mL/mg to about 4.25 ng·hr/mL/mg, or from about 2.0 ng·hr/mL/mg to about 3.0 ng·hr/mL/mg. In another embodiment, the AUC_{(Tmax+2SD of IR product)} for hydrocodone may be about 1.0, 1.25, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, or 3.5 ng·hr/mL/mg.

In one embodiment, the pharmaceutical composition, when orally administered to a subject, may produce a plasma profile characterized by an AUC₍₀₋₃ₕᵣ₎ for hydrocodone after a single dose from about 1.0 ng·hr/mL/mg to about 5.0 ng·hr/mL/mg, from about 1.50 ng·hr/mL/mg to about 4.25 ng·hr/mL/mg, or from about 2.0 ng·hr/mL/mg to about 3.0 ng·hr/mL/mg. In another embodiment, the AUC₍₀₋₃ₕᵣ₎ for hydrocodone may be about 1.0, 1.25, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, or 3.5 ng·hr/mL/mg.

In another embodiment, the pharmaceutical composition, when orally administered to a subject, may produce a plasma profile characterized by an AUC_{(0-2.44hr)} for hydrocodone after a single dose from about 0.5 ng·hr/mL/mg to about 5.0 ng·hr/mL/mg, from about 1.0 ng·hr/mL/mg to about 4.25 ng·hr/mL/mg, or from about 1.5 ng·hr/mL/mg to about 3.0 ng·hr/mL/mg. In another embodiment, the AUC_{(0-2.44hr)} for hydrocodone may be about 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0 ng·hr/mL/mg.

In another embodiment, the pharmaceutical composition, when orally administered to a subject, may produce a plasma profile characterized by an AUC₍₀₋₂ₕᵣ₎ for hydrocodone after a single dose from about 0.4 ng·hr/mL/mg to about 5.0 ng·hr/mL/mg, from about 0.5 ng·hr/mL/mg to about 4.25 ng·hr/mL/mg, or from about 1.0 ng·hr/mL/mg to about 3.5 ng·hr/mL/mg. In another embodiment, the AUC₍₀₋₂ₕᵣ₎ for hydrocodone may be about 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0 ng·hr/mL/mg.

In another embodiment, the pharmaceutical composition, when orally administered to a subject in a fasted state, may produce a plasma profile characterized by an AUC₍₀₋₂ₕᵣ₎ for hydrocodone after a single dose from about 0.4 ng·hr/mL/mg to about 5.0 ng·hr/mL/mg, from about 0.5 ng·hr/mL/mg to about 4.25 ng·hr/mL/mg, from about 0.75.0 ng·hr/mL/mg to about 3.5 ng·hr/mL/mg, from about 1.0 ng·hr/mL/mg to about 2.5 ng·hr/mL/mg, or from about 1.3 ng·hr/mL/mg to about 2.4 ng·hr/mL/mg. In another embodiment, the AUC₍₀₋₂ₕᵣ₎ for hydrocodone may be about 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, or 3.5 ng·hr/mL/mg. In still another embodiment, the AUC₍₀₋₂ₕᵣ₎ for hydrocodone may be about 1.8, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, or 2.0 ng·hr/mL/mg.

In another embodiment, the pharmaceutical composition, when orally administered to a subject in a fed state (high fat), may produce a plasma profile characterized by an AUC₍₀₋₂ₕᵣ₎ for hydrocodone after a single dose from about 0.4 ng·hr/mL/mg to about 5.0 ng·hr/mL/mg, from about 0.5 ng·hr/mL/mg to about 4.25 ng·hr/mL/mg, from about 0.75.0 ng·hr/mL/mg to about 3.5 ng·hr/mL/mg, from about 1.0 ng·hr/mL/mg to about 2.5 ng·hr/mL/mg, or from about 1.25 ng·hr/mL/mg to about 2.25 ng·hr/mL/mg. In another embodiment, the AUC₍₀₋₂ₕᵣ₎ for hydrocodone may be about 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, or 3.5 ng·hr/mL/mg. In still another embodiment, the AUC₍₀₋₂ₕᵣ₎ for hydrocodone may be about 1.6, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, or 2.0 ng·hr/mL/mg.

In another embodiment, the pharmaceutical composition, when orally administered to a subject in a fed state (low fat), may produce a plasma profile characterized by an AUC₍₀₋₂ₕᵣ₎ for hydrocodone after a single dose from about 0.25 ng·hr/mL/mg to about 4.0 ng·hr/mL/mg, from about 0.5 ng·hr/mL/mg to about 3.5 ng·hr/mL/mg, from about 0.75.0 ng·hr/mL/mg to about 3.0 ng·hr/mL/mg, from about 0.75 ng·hr/mL/mg to about 1.5 ng·hr/mL/mg, or from about 0.5 ng·hr/mL/mg to about 1.5 ng·hr/mL/mg. In another embodiment, the AUC₍₀₋₂ₕᵣ₎ for hydrocodone may be about 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, or 3.5 ng·hr/mL/mg. In still another embodiment, the AUC₍₀₋₂ₕᵣ₎ for hydrocodone may be about 1.0, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, or 1.30 ng·hr/mL/mg.

In one embodiment, the pharmaceutical composition, when orally administered to a subject, may produce a plasma profile characterized by an AUC₍₀₋₁₂ₕᵣ₎ for hydrocodone from about 5 ng·hr/mL/mg to about 25 ng·hr/mL/mg, from about 7.5 ng·hr/mL/mg to about 15.5 ng·hr/mL/mg, or from about 8.5 ng·hr/mL/mg to about 12.5 ng·hr/mL/mg. In other embodiments, the pharmaceutical composition, when orally administered to a subject, may produce a plasma profile characterized by an AUC₍₀₋₁₂ₕᵣ₎ for hydrocodone from about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 ng·hr/mL/mg.

In one embodiment, the pharmaceutical composition, when orally administered to a subject, may produce a plasma profile characterized by an AUC₍₁₋₁₂ₕᵣ₎ for hydrocodone from about 3 ng·hr/mL/mg to about 20 ng·hr/mL/mg, from about 7.5 ng·hr/mL/mg to about 15.0 ng·hr/mL/mg, or from about 8 ng·hr/mL/mg to about 12.5 ng·hr/mL/mg. In other embodiments, the pharmaceutical composition, when orally administered to a subject, may produce a plasma profile characterized by an AUC₍₁₋₁₂ₕᵣ₎ for hydrocodone from about 3.0, 4.0, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 ng·hr/mL/mg.

In one embodiment, the pharmaceutical composition, when orally administered to a subject, may produce a plasma profile characterized by an AUC₍₁₂₋₃₆ₕᵣ₎ for hydrocodone from about 2 ng·hr/mL/mg to about 10 ng·hr/mL/mg, from about 4 ng·hr/mL/mg to about 8 ng·hr/mL/mg, and from about 6 ng·hr/mL/mg to about 7 ng·hr/mL/mg. In other embodiments, the pharmaceutical composition, when orally administered to a subject, may produce a plasma profile characterized by an AUC₍₁₂₋₃₆ₕᵣ₎ for hydrocodone from about 2.0, 2.5, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In one embodiment, the pharmaceutical composition, when orally administered to a subject, may produce a plasma profile characterized by an AUC₍₈₋₁₂ₕᵣ₎ for hydrocodone from about 1 ng·hr/mL/mg to about 6 ng·hr/mL/mg, from about 2 ng·hr/mL/mg to about 5 ng·hr/mL/mg, or from about 3 ng·hr/mL/mg to about 4 ng·hr/mL/mg. In other embodiments, the pharmaceutical composition, when orally administered to a subject, may produce a plasma profile characterized by an AUC8-12 hr for hydrocodone from about 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject, may produce a plasma profile characterized by an AUC₍₈₋₁₀ₕᵣ₎ for hydrocodone from about 0.5 ng·hr/mL/mg to about 6 ng·hr/mL/mg, from about 1 ng·hr/mL/mg to about 5 ng·hr/mL/mg, or from about 1.5 ng·hr/mL/mg to about 4 ng·hr/mL/mg. In other embodiments, the pharmaceutical composition, when orally administered to a subject, may produce a plasma profile characterized by an AUC₍₈₋₁₀ₕᵣ₎ for hydrocodone from about 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject in a fasted state, may produce a plasma profile characterized by an AUC₍₈₋₁₀ₕᵣ₎ hydrocodone from about 0.5 ng·hr/mL/mg to about 6 ng·hr/mL/mg, from about 1 ng·hr/mL/mg to about 5 ng·hr/mL/mg, or from about 1.5 ng·hr/mL/mg to about 4 ng·hr/mL/mg. In other embodiments, the pharmaceutical composition, when orally administered to a subject, may produce a plasma profile characterized by an AUC₍₈₋₁₀ₕᵣ₎ for hydrocodone from about 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject in a fed state (high fat), may produce a plasma profile characterized by an AUC₍₈₋₁₀ₕᵣ₎ for hydrocodone from about 0.5 ng·hr/mL/mg to about 6 ng·hr/mL/mg, from about 1 ng·hr/mL/mg to about 5 ng·hr/mL/mg, or from about 1.5 ng·hr/mL/mg to about 4 ng·hr/mL/mg. In other embodiments, the pharmaceutical composition, when orally administered to a subject, may produce a plasma profile characterized by an AUC₍₈₋₁₀ₕᵣ₎ for hydrocodone from about 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject in a fed state (low fat), may produce a plasma profile characterized by an AUC₍₈₋₁₀ₕᵣ₎ for hydrocodone from about 0.5 ng·hr/mL/mg to about 6 ng·hr/mL/mg, from about 1 ng·hr/mL/mg to about 5 ng·hr/mL/mg, or from about 1.5 ng·hr/mL/mg to about 4 ng·hr/mL/mg. In other embodiments, the pharmaceutical composition, when orally administered to a subject, may produce a plasma profile characterized by an AUC₍₈₋₁₀ₕᵣ₎ for hydrocodone from about 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject, may produce a plasma profile characterized by an AUC₍₁₀₋₁₂ₕᵣ₎ for hydrocodone from about 0.25 ng·hr/mL/mg to about 5 ng·hr/mL/mg, from about 0.75 ng·hr/mL/mg to about 4.5 ng·hr/mL/mg, or from about 1.0 ng·hr/mL/mg to about 4 ng·hr/mL/mg. In other embodiments, the pharmaceutical composition, when orally administered to a subject, may produce a plasma profile characterized by an AUC₍₁₀₋₁₂ₕᵣ₎ for hydrocodone from about 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject in a fasted state, may produce a plasma profile characterized by an AUC₍₁₀₋₁₂ₕᵣ₎ for hydrocodone from about 0.25 ng·hr/mL/mg to about 5 ng·hr/mL/mg, from about 0.75 ng·hr/mL/mg to about 4.5 ng·hr/mL/mg, or from about 1.0 ng·hr/mL/mg to about 4 ng·hr/mL/mg. In other embodiments, the pharmaceutical composition, when orally administered to a subject, may produce a plasma profile characterized by an AUC₍₁₀₋₁₂ₕᵣ₎ for hydrocodone from about 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject in a fed state (low fat), may produce a plasma profile characterized by an AUC₍₁₀₋₁₂ₕᵣ₎ hydrocodone from about 0.25 ng·hr/mL/mg to about 5 ng·hr/mL/mg, from about 0.75 ng·hr/mL/mg to about 4.5 ng·hr/mL/mg, or from about 1.0 ng·hr/mL/mg to about 4 ng·hr/mL/mg. In other embodiments, the pharmaceutical composition, when orally administered to a subject, may produce a plasma profile characterized by an AUC₍₁₀₋₁₂ₕᵣ₎ hydrocodone from about 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject in a fed state (high fat), may produce a plasma profile characterized by an AUC₍₁₀₋₁₂ₕᵣ₎ for hydrocodone from about 0.25 ng·hr/mL/mg to about 5 ng·hr/mL/mg, from about 0.75 ng·hr/mL/mg to about 4.5 ng·hr/mL/mg, or from about 1.0 ng·hr/mL/mg to about 4 ng·hr/mL/mg. In other embodiments, the pharmaceutical composition, when orally administered to a subject, may produce a plasma profile characterized by an AUC₍₁₀₋₁₂ₕᵣ₎ hydrocodone from about 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3.0, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4.0, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5.0, 5.05, 5.1, 5.15, 5.2, 5.25, 5.3, 5.35, 5.4, 5.45, 5.5, 5.55, 5.6, 5.65, 5.7, or 5.75 ng·hr/mL/mg.

In one embodiment, the pharmaceutical composition, when orally administered to a subject, may produce a plasma profile characterized by an AUC_{Tmax+2SD-36hr} for hydrocodone from about 5 ng·hr/mL/mg to about 25 ng·hr/mL/mg, from about 10 ng·hr/mL/mg to about 20 ng·hr/mL/mg, or from about 13 ng·hr/mL/mg to about 17 ng·hr/mL/mg. In other embodiments, the pharmaceutical composition, when orally administered to a subject, may produce a plasma profile characterized by an AUC₍₃₋₃₆ₕᵣ₎ or hydrocodone from about 5.0, 5.25, 5.50, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, 14.0, 14.25, 14.5, 14.75, 15.0, 15.25, 15.5, 15.75, 16.0, 16.25, 16.50, 16.75, 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, or 25.0 ng·hr/mL/mg.

In one embodiment, the pharmaceutical composition, when orally administered to a subject, may produce a plasma profile characterized by an AUC_{(2.44-36hr)} for hydrocodone from about 5 ng·hr/mL/mg to about 25 ng·hr/mL/mg, from about 10 ng·hr/mL/mg to about 20 ng·hr/mL/mg, or from about 13 ng·hr/mL/mg to about 17 ng·hr/mL/mg. In other embodiments, the pharmaceutical composition, when orally administered to a subject, may produce a plasma profile characterized by an AUC_{(2.44-36hr)} for hydrocodone from about 5.0, 5.25, 5.50, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, 14.0, 14.25, 14.5, 14.75, 15.0, 15.25, 15.5, 15.75, 16.0, 16.25, 16.50, 16.75, 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, or 25.0 ng·hr/mL/mg.

In one embodiment, the pharmaceutical composition, when orally administered to a subject, may produce a plasma profile characterized by an AUC₍₂₋₄₈ₕᵣ₎ for hydrocodone from about 5 ng·hr/mL/mg to about 40 ng·hr/mL/mg, from about 10 ng·hr/mL/mg to about 35 ng·hr/mL/mg, from about 15 ng·hr/mL/mg to about 30 ng·hr/mL/mg, or from about 17.5 ng·hr/mL/mg to about 27 ng·hr/mL/mg. In other embodiments, the pharmaceutical composition, when orally administered to a subject, may produce a plasma profile characterized by an AUC₍₂₋₄₈ₕᵣ₎ for hydrocodone from about 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, 25.0, 25.25, 25.5, 25.75, 26.0, 26.25, 26.5, 26.75, 27.0, 27.25, 27.5, 28.75, 29.0, 29.25, 29.5, 29.75, 30 ng·hr/mL/mg.

In one embodiment, the pharmaceutical composition, when orally administered to a subject in a fasted state, may produce a plasma profile characterized by an AUC₍₂₋₄₈ₕᵣ₎ for hydrocodone from about 5 ng·hr/mL/mg to about 40 ng·hr/mL/mg, from about 10 ng·hr/mL/mg to about 35 ng·hr/mL/mg, from about 15 ng·hr/mL/mg to about 30 ng·hr/mL/mg, or from about 17.5 ng·hr/mL/mg to about 27 ng·hr/mL/mg. In other embodiments, the pharmaceutical composition, when orally administered to a subject, may produce a plasma profile characterized by an AUC₍₂₋₄₈ₕᵣ₎ for hydrocodone from about 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, 25.0, 25.25, 25.5, 25.75, 26.0, 26.25, 26.5, 26.75, 27.0, 27.25, 27.5, 28.75, 29.0, 29.25, 29.5, 29.75, 30 ng·hr/mL/mg.

In one embodiment, the pharmaceutical composition, when orally administered to a subject in a fed state (high fat), may produce a plasma profile characterized by an AUC₍₂₋₄₈ₕᵣ₎ for hydrocodone from about 5 ng·hr/mL/mg to about 40 ng·hr/mL/mg, from about 10 ng·hr/mL/mg to about 35 ng·hr/mL/mg, from about 15 ng·hr/mL/mg to about 30 ng·hr/mL/mg, or from about 17.5 ng·hr/mL/mg to about 27 ng·hr/mL/mg. In other embodiments, the pharmaceutical composition, when orally administered to a subject, may produce a plasma profile characterized by an AUC₍₂₋₄₈ₕᵣ₎ for hydrocodone from about 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, 25.0, 25.25, 25.5, 25.75, 26.0, 26.25, 26.5, 26.75, 27.0, 27.25, 27.5, 28.75, 29.0, 29.25, 29.5, 29.75, 30 ng·hr/mL/mg.

In one embodiment, the pharmaceutical composition, when orally administered to a subject in a fed state (low fat), may produce a plasma profile characterized by an AUC₍₂₋₄₈ₕᵣ₎ for hydrocodone from about 5 ng·hr/mL/mg to about 40 ng·hr/mL/mg, from about 10 ng·hr/mL/mg to about 35 ng·hr/mL/mg, from about 15 ng·hr/mL/mg to about 30 ng·hr/mL/mg, or from about 17.5 ng·hr/mL/mg to about 27 ng·hr/mL/mg. In other embodiments, the pharmaceutical composition, when orally administered to a subject, may produce a plasma profile characterized by an AUC₍₂₋₄₈ₕᵣ₎ for hydrocodone from about 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, 25.0, 25.25, 25.5, 25.75, 26.0, 26.25, 26.5, 26.75, 27.0, 27.25, 27.5, 28.75, 29.0, 29.25, 29.5, 29.75, 30 ng·hr/mL/mg.

In one embodiment, the pharmaceutical composition, when orally administered to a subject, may produce a plasma profile characterized by an AUC₀₋₁₂ hr for hydrocodone from about 50% to about 90% of the AUC0-t, from about 55% to about 80% of the AUC0-t, or from about 60% to about 70% of the AUC0-t. In other embodiments, the pharmaceutical composition, when orally administered to a subject, may produce a plasma profile characterized by an AUC₀₋₁₂ hr for hydrocodone that is about 50%, about 53%, about 55%, about 58%, about 60%, about 63%, about 65%, about 68%, about 70%, about 73%, about 75%, about 78%, about 80%, about 83%, about 85%, about 88%, or about 90% of the AUC0-t.

In one embodiment, the pharmaceutical composition, when orally administered to a subject, may produce a plasma profile characterized by an AUC1-12 hr for hydrocodone from about 40% to about 90% of the AUC0-t, from about 55% to about 80% of the AUC0-t, or from about 60% to about 70% of the AUC0-t. In other embodiments, the pharmaceutical composition, when orally administered to a subject, may produce a plasma profile characterized by an AUC1-12 hr for hydrocodone of about 40%, about 43%, about 45%, about 48%, about 50%, about 53%, about 55%, about 58%, about 60%, about 63%, about 65%, about 68%, about 70%, about 73%, about 75%, about 78%, about 80%, about 83%, about 85%, about 88%, or about 90% of the AUC0-t.

In one embodiment, the pharmaceutical composition, when orally administered to a subject, may produce a plasma profile characterized by an AUC12-36 hr for hydrocodone from about 20% to about 50% of the AUC0-t, from about 25% to about 45% of the AUC0-t, or from about 30% to about 40% of the AUC0-t. In other embodiments, the pharmaceutical composition, when orally administered to a subject, may produce a plasma profile characterized by an AUC1-12 hr for hydrocodone of about 20%, about 23%, about 25%, about 28%, about 30%, about 33%, about 35%, about 38%, about 40%, about 43%, about 45%, about 48%, about 50%, or about 53% of the AUC0-t.

In one embodiment, the pharmaceutical composition, when orally administered to a subject, may produce a plasma profile characterized by an AUC₈₋₁₂ hr for hydrocodone from about 5% to about 30% of the AUC0-t, from about 10% to about 25% of the AUC0-t, or from about 15% to about 20% of the AUC0-t. In other embodiments, the pharmaceutical composition, when orally administered to a subject, may produce a plasma profile characterized by an AUC₁₋₁₂ hr for hydrocodone of about 5%, about 8%, about 10%, about 13%, about 15%, about 18%, about 20%, about 23%, about 25%, about 28%, or about 30% of the AUC0-t.

In an alternate embodiment, the pharmaceutical composition, when orally administered to a subject, may provide a mean half-life of hydrocodone that ranges from about 3.5 hours to about 5.5 hours, or from about 4 hours to about 5 hours. In various embodiments, the mean half-life of hydrocodone may be about 3.8, 4.0, 4.2, 4.4, 4.6, 4.8, 5.0, or 5.2 hours.

In yet another embodiment, the pharmaceutical composition, when orally administered to a subject, produces a plasma profile characterized by an abuse quotient for hydrocodone from about 3 to about 5. In other embodiments, the abuse quotient for hydrocodone may be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

Moreover, upon oral administration, the pharmaceutical composition disclosed herein may maintain a therapeutic plasma concentration of acetaminophen of at least about 2 mg/mL from about 1 hour to about 6 hours after administration. In another embodiment, the pharmaceutical composition may maintain a therapeutic plasma concentration of acetaminophen of at least about 2 mg/mL from about 0.75 hour to about 6.5 hours after administration. In yet another embodiment, the composition may maintain a plasma concentration of acetaminophen of at least about 1 mg/mL from about 0.5 hour to about 12 hours after administration.

In another embodiment, the pharmaceutical composition, when orally administered to a subject, may produce a plasma profile characterized by a mean Cₘₐₓ for acetaminophen from about 4.0 ng/mL/mg to about 11.0 ng/mL/mg. In other embodiments, the mean Cₘₐₓ for acetaminophen may be from about 4.0, 4.5, 5.0, 5.5, 6.0, 6.5, 7.0, 7.5, 8.0, 8.5, 9.0, 9.5, 10.0, 10.5, or 11.0 ng/mL/mg. Moreover, the mean Cₘₐₓ for acetaminophen at steady state may range from about 6.0 ng/mL/mg to about 9.0 ng/mL/mg, from about 6.5 ng/mL/mg to about 8.5 ng/mL/mg, or from about 7.0 ng/mL/mg to about 8.0 ng/mL/mg.

In a further embodiment, the pharmaceutical composition, when orally administered to a subject, surprisingly may produce a blood plasma concentration profile characterized by a biphasic increase in blood plasma concentrations of acetaminophen. The biphasic absorption of acetaminophen may characterized by an initial rapid absorption resulting in first peak in plasma concentrations between about 0.5 hour and 2 hours, which contributes to the early onset on action, and a second peak in plasma concentrations between about 3 hours and 7 hours after administration of the composition, which contributes to the duration or maintenance of analgesia. In some instances, the second peak may correspond to the overall Cₘₐₓ of the composition. The biphasic increase in blood plasma concentrations of acetaminophen is characterized by a plasma concentration-time profile for acetaminophen in which the slope of a line drawn between 0 hour and 2 hour is greater than the slope of a line drawn between about 2 hours and 5 hours. See FIG. 24.

This biphasic increase in acetaminophen levels resulting from the composition has several benefits. For example, the initial rapid rise in plasma levels produce quick onset of analgesia and the slower absorption provides maintenance of analgesia for an extended period of time.

In a further embodiment, the pharmaceutical composition, when orally administered to a subject, may produce a plasma profile characterized by a mean AUC for acetaminophen from about 35.0 ng·hr/mL/mg to about 80.0 ng·hr/mL/mg. In a further embodiment, the mean AUC for acetaminophen may range from about 35.0 ng·hr/mL/mg to about 60.0 ng·hr/mL/mg. In other embodiments, the mean AUC for acetaminophen may be about 35.0, 40.0, 45.0, 50.0, 55.0, 60.0, 65.0, 70.0, 75.0, or 80.0 ng·hr/mL/mg. Additionally, the mean AUC for acetaminophen at steady state may range from about 40.0 ng·hr/mL/mg to about 50.0 ng·hr/mL/mg, from about 35.0 ng·hr/mL/mg to about 45.0 ng·hr/mL/mg, or from about 37.0 ng·hr/mL/mg to about 42.0 ng·hr/mL/mg.

In yet another embodiment, the pharmaceutical composition when orally administered to a subject, may produce a plasma profile characterized by a median Tₘₐₓ for acetaminophen from about 0.5 hours to about 6.0 hours. In another embodiment, the median Tₘₐₓ for acetaminophen may be from about 1.0 hour to about 5.0 hours. In a further embodiment, the median Tₘₐₓ for acetaminophen may range from about 0.5 hour to about 4.0 hours. In still another embodiment, the median Tₘₐₓ for acetaminophen may range from about 0.75 to about 1.5 hours. In other embodiments, the median Tₘₐₓ may be about 0.5, 0.6, 0.7, 0.8, 0.9, 1.0, 1.1, 1.2, 1.3, 1.4, 1.5, 1.6, 1.7 1.8, 1.9, 2.0, 2.2, 2.4, 2.6, 2.8, 3.0, 3.2, 3.4, 3.6, 3.8, 4.0, 4.2, 4.4, 4.6, 4.8, or 5.0 hours. Moreover, the median Tₘₐₓ for acetaminophen at steady state may range from about 0.5 hour to about 1.0 hour, or from about 0.5 hour to about 0.75 hour.

In a further embodiment, the pharmaceutical composition, when orally administered to a subject, may produce a plasma profile characterized by a median t_{lag} for acetaminophen from about 0 hour to about 0.5 hour. In an alternate embodiment, the median t_{lag} for acetaminophen may be from about 0 hour to about 0.25 hour. In one embodiment, the median t_{lag} for acetaminophen may be 0 hour. In another embodiment, the median t_{lag} for acetaminophen may be 0.25 hour.

In one embodiment, the pharmaceutical composition, when orally administered to a subject, may produce a plasma profile characterized by various partial AUCs for acetaminophen. The partial AUCs for acetaminophen are calculated as described above for hydrocodone. The pharmaceutical composition, when orally administered to a subject, may produce a plasma profile characterized by an AUC₀₋₁ₕᵣ for acetaminophen from about 1.25 ng·hr/mL/mg to about 3.25 ng·hr/mL/mg, from about 1.60 ng·hr/mL/mg to about 2.0 ng·hr/mL/mg, or from about 2.0 ng·hr/mL/mg to about 2.75 ng·hr/mL/mg. In another embodiment, the AUC₀₋₁ₕᵣ for acetaminophen may be about 1.25, 1.30, 1.40, 1.50, 1.55, 1.60, 1.65, 1.70, 1.75, 1.80, 1.85, 1.90, 1.95, 2.0, 2.05, 2.10, 2.15, 2.20, 2.25, 2.30, 2.35, 2.40, 2.45, 2.50, 2.55, 2.60, 2.65, 2.70, 2.75, 2.80, 2.85, or 2.90 or ng·hr/mL/mg.

In an additional embodiment, the pharmaceutical composition, when orally administered to a subject, may produce a plasma profile characterized by an AUC₀₋₂ₕᵣ for acetaminophen from about 1 ng·hr/mL/mg to about 40 ng·hr/mL/mg, from about 2.25 ng·hr/mL/mg to about 30 ng·hr/mL/mg, from about 4.0 ng·hr/mL/mg to about 27.75 ng·hr/mL/mg, from about 4.25 ng·hr/mL/mg to about 8.75 ng·hr/mL/mg, from about 5.50 ng·hr/mL/mg to about 6.0 ng·hr/mL/mg, or from about 6.0 ng·hr/mL/mg to about 7.25 ng·hr/mL/mg. In another embodiment, the AUC₀₋₂ₕᵣ for acetaminophen may be about 4.0, 4.25, 4.5, 4.75, 5.0, 5.25, 5.50, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, 14.0, 14.25, 14.5, 14.75, 15.0, 15.25, 15.5, 15.75, 16.0, 16.25, 16.50, 16.75, 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, or 25.0 ng·hr/mL/mg. In still another embodiment, the AUC₀₋₂ₕᵣ for acetaminophen may be about 4.25, 4.5, 4.75, 5.0, 5.25, 5.5, 5.75, 6.0, 6.25, 6.5, 6.75, 7.0, 7.25, 7.50, 7.75 or 8.0 ng·hr/mL/mg. In yet another embodiment, the AUC₀₋₂ₕᵣ for acetaminophen may be about 5.0, 5.25, 5.50, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, 14.0, 14.25, 14.5, 14.75, 15.0, 15.25, 15.5, 15.75, 16.0, 16.25, 16.50, 16.75, 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25 ng·hr/mL/mg.

In an additional embodiment, the pharmaceutical composition, when orally administered to a subject in a fasted state, may produce a plasma profile characterized by an AUC₀₋₂ₕᵣ for acetaminophen from about 1 ng·hr/mL/mg to about 40 ng·hr/mL/mg, from about 2.25 ng·hr/mL/mg to about 30 ng·hr/mL/mg, from about 4.0 ng·hr/mL/mg to about 27.75 ng·hr/mL/mg, from about 7.5 ng·hr/mL/mg to about 25 ng·hr/mL/mg, from about 10 ng·hr/mL/mg to about 22.5 ng·hr/mL/mg, or from about 12 ng·hr/mL/mg to about 22.5 ng·hr/mL/mg. In another embodiment, the AUC₀₋₂ₕᵣ for acetaminophen may be about 10.0, 10.25, 10.5, 10.75, 11.0, 11.25, 11.5, 11.75, 12.0, 12.25, 12.5, 12.75, 13.0, 13.25, 13.5, 13.75, 14.0, 14.25, 14.5, 14.75, 15.0, 15.25, 15.5, 15.75, 16.0, 16.25, 16.50, 16.75, 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, or 25.0 ng·hr/mL/mg.

In an additional embodiment, the pharmaceutical composition, when orally administered to a subject in a fed state (high fat), may produce a plasma profile characterized by an AUC₀₋₂ₕᵣ for acetaminophen from about 1 ng·hr/mL/mg to about 40 ng·hr/mL/mg, from about 2.25 ng·hr/mL/mg to about 30 ng·hr/mL/mg, from about 4.0 ng·hr/mL/mg to about 27.75 ng·hr/mL/mg, from about 5 ng·hr/mL/mg to about 25 ng·hr/mL/mg, from about 7.5 ng·hr/mL/mg to about 20 ng·hr/mL/mg, or from about 7.5 ng·hr/mL/mg to about 17.5 ng·hr/mL/mg. In another embodiment, the AUC₀₋₂ₕᵣ for acetaminophen may be about 4.0, 4.25, 4.5, 4.75, 5.0, 5.25, 5.50, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, 14.0, 14.25, 14.5, 14.75, 15.0, 15.25, 15.5, 15.75, 16.0, 16.25, 16.50, 16.75, 17.0, 17.25, 17.5, 17.75, 18.0, 18.25, 18.5, 18.75, 19.0, 19.25, 19.5, 19.75, 20.0, 20.25, 20.5, 20.75, 21.0, 21.25, 21.5, 21.75, 22.0, 22.25, 22.5, 22.75, 23.0, 23.25, 23.5, 23.75, 24.0, 24.25, 24.5, 24.75, or 25.0 ng·hr/mL/mg.

In an additional embodiment, the pharmaceutical composition, when orally administered to a subject in a fed state (low fat), may produce a plasma profile characterized by an AUC₀₋₂ₕᵣ for acetaminophen from about 1 ng·hr/mL/mg to about 40 ng·hr/mL/mg, from about 2 ng·hr/mL/mg to about 30 ng·hr/mL/mg, from about 3.0 ng·hr/mL/mg to about 25 ng·hr/mL/mg, from about 4 ng·hr/mL/mg to about 20 ng·hr/mL/mg, from about 4.5 ng·hr/mL/mg to about 15 ng·hr/mL/mg, or from about 5 ng·hr/mL/mg to about 10 ng·hr/mL/mg. In another embodiment, the AUC₀₋₂ₕᵣ for acetaminophen may be about 4.0, 4.25, 4.5, 4.75, 5.0, 5.25, 5.50, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, 10.25, 10.5, 10.75, 11.0, 11.25, 11.5, 11.75, 12.0, 12.25, 12.5, 12.75, 13.0, 13.25, 13.5, 13.75, 14.0, 14.25, 14.5, 14.75, 15.0, 15.25, 15.5, 15.75, 16.0, 16.25, 16.50, 16.75, 17.0, 17.25, 17.5, 17.75, 18.0, 18.25, 18.5, 18.75, 19.0, 19.25, 19.5, 19.75, or 20.0 ng·hr/mL/mg.

In a further embodiment, the pharmaceutical composition, when orally administered to a subject, may produce a plasma profile characterized by an AUC₀₋₄ₕᵣ for acetaminophen from about 10.0 ng·hr/mL/mg to about 20.0 ng·hr/mL/mg, from about 13.0 ng·hr/mL/mg to about 14.5 ng·hr/mL/mg, or from about 14.5 ng·hr/mL/mg to about 16.5 ng·hr/mL/mg. In another embodiment, the AUC₀₋₄ₕᵣ for acetaminophen may be about 10.0, 11.0, 12.0, 13.0, 13.5, 14.0, 14.5, 15.0, 15.5, 16.0, 16.5, or 17.0 ng·hr/mL/mg.

In yet another embodiment, the pharmaceutical composition, when orally administered to a subject, may produce a plasma profile characterized by an AUC_{Tmax-t} for acetaminophen from about 20.0 ng·hr/mL/mg to about 40.0 ng·hr/mL/mg, from about 23.5 ng·hr/mL/mg to about 36.0 ng·hr/mL/mg, or from about 29.0 ng·hr/mL/mg to about 31.0 ng·hr/mL/mg. In another embodiment, the AUC_{Tmax-t} for acetaminophen may be about 20.0, 21.0, 22.0, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0., 33.5, 34.0, 34.5, 35.0, 35.5 or 36.0 ng·hr/mL/mg.

In yet another embodiment, the pharmaceutical composition, when orally administered to a subject, may produce a plasma profile characterized by an AUC_{(0-(Tmax of IR product+2SD))} for acetaminophen after a single dose from about 3.0 ng·hr/mL/mg to about 13.0 ng·hr/mL/mg, from about 4.0 ng·hr/mL/mg to about 11.6 ng·hr/mL/mg, or from about 5.0 ng·hr/mL/mg to about 10.0 ng·hr/mL/mg. In still another embodiment, the pharmaceutical composition, when orally administered to a subject, may produce a plasma profile characterized by an AUC(0−(Tmax of IR product+2SD)) for acetaminophen after a single dose from about 5.0 ng·hr/mL/mg to about 13.0 ng·hr/mL/mg, from about 7.2 ng·hr/mL/mg to about 11.6 ng·hr/mL/mg, or from about 5.0 ng·hr/mL/mg to about 10.0 ng·hr/mL/mg. In another embodiment, the AUC(0−(Tmax of IR product+2SD)) for acetaminophen may be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13 ng·hr/mL/mg.

In still another embodiment, the pharmaceutical composition, when orally administered to a subject, may produce a plasma profile characterized by an AUC_{(0-1.7)} for acetaminophen after a single dose from about 5.0 ng·hr/mL/mg to about 13.0 ng·hr/mL/mg, from about 7.2 ng·hr/mL/mg to about 11.6 ng·hr/mL/mg, or from about 8.5 ng·hr/mL/mg to about 10.0 ng·hr/mL/mg. In another embodiment, the AUC_{(0-1.7)} for acetaminophen may be about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13 ng·ng·hr/mL/mg.

In yet a further embodiment, the pharmaceutical composition, when orally administered to a subject, may produce a plasma profile characterized by an AUC_{(1.7-48)} for acetaminophen after a single dose from about 25.0 ng·hr/mL/mg to about 75.0 ng·hr/mL/mg, from about 31.5 ng·hr/mL/mg to about 55.0 ng·hr/mL/mg, or from about 35.0 ng·hr/mL/mg to about 50.0 ng·hr/mL/mg. In another embodiment, the AUC_{(1.7-48)} for acetaminophen may be about 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0 ng·hr/mL/mg.

In yet a further embodiment, the pharmaceutical composition, when orally administered to a subject, may produce a plasma profile characterized by an AUC₍₂₋₄₈₎ for acetaminophen after a single dose from about 25.0 ng·hr/mL/mg to about 90.0 ng·hr/mL/mg, from about 30 ng·hr/mL/mg to about 80.0 ng·hr/mL/mg, or from about 45.0 ng·hr/mL/mg to about 85.0 ng·hr/mL/mg. In another embodiment, the AUC₍₂₋₄₈₎ for acetaminophen may be about 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0 ng·hr/mL/mg. In still another embodiment, the AUC₍₂₋₄₈₎ for acetaminophen may be about 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, 75.0, 75.5, 76.0, 76.5, 77.0, 77.5, 78.0, 78.5, 79.0, 79.5, 80.0 ng, 80.5, 81.0, 81.5, 82.0, 82.5, 83.0, 83.5, 84.0, 84.5, or 85.0·hr/mL/mg.

In yet a further embodiment, the pharmaceutical composition, when orally administered to a subject in a fasted state, may produce a plasma profile characterized by an AUC₍₂₋₄₈₎ for acetaminophen after a single dose from about 25.0 ng·hr/mL/mg to about 90.0 ng·hr/mL/mg, from about 30 ng·hr/mL/mg to about 80.0 ng·hr/mL/mg, or from about 45.0 ng·hr/mL/mg to about 85.0 ng·hr/mL/mg. In another embodiment, the AUC₍₂₋₄₈₎ for acetaminophen may be about 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0 ng·hr/mL/mg. In still another embodiment, the AUC₍₂₋₄₈₎ for acetaminophen may be about 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, 75.0, 75.5, 76.0, 76.5, 77.0, 77.5, 78.0, 78.5, 79.0, 79.5, 80.0 ng, 80.5, 81.0, 81.5, 82.0, 82.5, 83.0, 83.5, 84.0, 84.5, or 85.0·hr/mL/mg.

In yet a further embodiment, the pharmaceutical composition, when orally administered to a subject in a fed state (high fat), may produce a plasma profile characterized by an AUC₍₂₋₄₈₎ for acetaminophen after a single dose from about 25.0 ng·hr/mL/mg to about 90.0 ng·hr/mL/mg, from about 30 ng·hr/mL/mg to about 80.0 ng·hr/mL/mg, or from about 45.0 ng·hr/mL/mg to about 85.0 ng·hr/mL/mg. In another embodiment, the AUC₍₂₋₄₈₎ for acetaminophen may be about 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0 ng·hr/mL/mg. In still another embodiment, the AUC₍₂₋₄₈₎ for acetaminophen may be about 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, 75.0, 75.5, 76.0, 76.5, 77.0, 77.5, 78.0, 78.5, 79.0, 79.5, 80.0 ng, 80.5, 81.0, 81.5, 82.0, 82.5, 83.0, 83.5, 84.0, 84.5, or 85.0·hr/mL/mg.

In yet a further embodiment, the pharmaceutical composition, when orally administered to a subject in a fed state (low fat), may produce a plasma profile characterized by an AUC₍₂₋₄₈₎ for acetaminophen after a single dose from about 25.0 ng·hr/mL/mg to about 90.0 ng·hr/mL/mg, from about 30 ng·hr/mL/mg to about 80.0 ng·hr/mL/mg, or from about 45.0 ng·hr/mL/mg to about 85.0 ng·hr/mL/mg. In another embodiment, the AUC₍₂₋₄₈₎ for acetaminophen may be about 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0 ng·hr/mL/mg. In still another embodiment, the AUC₍₂₋₄₈₎ for acetaminophen may be about 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, 75.0, 75.5, 76.0, 76.5, 77.0, 77.5, 78.0, 78.5, 79.0, 79.5, 80.0 ng, 80.5, 81.0, 81.5, 82.0, 82.5, 83.0, 83.5, 84.0, 84.5, or 85.0·hr/mL/mg.

In another embodiment, the pharmaceutical composition, when orally administered to a subject, may produce a plasma profile characterized by an AUC(0-1.27) for acetaminophen after a single dose from about 3.0 ng·hr/mL/mg to about 21.0 ng·hr/mL/mg, from about 4.0 ng·hr/mL/mg to about 18.0 ng·hr/mL/mg, from about 10.0 ng·hr/mL/mg to about 16.0 ng·hr/mL/mg, or from about 5.0 ng·hr/mL/mg to about 15.0 ng·hr/mL/mg. In yet another embodiment, the pharmaceutical composition, when orally administered to a subject, may produce a plasma profile characterized by an AUC(0-1.27) for acetaminophen after a single dose from about 7.0 ng·hr/mL/mg to about 13.0 ng·hr/mL/mg, from about 9.0 ng·hr/mL/mg to about 11.6 ng·hr/mL/mg, or from about 12.0 ng·hr/mL/mg to about 10.0 ng·hr/mL/mg. In still another embodiment, the AUC(0-1.27) for acetaminophen may be about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, or 21.0 ng·hr/mL/mg.

In another embodiment, the AUC_{(1.27-36)} for acetaminophen may be about-15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, 55.0, 55.5, 56.0, 56.5, 57.0, 57.5, 58.0, 58.5, 59.0, 59.5, 60.0, 60.5, 61.0, 61.5, 62.0, 62.5, 63.0, 63.5, 64.0, 64.5, 65.0, 65.5, 66.0, 66.5, 67.0, 67.5, 68.0, 68.5, 69.0, 69.5, 70.0, 70.5, 71.0, 71.5, 72.0, 72.5, 73.0, 73.5, 74.0, 74.5, or 75.0 ng·hr/mL/mg.

In one embodiment, the pharmaceutical composition, when orally administered to a subject, may produce a plasma profile characterized by an AUC₀₋₁₂ₕᵣ for acetaminophen from about 20.0 ng·hr/mL/mg to about 60.0 ng·hr/mL/mg, from about 30 ng·hr/mL/mg to about 50 ng·hr/mL/mg, from about 35 to about 45 ng·hr/mL/mg, or from about 37.5 ng·hr/mL/mg to about 42.5 ng·hr/mL/mg. In another embodiment, the pharmaceutical composition, when orally administered to a subject, may produce a plasma profile characterized by an AUC₀₋₁₂ₕᵣ for acetaminophen from about 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, 50.0, 50.5, 51.0, 51.5, 52.0, 52.5, 53.0, 53.5, 54.0, 54.5, or 55.0. In a further embodiment, at AUC₀₋₁₂ₕᵣ between about 70%-95%, about 75%-92%, or about 77%-90% of the acetaminophen has been cleared. In still another embodiment, about 80% of the acetaminophen has been cleared.

In another embodiment, the pharmaceutical composition, when orally administered to a subject, may produce a plasma profile characterized by an AUC₁₋₁₂ₕᵣ for acetaminophen from about 15.0 ng·hr/mL/mg to about 55.0 ng·hr/mL/mg, from about 25.0 ng·hr/mL/mg to about 45.0 ng·hr/mL/mg, or from about 30.0 to about 40.0 ng·hr/mL/mg. In another embodiment, the pharmaceutical composition, when orally administered to a subject, may produce a plasma profile characterized by an AUC₁₋₁₂ₕᵣ for acetaminophen from about 15, 16, 17, 18, 19, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, or 50.0 ng·hr/mL/mg.

In yet another embodiment, the pharmaceutical composition, when orally administered to a subject, may produce a plasma profile characterized by an AUC₁₂₋₃₆ₕᵣ for acetaminophen from about 5.0 ng·hr/mL/mg to about 25.0 ng·hr/mL/mg, from about 7.5 ng·hr/mL/mg to about 20.0 ng·hr/mL/mg, or from about 10.0 ng·hr/mL/mg to about 15.0. In other embodiments, the pharmaceutical composition, when orally administered to a subject, may produce a plasma profile characterized by an AUC₁₂₋₃₆ₕᵣ for acetaminophen from about 5.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, or 15.0 ng·hr/mL/mg.

In another embodiment, the pharmaceutical composition, when orally administered to a subject, may produce a plasma profile characterized by an AUC₈₋₁₂ₕᵣ for acetaminophen from about 1.5 ng·hr/mL/mg to about 15.5 ng·hr/mL/mg, from about 2 ng·hr/mL/mg to about 12.25 ng·hr/mL/mg, from about 3.5 ng·hr/mL/mg to about 10 ng·hr/mL/mg, or from about 4.5 ng·hr/mL/mg to about 6.5 ng·hr/mL/mg. In other embodiments, the pharmaceutical composition, when orally administered to a subject, may produce a plasma profile characterized by an AUC₈₋₁₂ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject, may produce a plasma profile characterized by an AUC₈₋₁₀ₕᵣ for acetaminophen from about 1.5 ng·hr/mL/mg to about 20 ng·hr/mL/mg, from about 2 ng·hr/mL/mg to about 15 ng·hr/mL/mg, from about 3 ng·hr/mL/mg to about 12.5 ng·hr/mL/mg, or from about 4 ng·hr/mL/mg to about 10 ng·hr/mL/mg. In other embodiments, the pharmaceutical composition, when orally administered to a subject, may produce a plasma profile characterized by an AUC₈₋₁₀ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject in a fasted state, may produce a plasma profile characterized by an AUC₈₋₁₀ₕᵣ for acetaminophen from about 1.5 ng·hr/mL/mg to about 20 ng·hr/mL/mg, from about 2 ng·hr/mL/mg to about 15 ng·hr/mL/mg, from about 3 ng·hr/mL/mg to about 12.5 ng·hr/mL/mg, or from about 4 ng·hr/mL/mg to about 10 ng·hr/mL/mg. In other embodiments, the pharmaceutical composition, when orally administered to a subject, may produce a plasma profile characterized by an AUC₈₋₁₀ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject in a fed state (high fat), may produce a plasma profile characterized by an AUC₈₋₁₀ₕᵣ for acetaminophen from about 1.5 ng·hr/mL/mg to about 20 ng·hr/mL/mg, from about 2 ng·hr/mL/mg to about 15 ng·hr/mL/mg, from about 3 ng·hr/mL/mg to about 12.5 ng·hr/mL/mg, or from about 4 ng·hr/mL/mg to about 10 ng·hr/mL/mg. In other embodiments, the pharmaceutical composition, when orally administered to a subject, may produce a plasma profile characterized by an AUC₈₋₁₀ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject in a fed state (low fat), may produce a plasma profile characterized by an AUC₈₋₁₀ₕᵣ for acetaminophen from about 1.5 ng·hr/mL/mg to about 20 ng·hr/mL/mg, from about 2 ng·hr/mL/mg to about 15 ng·hr/mL/mg, from about 3 ng·hr/mL/mg to about 12.5 ng·hr/mL/mg, or from about 4 ng·hr/mL/mg to about 10 ng·hr/mL/mg. In other embodiments, the pharmaceutical composition, when orally administered to a subject, may produce a plasma profile characterized by an AUC₈₋₁₀ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject, may produce a plasma profile characterized by an AUC₁₀₋₁₂ₕᵣ for acetaminophen from about 0.5 ng·hr/mL/mg to about 20 ng·hr/mL/mg, from about 1.0 ng·hr/mL/mg to about 15 ng·hr/mL/mg, from about 1.5 ng·hr/mL/mg to about 12.5 ng·hr/mL/mg, or from about 2 ng·hr/mL/mg to about 10 ng·hr/mL/mg. In other embodiments, the pharmaceutical composition, when orally administered to a subject, may produce a plasma profile characterized by an AUC₁₀₋₁₂ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject in a fasted state, may produce a plasma profile characterized by an AUC₁₀₋₁₂ₕᵣ for acetaminophen from about 0.5 ng·hr/mL/mg to about 20 ng·hr/mL/mg, from about 1.0 ng·hr/mL/mg to about 15 ng·hr/mL/mg, from about 1.5 ng·hr/mL/mg to about 12.5 ng·hr/mL/mg, or from about 2 ng·hr/mL/mg to about 10 ng·hr/mL/mg. In other embodiments, the pharmaceutical composition, when orally administered to a subject, may produce a plasma profile characterized by an AUC₁₀₋₁₂ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject in a fed state (high fat), may produce a plasma profile characterized by an AUC₁₀₋₁₂ₕᵣ for acetaminophen from about 0.5 ng·hr/mL/mg to about 20 ng·hr/mL/mg, from about 1.0 ng·hr/mL/mg to about 15 ng·hr/mL/mg, from about 1.5 ng·hr/mL/mg to about 12.5 ng·hr/mL/mg, or from about 2 ng·hr/mL/mg to about 10 ng·hr/mL/mg. In other embodiments, the pharmaceutical composition, when orally administered to a subject, may produce a plasma profile characterized by an AUC₁₀₋₁₂ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In another embodiment, the pharmaceutical composition, when orally administered to a subject in a fed state (low fat), may produce a plasma profile characterized by an AUC₁₀₋₁₂ₕᵣ for acetaminophen from about 0.5 ng·hr/mL/mg to about 20 ng·hr/mL/mg, from about 1.0 ng·hr/mL/mg to about 15 ng·hr/mL/mg, from about 1.5 ng·hr/mL/mg to about 12.5 ng·hr/mL/mg, or from about 2 ng·hr/mL/mg to about 10 ng·hr/mL/mg. In other embodiments, the pharmaceutical composition, when orally administered to a subject, may produce a plasma profile characterized by an AUC₁₀₋₁₂ₕᵣ for acetaminophen from about 1.5, 1.75, 2.0, 2.25, 2.5, 2.75, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 ng·hr/mL/mg.

In one embodiment, the pharmaceutical composition, when orally administered to a subject, may produce a plasma profile characterized by an AUC₍₀₋₃ₕᵣ₎ for acetaminophen from about 5 ng·hr/mL/mg to about 30 ng·hr/mL/mg, from about 10 ng·hr/mL/mg to about 20 ng·hr/mL/mg, or from about 13 ng·hr/mL/mg to about 17 ng·hr/mL/mg. In other embodiments, the pharmaceutical composition, when orally administered to a subject, may produce a plasma profile characterized by an AUC₍₀₋₃ₕᵣ₎ for acetaminophen from about 5.0, 6.0, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 ng·hr/mL/mg.

In another embodiment, the pharmaceutical composition, when orally administered to a subject, may produce a plasma profile characterized by an AUC₍₃₋₃₆ₕᵣ₎ for acetaminophen from about 20 ng·hr/mL/mg to about 50 ng·hr/mL/mg, from about 20 ng·hr/mL/mg to about 40 ng·hr/mL/mg, or from about 25 ng·hr/mL/mg to about 35 ng·hr/mL/mg. In other embodiments, the pharmaceutical composition, when orally administered to a subject, may produce a plasma profile characterized by an AUC₍₃₋₃₆ₕᵣ₎ for acetaminophen from about 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng·hr/mL/mg.

In one embodiment, the pharmaceutical composition, when orally administered to a subject, may produce a plasma profile characterized by an AUC₀₋₁₂ₕᵣ for acetaminophen from about 50% to about 90% of the AUC₀₋ₜ, from about 55% to about 85% of the AUC₀₋ₜ, or from about 75% to about 85% of the AUC₀₋ₜ. In other embodiments, the pharmaceutical composition, when orally administered to a subject, may produce a plasma profile characterized by an AUC₀₋₁₂ₕᵣ for acetaminophen that is about 50%, 55%, 60%, 65%, 70%, 75%, 76%, 77%, 78%, 79%, 80%, 81%, 82%, 83%, 84% or 85% of the AU C₀₋ₜ.

In one embodiment, the pharmaceutical composition, when orally administered to a subject, may produce a plasma profile characterized by an AUC₁₋₁₂ₕᵣ for acetaminophen from about 40% to about 90% of the AUC₀₋ₜ, from about 55% to about 85% of the AUC₀₋ₜ, or from about 60% to about 75% of the AUC₀₋ₜ. In other embodiments, the pharmaceutical composition, when orally administered to a subject, may produce a plasma profile characterized by an AUC₁₋₁₂ₕᵣ for acetaminophen of about 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, or 80% of the AUC₀₋ₜ.

In one embodiment, the pharmaceutical composition, when orally administered to a subject, may produce a plasma profile characterized by an AUC₁₂₋₃₆ₕᵣ for acetaminophen from about 10% to about 40% of the AUC₀₋ₜ, from about 15% to about 35% of the AUC₀₋ₜ, or from about 20% to about 30% of the AUC₀₋ₜ. In other embodiments, the pharmaceutical composition, when orally administered to a subject, may produce a plasma profile characterized by an AUC₁₂₋₃₆ₕᵣ for acetaminophen of about 10%, 12%, 14%, 16%, 18%, 20%, 21%, 22%, 23%, 24%, 25%, 26%, 27%, 28%, 29%, or 30% of the AUC₀₋ₜ.

In one embodiment, the pharmaceutical composition, when orally administered to a subject, may produce a plasma profile characterized by an AUC₈₋₁₂ₕᵣ for acetaminophen from about 5% to about 30% of the AUC₀₋ₜ, from about 7% to about 25% of the AUC₀₋ₜ, or from about 10% to about 20% of the AUC₀₋ₜ. In other embodiments, the pharmaceutical composition, when orally administered to a subject, may produce a plasma profile characterized by an AUC₈₋₁₂ₕᵣ for acetaminophen of about 5%, 6%, 7%, 8%, 9%, 10%, 11%, 12%, 13%, 14%, 15%, 16%, 17%, 18%, 19%, 20%, 21%, 22%, 23%, 24%, or 25% of the AUC₀₋ₜ.

In an alternate embodiment, the pharmaceutical composition, when orally administered to a subject, may have a mean half-life of acetaminophen that ranges from about 2 hours to about 10 hours, or from about 3 hours to about 6 hours. In another embodiment, the pharmaceutical composition, when orally administered to a subject, may have a mean half-life of acetaminophen that ranges from about 3 hours to about 5 hours. In still another embodiment, the pharmaceutical composition, when orally administered to a subject, may have a mean half-life of acetaminophen that ranges from about 4 hours to about 5 hours. In various embodiments, the mean half-life of acetaminophen may be about 2.5, 3.0, 3.5, 4.0, 4.5, 5.0, 5.5, 6.0, 6.5, 6.0, 7.0, 7.5, or 8 hours. In additional embodiments, the pharmaceutical composition, when orally administered to a subject, has a mean observed half-life of acetaminophen that is more than the mean half-life of commercially available immediate release acetaminophen products.

In another embodiment, upon administration of the pharmaceutical composition to a subject, the composition may provide at least about 4 hours to about 12 hours of drug delivery to the upper gastrointestinal tract, which includes the duodenum, jejunum, and ileum of the small intestine. In another embodiment, the composition may provide at least about 6 hours of drug delivery to the upper gastrointestinal tract. In yet a further embodiment, the composition may provide at least about 8 hours of drug delivery to the upper gastrointestinal tract. In yet a further embodiment, the composition may provide at least about 9 hours, or at least about 10 hours of drug delivery to the upper gastrointestinal tract.

In yet another embodiment, upon administration of the pharmaceutical composition to a subject, APAP undergoes presystemic metabolism in the gut and/or liver allowing only a fraction of the drug to reach the systemic circulation. The fraction of drug that is originally absorbed prior to pre-systemic metabolism is referred to as the fraction absorbed and denoted “Fab.” This is different from the fraction bioavailable “F,” which is the fraction that reaches the systemic circulation after the metabolism in the gut and liver.

In another embodiment, 60-90% of the acetaminophen in the pharmaceutical composition, which is available for absorption into the systemic circulation, is absorbed in the upper gastrointestinal tract. In still another embodiment, 60-85% of acetaminophen in the pharmaceutical composition, which is available for absorption into the systemic circulation, is absorbed in the duodenum and jejunum. Greater than 50% absorption of acetaminophen in the upper gastrointestinal tract is beneficial to a human subject because acetaminophen is poorly absorbed in the stomach and well absorbed in the small intestine and particularly, the upper segment of the gastrointestinal tract. It is therefore critical that acetaminophen is available in upper small intestine for its absorption. In one embodiment acetaminophen is released in stomach and reaches quickly into upper part of the small intestine for the absorption to take place.

In another embodiment, when about 60% to about 75% of the acetaminophen is released from the dosage form in the stomach within 2 hours following oral administration, about 10% to about 25% of the total amount of the acetaminophen in the dosage form, which is available for absorption into the systemic circulation, is absorbed in the duodenum, about 25% to about 40% is absorbed in the proximal jejunum, about 15% to about 20% is absorbed in the distal jejunum, and about 5% to about 15% is absorbed in the ileum.

In another embodiment, when about 70% to about 90% of the acetaminophen is released from the dosage form in the stomach within 4 hours following oral administration, about 10% to about 25% of the total amount of the acetaminophen in the dosage form, which is available for absorption into the systemic circulation, is absorbed in the duodenum, about 25% to about 40% is absorbed in the proximal jejunum, about 15% to about 20% is absorbed in the distal jejunum, and about 5% to about 15% is absorbed in the ileum.

In yet another embodiment, when at least about 55% of the total amount of the acetaminophen is released from the dosage form in the stomach within 1 hour after oral administration and when at least about 60% of the acetaminophen is released in the stomach after 2 hours, about 15% to about 20% of the total amount of the acetaminophen in the dosage form, which is available for absorption into the systemic circulation, is absorbed in the duodenum, about 30% to about 37% is absorbed in the proximal jejunum, about 15% to about 18% is absorbed in the distal jejunum, and about 8% to about 10% is absorbed in the ileum.

In still another embodiment, upon administration of the pharmaceutical composition to a subject, the opioid undergoes presystemic metabolism in the gut and/or liver allowing only a fraction of the drug to reach the systemic circulation. The fraction of drug that is originally absorbed prior to pre-systemic metabolism is referred to as the fraction absorbed and denoted “Fab.” In one embodiment, the opioid is hydrocodone. This is different from the fraction bioavailable “F,” which is the fraction that reaches the systemic circulation after metabolism in the gut and liver.

In a further embodiment, 70-95% of the hydrocodone in the pharmaceutical composition, which is available for absorption into the systemic circulation, is absorbed in the upper gastrointestinal tract. In still another embodiment, 80-95% of hydrocodone in the pharmaceutical composition, which is available for absorption into the systemic circulation, is absorbed in the duodenum and jejunum.

In one embodiment, the composition releases the opioid and other API in the stomach to optimize drug absorption in the duodenum and jejunum. For example, when about 25% to about 50% of hydrocodone is released from the dosage form in the stomach within 1 hour following oral administration, about 10% to about 45% of the total amount of the hydrocodone in the dosage form, which is available for absorption into the systemic circulation, is absorbed in the duodenum, about 25% to about 50% is absorbed in the proximal jejunum, about 7% to about 20% is absorbed in the distal jejunum, and about 2% to about 15% is absorbed in the ileum.

In another embodiment, when about 45% to about 65% of hydrocodone is released from the dosage form in the stomach within 2 hours following oral administration, about 10% to about 50% of the total amount of the hydrocodone in the dosage form, which is available for absorption into the systemic circulation, is absorbed in the duodenum, about 25% to about 55% is absorbed in the proximal, about 5% to about 25% is absorbed in the distal jejunum, and about 2% to about 15% is absorbed in the ileum.

In another embodiment, when about 60% to about 85% of hydrocodone is released from the dosage form in the stomach within 4 hours following oral administration, about 10% to about 55% of the total amount of the hydrocodone in the dosage form, which is available for absorption into the systemic circulation, is absorbed in the duodenum, about 30% to about 60% is absorbed in the proximal, about 10% to about 30% is absorbed in the distal jejunum, and about 2% to about 20% is absorbed in the ileum.

In yet another embodiment, when at least 25% of the total amount of the hydrocodone is released from the dosage form in the stomach within 1 hour after oral administration and when at least 45% of the hydrocodone is released in the stomach after 2 hours, about 30% to about 45% of the total amount of hydrocodone in the dosage form, which is available for absorption into the systemic circulation, is absorbed in the duodenum, about 37% to about 43% is absorbed in the proximal jejunum, about 10% to about 15% is absorbed in the distal jejunum, and about 2% to about 8% is absorbed in the ileum.

In another embodiment, about 90% to about 100% of the IR dose of acetaminophen is released within about 15 minutes, 30 minutes, 45 minutes or 60 minutes after oral administration. In one embodiment, the dosage form provides a dissolution profile wherein about 20% to about 65%, about 35% to about 55% or about 40% to about 50% of the ER dose of acetaminophen remains in the ER layer between about 1 and 2 hours after administration. In one embodiment, not more than 50% of the ER dose of acetaminophen is released within about the first hour. In a further embodiment, not more than 45% or not more than 40% of the ER dose of acetaminophen is released within about the first hour. In another embodiment, not more than 85% of the ER dose of acetaminophen is released within about 4 hours. In yet another embodiment, not less than 50% is released after about 6 hours. In yet another embodiment, not less than 60% is released after about 6 hours. In one embodiment, the ER dose of acetaminophen is released over a time period of about 6 to 12, about 8 to 10, or about 9 to 10 hours in vitro. In another embodiment, the ER dose of acetaminophen is released over a time period of about 7 hours, 8 hours, 9 hours, 10 hours, 11 hours or 12 hours in vitro. In another embodiment, at least 90% or 95% of the ER dose of acetaminophen is released over a time period of about 7 hours, 8 hours, 9 hours, 10 hours, 11 hours or 12 hours in vitro.

In one embodiment, the pharmaceutical compositions disclosed herein rapidly achieve therapeutic plasma drug levels of hydrocodone and acetaminophen similar to an immediate release product, which provides an early onset of action within about the first 5 minutes, 10 minutes, 15 minutes, 20 minutes, 25 minutes, 30 minutes, 35 minutes, 40 minutes, 45 minutes, 50 minutes, 55 minutes or 60 minutes after administration of the composition, but unlike an immediate release product, the pharmaceutical composition is able to maintain those therapeutic plasma drug levels of hydrocodone and acetaminophen over an extended period of time (e.g., up to 12 hours). Currently, there is no pharmaceutical composition available comprising hydrocodone and acetaminophen which is able to provide a patient with quick onset of analgesia and maintenance of analgesia for an extended period of time.

In yet another embodiment, upon average, within one hour of administration to a subject, the pharmaceutical composition achieves a Cmax for acetaminophen. The Cmax achieved by the pharmaceutical composition disclosed herein is comparable to the Cmax obtained from a commercially-available immediate release product containing acetaminophen formulated at half the strength of the commercially-available immediate release product. The acetaminophen continues to be released from the pharmaceutical composition at a rate less than the clearance rate for the acetaminophen, so that the acetaminophen levels fall smoothly until all of the acetaminophen is absorbed. Stated another way, the acetaminophen released by the pharmaceutical composition is eliminated by the body faster than it is being absorbed. The absorption of the acetaminophen released from the pharmaceutical composition is complete in about 8 to about 10 hours so that for one half life of acetaminophen the blood supply reaching the subject's liver via the portal vein contains no additional amounts of acetaminophen beyond the amounts present in the subject's general circulation.

These additional amounts of acetaminophen delivered to the liver from the subject's portal vein are frequently caused by the absorption of acetaminophen in the subject's gastrointestinal tract. Indeed, blood from the subject's intestines passes through the liver and then on to the general circulation. When acetaminophen is undergoing absorption, blood containing acetaminophen from the absorption process passes through the subject's liver prior to entering the general circulation where the acetaminophen is diluted by the distribution and clearance processes. The metabolism of these higher acetaminophen concentrations in blood coming into the subject's liver is termed the “first pass effect.” Hence, the absorption process for acetaminophen taxes a subject's metabolic systems in the liver due to these higher “first pass” concentrations. Once the absorption process is complete, the concentration of acetaminophen in the blood reaching the subject's liver through the portal vein will be the same concentration of acetaminophen as found in blood throughout the rest of the subject's body. Thus, the pharmaceutical compositions disclosed herein provide a Cmax comparable to a commercially-available immediate-release acetaminophen product (dosed at half strength) while providing a less taxing burden on the subject's metabolic systems in the liver because the acetaminophen released by the pharmaceutical composition is eliminated by the subject's body faster than it is being absorbed. This results in decreased levels of acetaminophen in a subject's liver as compared to an immediate release dosage form of acetaminophen dosed every 6 hours.

### (i) the Pharmacokinetic Profiles of the Pharmaceutical Compositions of the Invention are not Affected by the Fed or Fasted State of the Subject

Food can play a significant role in both the rate and extent of absorption of a drug. As is known, the primary function of the small intestine is to absorb food. During and after a meal, the intestine normally shows very irregular or unsynchronized contractions that move the food content back and forth and mix it with the digestive enzymes that are secreted into the intestine. However, these contractions are not entirely unsynchronized; they move the contents of the intestine slowly towards the large intestine. It normally takes about 90-120 minutes for the first part of a meal to reach the large intestine, and the last portion of the meal may not reach the large intestine for five (5) hours. Between meals, the intestine shows cycles of activity that repeat about every 90-120 minutes. The cycle consists of a short period of very few contractions (Phase I), followed by a long period of unsynchronized contractions that appear similar to the fed pattern (pre-burst, Phase II), and then a burst of strong, regular contractions that move down the intestine in a peristaltic fashion (Phase III). Phase III represents a continuation of the “housekeeper waves” that start in the stomach; its function is to sweep undigested food particles and bacteria out of the small intestine and ultimately into the large intestine.

Because non-opioid GR dosage forms of the prior art, as well as prior art extended release opioid formulations, demonstrate food effects, Applicants expected to likewise see a food effect with the pharmaceutical compositions of the present invention. Here, however, Applicants have surprisingly discovered that the pharmacokinetic profiles of a pharmaceutical composition that comprises hydrocodone and acetaminophen are not substantially affected by the fed or fasted state of a human subject ingesting the composition.

In general, a fed state is defined as having consumed food within about 30 min prior to administration of the composition. The food may be a high fat meal, a low fat meal, a high calorie meal, or a low calorie meal. A fasted state may be defined as not having ingested food for at least 10 hours prior to administration of the composition. In some embodiments, the subject may have fasted for at least 10 hours prior to the first dose and refrains from ingesting food for at least one hour prior to administration of subsequent doses. In other embodiments, the fasted subject may not have ingested food for at least 1 hour, 2 hours, 3 hours, 4 hours, 5 hours, 6 hours, 7 hours, 8 hours, 9 hours, or 10 hours prior to administration of each dose of the composition.

As the pharmacokinetic profiles of a pharmaceutical composition that comprises hydrocodone and acetaminophen are not substantially affected by the fed or fasted state of a human subject, there is no substantial difference in the quantity of drug absorbed or the rate of drug absorption when the hydrocodone/acetaminophen compositions are administered in the fed versus the fasted state. Without being bound to theory, Applicants believe that in a fasted state the opioid acts to reduce gastric motility in an amount sufficient to retain the dosage form in the stomach thereby mitigating the “housekeeper waves” described above.

As shown herein, the pharmacokinetic parameters of the compositions of the invention are similar when the composition is administered in the fed and fasted states. Benefits of a dosage form, which substantially eliminates the effect of food, include an increase in convenience, thereby increasing patient compliance, as the patient does not need to ensure that they are taking a dose either with or without food. This is significant because poor patient compliance can lead to adverse therapeutic outcomes.

The invention also encompasses an hydrocodone/APAP pharmaceutical composition in which administration of the composition to a human subject in a fasted state is bioequivalent to administration of the composition to a human subject in a fed state wherein bioequivalence is established by: (1) a 90% Confidence Interval (CI) for AUC which is between 80% and 125%, and (2) a 90% CI for Cmax, which is 80% and 125%. In a further embodiment, the compositions disclosed herein may by administered to a subject in need thereof without regard to food.

other embodiments, the difference in absorption of either the opioids and/or the APIs of the invention, when administered in the fed versus the fasted state, is less than about 35%, less than about 30%, less than about 25%, less than about 20%, less than about 15%, less than about 10%, less than about 5%, or less than about 3%. The pharmacokinetic parameter of the other API(s) that is independent of food may be, but is not limited to, Cmax, C1 hr, C2 hr, AUC, partial AUC, Tmax, and Tlag. Additionally, the opioid(s) in the composition produce a plasma profile characterized by at least one pharmacokinetic parameter that differs by less than about 30% under fed and fasted conditions. In various embodiments, the pharmacokinetic parameter may vary by less than about 25%, 20%, 15%, 10%, or 5% under fed and fasted conditions. In one embodiment, the pharmacokinetic parameter of the opioid that is independent of food may be, but is not limited to, Cmax, C1 hr, C2 hr, AUC, partial AUC, Tmax, and Tlag.

### (j) Exemplary Compositions

In one embodiment, the pharmaceutical composition for extended release of hydrocodone and acetaminophen comprises at least one extended release portion comprising acetaminophen, hydrocodone or a combination thereof, and at least one extended release component; and at least one immediate release portion comprising hydrocodone, acetaminophen, or combinations thereof. In yet another embodiment, the pharmaceutical composition comprises an immediate release portion comprising hydrocodone and acetaminophen and an extended release portion comprising hydrocodone, acetaminophen and an extended release component. In still yet another embodiment, the composition comprises two extended release portions, each comprising an extended release component and one of the hydrocodone or the acetaminophen, and an immediate release portion comprising the hydrocodone and the acetaminophen. In another embodiment, the composition comprises two extended release portions, each comprising an extended release component and one of hydrocodone or acetaminophen, and two immediate release portions, each comprising one of hydrocodone or acetaminophen. In one embodiment, the extended release component comprises at least one extended release polymer. In another one embodiment, the extended release polymer comprises a polyethylene oxide. The molecular weight of the polyethylene oxide may be from about 500,000 Daltons to about 10,000,000 Daltons.

In another embodiment, the pharmaceutical composition may comprise from about 5 mg to about 30 mg of hydrocodone and from about 250 mg to about 1300 mg of acetaminophen. In one exemplary embodiment, the pharmaceutical composition may comprise about 15 mg of hydrocodone and about 650 mg of acetaminophen. In another exemplary embodiment, the composition may comprise about 15 mg of hydrocodone and about 500 mg of acetaminophen. In yet another exemplary embodiment, the composition may comprise about 15 mg of hydrocodone and about 325 mg of acetaminophen. In still another exemplary embodiment, the pharmaceutical composition may comprise about 10 mg of hydrocodone and about 325 mg of acetaminophen. In yet another exemplary embodiment, the pharmaceutical composition may comprise about 7.5 mg of hydrocodone and about 325 mg of acetaminophen. In still another exemplary embodiment, the pharmaceutical composition may comprise about 5 mg of hydrocodone and about 325 mg of acetaminophen. In a further exemplary embodiment, the pharmaceutical composition may comprise about 20 mg of hydrocodone and about 650 mg of acetaminophen. In another exemplary embodiment, the composition may comprise about 30 mg of hydrocodone and about 650 mg of acetaminophen.

In another embodiment, the composition may comprise from about 5 mg to about 30 mg of opioid and from about 250 mg to about 1300 mg of at least one other API. In one embodiment, the composition may comprise about 15 mg of opioid and about 650 mg of at least one other API. In another embodiment, the composition may comprise about 15 mg of opioid and about 500 mg of at least one other API. In a further embodiment, the composition may comprise about 30 mg of opioid and about 500 mg of at least one other API. In still another embodiment, the composition may comprise about 15 mg of opioid and about 325 mg of at least one other API. In yet another exemplary embodiment, the composition may comprise about 7.5 mg of opioid and about 325 mg of at least one other API. In still another exemplary embodiment, the pharmaceutical composition may comprise about 10 mg of opioid and about 325 mg of at least one other API. In yet another exemplary embodiment, the pharmaceutical composition may comprise about 5 mg of opioid and about 325 mg of at least one other API. In a further exemplary embodiment, the pharmaceutical composition may comprise about 20 mg of opioid and about 650 mg of at least one other API. In another exemplary embodiment, the composition may comprise about 30 mg of opioid and about 650 mg of at least one other API. In yet another exemplary embodiment, the composition may comprise about 22.5 mg of opioid and about 925 mg of at least one other API.

In a further embodiment, a single dosage form of the pharmaceutical composition disclosed herein (e.g., one tablet) will provide a subject with approximately the same therapeutic benefit and pharmacokinetic profile as either two dosage forms (e.g., two tablets) of the composition formulated at half the strength, or three dosage forms (e.g., three tablets) of the composition formulated at a third of the strength. In yet another exemplary embodiment, the pharmaceutical composition comprising 15 mg of hydrocodone and 650 mg of acetaminophen in a single dosage form (e.g., one tablet) will provide a subject with approximately the same therapeutic benefit and pharmacokinetic profile as two dosage forms of the pharmaceutical composition formulated at half the strength (e.g., each tablet comprising 7.5 mg of hydrocodone and 325 mg of acetaminophen). In still another exemplary embodiment, the pharmaceutical composition comprising 15 mg of hydrocodone and 650 mg of acetaminophen in a single dosage form (e.g., one tablet) will provide a subject with approximately the same therapeutic benefit and pharmacokinetic profile as three dosage forms of the pharmaceutical composition formulated at a third of the strength (e.g., each tablet comprising 5 mg of hydrocodone and about 216.7 mg of acetaminophen). In yet another embodiment, the pharmaceutical composition comprising 15 mg of hydrocodone and 325 mg of acetaminophen in a single dosage form (e.g., one tablet) taken together with another tablet comprising 7.5 mg of hydrocodone and 325 mg of acetaminophen in a single dosage form will provide a subject with approximately the same therapeutic benefit and pharmacokinetic profile as a single tablet comprising 22.5 mg of hydrocodone and 650 mg of acetaminophen. In still another exemplary embodiment, the pharmaceutical composition comprising 15 mg of hydrocodone and 325 mg of acetaminophen in a single dosage form (e.g., one tablet) taken together with another tablet comprising 15 mg of hydrocodone and 325 mg of acetaminophen in a single dosage form will provide a subject with approximately the same therapeutic benefit and pharmacokinetic profile as a single tablet configuration totaling 30 mg of hydrocodone and 650 mg of acetaminophen. In yet a further exemplary embodiment, a pharmaceutical composition comprising 21 mg of hydrocodone and 650 mg of acetaminophen in a single dosage form (e.g., one tablet) will provide a subject with approximately the same therapeutic benefit and pharmacokinetic profile as two dosage forms of the pharmaceutical composition formulated at half the strength (e.g., each tablet comprising 10.5 mg of hydrocodone and 325 mg of acetaminophen).). In yet another exemplary embodiment, a pharmaceutical composition comprising 22.5 mg of hydrocodone and 925 mg of acetaminophen in a single dosage form (e.g., one tablet) will provide a subject with approximately the same therapeutic benefit and pharmacokinetic profile as three dosage forms of the pharmaceutical composition formulated at a third of the strength (e.g., each tablet comprising 7.5 mg of hydrocodone and 325 mg of acetaminophen).

In yet another embodiment, the at least one extended release portion of the composition may comprise from about 40% to about 60% (w/w) of the total amount of acetaminophen in the composition and from about 70% to about 80% (w/w) of the total amount of hydrocodone in the composition, whereas the at least one immediate release portion may comprise from about 40% to about 60% (w/w) of the total amount of acetaminophen in the composition and from about 20% to about 30% (w/w) of the total amount of hydrocodone in the composition. In still another embodiment, the at least one extended release portion may comprise about 50% (w/w) of the total amount of acetaminophen in the composition and about 75% (w/w) of the total amount of hydrocodone in the composition; and the at least one immediate release portion may comprise about 50% (w/w) of total amount of acetaminophen in the composition and about 25% (w/w) of the total amount of hydrocodone in the composition.

In another embodiment, an extended release portion of the composition may comprise, by weight of such extended release portion, from about 30% to about 50% of the extended release polymer, from about 20% to about 40% of acetaminophen, and from about 0.5% to about 2% of hydrocodone; and an immediate release portion may comprise, by weight of such immediate release portion, from about 70% to about 80% acetaminophen and from about 0.5% to about 1% of hydrocodone.

In yet another embodiment, the pharmaceutical composition may comprise from about 7.5 mg to about 30 mg of hydrocodone and from about 325 mg to about 650 mg of acetaminophen, wherein the at least one immediate release portion may comprise about 25% (w/w) of the total amount of hydrocodone in the composition and about 50% (w/w) of the total amount of acetaminophen in the composition, and the at least one extended release portion may comprise about 75% (w/w) of the total amount of hydrocodone in the composition, about 50% (w/w) of the total amount of acetaminophen in the composition, and about 35% to about 45%, by weight of the at least one extended release portion, of an extended release polymer comprising a polyethylene oxide.

In yet another embodiment, the pharmaceutical composition may comprise about 5 mg of hydrocodone and about 325 mg of acetaminophen, wherein the at least one immediate release portion may comprise about 25% (w/w) of the total amount of hydrocodone in the composition and about 50% (w/w) of the total amount of acetaminophen in the composition, and the at least one extended release portion may comprise about 75% (w/w) of the total amount of hydrocodone in the composition, about 50% (w/w) of the total amount of acetaminophen in the composition.

In a further embodiment, the pharmaceutical composition may comprise about 5 mg of hydrocodone and about 325 mg of acetaminophen, wherein the at least one immediate release portion may comprise about 20% (w/w) to about 30% (w/w) of the total amount of hydrocodone in the composition, and about 40% (w/w) to about 60% (w/w) of the total amount of acetaminophen in the composition; and the at least one extended release portion may comprise about 70% (w/w) to about 80% (w/w) of the total amount of hydrocodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 5 mg of hydrocodone and about 325 mg of acetaminophen, wherein the at least one immediate release portion may comprise about 1.25 mg of hydrocodone and about 162.5 mg of acetaminophen, and the at least one extended release portion may comprise about 3.75 mg of hydrocodone and about 162.5 mg of acetaminophen.

In still another embodiment, the pharmaceutical composition may comprise about 5 mg of hydrocodone and about 325 mg of acetaminophen, wherein the at least one immediate release portion may comprise about 0.75 mg to about 2 mg of hydrocodone and about 125 mg to about 325 mg of acetaminophen; and the at least one extended release portion may comprise about 3 mg to about 4.5 mg of hydrocodone and about 125 mg to about 325 mg of acetaminophen.

In yet another embodiment, the pharmaceutical composition may comprise about 7.5 mg of hydrocodone and about 325 mg of acetaminophen, wherein the at least one immediate release portion may comprise about 25% (w/w) of the total amount of hydrocodone in the composition and about 50% (w/w) of the total amount of acetaminophen in the composition, and the at least one extended release portion may comprise about 75% (w/w) of the total amount of hydrocodone in the composition, about 50% (w/w) of the total amount of acetaminophen in the composition.

In a further embodiment, the pharmaceutical composition may comprise about 7.5 mg of hydrocodone and about 325 mg of acetaminophen, wherein the at least one immediate release portion may comprise about 20% (w/w) to about 30% (w/w) of the total amount of hydrocodone in the composition, and about 40% (w/w) to about 60% (w/w) of the total amount of acetaminophen in the composition; and the at least one extended release portion may comprise about 70% (w/w) to about 80% (w/w) of the total amount of hydrocodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 7.5 mg of hydrocodone and about 325 mg of acetaminophen, wherein the at least one immediate release portion may comprise about 1.875 mg of hydrocodone and about 162.5 mg of acetaminophen, and the at least one extended release portion may comprise about 5.625 mg of hydrocodone and about 162.5 mg of acetaminophen.

In still another embodiment, the pharmaceutical composition may comprise about 7.5 mg of hydrocodone and about 325 mg of acetaminophen, wherein the at least one immediate release portion may comprise about 1 mg to about 3 mg of hydrocodone and about 125 mg to about 325 mg of acetaminophen; and the at least one extended release portion may comprise about 4.75 mg to about 6.5 mg of hydrocodone and about 125 mg to about 325 mg of acetaminophen.

In yet another embodiment, the pharmaceutical composition may comprise about 10 mg of hydrocodone and about 325 mg of acetaminophen, wherein the at least one immediate release portion may comprise about 25% (w/w) of the total amount of hydrocodone in the composition and about 50% (w/w) of the total amount of acetaminophen in the composition, and the at least one extended release portion may comprise about 75% (w/w) of the total amount of hydrocodone in the composition, about 50% (w/w) of the total amount of acetaminophen in the composition.

In a further embodiment, the pharmaceutical composition may comprise about 10 mg of hydrocodone and about 325 mg of acetaminophen, wherein the at least one immediate release portion may comprise about 20% (w/w) to about 30% (w/w) of the total amount of hydrocodone in the composition, and about 40% (w/w) to about 60% (w/w) of the total amount of acetaminophen in the composition; and the at least one extended release portion may comprise about 70% (w/w) to about 80% (w/w) of the total amount of hydrocodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 10 mg of hydrocodone and about 325 mg of acetaminophen, wherein the at least one immediate release portion may comprise about 2.5 mg of hydrocodone and about 162.5 mg of acetaminophen, and the at least one extended release portion may comprise about 7.5 mg of hydrocodone and about 162.5 mg of acetaminophen.

In still another embodiment, the pharmaceutical composition may comprise about 10 mg of hydrocodone and about 325 mg of acetaminophen, wherein the at least one immediate release portion may comprise about 1.5 mg to about 3.5 mg of hydrocodone and about 125 mg to about 325 mg of acetaminophen; and the at least one extended release portion may comprise about 6.25 mg to about 8.75 mg of hydrocodone and about 125 mg to about 325 mg of acetaminophen.

In yet another embodiment, the pharmaceutical composition may comprise about 15 mg of hydrocodone and about 325 mg of acetaminophen, wherein the at least one immediate release portion may comprise about 25% (w/w) of the total amount of hydrocodone in the composition and about 50% (w/w) of the total amount of acetaminophen in the composition, and the at least one extended release portion may comprise about 75% (w/w) of the total amount of hydrocodone in the composition, about 50% (w/w) of the total amount of acetaminophen in the composition.

In a further embodiment, the pharmaceutical composition may comprise about 15 mg of hydrocodone and about 325 mg of acetaminophen, wherein the at least one immediate release portion may comprise about 20% (w/w) to about 30% (w/w) of the total amount of hydrocodone in the composition, and about 40% (w/w) to about 60% (w/w) of the total amount of acetaminophen in the composition; and the at least one extended release portion may comprise about 70% (w/w) to about 80% (w/w) of the total amount of hydrocodone in the composition and about 40% (w/w) to about 60% (w/w) of the total amount of acetaminophen in the composition. The at least one extended release portion may also comprise about 35% to about 45%, by weight of an extended release polymer, such as a polyethylene oxide.

In an additional embodiment, the pharmaceutical composition may comprise about 15 mg of hydrocodone and about 325 mg of acetaminophen, wherein the at least one immediate release portion may comprise about 3.75 mg of hydrocodone and about 162.5 mg of acetaminophen, and the at least one extended release portion may comprise about 11.25 mg of hydrocodone and about 162.5 mg of acetaminophen.

In still another embodiment, the pharmaceutical composition may comprise about 15 mg of hydrocodone and about 325 mg of acetaminophen, wherein the at least one immediate release portion may comprise about 2.5 mg to about 5 mg of hydrocodone and about 125 mg to about 325 mg of acetaminophen; and the at least one extended release portion may comprise about 10 mg to about 12.5 mg of hydrocodone and about 125 mg to about 325 mg of acetaminophen.

Other exemplary formulations are set forth in Charts 1-2 below:

CHART 1
Representative Hydrocodone/Acetaminophen Formulations.

| | | Formulation No. | | | | | | | | | |
|---|---|---|---|---|---|---|---|---|---|---|---|
| | | 1 | 2 | 3 | 4 | 5 | 6 | 7 | 8 | 9 | 10 |
| Immediate | APAP | 185.3 | 175.0 | 180.0 | 160.4 | 200.0 | 193.4 | 118.8 | 162.5 | 139.0 | 150.0 |
| Release | Hydrocodone bitartrate | 1.100 | 1.75 | 2.00 | 2.50 | 1.25 | 1.00 | 2.75 | 1.875 | 1.75 | 1.875 |
| Layer | Microcrystalline cellulose | 23.0 | 17.0 | 19.0 | 27.0 | 16.0 | 18.0 | 18.0 | 14.0 | 21.0 | 24.0 |
| | Pregelatinized starch | 0.05 | 0.15 | 0.25 | 0.10 | 0.05 | 0.30 | 0.20 | 0.25 | 0.15 | 0.20 |
| | Citric Acid Anhydrous | 0.08 | 0.08 | 0.08 | 0.11 | 0.11 | 0.14 | 0.07 | 0.13 | 0.15 | 0.17 |
| | EDTA disodium salt, | 0.087 | 0.106 | 0.075 | 0.03 | 0.050 | 0.055 | 0.033 | 0.025 | 0.045 | 0.018 |
| | dihydrate | | | | | | | | | | |
| | Hydroxypropyl cellulose | 14.1 | 17.8 | — | — | 17.3 | — | 16.7 | 16.1 | 21.5 | — |
| | Hypromellose | 2.5 | — | 3.2 | — | — | — | — | — | 8.9 | 19.5 |
| | Hydroxypropyl methyl | — | — | 21.7 | 18.3 | — | 19.3 | — | — | — | 3.0 |
| | cellulose | | | | | | | | | | |
| | Croscarmellose sodium | 10.0 | 11.0 | 11.5 | 11.5 | 13.0 | 14.5 | 14.5 | 12.5 | 14.0 | 12.5 |
| | Silicon dioxide | 0.97 | 0.75 | 1.14 | 1.02 | 1.10 | 1.03 | 0.88 | 1.05 | 0.93 | 2.30 |
| | Magnesium stearate | 1.5 | 1.0 | 1.0 | 0.5 | 0.5 | 2.0 | 2.0 | 0.5 | 1.5 | 2.5 |
| Extended | APAP | 185.3 | 150.0 | 145.0 | 155.2 | 125.0 | 100.5 | 146.9 | 162.5 | 207.4 | 150.0 |
| Release | Hydrocodone bitartrate | 6.900 | 5.75 | 5.50 | 5.00 | 6.25 | 6.50 | 7.25 | 5.625 | 4.75 | 6.625 |
| | Microcrystalline cellulose | 175.4 | 180.0 | 302.2 | 275.0 | 214.8 | 250.0 | 245.7 | 203.6 | 288.3 | 200.5 |
| | Pregelatinized starch | 0.60 | 0.60 | 0.70 | 0.70 | 0.70 | 0.75 | 0.75 | 0.75 | 0.85 | 0.85 |
| | Citric Acid Anhydrous | 0.24 | 0.16 | 0.24 | 0.22 | 0.33 | 0.28 | 0.07 | 0.38 | 0.45 | 0.34 |
| | EDTA disodium salt, | 0.160 | 0.085 | 0.095 | 0.055 | 0.130 | 0.065 | 0.065 | 0.075 | 0.130 | 0.125 |
| | dihydrate | | | | | | | | | | |
| | Hydroxypropyl cellulose | 30.0 | 275.8 | 95.5 | 210.6 | 13.2 | 40.7 | 32.9 | 9.6 | — | — |
| | Polyox N12K | 292.8 | — | — | — | 287.7 | — | — | 321.8 | 155.5 | — |
| | Polyox 303 | — | — | 244.2 | — | — | — | 275.5 | — | — | 189.2 |
| | Hydroxypropyl methyl | — | 103.2 | — | 134.2 | — | 182.2 | — | — | 155.5 | 210.2 |
| | cellulose | | | | | | | | | | |
| | Silicon Dioxide | 1.8 | 1.3 | 1.5 | 2.3 | 2.4 | 3.0 | 3.5 | 3.6 | 2.0 | 2.5 |
| | Magnesium Stearate | 7.5 | 8.0 | 7.4 | 8.1 | 7.5 | 10.2 | 9.9 | 7.2 | 10.3 | 10.3 |
| | | Formulation No. | | | | | | | | | |
| | | 11 | 12 | 13 | 14 | 15 | 16 | 17 | 18 | 19 | 20 |
| Immediate | APAP | 300.0 | 150.0 | 200.0 | 150.0 | 100.0 | 160.0 | 190.0 | 75.0 | 90.0 | 125.0 |
| Release | Hydrocodone bitartrate | 2.00 | 1.00 | 1.50 | 3.50 | 2.75 | 1.25 | 1.25 | 2.50 | 1.75 | 3.00 |
| Layer | Microcrystalline cellulose | 21.5 | 18.5 | 25.3 | 35.0 | 15.7 | 27.1 | 9.9 | 13.9 | 24.2 | 16.9 |
| | Pregelatinized starch | 0.03 | 0.30 | 0.25 | 0.27 | 0.08 | 0.35 | 0.75 | 0.09 | 0.15 | 0.26 |
| | Citric Acid Anhydrous | 0.12 | 0.08 | 0.09 | 0.16 | 0.07 | 0.24 | 0.14 | 0.26 | 0.15 | 0.20 |
| | EDTA disodium salt, | 0.04 | 0.175 | 0.1 | 0.06 | 0.1 | 0.09 | 0.06 | 0.08 | 0.063 | 0.09 |
| | dihydrate | | | | | | | | | | |
| | Hydroxypropyl cellulose | — | 21.5 | 1.8 | 9.8 | 14.8 | — | 20.8 | 19.2 | 25.4 | — |
| | Hypromellose | 2.5 | — | — | — | — | — | — | — | 10.3 | 22.5 |
| | Hydroxypropyl methyl | 16.3 | 11.4 | 17.5 | 8.7 | — | 29.3 | — | — | — | 4.4 |
| | cellulose | | | | | | | | | | |
| | Croscarmellose sodium | 6.8 | 11.0 | 12.8 | 7.9 | 19.0 | 9.6 | 13.3 | 15.6 | 15.1 | 14.7 |
| | Silicon dioxide | 0.86 | 0.80 | 2.25 | 1.24 | .95 | 1.34 | 0.80 | 1.66 | 0.79 | 2.37 |
| | Magnesium stearate | 1.75 | 1.0 | 0.75 | 0.6 | 0.5 | 2.5 | 1.9 | 0.8 | 1.2 | 2.8 |
| Extended | APAP | 150.0 | 150.0 | 125.0 | 75.0 | 100.0 | 165.0 | 135.0 | 225.0 | 210.0 | 150.0 |
| Release | Hydrocodone bitartrate | 8.00 | 6.50 | 6.00 | 6.50 | 3.25 | 6.25 | 6.25 | 5.00 | 6.25 | 5.50 |
| | Microcrystalline cellulose | 182.2 | 197.6 | 300.4 | 269.6 | 210.0 | 275.5 | 283.2 | 310.2 | 240.8 | 210.0 |
| | Pregelatinized starch | 0.75 | 0.73 | 0.46 | 0.89 | 0.55 | 0.78 | 0.55 | 0.65 | 0.67 | 0.64 |
| | Citric Acid Anhydrous | 0.25 | 0.36 | 0.38 | 0.34 | 0.37 | 0.23 | 0.14 | 0.40 | 0.70 | 0.70 |
| | EDTA disodium salt, | 0.23 | 0.09 | 0.14 | 0.06 | 0.183 | 0.035 | 0.049 | 0.03 | 0.105 | 0.075 |
| | dihydrate | | | | | | | | | | |
| | Hydroxypropyl cellulose | 34.7 | 321.9 | 88.4 | 212.9 | 11.9 | 37.7 | 34.2 | 17.4 | — | — |
| | Polyox N12K | — | — | 252.4 | — | 290.3 | — | 248.2 | 279.2 | 175.2 | — |
| | Polyox 303 | 275.8 | — | — | — | — | — | — | — | — | 224.5 |
| | Hydroxypropyl methyl | — | 101.1 | — | 110.5 | — | 192.1 | — | — | 140.9 | 185.6 |
| | cellulose | | | | | | | | | | |
| | Silicon Dioxide | 1.3 | 1.3 | 1.2 | 2.4 | 2.1 | 3.2 | 4.0 | 4.0 | 2.0 | 3.8 |
| | Magnesium Stearate | 5.7 | 9.4 | 6.6 | 5.5 | 7.7 | 9.4 | 6.4 | 5.2 | 9.9 | 7.2 |
| | | Formulation No. | | | | | | | | | |
| | | 21 | 22 | 23 | 24 | 25 | 26 | 27 | 28 | 29 | 30 |
| Immediate | APAP | 185.3 | 175.0 | 180.0 | 160.4 | 200.0 | 193.4 | 118.8 | 162.5 | 139.0 | 150.0 |
| Release | Hydrocodone bitartrate | 1.100 | 1.75 | 2.00 | 2.50 | 1.25 | 1.00 | 2.75 | 1.875 | 1.75 | 1.875 |
| Layer | Microcrystalline cellulose | 23.0 | 17.0 | 19.0 | 27.0 | 16.0 | 18.0 | 18.0 | 14.0 | 21.0 | 24.0 |
| | Pregelatinized starch | 0.05 | 0.15 | 0.25 | 0.10 | 0.05 | 0.30 | 0.20 | 0.25 | 0.15 | 0.20 |
| | Citric Acid Anhydrous | 0.08 | 0.08 | 0.08 | 0.11 | 0.11 | 0.14 | 0.07 | 0.13 | 0.15 | 0.17 |
| | EDTA disodium salt, | 0.087 | 0.106 | 0.075 | 0.03 | 0.050 | 0.055 | 0.033 | 0.025 | 0.045 | 0.018 |
| | dihydrate | | | | | | | | | | |
| | Hydroxypropyl cellulose | 14.1 | 17.8 | — | — | 17.3 | — | 16.7 | 16.1 | 21.5 | — |
| | Hypromellose | 2.5 | — | 3.2 | — | — | — | — | — | 8.9 | 19.5 |
| | Hydroxypropyl methyl | — | — | 21.7 | 18.3 | — | 19.3 | — | — | — | 3.0 |
| | cellulose | | | | | | | | | | |
| | Croscarmellose sodium | 10.0 | 11.0 | 11.5 | 11.5 | 13.0 | 14.5 | 14.5 | 12.5 | 14.0 | 12.5 |
| | Silicon dioxide | 0.97 | 0.75 | 1.14 | 1.02 | 1.10 | 1.03 | 0.88 | 1.05 | 0.93 | 2.30 |
| | Magnesium stearate | 1.5 | 1.0 | 1.0 | 0.5 | 0.5 | 2.0 | 2.0 | 0.5 | 1.5 | 2.5 |
| Extended | APAP | 185.3 | 150.0 | 145.0 | 155.2 | 125.0 | 100.5 | 146.9 | 162.5 | 207.4 | 150.0 |
| Release | Hydrocodone bitartrate | 6.900 | 5.75 | 5.50 | 5.00 | 6.25 | 6.50 | 7.25 | 5.625 | 4.75 | 6.625 |
| Layer | Microcrystalline cellulose | 175.4 | 180.0 | 302.2 | 275.0 | 214.8 | 250.0 | 245.7 | 203.6 | 288.3 | 200.5 |
| | Pregelatinized starch | 0.60 | 0.60 | 0.70 | 0.70 | 0.70 | 0.75 | 0.75 | 0.75 | 0.85 | 0.85 |
| | Citric Acid Anhydrous | 0.24 | 0.16 | 0.24 | 0.22 | 0.33 | 0.28 | 0.07 | 0.38 | 0.45 | 0.34 |
| | EDTA disodium salt, | 0.160 | 0.085 | 0.095 | 0.055 | 0.130 | 0.065 | 0.065 | 0.075 | 0.130 | 0.125 |
| | dihydrate | | | | | | | | | | |
| | Hydroxypropyl cellulose | 30.0 | 275.8 | 95.5 | 210.6 | 13.2 | 40.7 | 32.9 | 9.6 | — | — |
| | Polyox N60K | 292.8 | — | — | — | 287.7 | — | — | 321.8 | 155.5 | — |
| | Polyox 205 | — | — | 244.2 | — | — | — | 275.5 | — | — | 189.2 |
| | Hydroxypropyl methyl | — | 103.2 | — | 134.2 | — | 182.2 | — | — | 155.5 | 210.2 |
| | cellulose | | | | | | | | | | |
| | Silicon Dioxide | 1.8 | 1.3 | 1.5 | 2.3 | 2.4 | 3.0 | 3.5 | 3.6 | 2.0 | 2.5 |
| | Magnesium Stearate | 7.5 | 8.0 | 7.4 | 8.1 | 7.5 | 10.2 | 9.9 | 7.2 | 10.3 | 10.3 |
| | | Formulation No. | | | | | | | | | |
| | | 31 | 32 | 33 | 34 | 35 | 36 | 37 | 38 | 39 | 40 |
| Immediate | APAP | 300.0 | 150.0 | 200.0 | 150.0 | 100.0 | 160.0 | 190.0 | 75.0 | 90.0 | 125.0 |
| Release | Hydrocodone bitartrate | 2.00 | 1.00 | 1.50 | 3.50 | 2.75 | 1.25 | 1.25 | 2.50 | 1.75 | 3.00 |
| Layer | Microcrystalline cellulose | 21.5 | 18.5 | 25.3 | 35.0 | 15.7 | 27.1 | 9.9 | 13.9 | 24.2 | 16.9 |
| | Pregelatinized starch | 0.03 | 0.30 | 0.25 | 0.27 | 0.08 | 0.35 | 0.75 | 0.09 | 0.15 | 0.26 |
| | Citric Acid Anhydrous | 0.12 | 0.08 | 0.09 | 0.16 | 0.07 | 0.24 | 0.14 | 0.26 | 0.15 | 0.20 |
| | EDTA disodium salt, | 0.04 | 0.175 | 0.1 | 0.06 | 0.1 | 0.09 | 0.06 | 0.08 | 0.063 | 0.09 |
| | dihydrate | | | | | | | | | | |
| | Hydroxypropyl cellulose | — | 21.5 | 1.8 | 9.8 | 14.8 | — | 20.8 | 19.2 | 25.4 | — |
| | Hypromellose | 2.5 | — | — | — | — | — | — | — | 10.3 | 22.5 |
| | Hydroxypropyl methyl | 16.3 | 11.4 | 17.5 | 8.7 | — | 29.3 | — | — | — | 4.4 |
| | cellulose | | | | | | | | | | |
| | Croscarmellose sodium | 6.8 | 11.0 | 12.8 | 7.9 | 19.0 | 9.6 | 13.3 | 15.6 | 15.1 | 14.7 |
| | Silicon dioxide | 0.86 | 0.80 | 2.25 | 1.24 | .95 | 1.34 | 0.80 | 1.66 | 0.79 | 2.37 |
| | Magnesium stearate | 1.75 | 1.0 | 0.75 | 0.6 | 0.5 | 2.5 | 1.9 | 0.8 | 1.2 | 2.8 |
| Extended | APAP | 150.0 | 150.0 | 125.0 | 75.0 | 100.0 | 165.0 | 135.0 | 225.0 | 210.0 | 150.0 |
| Release | Hydrocodone bitartrate | 8.00 | 6.50 | 6.00 | 6.50 | 3.25 | 6.25 | 6.25 | 5.00 | 6.25 | 5.50 |
| Layer | Microcrystalline cellulose | 182.2 | 197.6 | 300.4 | 269.6 | 210.0 | 275.5 | 283.2 | 310.2 | 240.8 | 210.0 |
| | Pregelatinized starch | 0.75 | 0.73 | 0.46 | 0.89 | 0.55 | 0.78 | 0.55 | 0.65 | 0.67 | 0.64 |
| | Citric Acid Anhydrous | 0.25 | 0.36 | 0.38 | 0.34 | 0.37 | 0.23 | 0.14 | 0.40 | 0.70 | 0.70 |
| | EDTA disodium salt, | 0.23 | 0.09 | 0.14 | 0.06 | 0.183 | 0.035 | 0.049 | 0.03 | 0.105 | 0.075 |
| | dihydrate | | | | | | | | | | |
| | Hydroxypropyl cellulose | 34.7 | 321.9 | 88.4 | 212.9 | 11.9 | 37.7 | 34.2 | 17.4 | — | — |
| | Polyox N60K | — | — | 252.4 | — | 290.3 | — | 248.2 | 279.2 | 175.2 | — |
| | Polyox 205 | 275.8 | — | — | — | — | — | — | — | — | 224.5 |
| | Hydroxypropyl methyl | — | 101.1 | — | 110.5 | — | 192.1 | — | — | 140.9 | 185.6 |
| | cellulose | | | | | | | | | | |
| | Silicon Dioxide | 1.3 | 1.3 | 1.2 | 2.4 | 2.1 | 3.2 | 4.0 | 4.0 | 2.0 | 3.8 |
| | Magnesium Stearate | 5.7 | 9.4 | 6.6 | 5.5 | 7.7 | 9.4 | 6.4 | 5.2 | 9.9 | 7.2 |
| | | Formulation No. | | | | | | | | | |
| | | 41 | 42 | 43 | 44 | 45 | 46 | 47 | 48 | 49 | 50 |
| Immediate | APAP | 185.3 | 175.0 | 180.0 | 160.4 | 200.0 | 193.4 | 118.8 | 162.5 | 139.0 | 150.0 |
| Release | Hydrocodone bitartrate | 1.100 | 1.75 | 2.00 | 2.50 | 1.25 | 1.00 | 2.75 | 1.875 | 1.75 | 1.875 |
| Layer | Microcrystalline cellulose | 23.0 | 17.0 | 19.0 | 27.0 | 16.0 | 18.0 | 18.0 | 14.0 | 21.0 | 24.0 |
| | Pregelatinized starch | 0.05 | 0.15 | 0.25 | 0.10 | 0.05 | 0.30 | 0.20 | 0.25 | 0.15 | 0.20 |
| | Citric Acid Anhydrous | 0.08 | 0.08 | 0.08 | 0.11 | 0.11 | 0.14 | 0.07 | 0.13 | 0.15 | 0.17 |
| | EDTA disodium salt, | 0.087 | 0.106 | 0.075 | 0.03 | 0.050 | 0.055 | 0.033 | 0.025 | 0.045 | 0.018 |
| | dihydrate | | | | | | | | | | |
| | Hydroxypropyl cellulose | 14.1 | 17.8 | — | — | 17.3 | — | 16.7 | 16.1 | 21.5 | — |
| | Hypromellose | 2.5 | — | 3.2 | — | — | — | — | — | 8.9 | 19.5 |
| | Hydroxypropyl methyl | — | — | 21.7 | 18.3 | — | 19.3 | — | — | — | 3.0 |
| | cellulose | | | | | | | | | | |
| | Croscarmellose sodium | 10.0 | 11.0 | 11.5 | 11.5 | 13.0 | 14.5 | 14.5 | 12.5 | 14.0 | 12.5 |
| | Silicon dioxide | 0.97 | 0.75 | 1.14 | 1.02 | 1.10 | 1.03 | 0.88 | 1.05 | 0.93 | 2.30 |
| | Magnesium stearate | 1.5 | 1.0 | 1.0 | 0.5 | 0.5 | 2.0 | 2.0 | 0.5 | 1.5 | 2.5 |
| Extended | APAP | 185.3 | 150.0 | 145.0 | 155.2 | 125.0 | 100.5 | 146.9 | 162.5 | 207.4 | 150.0 |
| Release | Hydrocodone bitartrate | 6.900 | 5.75 | 5.50 | 5.00 | 6.25 | 6.50 | 7.25 | 5.625 | 4.75 | 6.625 |
| Layer | Microcrystalline cellulose | 175.4 | 180.0 | 302.2 | 275.0 | 214.8 | 250.0 | 245.7 | 203.6 | 288.3 | 200.5 |
| | Pregelatinized starch | 0.60 | 0.60 | 0.70 | 0.70 | 0.70 | 0.75 | 0.75 | 0.75 | 0.85 | 0.85 |
| | Citric Acid Anhydrous | 0.24 | 0.16 | 0.24 | 0.22 | 0.33 | 0.28 | 0.07 | 0.38 | 0.45 | 0.34 |
| | EDTA disodium salt, | 0.160 | 0.085 | 0.095 | 0.055 | 0.130 | 0.065 | 0.065 | 0.075 | 0.130 | 0.125 |
| | dihydrate | | | | | | | | | | |
| | Hydroxypropyl cellulose | 30.0 | 275.8 | 95.5 | 210.6 | 13.2 | 40.7 | 32.9 | 9.6 | — | — |
| | Polyox 1105 | 262.2 | — | — | — | 301.6 | — | — | 250.3 | 188.3 | — |
| | Polyox N-750 | — | — | 244.2 | — | — | — | 275.5 | — | — | 189.2 |
| | Hydroxypropyl methyl | — | 103.2 | — | 134.2 | — | 182.2 | — | — | 155.5 | 210.2 |
| | cellulose | | | | | | | | | | |
| | Silicon Dioxide | 1.8 | 1.3 | 1.5 | 2.3 | 2.4 | 3.0 | 3.5 | 3.6 | 2.0 | 2.5 |
| | Magnesium Stearate | 7.5 | 8.0 | 7.4 | 8.1 | 7.5 | 10.2 | 9.9 | 7.2 | 10.3 | 10.3 |
| | | Formulation No. | | | | | | | | | |
| | | 51 | 52 | 53 | 54 | 55 | 56 | 57 | 58 | 59 | 60 |
| Immediate | APAP | 300.0 | 150.0 | 200.0 | 150.0 | 100.0 | 160.0 | 190.0 | 75.0 | 90.0 | 125.0 |
| Release | Hydrocodone bitartrate | 2.00 | 1.00 | 1.50 | 3.50 | 2.75 | 1.25 | 1.25 | 2.50 | 1.75 | 3.00 |
| Layer | Microcrystalline cellulose | 21.5 | 18.5 | 25.3 | 35.0 | 15.7 | 27.1 | 9.9 | 13.9 | 24.2 | 16.9 |
| | Pregelatinized starch | 0.03 | 0.30 | 0.25 | 0.27 | 0.08 | 0.35 | 0.75 | 0.09 | 0.15 | 0.26 |
| | Citric Acid Anhydrous | 0.12 | 0.08 | 0.09 | 0.16 | 0.07 | 0.24 | 0.14 | 0.26 | 0.15 | 0.20 |
| | EDTA disodium salt, | 0.04 | 0.175 | 0.1 | 0.06 | 0.1 | 0.09 | 0.06 | 0.08 | 0.063 | 0.09 |
| | dihydrate | | | | | | | | | | |
| | Hydroxypropyl cellulose | — | 21.5 | 1.8 | 9.8 | 14.8 | — | 20.8 | 19.2 | 25.4 | — |
| | Hypromellose | 2.5 | — | — | — | — | — | — | — | 10.3 | 22.5 |
| | Hydroxypropyl methyl | 16.3 | 11.4 | 17.5 | 8.7 | — | 29.3 | — | — | — | 4.4 |
| | cellulose | | | | | | | | | | |
| | Croscarmellose sodium | 6.8 | 11.0 | 12.8 | 7.9 | 19.0 | 9.6 | 13.3 | 15.6 | 15.1 | 14.7 |
| | Silicon dioxide | 0.86 | 0.80 | 2.25 | 1.24 | .95 | 1.34 | 0.80 | 1.66 | 0.79 | 2.37 |
| | Magnesium stearate | 1.75 | 1.0 | 0.75 | 0.6 | 0.5 | 2.5 | 1.9 | 0.8 | 1.2 | 2.8 |
| Extended | APAP | 150.0 | 150.0 | 125.0 | 75.0 | 100.0 | 165.0 | 135.0 | 225.0 | 210.0 | 150.0 |
| Release | Hydrocodone bitartrate | 8.00 | 6.50 | 6.00 | 6.50 | 3.25 | 6.25 | 6.25 | 5.00 | 6.25 | 5.50 |
| Layer | Microcrystalline cellulose | 182.2 | 197.6 | 300.4 | 269.6 | 210.0 | 275.5 | 283.2 | 310.2 | 240.8 | 210.0 |
| | Pregelatinized starch | 0.75 | 0.73 | 0.46 | 0.89 | 0.55 | 0.78 | 0.55 | 0.65 | 0.67 | 0.64 |
| | Citric Acid Anhydrous | 0.25 | 0.36 | 0.38 | 0.34 | 0.37 | 0.23 | 0.14 | 0.40 | 0.70 | 0.70 |
| | EDTA disodium salt, | 0.23 | 0.09 | 0.14 | 0.06 | 0.183 | 0.035 | 0.049 | 0.03 | 0.105 | 0.075 |
| | dihydrate | | | | | | | | | | |
| | Hydroxypropyl cellulose | 34.7 | 321.9 | 88.4 | 212.9 | 11.9 | 37.7 | 34.2 | 17.4 | — | — |
| | Polyox 1105 | — | — | 252.4 | — | 290.3 | — | 248.2 | 279.2 | 175.2 | — |
| | Polyox N-750 | 275.8 | — | — | — | — | — | — | — | — | 224.5 |
| | Hydroxypropyl methyl | — | 101.1 | — | 110.5 | — | 192.1 | — | — | 140.9 | 185.6 |
| | cellulose | | | | | | | | | | |
| | Silicon Dioxide | 1.3 | 1.3 | 1.2 | 2.4 | 2.1 | 3.2 | 4.0 | 4.0 | 2.0 | 3.8 |
| | Magnesium Stearate | 5.7 | 9.4 | 6.6 | 5.5 | 7.7 | 9.4 | 6.4 | 5.2 | 9.9 | 7.2 |
| | | Formulation No. | | | | | | | | | |
| | | 61 | 62 | 63 | 64 | 65 | 66 | 67 | 68 | 69 | 70 |
| Immediate | APAP | 185.3 | 175.0 | 180.0 | 160.4 | 200.0 | 193.4 | 118.8 | 162.5 | 139.0 | 150.0 |
| Release | Hydrocodone bitartrate | 1.100 | 1.75 | 2.00 | 2.50 | 1.25 | 1.00 | 2.75 | 1.875 | 1.75 | 1.875 |
| Layer | Microcrystalline cellulose | 23.0 | 17.0 | 19.0 | 27.0 | 16.0 | 18.0 | 18.0 | 14.0 | 21.0 | 24.0 |
| | Pregelatinized starch | 0.05 | 0.15 | 0.25 | 0.10 | 0.05 | 0.30 | 0.20 | 0.25 | 0.15 | 0.20 |
| | Citric Acid Anhydrous | 0.08 | 0.08 | 0.08 | 0.11 | 0.11 | 0.14 | 0.07 | 0.13 | 0.15 | 0.17 |
| | EDTA disodium salt, | 0.087 | 0.106 | 0.075 | 0.03 | 0.050 | 0.055 | 0.033 | 0.025 | 0.045 | 0.018 |
| | dihydrate | | | | | | | | | | |
| | Hydroxypropyl cellulose | 14.1 | 17.8 | — | — | 17.3 | — | 16.7 | 16.1 | 21.5 | — |
| | Hypromellose | 2.5 | — | 3.2 | — | — | — | — | — | 8.9 | 19.5 |
| | Hydroxypropyl methyl | — | — | 21.7 | 18.3 | — | 19.3 | — | — | — | 3.0 |
| | cellulose | | | | | | | | | | |
| | Croscarmellose sodium | 10.0 | 11.0 | 11.5 | 11.5 | 13.0 | 14.5 | 14.5 | 12.5 | 14.0 | 12.5 |
| | Silicon dioxide | 0.97 | 0.75 | 1.14 | 1.02 | 1.10 | 1.03 | 0.88 | 1.05 | 0.93 | 2.30 |
| | Magnesium stearate | 1.5 | 1.0 | 1.0 | 0.5 | 0.5 | 2.0 | 2.0 | 0.5 | 1.5 | 2.5 |
| Extended | APAP | 185.3 | 150.0 | 145.0 | 155.2 | 125.0 | 100.5 | 146.9 | 162.5 | 207.4 | 150.0 |
| Release | Hydrocodone bitartrate | 6.900 | 5.75 | 5.50 | 5.00 | 6.25 | 6.50 | 7.25 | 5.625 | 4.75 | 6.625 |
| Layer | Microcrystalline cellulose | 175.4 | 180.0 | 302.2 | 275.0 | 214.8 | 250.0 | 245.7 | 203.6 | 288.3 | 200.5 |
| | Pregelatinized starch | 0.60 | 0.60 | 0.70 | 0.70 | 0.70 | 0.75 | 0.75 | 0.75 | 0.85 | 0.85 |
| | Citric Acid Anhydrous | 0.24 | 0.16 | 0.24 | 0.22 | 0.33 | 0.28 | 0.07 | 0.38 | 0.45 | 0.34 |
| | EDTA disodium salt, | 0.160 | 0.085 | 0.095 | 0.055 | 0.130 | 0.065 | 0.065 | 0.075 | 0.130 | 0.125 |
| | dihydrate | | | | | | | | | | |
| | Hydroxypropyl cellulose | 30.0 | 275.8 | 95.5 | 210.6 | 13.2 | 40.7 | 32.9 | 9.6 | — | — |
| | Polyox 301 | 292.8 | — | — | — | 287.7 | — | — | 321.8 | 155.5 | — |
| | Polyox N-80 | — | — | 244.2 | — | — | — | 275.5 | — | — | 189.2 |
| | Hydroxypropyl methyl | — | 103.2 | — | 134.2 | — | 182.2 | — | — | 155.5 | 210.2 |
| | cellulose | | | | | | | | | | |
| | Silicon Dioxide | 1.8 | 1.3 | 1.5 | 2.3 | 2.4 | 3.0 | 3.5 | 3.6 | 2.0 | 2.5 |
| | Magnesium Stearate | 7.5 | 8.0 | 7.4 | 8.1 | 7.5 | 10.2 | 9.9 | 7.2 | 10.3 | 10.3 |
| | | Formulation No. | | | | | | | | | |
| | | 71 | 72 | 73 | 74 | 75 | 76 | 77 | 78 | 79 | 80 |
| Immediate | APAP | 300.0 | 150.0 | 200.0 | 150.0 | 100.0 | 160.0 | 190.0 | 75.0 | 90.0 | 125.0 |
| Release | Hydrocodone bitartrate | 2.00 | 1.00 | 1.50 | 3.50 | 2.75 | 1.25 | 1.25 | 2.50 | 1.75 | 3.00 |
| Layer | Microcrystalline cellulose | 21.5 | 18.5 | 25.3 | 35.0 | 15.7 | 27.1 | 9.9 | 13.9 | 24.2 | 16.9 |
| | Pregelatinized starch | 0.03 | 0.30 | 0.25 | 0.27 | 0.08 | 0.35 | 0.75 | 0.09 | 0.15 | 0.26 |
| | Citric Acid Anhydrous | 0.12 | 0.08 | 0.09 | 0.16 | 0.07 | 0.24 | 0.14 | 0.26 | 0.15 | 0.20 |
| | EDTA disodium salt, | 0.04 | 0.175 | 0.1 | 0.06 | 0.1 | 0.09 | 0.06 | 0.08 | 0.063 | 0.09 |
| | dihydrate | | | | | | | | | | |
| | Hydroxypropyl cellulose | — | 21.5 | 1.8 | 9.8 | 14.8 | — | 20.8 | 19.2 | 25.4 | — |
| | Hypromellose | 2.5 | — | — | — | — | — | — | — | 10.3 | 22.5 |
| | Hydroxypropyl methyl | 16.3 | 11.4 | 17.5 | 8.7 | — | 29.3 | — | — | — | 4.4 |
| | cellulose | | | | | | | | | | |
| | Croscarmellose sodium | 6.8 | 11.0 | 12.8 | 7.9 | 19.0 | 9.6 | 13.3 | 15.6 | 15.1 | 14.7 |
| | Silicon dioxide | 0.86 | 0.80 | 2.25 | 1.24 | .95 | 1.34 | 0.80 | 1.66 | 0.79 | 2.37 |
| | Magnesium stearate | 1.75 | 1.0 | 0.75 | 0.6 | 0.5 | 2.5 | 1.9 | 0.8 | 1.2 | 2.8 |
| Extended | APAP | 150.0 | 150.0 | 125.0 | 75.0 | 100.0 | 165.0 | 135.0 | 225.0 | 210.0 | 150.0 |
| Release | Hydrocodone bitartrate | 8.00 | 6.50 | 6.00 | 6.50 | 3.25 | 6.25 | 6.25 | 5.00 | 6.25 | 5.50 |
| Layer | Microcrystalline cellulose | 182.2 | 197.6 | 300.4 | 269.6 | 210.0 | 275.5 | 283.2 | 310.2 | 240.8 | 210.0 |
| | Pregelatinized starch | 0.75 | 0.73 | 0.46 | 0.89 | 0.55 | 0.78 | 0.55 | 0.65 | 0.67 | 0.64 |
| | Citric Acid Anhydrous | 0.25 | 0.36 | 0.38 | 0.34 | 0.37 | 0.23 | 0.14 | 0.40 | 0.70 | 0.70 |
| | EDTA disodium salt, | 0.23 | 0.09 | 0.14 | 0.06 | 0.183 | 0.035 | 0.049 | 0.03 | 0.105 | 0.075 |
| | dihydrate | | | | | | | | | | |
| | Hydroxypropyl cellulose | 34.7 | 321.9 | 88.4 | 212.9 | 11.9 | 37.7 | 34.2 | 17.4 | — | — |
| | Polyox 301 | — | — | 252.4 | — | 290.3 | — | 248.2 | 279.2 | 175.2 | — |
| | Polyox N-80 | 275.8 | — | — | — | — | — | — | — | — | 224.5 |
| | Hydroxypropyl methyl | — | 101.1 | — | 110.5 | — | 192.1 | — | — | 140.9 | 185.6 |
| | cellulose | | | | | | | | | | |
| | Silicon Dioxide | 1.3 | 1.3 | 1.2 | 2.4 | 2.1 | 3.2 | 4.0 | 4.0 | 2.0 | 3.8 |
| | Magnesium Stearate | 5.7 | 9.4 | 6.6 | 5.5 | 7.7 | 9.4 | 6.4 | 5.2 | 9.9 | 7.2 |

*All weights in mg.

CHART 2
Additional Hydrocodone/Acetaminophen Formulations.

| | | 81 | 82 | 83 | 84 | 85 | 86 | 87 | 88 | 89 | 90 |
|---|---|---|---|---|---|---|---|---|---|---|---|
| Immediate | APAP | 325.0 | 325 | 325 | 325 | 325.0 | 325.0 | 325.0 | 325.0 | 325.0 | 325.0 |
| Release | Hydrocodone bitartrate | 3.75 | 3.75 | 3.75 | 3.75 | 3.75 | 3.75 | 3.75 | 3.75 | 3.75 | 3.75 |
| Layer | Microcrystalline cellulose | 28.10 | 28.10 | 28.10 | 28.10 | 28.1 | 28.1 | 28.1 | 28.1 | 28.1 | 28.1 |
| | Pregelatinized starch | 0.50 | 0.50 | 0.50 | 0.50 | 0.50 | 0.50 | 0.50 | 0.50 | 0.50 | 0.50 |
| | Citric Acid Anhydrous | 0.25 | 0.25 | 0.25 | 0.25 | 0.25 | 0.25 | 0.25 | 0.25 | 0.25 | 0.25 |
| | EDTA disodium salt, | 0.05 | 0.05 | 0.05 | 0.05 | 0.05 | 0.05 | 0.05 | 0.05 | 0.05 | 0.05 |
| | dihydrate | | | | | | | | | | |
| | Hydroxypropyl cellulose | 32.23 | 32.24 | 32.24 | 32.24 | 32.24 | 32.24 | 32.24 | 32.24 | 32.24 | 32.24 |
| | Croscarmellose sodium | 25.087 | 25.09 | 25.09 | 25.09 | 25.09 | 25.09 | 25.09 | 25.09 | 25.09 | 25.09 |
| | Silicon dioxide | 2.09 | 2.09 | 2.09 | 2.09 | 2.09 | 2.09 | 2.09 | 2.09 | 2.09 | 2.09 |
| | Magnesium stearate | 1.045 | 1.05 | 1.05 | 1.05 | 1.045 | 1.045 | 1.045 | 1.045 | 1.045 | 1.045 |
| Extended | APAP | 325.0 | 325 | 325 | 325 | 325.0 | 325.0 | 325.0 | 325.0 | 325.0 | 325.0 |
| Release | Hydrocodone bitartrate | 11.25 | 11.25 | 11.25 | 11.25 | 11.25 | 11.25 | 11.25 | 11.25 | 11.25 | 11.25 |
| Layer | Microcrystalline cellulose | 23.85 | 95.19 | 23.85 | 23.85 | 23.85 | 95.19 | 23.85 | 23.85 | 23.85 | 95.19 |
| | Pregelatinized starch | 1.50 | 1.50 | 1.50 | 1.50 | 1.5 | 1.5 | 1.5 | 1.5 | 1.5 | 1.5 |
| | Citric Acid Anhydrous | 0.75 | 0.75 | 0.75 | 0.75 | 0.75 | 0.75 | 0.75 | 0.75 | 0.75 | 0.75 |
| | EDTA disodium salt, | 0.15 | 0.15 | 0.15 | 0.15 | 0.15 | 0.15 | 0.15 | 0.15 | 0.15 | 0.15 |
| | dihydrate | | | | | | | | | | |
| | Hydroxypropyl cellulose | 19.16 | 19.16 | 19.16 | 19.16 | 19.16 | 19.16 | 19.16 | 19.16 | 19.16 | 19.16 |
| | Polyox 1105 | 321.02 | 249.68 | — | 321.02 | — | 249.68 | 321.02 | — | — | — |
| | Polyox N12K | — | — | 321.02 | — | — | — | — | 321.02 | — | — |
| | Polyox N60K | — | — | — | — | 321.02 | — | — | — | 321.02 | 249.68 |
| | Silicon Dioxide | — | — | — | — | | 3.57 | 3.57 | 3.57 | 3.57 | 3.57 |
| | Magnesium Stearate | 3.57 | 3.57 | 3.57 | 3.57 | 3.57 | 7.13 | 7.13 | 7.13 | 7.13 | 7.13 |

*All weights in mg.

### III. METHODS FOR PREPARING SOLID DOSAGE FORMS OF THE PHARMACEUTICAL COMPOSITION

Another aspect of the disclosure provides methods for preparing solid dosage forms of the pharmaceutical composition that provide extended release of hydrocodone and acetaminophen. Solid dosage compositions in the form of tablets may be produced using any suitable method known in the art including but not limited to wet granulation, dry granulation, direct compression, and combinations thereof.

Granulation is a manufacturing process which increases the size and homogeneity of active pharmaceutical ingredients and excipients that comprise a solid dose composition. The granulation process, which is often referred to as agglomeration, changes important physical characteristics of the dry composition, with the aim of improving manufacturability and, thereby, product quality, as well as providing desired release kinetics. Wet granulation is by far the more prevalent agglomeration process utilized within the pharmaceutical industry. Most wet granulation procedures follow some basic steps; the active agent(s) and excipients are mixed together, and a binder solution is prepared and added to the powder mixture to form a wet mass. The moist particles are then dried and sized by milling or by screening through a sieve. In some cases, the wet granulation is “wet milled” or sized through screens before the drying step. The wet granulation process may be a high shear granulation process or a fluid bed granulation process. Several methods of granulation are described in co-pending application U.S. application Ser. No. 13/166,770, filed Jun. 22, 2011, which is incorporated herein by reference in its entirety.

After granulation and drying of the resultant particles, batches are characterized with respect to properties such as final Loss on Drying (LOD), bulk density, tap density, and particle size. Loss on Drying (LOD) typically is determined after each granulation using the Moisture Analyzer. Several 1 g samples may be taken and loaded into the moisture analyzer. The samples may be run for 5 minutes at a temperature of 105° C. In another embodiment, the samples may be run at 105° C. until there is no weight fluctuation in order to determine the LOD.

Bulk and tap densities may be determined as follows. A graduated cylinder is filled with a certain amount of material (e.g., 30-40 g or 82-88 g), and the volume recorded to determine the material bulk density. Tap density can be determined with a help of a Tap Density Tester by exposing the material to 100 taps per test and recording the new volume.

Particle size determination generally is performed immediately after granulation, after sieving through 20 mesh screen to remove agglomerates. Particle diameter may be determined with a sieve-type particle diameter distribution gauge using sieves with openings of 30, 40, 60, 80, 120, and 325 mesh. Fractions may be weighed on a Mettler balance to estimate size distribution. This provides determination of the quantitative ratio by particle diameter of composition comprising extended release particles. Sieve analysis according to standard United States Pharmacopoeia methods (e.g., USP-23 NF 18), may be done such as by using a Meinzer II Sieve Shaker.

In one embodiment, the method for preparing dosage forms of the pharmaceutical composition may comprise wet granulating a first mixture comprising the opioid, such as hydrocodone, the API, such as acetaminophen, and a binder to produce a first granulation mixture. The wet granulation process may be a fluid bed granulation process. In additional embodiments, the first mixture may further comprise at least one additional excipient selected from the group consisting of fillers, lubricants, antioxidants, chelating agents, and color agents. The first granulation mixture may be blended with an extended release polymer and one or more excipients, as listed above, to form at least one extended release portion of a dosage form. In certain embodiments, the extended release polymer may be a polyethylene oxide.

In another embodiment, the method further comprises wet granulating a second mixture comprising the opioid, such as hydrocodone, the API, such as acetaminophen, and a binder to form a second granulation mixture. The wet granulation process may be a fluid bed granulation process. In some embodiments, the second mixture may further comprise at least one additional excipient selected from the group consisting of fillers, lubricants, disintegrants, antioxidants, chelating agents, and color agents. The second granulation mixture may be blended with one or more excipients, as listed above, to form an immediate release portion of a dosage form.

In an additional embodiment, the method may further comprise compressing the at least one extended release portion and the at least one immediate release portion into a tablet. The tablet may be a bilayer tablet. The tablet may be coated with a tablet coating.

In another embodiment, the method may comprise granulating via a high shear wet granulation process a mixture comprising the opioid (e.g., hydrocodone) and at least one excipient to form opioid (e.g., hydrocodone) particles. The opioid particles may be dried at a suitable temperature. The opioid particles comprising hydrocodone may be granulated via a fluid bed granulation process with the API (e.g., acetaminophen), a binder, and an optional excipient to form the granulation mixture. The granulation mixture may be blended with an extended release polymer and at least one excipient to form an extended release portion of a solid dosage form.

In a further embodiment, the method may further comprise granulating via a fluid bed granulation process opioid particles comprising hydrocodone with the API, a binder, and an optional excipient to form another granulation mixture. This granulation mixture may be blended with one or more excipients to form an immediate release portion of a solid dosage form.

In an additional embodiment, the method may further comprise compressing the at least one extended release portion comprising opioid particles and the at least one immediate release portion comprising opioid particles into a tablet. In one embodiment, the method comprises compressing one extended release portion comprising opioid particles and one immediate release portion comprising opioid particles into a bilayer tablet. The tablet may be coated with a tablet coating.

In another embodiment, wet granulation of either mixture may produce particles with a bulk density ranging from about 0.30 to 0.40 grams/milliliter (g/mL). In other aspects, the wet granulation may produce particles with a tap density ranging from about 0.35 g/mL to about 0.45 g/mL. In other embodiments, the wet granulation may produce particles, wherein at least about 50% of the particles have a size greater than 125 microns. In still other embodiments, the wet granulation may produce particles wherein about 20% to about 65% of the particles have a size greater than about 125 microns and less than about 250 microns.

Tablets generally are characterized with respect to disintegration and dissolution release profiles as well as tablet hardness, friability, and content uniformity.

In vitro dissolution profiles for the tablets may be determined using a USP Type II apparatus, with a paddle speed of either about 100 rpm or 150 rpm, in 0.1 N HCl, at 37° C. Samples of 5 mL at each time-point, may be taken without media replacement at 0.08, 0.25, 0.5, 1, 2, 4, 6, 8 and 12 hours, for example. In some embodiments, the dissolution profiles may be determined at varying pH values, such as at a pH of about 3.0, 3.5, 4.0, 4.5, 5.0, 5.5, 6.0 or 6.5. The fluid used may be, for example, HCl, phosphate buffer, or simulated gastric fluid. The resulting cumulative dissolution profiles for the tablets are based upon a theoretical percent active added to the pharmaceutical compositions.

A tablet preferably disintegrates before it dissolves. A disintegration tester measures the time it takes a tablet to break apart in solution. The tester suspends tablets in a solution bath for visual monitoring of the disintegration rate. Both the time to disintegration and the disintegration consistency of all tablets may be measured. The disintegration profile may be determined in a USP Disintegration Tester in pH 5.8 phosphate buffer or 0.1 N HCl of pH 1.2. The fluid used may be, for example, HCl, phosphate buffer, or simulated gastric fluid. Samples, 1-5 mL at each time-point, may be taken, for example, without media replacement at 0.5, 1, 2, 3, 4, 5, 6, 7 and 8 hours. The resulting cumulative disintegration profiles are based upon a theoretical percent active added to the pharmaceutical compositions.

After tablets are formed by compression, it is desired that the tablets have a strength of at least 9-25 Kiloponds (kp), or at least about 12-20 kp. A hardness tester generally is used to determine the load required to diametrically break the tablets (crushing strength) into two equal halves. The fracture force may be measured using a Venkel Tablet Hardness Tester, using standard USP protocols.

Friability is a well-known measure of a tablet's resistance to surface abrasion that measures weight loss in percentage after subjecting the tablets to a standardized agitation procedure. Friability properties are especially important during any transport of the dosage form as any fracturing of the final dosage form may result in a subject receiving less than the prescribed medication. Friability may be determined using a Roche Friability Drum according to standard USP guidelines which specifies the number of samples, the total number of drum revolutions, and the drum rpm to be used. Friability values of from 0.8 to 1.0% generally are regarded as constituting the upper limit of acceptability.

The prepared tablets generally are tested for content uniformity to determine if they meet the pharmaceutical requirement of an acceptance value of 15 or less. Each tablet may be placed in a solution of 60% methanol/40% isopropanol and stirred at room temperature until the tablet disintegrates. The solution containing the dissolved tablet may be further diluted in 90% water/10% isopropanol/0.1% heptafluorobutyric acid and generally is analyzed by HPLC.

### IV. METHOD FOR REDUCING THE RISK OF ACETAMINOPHEN-INDUCED HEPATIC DAMAGE

The present disclosure also provides methods for reducing the risk of acetaminophen-induced hepatic damage in a subject being treated for pain with a dosage regimen that comprises administering to the subject at least two consecutive doses of a pharmaceutical composition comprising hydrocodone and acetaminophen. The method comprises administering a first dose of a pharmaceutical composition comprising at least one extended release portion comprising the acetaminophen, the hydrocodone or a combination thereof, and an extended release component to the subject, wherein the composition maintains a therapeutic blood plasma concentration of hydrocodone of at least 5 ng/mL from about 0.75 hours to about 10 hours after administration of the composition, and wherein at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration. The method further comprises administering a second dose of the pharmaceutical composition to the subject at about 12 hours after administration of the first dose.

Avoiding toxic intermediate formation is an important strategy in addressing product safety. Indeed, acetaminophen is absorbed from the stomach and small intestine and primarily metabolized by conjugation in the liver to nontoxic, water-soluble compounds that are eliminated in the urine. When the maximum daily dose (“MDD”) is exceeded over a prolonged period, metabolism by conjugation becomes saturated, and excess acetaminophen is oxidatively metabolized by the CYP enzymes (CYP2E1, 1A2, 2A6, 3A4) to a reactive metabolite, N-acetyl-p-benzoquinone-imine (NAPQI). NAPQI has an extremely short half-life, and rapidly conjugates with available glutathione, which acts as a sulfhydryl donor. The reduced NAPQI is then renally excreted. The liver plays a central role in the turnover of glutathione in the body. Given that toxicity due to NAPQI formation occurs via necrosis of the liver following the formation of toxic adducts, minimizing glutathione depletion and enhancing glutathione regeneration in the liver is an important concern.

Human erythrocyte data resulting from hepatic turnover demonstrate a time-delayed response to redox and free radical insults via glutathione depletion and regeneration. The hepatic dynamics of glutathione formation and depletion in animal data using hepatic models can also be reviewed. In Swiss mice, the dynamics of glutathione depletion was investigated in detail for acetaminophen doses ranging from (100 mg/kg to 600 mg/kg) in work done by Brzeznicka and Piotrowski (1989). Under one embodiment of the present invention, the intended dosage for patients with acute pain is 1.3 g/day of acetaminophen. Assuming a subject's weight of 70 kg, this is 1.229×10⁻⁴ moles/kg/day in human subjects. In Swiss mice, 400 mg/kg and 600 mg/kg are 2.65×10⁻³ moles/kg/day and 3.97×10⁻³ moles/kg/day, respectively, resulting in a 22-fold and a 32-fold safety exposure ratio, as compared with human levels. The bioequivalence level is 95%. Brzeiznicka and Piotrowski report that circulating hepatic GSH changes in mice began within 15 min after acetaminophen administration, and depletion followed a pattern that was strictly dose dependent, reaching a minimum GSH level 2 hrs after injection for the all dose groups, rebounding to initial levels between hours 8 and 12. Taken together, these results support the hypothesis that exposing subjects to the lower end of the therapeutic window of acetaminophen may provide benefit in terms of the patient's ability to regenerate physiologically protective levels of glutathione. Thus, the pharmaceutical formulations disclosed herein, which are designed to allow for a two hour break in acetaminophen exposure in each twelve hour exposure window allows for restorative hepatic regeneration of the subject's glutathione levels during that period when the acetaminophen concentrations are at their lowest or absent, while still preserving the considerable benefits of the potentiating effects of combination analgesia.

As mentioned above, acetaminophen is primarily metabolized via conjugation reactions, e.g., glucuronidation and sulfation, in the liver to nontoxic, water-soluble compounds that are rapidly eliminated from the body. A small proportion of acetaminophen is metabolized by the cytochrome P450 system to the reactive metabolite, NAPQI. Generally, this toxic metabolite is rapidly detoxified by conjugation to glutathione to form a non-toxic metabolite that is renally excreted. However, if the conjugation pathways become saturated and more acetaminophen is metabolized via the cytochrome P450 pathway, the pool of available glutathione may become depleted. With insufficient glutathione to bind to and inactivate NAPQI, this toxic metabolite is able to react with the sulfhydryl groups of cellular proteins initiating a cascade of cellular damage, which may lead to liver necrosis, and, ultimately, liver failure.

The method disclosed herein addresses the problem of depleted stores of glutathione by providing a period of time during the later part of the dosing interval during which the release of acetaminophen is low because most of the acetaminophen has already been released from the composition. The period of time during which the release of acetaminophen is low is called the acetaminophen “time-off” period. As a consequence of this acetaminophen time-off period, the plasma levels of acetaminophen fall to sufficiently low levels such that the metabolic burden on the liver is reduced, thereby allowing the depleted stores of glutathione to be replenished via the continuous glutathione manufacturing pathway comprising the glutathione synthase pathway. Because the levels of glutathione are able to be restored before the next dose, the risk of acetaminophen-induced hepatic damage is significantly reduced.

Additionally, the acetaminophen time-off period provided by the compositions disclosed herein may provide an added and beneficial precaution for any subject undergoing acetaminophen therapy to avoid an inadvertent reduction in glutathione stores and any potential acetaminophen-induced hepatic damage. In particular, the acetaminophen time-off period provided by the compositions disclosed herein may be especially useful during chronic administration of analgesic compositions comprising acetaminophen. The subject may be at increased risk for developing acetaminophen-induced hepatic damage because of frequent and regular user of alcohol (i.e., ethanol), concurrent administration of acetaminophen from another source (e.g., an over-the-counter medication), poor diet, and/or compromised liver function.

In general, the compositions disclosed herein are formulated such that the rate of release of acetaminophen is high during the first several hours of the dosing interval and the rate of release of acetaminophen is low during the last several hours of the dosing interval. More specifically, the compositions are formulated to release from about 40% to about 65% of the acetaminophen in about 30 minutes, from about 55% to about 80% of the acetaminophen in about 2 hours, from about 65% to about 92% of the acetaminophen in about 4 hours, and from about 67% to about 95% of the acetaminophen in about 8 hours, wherein the dosing interval is about 12 hours. In another, the compositions are formulated to release from about 45% to about 60% of the acetaminophen in about 30 minutes, from about 57% to about 75% of the acetaminophen in about 2 hours, from about 67% to about 90% of the acetaminophen in about 4 hours, and from about 70% to about 95% of the acetaminophen in about 8 hours, wherein the dosing interval is about 12 hours. In yet another embodiment, during the final 4 hours of a 12 hour dosing interval, only about 5% of the acetaminophen remains to be released from the composition.

The subject may be a mammal, and in certain embodiments, the subject may be a human. In various embodiments, the at least two consecutive doses of the analgesic composition may be administered to the subject at 8 hour intervals, 10 hour intervals, 12 hour intervals, 18 hour intervals, or 24 hour intervals.

The method for reducing the risk of acetaminophen-induced hepatic damage disclosed herein may further comprise administering additional doses of the pharmaceutical composition at regular dosing intervals, such as e.g., at 12 hour intervals. During the latter part of each dosing interval, therefore, the acetaminophen time-off period allows depleted stores of glutathione to be replenished, thereby reducing the risk of acetaminophen-induced hepatic damage in subjects being treated for pain with a composition comprising acetaminophen.

### V. METHOD FOR TREATING PAIN

Also provided is a method for treating pain in a subject in need of such treatment with a pharmaceutical composition that comprises an opioid, such as hydrocodone, and an additional API, such as acetaminophen, wherein the method comprises administering an effective amount of any of the pharmaceutical compositions disclosed herein. For example, the method comprises orally administering to the subject an effective amount of a pharmaceutical composition comprising at least one extended release portion comprising hydrocodone, acetaminophen and combination thereof, and an extended release component,

wherein the composition maintains a therapeutic plasma concentration of hydrocodone of at least about 5 ng/mL from about 0.75 hour to about 10 hours after administration of the composition, and wherein at least about 90% of the acetaminophen is released from the composition by about 8 hours after administration of the composition such that, by about 10 hours after administration of the composition, acetaminophen has a blood plasma concentration that is less than about 30% of acetaminophen's maximum plasma concentration.

In some embodiments, the subject may be suffering from or diagnosed with chronic pain. In yet another embodiment, the subject may be suffering from or diagnosed with acute pain. In still another embodiment, the subject may be suffering from or diagnosed with moderate to severe acute pain. In yet other embodiments, the subject may be suffering from or diagnosed with both chronic and acute pain. The subject may be a mammal, and in certain embodiments, the subject may be a human.

In additional embodiments, the method comprises orally administering to the subject an effective amount of a gastric retentive pharmaceutical composition to the subject, wherein the subject is in a fasted state. Moreover, upon administration of the pharmaceutical composition, the opioid in the composition produces a plasma profile characterized by at least one pharmacokinetic parameter that differs by less than about 30% when the subject is in a fasted state as compared to a fed state.

The pharmacokinetic parameter of the active agent(s) of the pharmaceutical composition that differs by less that about 30% under fed and fasted conditions may be, but is not limited to, Cmax, C1 hr, C2 hr, AUC, partial AUC, Tmax, and Tlag. In various embodiments, the pharmacokinetic parameter may vary by less than about 25%, 20%, 15%, 10%, or 5% under fed and fasted conditions.

In embodiments in which the pharmaceutical composition comprises hydrocodone and acetaminophen, the Cmax or AUC of hydrocodone and the Cmax or AUC of acetaminophen may each individually vary by less than about 30%, 29%, 28%, 27%, 26%, 25%, 24%, 23%, 22%, 21%, 20%, 19%, 18%, 17%, 16%, 15%, 14%. 13%, 12%, 11%, 10%, 9%, 8%, 7%, 6%, 5%, 4%, 3%, 2%, or 1% under fed and fasted conditions.

In some embodiments, an effective amount of the pharmaceutical composition may be administered to a subject in a fed state. In general, a fed state is defined as having consumed food within about 30 min prior to administration of the pharmaceutical composition. The food may be a high fat meal, a low fat meal, a high calorie meal, or a low calorie meal. In other embodiments, an effective amount of the pharmaceutical composition may be administered to a subject in a fasted state. In general, a fasted state is defined as not having ingested food for at least 10 hours prior to administration of the pharmaceutical composition. In some embodiments, the pharmaceutical composition may be administered to a subject who has fasted for at least 10 hours prior to the first dose and who fasts for at least one hour prior to administration of subsequent doses. In other embodiments, the pharmaceutical composition may be administered to a subject who has fasted for at least 1 hour, 2 hours, 3 hours, 4 hours, 5 hours, 6 hours, 7 hours, 8 hours, 9 hours, or 10 hours prior to administration of each dose.

An effective amount of the pharmaceutical composition may comprise from about 5 mg to about 300 mg of the opioid and from about 100 mg to about 1300 mg of the other API. In embodiments in which the opioid is hydrocodone and the API is acetaminophen, the pharmaceutical composition may comprise from about 7.5 mg to about 30 mg of hydrocodone and from about 250 mg to about 1300 mg of acetaminophen.

In one embodiment, an effective amount of a pharmaceutical composition may be 15 mg of hydrocodone and 650 mg of acetaminophen. For example, one solid dosage form comprising 15 mg of hydrocodone and 650 mg of acetaminophen may be administered. Alternatively, two solid dosage forms with each comprising 7.5 mg of hydrocodone and 325 mg of acetaminophen may be administered. In another embodiment, the effective amount of a pharmaceutical composition may be 7.5 mg of hydrocodone and 325 mg of acetaminophen, wherein one solid dosage form comprising 7.5 mg of hydrocodone and 325 mg of acetaminophen may be administered. In yet another embodiment, the effective amount of a pharmaceutical composition may be 20 mg of hydrocodone and 650 mg of acetaminophen. For example, one solid dosage form comprising 20 mg of hydrocodone and 650 mg of acetaminophen may be administered. Alternatively, two solid dosage forms with each comprising 10 mg of hydrocodone and 325 mg of acetaminophen may be administered. In another embodiment, the effective amount of a pharmaceutical composition may be 10 mg of hydrocodone and 325 mg of acetaminophen, wherein one solid dosage form comprising 10 mg of hydrocodone and 325 mg of acetaminophen may be administered. In still yet another embodiment, the effective amount of a pharmaceutical composition may be 30 mg of hydrocodone and 650 mg of acetaminophen. For example, one solid dosage form comprising 30 mg of hydrocodone and 650 mg of acetaminophen may be administered. Alternatively, two solid dosage forms with each comprising 15 mg of hydrocodone and 325 mg of acetaminophen may be administered. In another embodiment, the effective amount of a pharmaceutical composition may be 15 mg of hydrocodone and 325 mg of acetaminophen, wherein one solid dosage form comprising 15 mg of hydrocodone and 325 mg of acetaminophen may be administered.

The dosing intervals of the effective amount of the pharmaceutical composition can and will vary. For example, an effective amount of the pharmaceutical composition may be administered once a day, twice a day, or three times a day. In another embodiment, an effective amount of the pharmaceutical composition may be administered twice a day.

In general, therapeutic plasma concentrations of the opioid (e.g., hydrocodone) and the additional API (e.g., acetaminophen) are attained within about 5 minutes, 10 minutes, 15 minutes, 20 minutes, 25 minutes, 30 minutes, 35 minutes, 40 minutes, 45 minutes, 50 minutes, 55 minutes, or 60 minutes after administration of the first dose of the pharmaceutical composition. Accordingly, depending upon the severity of the pain, onset on analgesia may be attained within about 5 minutes, 10 minutes, 15 minutes, 20 minutes, 25 minutes, 30 minutes, 35 minutes, 40 minutes, 45 minutes, 50 minutes, 55 minutes, or 60 minutes after administration of the composition. Onset of analgesia may be measured by the double stopwatch method or other pain assessments such as measurements of duration of the pain and pain intensity. Generally, analgesia or pain relief will be maintained throughout the duration of the dosing interval. For example, in one embodiment, analgesia or pain relief will be maintained for 12 hours. Upon administration of the next dose of the pharmaceutical composition, therefore, analgesia or pain relief may be maintained. Accordingly, analgesia or pain relief will be maintained as long as therapeutic amounts of the pharmaceutical composition are administered at regular dosing intervals. Moreover, pain relief may be managed such that no break-through episodes of pain occur.

In some embodiments, an effective amount of the pharmaceutical composition may be administered to a subject in a fed state. In general, a fed state is defined as having consumed food within about 30 min prior to administration of the pharmaceutical composition. The food may be a high fat meal, a low fat meal, a high calorie meal, or a low calorie meal. In other embodiments, an effective amount of the pharmaceutical composition may be administered to a subject in a fasted state. In general, a fasted state is defined as not having ingested food for at least 10 hours prior to administration of the pharmaceutical composition. In some embodiments, the pharmaceutical composition may be administered to a subject who has fasted for at least 10 hours prior to the first dose and who fasts for at least one hour prior to administration of subsequent doses. In other embodiments, the pharmaceutical composition may be administered to a subject who has fasted for at least 1 hour, 2 hours, 3 hours, 4 hours, 5 hours, 6 hours, 7 hours, 8 hours, 9 hours, or 10 hours prior to administration of each dose.

The method of the present invention is useful for treating numerous pain states that are currently being treated with conventional immediate release compositions comprising acetaminophen and hydrocodone. These and additional pain states include, by way of illustration and not limitation, headache pain, pain associated with migraine, neuropathic pain selected from the group consisting of diabetic neuropathy, HIV sensory neuropathy, post-herpetic neuralgia, post-thoracotomy pain, trigeminal neuralgia, radiculopathy, neuropathic pain associated with chemotherapy, reflex sympathetic dystrophy, back pain, peripheral neuropathy, entrapment neuropathy, phantom limb pain, and complex regional pain syndrome, dental pain, pain associated with a surgical procedure and or other medical intervention, bone cancer pain, joint pain associated with psoriatic arthritis, osteoarthritic pain, rheumatoid arthritic pain, juvenile chronic arthritis associated pain, juvenile idiopathic arthritis associated pain, Spondyloarthropathies (such as ankylosing spondylitis (Mb Bechterew) and reactive arthritis (Reiter's syndrome) associated pain), pain associated with psoriatic arthritis, gout pain, pain associated with pseudogout (pyrophosphate arthritis), pain associated with systemic lupus erythematosus (SLE), pain associated with systemic sclerosis (scleroderma), pain associated with Behcet's disease, pain associated with relapsing polychondritis, pain associated with adult Still's disease, pain associated with transient regional osteoporosis, pain associated with neuropathic arthropathy, pain associated with sarcoidosis, arthritic pain, rheumatic pain, joint pain, osteoarthritic joint pain, rheumatoid arthritic joint pain, juvenile chronic arthritis associated joint pain, juvenile idiopathic arthritis associated joint pain, Spondyloarthropathies (such as ankylosing spondylitis (Mb Bechterew) and reactive arthritis (Reiter's syndrome) associated joint pain), gout joint pain, joint pain associated with pseudogout (pyrophosphate arthritis), joint pain associated with systemic lupus erythematosus (SLE), joint pain associated with systemic sclerosis (scleroderma), joint pain associated with Behcet's disease, joint pain associated with relapsing polychondritis, joint pain associated with adult Still's disease, joint pain associated with transient regional osteoporosis, joint pain associated with neuropathic arthropathy, joint pain associated with sarcoidosis, arthritic joint pain, rheumatic joint pain, acute pain, acute joint pain, chronic pain, chronic joint pain, inflammatory pain, inflammatory joint pain, mechanical pain, mechanical joint pain, pain associated with the fibromyalgia syndrome (FMS), pain associated with polymyalgia rheumatica, monarticular joint pain, polyarticular joint pain, nociceptive pain, psychogenous pain, pain of unknown etiology, pain mediated by IL-6, IL-6 soluble receptor, or IL-6 receptor, pain associated with a surgical procedure in a patient with a clinical diagnosis of OA, pain like static allodynia, pain like dynamic allodynia, and/or pain associated with Crohn's disease.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

### VI. ILLUSTRATIVE BENEFITS FOR THE PRESENT INVENTION

A non-exhaustive description of certain advantages of the present invention is described below. For example, one goal of the present invention was to develop an opioid/API formulation, such as an hydrocodone/acetaminophen formulation, that has, among other things, the following characteristics:
Rapid onset of analgesia (e.g., within approximately 30 minutes);
Extended duration of analgesia for 12 hours;
Use in the treatment of acute pain;
Administration of the dosage form without regard to food; Acetaminophen absorption primarily in a patient's upper gastrointestinal tract (upper part of small intestine, e.g., duodenum, jejunum), where acetaminophen is best absorbed;
Prolonged retention of the dosage form in the stomach;
Obtain the optimal amount and release of hydrocodone in the dosage form in order to prevent inhibition of gastric emptying;
Minimize hydrocodone's effect on gastric emptying, which can blunt acetaminophen's absorption, by finding the desirable dosing splits of each agent;
Achieve concentrations of acetaminophen in the latter part of the dosing cycle that are comparable to pre-dose concentrations of acetaminophen from immediate-release tablets (in a multiple-dose setting), allowing a patient's glutathione synthase enzyme cycle to replenish its levels of glutathione to avoid the formation of toxic intermediates with subsequent or concomitant doses of acetaminophen; and
Formulate an acetaminophen/hydrocodone product that achieves acute and prolonged analgesia with low amounts of acetaminophen and hydrocodone.

While these characteristics provided a general road map for the development work, several of these characteristics appeared to be irreconcilable. For example, administration of the dosage form to patients without regard to food was a very important characteristic, as patients suffering from acute pain often are unable to eat and retain food. Yet, in order to achieve prolonged retention of the dosage form in the stomach, one of skill in the art would administer the dosage form with food because the presence of food in the stomach decreases the stomach's migrating motor complex or “housekeeping wave.”

Nevertheless, the inventors surprisingly found that they were able to formulate an extended-release hydrocodone/acetaminophen formulation with all of the desired characteristics set forth above and with lower amounts of hydrocodone. Indeed, they developed improved extended-release hydrocodone/acetaminophen formulations that possess the following unexpected characteristics: (1) the formulations may be administered without regard to food; (2) the formulations achieve the desired pharmacokinetic parameters, such as, a rapid onset of analgesia, an extended duration of pain relief, and low plasma concentrations of acetaminophen in the latter part of the dosing cycle; and (3) the formulations provide sufficient an uninterrupted acute pain relief for 12 hours.

Accordingly, the formulations disclosed herein yield several unexpected results that are not taught or disclosed by the teachings of the art.

Having described the invention in detail, it will be apparent that modifications and variations are possible without departing from the scope of the invention defined in the appended claims.

### EXAMPLES

The following examples are included to demonstrate certain embodiments of the invention. Those of skill in the art should, however, in light of the present disclosure, appreciate that modifications can be made in the specific embodiments that are disclosed and still obtain a like or similar result without departing from the spirit and scope of the invention, therefore all matter set forth is to be interpreted as illustrative and not in a limiting sense.

### Example 1

### Pharmacokinetic Study Involving Hydrocodone and Acetaminophen

A four-way crossover pharmacokinetic study was conducted. In a first trial (Treatment A), thirty-five subjects in a fasted state were administered a single, two-tablet dose of hydrocodone/acetaminophen, each tablet containing 7.5 mg hydrocodone, 325 mg acetaminophen, and having slow release properties as compared to an immediate release formulation. (See selected examples from Chart No. 1). In a second trial (Treatment B), thirty-five subjects in a fasted state were administered a single, two-tablet dose of hydrocodone/acetaminophen, each tablet containing 7.5 mg hydrocodone, 325 mg acetaminophen, and having medium release properties as compared to an immediate release formulation. (See selected examples from Chart No. 1). In a third trial (Treatment C), thirty-five subjects in a fed state were administered a single, two-tablet medium-release dose of hydrocodone/acetaminophen, each tablet containing 7.5 mg hydrocodone, 325 mg acetaminophen, and having medium release properties as compared to an immediate release formulation. (See selected examples from Chart Nos. 1 and 2). In a fourth trial (Treatment D), thirty-five subjects were administered a single, two-tablet dose of an immediate release tablet containing 7.5 mg hydrocodone and 325 mg acetaminophen.

The pharmacokinetic profiles from time 0 to 36 hours for hydrocodone and acetaminophen in each of these trials are shown in FIGS. 1 and 2, respectively. The pharmacokinetic profiles from time 0 to 12 hours for hydrocodone and acetaminophen in each of these trials are shown in FIGS. 3 and 4, respectively. The pharmacokinetic parameters of hydrocodone and acetaminophen are summarized in Tables 1 and 2, respectively. The simulated pharmacokinetic profiles from time 0 to 144 hours for hydrocodone and acetaminophen in each of these trials are shown in FIGS. 5 and 6, respectively.

TABLE 1
Pharmacokinetic parameters for hydrocodone

| | Treatment | Treatment | Treatment | Treatment |
|---|---|---|---|---|
| | A, | B, | C, | D, |
| | Mean (SD) | Mean (SD) | Mean (SD) | Mean (SD) |
| Parameter | (N = 35) | (N = 35) | (N = 35) | (N = 35) |
| AUC0-t | 254.12 | 243.88 | 265.08 | 261.60 |
| (ng · h/mL) | (71.48) | (67.86) | (73.62) | (72.55) |
| AUC0-inf | 264.47 | 251.04 | 268.73 | 264.79 |
| (ng · h/mL) | (72.66) | (69.72) | (75.25) | (73.55) |
| Cmax (ng/mL) | 18.62 (5.38) | 18.93 (5.58) | 19.73 (4.06) | 22.84 (6.51) |
| Tmax (h)a | 4.05 | 4.00 | 5.92 | 8.00 |
| | (2.00-7.00) | (1.00-7.00) | (2.00-12.08) | (0.67-10.02) |
| tlag (h)a | 0.17 | 0.17 | 0.17 | 0.17 |
| | (0.00-0.37) | (0.00-0.48) | (0.00-0.67) | (0.00-0.33) |
| t1/2 (h) | 7.14 (2.55) | 6.70 (1.56) | 4.91 (0.59) | 4.87 (0.57) |
| Kel (h − 1) | 0.1087 | 0.1087 | 0.1431 | 0.1442 |
| | (0.0351) | (0.0238) | (0.0174) | (0.0171) |

Median (minimum-maximum).

TABLE 2
Pharmacokinetic parameters for acetaminophen

| | Treatment A, | Treatment B, | Treatment C, | Treatment D |
|---|---|---|---|---|
| | Mean (SD) | Mean (SD) | Mean (SD) | Mean (SD) |
| Parameter | (N = 35) | (N = 35) | (N = 35) | (N = 35) |
| AUC0-t | 30578 (9205) | 28939 (8364) | 29900 (8544) | 30771 (9518) |
| (ng · h/mL) | | | | |
| AUC0-inf | 33417 (9306) | 31073 (8688) | 31512 (8943) | 31833 (9831) |
| (ng · h/mL) | | | | |
| Cmax | 5030 (1678) | 4950 (1586) | 3343 (847) | 4755 (1673) |
| (ng/mL) | | | | |
| Tmax (h)a | 0.67 | 0.67 | 2.00 | 0.67 |
| | (0.33-2.00) | (0.22-1.03) | (0.33-5.92) | (0.33-7.00) |
| tlag (h)a | 0.00 | 0.00 | 0.17 | 0.00 |
| | (0.00-0.33) | (0.00-0.17) | (0.00-0.50) | (0.00-0.33) |
| t1/2 (h) | 8.05 | 6.57 | 5.10 | 4.36 |
| | (3.33) | (2.11) | (2.24) | (1.32) |
| Kel (h − 1) | 0.1030 | 0.1196 | 0.1529 | 0.1718 |
| | (0.0478) | (0.0504) | (0.0498) | (0.0509) |

Median (minimum-maximum).

These results indicate that the subjects exhibited an initial rapid rise in hydrocodone concentrations to provide early onset of action with the concentrations falling slowly over a period of twelve hours. The median Tlag was unaffected by the formulations in comparison to an immediate release tablet containing 7.5 mg hydrocodone and 325 mg acetaminophen. Subjects also exhibited an initial rapid rise in acetaminophen concentrations to provide the desired early onset of action with the concentrations reaching levels that were lower than an immediate release tablet containing 7.5 mg hydrocodone and 325 mg acetaminophen at around twelve hours. Accordingly, the pharmaceutical compositions administered in Treatments A-C exhibited the desired APAP “time-off” feature in their pharmacokinetic profiles.

Administration of the pharmaceutical formulations with food had no effect on Cmax and AUC of hydrocodone, although the Tmax was delayed by two hours. Tlag was unaffected. The Cmax of APAP decreased by about 31% when administered with food, but there was no change in AUC. Tmax of APAP was delayed by a little more than one hour. No dose dumping was observed from any of the formulations.

The pharmacokinetic profiles of both the pharmaceutical formulations having slow and medium release properties as compared to an immediate release formulation satisfy the desired pharmacokinetic parameters for both hydrocodone and acetaminophen. The observed Cmax and AUC values were suitable for hydrocodone/acetaminophen formulations containing an immediate release and extended release portion.

### Example 2

### Clinical Pharmacokinetic Analysis of an Extended Release Formulation of Hydrocodone/Acetaminophen Administered Under Fed and Fasted Conditions

An open-label, randomized, three-period crossover study was conducted to evaluate the pharmacokinetics (PK), bioavailability, and safety of two tablets of a multi-layer extended-release formulation (7.5 mg hydrocodone bitartrate (HB)/325 mg acetaminophen (APAP)), administered as a single dose in normal, healthy subjects under fed (high-fat or and low-fat meal) and fasted conditions (i.e., 10 hr fast).

This single center, open-label, randomized, 3-period, 6-sequence crossover study in normal, healthy subjects was designed to evaluate the effect of a high-fat and low-fat meal on the PK, bioavailability, and safety of a multilayer ER tablet formulation of 7.5 mg HB/325 mg APAP (see selected example from Chart No. 1). The formulation was orally administered as 2 tablets (15 mg HB/650 mg APAP total dose) under 2 types of fed (high-fat and low-fat) and fasted conditions. Forty-eight subjects were enrolled and 40 subjects completed the study. Only subjects that completed all 3 study periods have been included in the PK evaluation.

Following a 10 hour overnight fast, subjects randomized to Treatment A consumed an entire standardized FDA high-fat breakfast (approximately 1,000±100 calories and approximately 50% from fat); those receiving Treatment B consumed an entire low-fat breakfast (approximately 800±80 calories and approximately 25% to 30% from fat). Breakfasts were consumed within 30 minutes prior to Hour 0 study drug administration. Subjects who could not consume the entire breakfast in the allotted time were dropped from the study. Subjects randomized to Treatment C were administered study drug under fasted conditions following an overnight fast of at least 10 hours. No food was allowed for the first 4 hours post dose. Blood samples were collected pre-dose (up to 60 minutes prior to dose), and at 15 min, 30 min, 45 min and 1, 2, 3, 4, 5, 6, 7, 8, 9, 10, 11, 12, 16, 18, 20, 24, 36 and 48 hours post-dose, and the resulting plasma samples were analyzed for Hydrocodone (HC) and APAP using a validated liquid chromatography-tandem mass spectrometry assay with a linear range of 0.100 to 50 ng/mL for HC and 100 to 15,000 ng/mL for APAP. The following PK parameters were calculated for hydrocodone and acetaminophen using standard non-compartmental methods:
area under the plasma concentration curve to last quantifiable concentration AUC₍₀₋ₜ₎
area under the plasma concentration curve to infinite time AUC_{(0-inf)}
maximum observed plasma concentration _{(Cmax)}
time observed maximum plasma concentration ₍ₜₘₐₓ₎
lag time _{(tlag)}
apparent first-order terminal elimination rate constant ₍ₖₑₗ₎
apparent plasma terminal elimination half-life _{(t1/2)}

Tables 3 and 4 presents PK parameters for HC under the three treatment conditions, and FIG. 7 presents plasma HC concentration-time profiles for the treatments. Mean plasma concentration profiles of HC revealed that HC was rapidly absorbed under both fed (high and low fat meal) and fasted conditions. There was a slight lag (median 0.25 hours) when the formulation was administered after a meal. The median of the time of observed maximum plasma concentrations (Tmax) was 4 hours after administration under both the low fat and fasted conditions. Median Tmax for HC under high fat conditions was significantly delayed, as compared to fasted conditions (6 hr vs. 4 hr; P<0.05). Average maximum plasma HC concentrations (Cmax) were 23.09 ng/mL after a low fat breakfast, 21.66 ng/mL after a high fat breakfast, and 20.33 ng/mL under fasted conditions.

TABLE 3
Hydrocodone Pharmacokinetic Estimates (2 tablets of 7.5/325)

| | Treatment A | Treatment B | Treatment C |
|---|---|---|---|
| | Fed (High Fat) | Fed (Low Fat) | Fasted |
| | Mean (SD) | Mean (SD) | Mean (SD) |
| Parameter | (N = 31) | (N = 31) | (N = 31) |
| AUC0-t | 301.50 | 299.72 | 280.10 |
| (ng · hr/mL) | (52.81) | (57.01) | (58.80) |
| AUC0-inf | 303.66 | 301.95 | 282.94 |
| (ng · hr/mL) | (53.13) | (57.83) | (59.86) |
| Cmax (ng/mL) | 21.66 | 23.09 | 20.33 |
| | (4.88) | (3.79) | (4.33) |
| Tmax (hr)a | 6 (2-11) | 4 (2-7) | 4 (2-7) |
| Kel (1/hr) | 0.1273 | 0.1218 | 0.1110 |
| | (0.0207) | (0.0202) | (0.0194) |
| tlag (hr)a | 0 | 0.25 | 0 |
| | (0-1.07) | (0-0.75) | (0-0.50) |
| t1/2 (hr) | 5.58 (0.85) | 5.85 (1.00) | 6.43 (1.11) |

aMedian (minimum-maximum).

A comparison of Cmax showed that HC concentrations were 6% and 14% higher when the formulation was given under high fat (Treatment A) and low fat (Treatment B) conditions, compared to fasted conditions (Treatment C; see Table 3). The Cmax for Treatment A was bioequivalent to both Treatments B (88%-99%) and C (101%-113%) as the 90% CIs for the geometric ratios were contained within 80% to 125% (see Table 4). The Cmax observed for Treatment B was also bioequivalent to Treatment C (108%-122%). AUCs were approximately 7% higher when the formulation was administered under fed conditions (high and low fat), as compared to fasted conditions (Table 3). AUC for both Treatments A and B (high fat and low fat) were bioequivalent to Treatment C (fasted; 104%-112% and 103%-111% for AUC0-t and 104%-112% and 103%-111% for AUC(0-inf) (Table 4). The apparent plasma terminal elimination half-life (t½) for HC was similar when the formulation was administered under fed (5.58 hours) and fasted conditions (6.43 hours).

TABLE 4
Hydrocodone Geometric LSMEANS Ratio (%) (90% Cl)

| | Treatment A/C | Treatment B/C | Treatment A/B |
|---|---|---|---|
| | Fed (High | Fed (Low | Fed (High Fat)/ |
| Parameter | Fat)/Fasted | Fat)/Fasted | Fed (Low Fat) |
| AUC₀₋ₜ | 108.40 | 107.45 | 100.89 |
| (ng · hr/mL)^{a} | (104.44-112.51) | (103.53-111.52) | (97.22-104.69) |
| AUC_{0-inf} | 108.11 | 107.17 | 100.88 |
| (ng · hr/mL)^{a} | (104.14-112.24) | (103.23-111.26) | (97.19-104.72) |
| Cₘₐₓ (ng/mL)^{a} | 106.66 | 114.75 | 92.95 |
| | (100.54-113.15) | (108.16-121.73) | (87.64-98.59) |

PK parameters for APAP are presented in Tables 5 and 6 and the plasma APAP concentration-time profiles are presented in FIG. 8. APAP was rapidly absorbed following administration under fed (high and low fat meals) and fasted conditions. There was a slight lag when the formulation was administered after a low fat breakfast (median lag time [flag] 0.25 hours). There was no lag in the absorption of APAP when administered following a high fast breakfast or after fasting. The time to Cₘₐₓ was significantly (P<0.05) longer when administered after a meal (high and low fat; median Tmax=2 hours) than when administered under fasted conditions (median Tmax=0.75 hour). Average Cmax values for APAP were lower after a high (4.317 ng/mL) and low fat (4,122 ng/mL) meal than when administered under fasted conditions (5307 ng/mL). Geometric mean ratios for Cmax following Treatments A and B were 20% to 22% lower than for Treatment C (Table 6). The 90% CIs for Cmax following Treatment A (75%-88%) and Treatment B (73%-83%) with reference to fasted state were outside the bioequivalent range of 80%-125%. The AUCs for APAP were almost identical when the formulation was administered under high fat, low fat, or fasting conditions. (Comparison of geometric mean ratios of AUC0-t and AUC0 inf for Treatments A (90% CI 100%-105% and 98%-103%) and B (90% CI 96%-101% and 97% to 103%) with those for Treatment C showed that treatments were bioequivalent. The t½ for APAP after the formulation was administered after a high or low fat meal (5 hours) was slightly shorter than when administered under fasted conditions (7 hours).

TABLE 5
APAP Pharmacokinetic Estimates (2 tablets of 7.5/325)

| | Treatment A | | Treatment B | | Treatment C | |
|---|---|---|---|---|---|---|
| | Fed (High Fat) | | Fed (Low Fat) | | Fasted | |
| | Mean (SD) | | Mean (SD) | | Mean (SD) | |
| Parameter | (N = 31) | | (N = 31) | | (N = 31) | |
| AUC₀₋ₜ | 33210.39 | | 32415.11 | | 32149.34 | |
| (ng · hr/mL) | (10402.75) | | (9586.52) | | (9431.97) | |
| AUC_{0-inf} | 34689.91 | | 34092.21 | | 34803.59 | |
| (ng · hr/mL) | (10672.37) | | (9949.21) | | (9635.34) | |
| Cₘₐₓ (ng/mL) | 4317.00 | | 4122.25 | | 5307.00 | |
| | (1185.08) | | (877.19) | | (1419.43) | |
| Tₘₐₓ (hr)^{a} | 2 | | 2 | | 0.75 | |
| | (0.25-6.05) | | (0.75-7.00) | | (0.25-5.00) | |
| Kₑₗ (1/hr) | 0.1444 | | 0.1317 | | 0.1072 | |
| | (0.0470) | | (0.0356) | | (0.0402) | |
| t_{lag} (hr)^{a} | 0 | (0-0.63) | 0.25 | (0-0.50) | 0.00 | (0-0.25) |
| t_{1/2} (hr) | 5.37 | (2.02) | 5.68 | (1.68) | 7.37 | (2.77) |

^{a}Median (minimum-maximum).

TABLE 6
APAP Geometric LSMEANS Ratio (%) (90% Cl)

| | Treatment A/C | Treatment B/C | Treatment A/B |
|---|---|---|---|
| | Fed (High | Fed (Low | Fed (High Fat)/ |
| Parameter | Fat)/Fasted | Fat)/Fasted | Fed (Low Fat) |
| AUC₀₋ₜ | 102.70 | 100.74 | 101.94 |
| (ng · hr/mL)^{a} | (100.05-105.42) | (98.15-103.41) | (99.32-104.63) |
| AUC_{0-inf} | 100.32 | 98.66 | 101.69 |
| (ng · hr/mL)^{a} | (97.71-103.01) | (96.09-101.30) | (99.04-104.40) |
| Cₘₐₓ (ng/mL)^{a} | 80.49 | 78.10 | 103.06 |
| | (75.44-85.88) | (73.20-83.33) | (96.62-109.93) |

In summary, total exposure (AUC) for HC was slightly increased (by about 7%) when the formulation was administered with food (after high- or low-fat meal); however, AUCs for HC were equivalent between all treatments (high fat vs. fasted, low fat vs. fasted and high fat vs. low fat). Peak exposure (Cmax) for HC was 6% and 14% higher under high fat and low-fat conditions, respectively, compared to fasted conditions. The Cmax for HC after a high-fat meal and low fat meal were bioequivalent to fasted conditions. The AUCs for APAP were equivalent between all treatments (high fat vs. fasted, low fat vs. fasted, and high fat vs. low fat). The peak exposure (Cmax) for APAP was decreased by about 20% in fed (high- and low-fat) states as compared to the fasted state.

A similar trial was conducted to evaluate the pharmacokinetics and bioavailability following administration of a single-dose of three tablets of the multi-layer extended release formulation comprising 7.5 mg hydrocodone and 325 mg APAP per tablet under the same fed and fasted conditions. Again, plasma hydrocodone and APAP concentrations were rapidly obtained after administration, with plasma hydrocodone concentrations sustained over a 12-hour dosing interval. Plasma APAP concentrations were sufficiently low after 10-12 hours to permit the APAP “time-off” period before the next dose. The confidence intervals for the total exposure (AUC) demonstrated that ingestion of a low-fat meal versus a high-fat meal did not affect the AUC for either hydrocodone or APAP. Peak exposure (Cmax) for hydrocodone administered after ingestion of a high-fat meal did not differ from that under fasted conditions but increased slightly when administered after a low-fat meal. Peak exposure (Cmax) was lower under both fed conditions than fasted conditions.

These findings support the safe and appropriate administration of the multi-layer extended release formulation without regard to food.

### Example 3

### Clinical Pharmacokinetic Analysis of an Extended Release Formulation of 7.5 Mg Hydrocodone/325 mg Acetaminophen—Single and Multiple Doses

An open-label, randomized, 3-period crossover study was performed to evaluate single and multiple dose pharmacokinetics, bioavailability, and safety of an extended release formulation containing 7.5 mg hydrocodone/325 mg acetaminophen under fasted conditions in normal, healthy subjects. (See example in Chart 1). The pharmacokinetics (PK) and bioavailability following administration of a 7.5 mg hydrocodone/325 mg acetaminophen tablet disclosed herein administered as either 1 or 2 tablets every 12 hours was compared to 1 immediate release tablet containing 7.5 mg hydrocodone/325 mg acetaminophen and administered every 6 hours (Q6h). The study also assessed the PK proportionality between the 1 tablet and 2 tablet dosing configurations of the 7.5 mg hydrocodone/325 mg acetaminophen tablet disclosed herein. In addition, the study evaluated the safety of the 1 tablet and 2 tablet dosing configurations of the 7.5 mg hydrocodone/325 mg acetaminophen tablet disclosed herein compared with immediate release tablet containing 7.5 mg hydrocodone/325 mg acetaminophen.

The subjects were randomly divided into three treatment options:
Treatment A: One tablet of the 7.5 mg hydrocodone/325 mg acetaminophen tablet disclosed herein administered orally under fasted conditions on Day 1 followed by 1 tablet given Q12h (beginning on Day 3 for a total of 9 doses).
Treatment B: Two tablets of the 7.5 mg hydrocodone/325 mg acetaminophen tablet disclosed herein administered orally under fasted conditions on Day 1 followed by 2 tablets given Q12h (beginning on Day 3 for a total of 9 doses).
Treatment C: One tablet of an immediate release tablet containing 7.5 mg hydrocodone/325 mg acetaminophen administered orally Q6h for 2 doses under fasted conditions on Day 1 followed by 1 tablet given Q6h (beginning on Day 3 for a total of 18 doses).

The pharmacokinetic (PK) parameters of a single dose of hydrocodone and acetaminophen are presented below in Tables 7 and 8 respectively. The plasma concentrations of hydrocodone and acetaminophen are presented in FIGS. 9 and 10, respectively. The pharmacokinetic parameters demonstrate that the formulations disclosed herein containing 7.5 mg hydrocodone/325 mg acetaminophen are dose proportional for both hydrocodone and acetaminophen. The pharmacokinetic profiles for hydrocodone showed an initial rapid rise in the concentrations of hydrocodone to provide a subject with the desired early onset of action (the median tlag for the formulation disclosed herein is 0.08 hour and for the immediate release tablet it is 0.04 hour). The hydrocodone concentrations in the subjects that took the formulations disclosed herein containing 7.5 mg hydrocodone/325 mg acetaminophen also fell slowly over a period of 12 hours.

Similarly, the pharmacokinetic profiles for acetaminophen showed an initial rapid rise in the concentrations of acetaminophen to provide the desired early onset of action (the median tlag=0 hr for the formulation disclosed herein containing 7.5 mg hydrocodone/325 mg acetaminophen, which is the same as the immediate release tablet containing 7.5 mg hydrocodone/325 mg acetaminophen). The acetaminophen concentrations achieved by the formulation disclosed herein containing 7.5 mg hydrocodone/325 mg acetaminophen fell slowly, reaching levels that were lower than the immediate release tablet at around 12 hours, showing the desired “time off” from acetaminophen.

TABLE 7
Single Dose PK Parameters for Hydrocodone (Mean ± SD)

| | | | | | Commercially- | |
|---|---|---|---|---|---|---|
| | | | | | available | |
| | | | | | immediate | |
| | | | | | release | |
| | One Tablet | | Two Tablets | | product | |
| | (7.5 HC/325 | | (7.5 HC/325 | | (7.5 HC/325 | |
| PK | APAP) | | APAP) | | APAP) | |
| Parameters | Treatment A | | Treatment B | | Treatment C | |
| Cₘₐₓ | 8.98 | (2.02) | 17.53 | (3.69) | 24.48 | (5.69) |
| (ng/mL) | | | | | | |
| AUCₜ | 122.43 | (30.53) | 248.48 | (63.22) | 254.39 | (63.21) |
| (ng · hr/mL) | | | | | | |
| AUC_{inf} | 124.24 | (30.63) | 251.20 | (63.57) | 256.24 | (63.71) |
| (ng · hr/mL) | | | | | | |
| t_{lag} (hr) | 0.12 | (0.14) | 0.08 | (0.13) | 0.04 | (0.09) |
| Tₘₐₓ* (hr) | 8.00 | | 3.00 | | 3.00 | |
| | (0.25-6.00) | | (0.25-2.00) | | (0.25-8.00) | |
| T_{1/2} (hr) | 6.26 | (1.41) | 6.41 | (1.09) | 5.37 | (0.83) |

TABLE 8
Single Dose PK Parameters for Acetaminophen (Mean ± SD)

| | | | Commercially- |
|---|---|---|---|
| | | | available |
| | | | immediate |
| | | | release |
| | One Tablet | Two Tablets | product |
| | (7.5 HC/325 | (7.5 HC/325 | (7.5 HC/325 |
| PK | APAP) | APAP) | APAP) |
| Parameters | Treatment A | Treatment B | Treatment C |
| Cₘₐₓ | 2604.55 | 5432.05 | 4912.05 |
| (ng/mL) | (925.57) | (1793.44) | (1647.69) |
| AUCₜ | 13248.82 | 28593.91 | 27928.74 |
| (ng · hr/mL) | (3889.65) | (8400.40) | (9086.86) |
| AUC_{inf} | 15124.65 | 31049.83 | 28993.63 |
| (ng · hr/mL) | (4179.45) | (8899.91) | (9243.85) |
| t_{lag} (hr) | 0.06 (0.11) | 0.02 (0.07) | 0.01 (0.04) |
| Tₘₐₓ* (hr) | 0.50 | 0.50 | 0.50 |
| | (0.25-6.00) | (0.25-2.00) | (0.25-8.00) |
| T_{1/2} (hr) | 7.73 (2.88) | 8.32 (4.34) | 4.29 (1.40) |

*Tmax values: median (range)

As shown below in Table 9, the 90% Confidence Intervals for all the pharmacokinetic parameters for hydrocodone dosed as a single dose of either one tablet or two tablets of the formulations disclosed herein containing 7.5 mg hydrocodone/325 mg acetaminophen were contained within the 80-125% bioequivalence range for the immediate release tablet containing 7.5 mg hydrocodone/325 mg acetaminophen. Similarly, as shown below in Table 10, the 90% Confidence Intervals for all the pharmacokinetic parameters for acetaminophen dosed as a single dose of either one tablet or two tablets of the formulations disclosed herein containing 7.5 mg hydrocodone/325 mg acetaminophen were contained within the 80-125% bioequivalence range for the immediate release tablet containing 7.5 mg hydrocodone/325 mg acetaminophen. However, the cmax values for both dosing configurations for the formulations disclosed herein were lower as compared to the immediate release product.

TABLE 9
Single Dose Bioequivalence Parameters for Hydrocodone

| | | Immediate Release Tablet | | |
|---|---|---|---|---|
| | | | 90% Cl | |
| Test | PK Parameter | LSM | Lower | Upper |
| One Tablet | Cmax | 73.53 | 70.46 | 76.74 |
| | AUCt | 96.2 | 92.33 | 100.23 |
| | AUCinf | 96.97 | 93.10 | 100.99 |
| Two Tablets | Cmax | 71.86 | 68.85 | 74.99 |
| | AUCt | 97.59 | 93.66 | 101.68 |
| | AUCinf | 97.96 | 94.06 | 102.03 |
| | | Reference: Two tablets of the | | |
| | | 7.5 mg HC/325 mg APAP | | |
| One Tablet | Cmax | 102.33 | 98.05 | 106.80 |
| | AUCt | 98.58 | 94.61 | 102.71 |
| | AUCinf | 98.98 | 95.04 | 103.09 |

TABLE 10
Single Dose Bioequivalence Parameters for Acetaminophen

| | | Immediate Release Tablet | | |
|---|---|---|---|---|
| | | | 90% Cl | |
| Test | PK Parameter | LSM | Lower | Upper |
| One Tablet | Cmax | 105.34 | 97.98 | 113.25 |
| | AUCt | 95.16 | 91.89 | 98.55 |
| | AUCinf | 104.99 | 101.17 | 108.95 |
| Two Tablets | Cmax | 110.78 | 103.04 | 119.10 |
| | AUCt | 102.62 | 99.10 | 106.28 |
| | AUCinf | 107.42 | 103.52 | 111.48 |
| | | Reference: Two tablets of the | | |
| | | 7.5 mg HC/325 mg APAP | | |
| One Tablet | Cmax | 95.09 | 88.45 | 102.24 |
| | AUCt | 92.73 | 89.54 | 96.03 |
| | AUCinf | 97.73 | 94.18 | 101.42 |

The pharmacokinetic (PK) parameters of multiple doses of hydrocodone and acetaminophen are presented below in Tables 11 and 12, respectively. The plasma concentrations of hydrocodone and acetaminophen are presented in FIGS. 11 and 12, respectively. The pharmacokinetic parameters demonstrate that the formulations disclosed herein containing 7.5 mg hydrocodone/325 mg acetaminophen are dose proportional for both hydrocodone and acetaminophen. No differences were observed in dose-normalized Cmaxss, Cminss, AUC0-12sshr, Cavss, and fluctuation of hydrocodone and acetaminophen following administration of the formulation disclosed herein containing 7.5 mg hydrocodone/325 mg acetaminophen as either 1-tablet or 2-tablet dosing configurations, or the immediate release tablet containing 7.5 mg hydrocodone/325 mg acetaminophen. For acetaminophen, the 1-tablet, 2-tablet and immediate release tablet had the same (0.5 hr) median Tmaxss. For hydrocodone, the median Tmaxss of the formulation disclosed herein containing 7.5 mg hydrocodone/325 mg acetaminophen (dosed as either 1 and 2 tablets) was 2 hours while the median Tmaxss for the immediate release tablet containing 7.5 mg hydrocodone/325 mg acetaminophen was 1 hour. No difference was observed in the swing of hydrocodone for the formulation disclosed herein containing 7.5 mg hydrocodone/325 mg acetaminophen dosed as either 1 and 2-tablet and the immediate release tablet containing 7.5 mg hydrocodone/325 mg acetaminophen. In the case of acetaminophen, the swing was partially out of the range to demonstrate no difference (107.33-132.50).

TABLE 11
Multiple Dose PK Parameters for Hydrocodone (Mean ± SD)

| | | | | | Commercially- | |
|---|---|---|---|---|---|---|
| | | | | | available | |
| | | | | | immediate | |
| | | | | | release | |
| | One Tablet | | Two Tablets | | product | |
| | (7.5 HC/325 | | (7.5 HC/325 | | (7.5 HC/325 | |
| | APAP) | | APAP) | | APAP) | |
| PK Parameters | Treatment A | | Treatment B | | Treatment C | |
| Cmax(ss) | 15.57 | (3.85) | 30.54 | (6.87) | 33.80 | (8.74) |
| (ng/mL) | | | | | | |
| Cmin(ss) | 6.49 | (2.41) | 13.38 | (4.12) | 14.13 | (4.67) |
| (ng/mL) | | | | | | |
| Cavg(ss) | 11.20 | (3.16) | 22.54 | (5.36) | 22.38 | (5.82) |
| (ng/mL) | | | | | | |
| AUC0-12(ss) | 134.36 | (37.91) | 270.52 | (64.29) | 268.57 | (69.87) |
| (ng · hr/mL) | | | | | | |
| Tmax(SS) | 146.00 | | 146.00 | | 145.00 | |
| (hr) | (144.50-150.00) | | (144.50-150.00) | | (144.50-142.00) | |
| Swing | 1.53 | (1.00) | 1.37 | (1.42) | 1.53 | (3.16) |
| Fluctuation | 83.76 | (19.00) | 77.54 | (16.70) | 90.60 | (3054) |
| (%) | | | | | | |
| Accumulation | 1.43 | (0.20) | 1.43 | (0.20) | 1.23 | (0.29) |

Index

* Tmax values: median (range)

TABLE 12
Multiple Dose PK Parameters for Acetaminophen (Mean ± SD)

| | | | Commercially- |
|---|---|---|---|
| | | | available |
| | | | immediate |
| | One Tablet | Two Tablets | release product |
| | (7.5 HC/ | (7.5 HC/ | (7.5 HC/ |
| | 325 APAP) | 325 APAP) | 325 APAP) |
| PK Parameters | Treatment A | Treatment B | Treatment C |
| Cmin (ss) | 464.89 | 844.68 | 893.86 |
| (ng/mL) | (166.72) | (293.41) | (310.09) |
| Cavg (ss) | 1169.43 | 2238.75 | 2267.63 |
| (ng/mL) | (316.86) | (577.32) | (592.95) |
| AUC0-12 (ss) | 14033.21 | 26864.94 | 27211.55 |
| (ng.hr/mL) | (3802.32) | (6927.89) | (7115.37) |
| Tmax (SS) | 144.5 | 144.5 | 144.5 |
| (hr) | (144.25-148.00) | (144.25-146.00) | (144.25-152.00) |
| Swing | 6.43 | 6.78 | 5.76 |
| | (0.96) | (0.44) | (2.80) |
| Fluctuation | 237.58 | 237.66 | 211.43 |
| (%) | (85.70) | (74.05) | (76.05) |
| Accumulation | 1.28 | 1.21 | 1.05 |
| Index | (0.21) | (0.17) | (0.06) |

* Tmax values: median (range)

As shown below in Table 13, the 90% Confidence Intervals for all the pharmacokinetic parameters for hydrocodone dosed as multiple doses of either one tablet or two tablets of the formulations disclosed herein containing 7.5 mg hydrocodone/325 mg acetaminophen were contained within the 80-125% bioequivalence range for the immediate release tablet containing 7.5 mg hydrocodone/325 mg acetaminophen. Similarly, as shown below in Table 14, the 90% Confidence Intervals for all the pharmacokinetic parameters (except the swing) for acetaminophen dosed as multiple doses of either one tablet or two tablets of the formulations disclosed herein containing 7.5 mg hydrocodone/325 mg acetaminophen were contained within the 80-125% bioequivalence range for the immediate release tablet containing 7.5 mg hydrocodone/325 mg acetaminophen.

TABLE 13
Multiple Dose Bioequivalence Parameters for Hydrocodone

| | | Immediate Release Tablet | | |
|---|---|---|---|---|
| | | | 90% CI | |
| Test | PK Parameter | LSM | Lower | Upper |
| One Tablet | Cmax (ss) | 92.46 | 89.07 | 95.98 |
| | Cmin (ss) | 91.22 | 87.61 | 94.98 |
| | Cavg (ss) | 99.7 | 96.85 | 102.64 |
| | AUC0-12 (ss) | 99.71 | 96.85 | 102.65 |
| | Swing | 103.98 | 97.05 | 111.4 |
| | Fluctuation | 95.13 | 90.54 | 99.94 |
| Two Tablets | Cmax (ss) | 90.91 | 87.58 | 94.38 |
| | Cmin (ss) | 95.33 | 91.56 | 99.26 |
| | Cavg (ss) | 101.15 | 98.25 | 104.14 |
| | AUC0-12 (ss) | 101.15 | 98.26 | 104.14 |
| | Swing | 93.88 | 87.62 | 100.58 |
| | Fluctuation | 88.47 | 84.21 | 92.95 |
| | | Reference: Two tablets of the | | |
| | | 7.5 mg HC/325 mg APAP | | |
| One Tablet | Cmax (ss) | 101.7 | 97.97 | 105.57 |
| | Cmin (ss) | 95.69 | 91.9 | 99.64 |
| | Cavg (ss) | 98.57 | 95.74 | 101.48 |
| | AUC0-12 (ss) | 98.57 | 95.75 | 101.48 |
| | Swing | 110.76 | 103.38 | 118.66 |
| | Fluctuation | 107.52 | 102.34 | 112.96 |

TABLE 14
Multiple Dose Bioequivalence Parameters for Acetaminophen

| | | Immediate Release Tablet | | |
|---|---|---|---|---|
| | | | 90% CI | |
| Test | PK Parameter | LSM | Lower | Upper |
| One Tablet | Cmax (ss) | 113.39 | 105.69 | 121.66 |
| | Cmin (ss) | 102.92 | 98.12 | 107.95 |
| | Cavg (ss) | 102.81 | 100.19 | 105.49 |
| | AUC0-12 (ss) | 102.81 | 100.19 | 105.49 |
| | Swing | 112.44 | 101.2 | 124.93 |
| | Fluctuation | 112.56 | 103.72 | 122.17 |
| Two Tablets | Cmax (ss) | 109.25 | 101.83 | 117.21 |
| | Cmin (ss) | 94.27 | 89.87 | 98.88 |
| | Cavg (ss) | 98.76 | 96.25 | 101.33 |
| | AUC0-12 (ss) | 98.76 | 96.25 | 101.33 |
| | Swing | 119.25 | 107.33 | 132.5 |
| | Fluctuation | 113.83 | 104.88 | 123.54 |
| | | Reference: Two tablets of the | | |
| | | 7.5 mg HC/325 mg APAP | | |
| One Tablet | Cmax (ss) | 103.79 | 96.74 | 111.36 |
| | Cmin (ss) | 109.17 | 104.08 | 114.51 |
| | Cavg (ss) | 104.1 | 101.45 | 106.82 |
| | AUC0-12 (ss) | 104.1 | 101.45 | 106.82 |
| | Swing | 94.29 | 84.86 | 104.76 |
| | Fluctuation | 98.89 | 91.11 | 107.32 |

### Example 4

### Partial Areas Under the Curve for Hydrocodone and Acetaminophen

A cross-study comparison of the partial AUCs for acetaminophen following oral administration of the pharmaceutical compositions described in Treatment A of Example 1, and Treatment C of Example 2 was performed. These results are summarized in Tables 15 and 16. Additionally, the partial AUCs for hydrocodone were determined and are summarized in Tables 17.

TABLE 15
Mean (SD) Parameter Estimates for Partial AUCs for Acetaminophen.

| Study | AUC_{0-1 h} | AUC_{1-12 h} | AUC_{12-36 h} | AUC_{0-1.27 h} | AUC_{1.27-36 h} | AUC₀₋ₜ |
|---|---|---|---|---|---|---|
| Treatment A | 3276.62 | 20624.53 | 7774.64 | 3816.89 | 27858.88 | 30618.62 |
| (Ex. 1) | | | | | | |
| Treatment C | 3264.68 | 22299.56 | 8284.15 | 4428.19 | 20420.21 | 32441.45 |
| (Ex. 2) | | | | | | |

TABLE 16
Percent of the partial AUC compared to AUC₀₋ₜ.

| Study | AUC_{1-12 h} | AUC_{12-36 h} |
|---|---|---|
| Treatment A | 67% | 25% |
| (Ex. 1) | | |
| Treatment C | 69% | 26% |
| (Ex. 2) | | |

TABLE 17
Mean (SD) Parameter Estimates for Partial AUCs for Hydrocodone.

| | AUC₀₋ₜ | AUC_{0-2.44 h} | AUC_{2.44-36 h} |
|---|---|---|---|
| Study | (ng · h/mL) | (ng · h/mL) | (ng · h/mL) |
| Treatment A | 254.16 (71.57) | 26.33 (8.70) | 227.93 (65.32) |
| (Ex. 1) | | | |
| Treatment C | 280.08 (59.58) | 27.41 (9.34) | 246.25 (52.99) |
| (Ex. 2) | | | |

Further, it was determined that Tmax for an immediate release tablet containing 7.5 mg hydrocodone and 325 mg acetaminophen plus two standard deviations was about 3 hr. Some of the partial AUCs of the pharmaceutical formulation described herein were determined in accordance with this time interval.

The bioequivalence determinations between two tablets of a pharmaceutical composition described herein, each containing 7.5 mg hydrocodone and 325 mg acetaminophen (in fed and fasted states) and an immediate release tablet containing 7.5 mg hydrocodone and 325 mg acetaminophen can be found in Tables 18 and 19.

TABLE 18
Bioequivalence Determination for Acetaminophen

| | LSM | 90% CI | |
|---|---|---|---|
| Parameter | Ratio | Lower | Upper |
| Ln (AUC_{0-1.27 h}) | 103.62 | 87.18 | 123.16 |
| Ln (AUC_{1.27-36 h}) | 93.32 | 83.11 | 104.78 |
| Ln (AUC₀₋ₜ) | 94.52 | 84.34 | 105.93 |

TABLE 19
Bioequivalence Determination for Hydrocodone

| | LSM | 90% CI | |
|---|---|---|---|
| Parameter | Ratio | Lower | Upper |
| Ln (AUC_{0-2.44 h}) | 91.49 | 82.77 | 101.13 |
| Ln (AUC_{2.44-36 h}) | 96.78 | 83.19 | 112.61 |
| Ln (AUC₀₋ₜ) | 89.71 | 81.51 | 98.74 |

### Example 5

### In Vitro Dissolution of Controlled-Release Bilayer Tablets Comprising 7.5 mg Hydrocodone and 325 mg Acetaminophen Performed at a 100 rpm Paddle Speed

Three batches of bilayer formulations described herein were prepared, each containing a total of 7.5 mg of hydrocodone bitartrate and a total of 325 mg of acetaminophen. 50% of the acetaminophen was contained in the immediate release portion, and the other 50% was contained in the ER layer. 25% of the hydrocodone bitartrate was contained in the immediate release portion of the formulation, and the other 75% was contained in the ER layer. POLYOX® N-60K was employed as the extended release component in an amount of 45% by weight of the ER portion.

Dissolution profiles for the formulations of each batch were determined in a USP Type II apparatus. Six tablets from each batch were weighed, placed in a sinker, and dropped into an equilibrated dissolution bath vessel containing 900 mL of (helium sparged) 0.1 N HCl heated to 37° C.±0.5° C. The mixture was stirred at 100±4 rpm, and the temperature was maintained at 37° C.±0.5° C. for 12 hr. The bath vessel was covered with a low evaporation vessel cover. Samples (5 mL) were removed at 0.25 hr, 0.5 hr, 1 hr, 2 hr, 4 hr, 6, hr, 8 hr, and 12 hr. Each sample was filtered through a 0.45 μm filter and analyzed by HPLC using standard procedures.

The cumulative percent release of acetaminophen and hydrocodone from each batch are described in Table 20.

TABLE 20
Release rate data of bilayer tablets (7.5 mg hydrocodone bitartrate;
325 mg acetaminophen) using a 100 rpm dissolution method.

| | Hydrocodone Bitartrate | | | | Acetaminophen | | | |
|---|---|---|---|---|---|---|---|---|
| Time | Mean | | Min | Max | Mean | | Min | Max |
| (Hours) | (%) | RSD | (%) | (%) | (%) | RSD | (%) | (%) |

Batch 1

| | | | | | | | | |
|---|---|---|---|---|---|---|---|---|
| 0.25 | 31.4 | 6.3 | 27.1 | 33.6 | 52.7 | 5.1 | 46.7 | 54.6 |
| 0.5 | 36.5 | 3.6 | 34.1 | 38.6 | 55.5 | 2.5 | 52.2 | 56.8 |
| 1.0 | 43.7 | 2.1 | 42.2 | 45.2 | 59.1 | 1.4 | 57.3 | 60.1 |
| 2.0 | 54.5 | 1.7 | 53.0 | 56.6 | 64.7 | 0.9 | 63.8 | 65.8 |
| 4.0 | 70.9 | 1.3 | 69.4 | 72.9 | 74.0 | 0.8 | 73.1 | 75.2 |
| 6.0 | 83.0 | 1.5 | 81.1 | 85.3 | 81.8 | 0.9 | 80.6 | 83.3 |
| 8.0 | 91.9 | 1.5 | 89.7 | 93.8 | 88.4 | 0.9 | 87.4 | 89.9 |
| 12.0 | 100.5 | 1.4 | 98.1 | 102.5 | 96.1 | 0.8 | 94.9 | 97.2 |

Batch 2

| | | | | | | | | |
|---|---|---|---|---|---|---|---|---|
| 0.25 | 30.8 | 3.0 | 29.6 | 32.5 | 53.6 | 1.7 | 52.4 | 55.1 |
| 0.5 | 35.6 | 2.1 | 34.5 | 37.0 | 55.8 | 1.4 | 54.9 | 57.1 |
| 1.0 | 42.4 | 2.3 | 40.7 | 44.5 | 59.1 | 1.3 | 58.4 | 60.6 |
| 2.0 | 52.7 | 2.1 | 51.6 | 54.8 | 64.6 | 1.3 | 63.9 | 66.5 |
| 4.0 | 69.0 | 2.0 | 67.4 | 71.5 | 73.9 | 1.3 | 72.8 | 76.2 |
| 6.0 | 81.8 | 1.7 | 79.5 | 83.5 | 82.4 | 1.4 | 80.9 | 85.1 |
| 8.0 | 90.3 | 1.5 | 87.9 | 92.5 | 88.6 | 1.6 | 86.6 | 91.9 |
| 12.0 | 98.9 | 1.6 | 96.0 | 101.0 | 96.5 | 1.5 | 94.4 | 99.8 |

Batch 3

| | | | | | | | | |
|---|---|---|---|---|---|---|---|---|
| 0.25 | 31.7 | 3.2 | 29.7 | 33.6 | 52.7 | 2.5 | 49.9 | 54.9 |
| 0.5 | 36.4 | 2.8 | 34.7 | 38.2 | 55.1 | 2.0 | 53.1 | 56.9 |
| 1.0 | 43.5 | 2.3 | 42.1 | 45.1 | 58.7 | 1.8 | 57.5 | 60.7 |
| 2.0 | 54.5 | 2.4 | 52.9 | 56.5 | 64.5 | 1.7 | 63.3 | 66.8 |
| 4.0 | 70.2 | 2.5 | 68.2 | 72.7 | 73.7 | 1.7 | 72.1 | 76.4 |
| 6.0 | 81.8 | 2.2 | 79.8 | 85.6 | 81.3 | 1.6 | 79.2 | 84.3 |
| 8.0 | 90.5 | 2.3 | 88.0 | 95.1 | 87.8 | 1.6 | 85.5 | 91.0 |
| 12.0 | 98.9 | 1.9 | 97.1 | 103.0 | 95.2 | 1.4 | 92.9 | 98.2 |

### Example 6

### In Vitro Dissolution of Controlled-Release Bilayer Tablets Comprising 7.5 mg Hydrocodone and 325 mg Acetaminophen Performed at a 150 Rpm Paddle Speed

Dissolution studies were performed on fast-release, medium-release, and slow-release pharmaceutical formulations described herein containing 7.5 mg hydrocodone and 325 mg acetaminophen.

Dissolution profiles for the three above-described compositions were determined in USP Type II apparatus. Six tablets of each composition were weighed, placed in a sinker, and dropped into an equilibrated dissolution bath vessel that contained 900 mL of (helium sparged) 0.1 N HCl that was heated to 37° C.±0.5° C. The mixture was stirred at 150±6 rpm and the temperature was maintained at 37° C.±0.5° C. for 12 hr. The bath vessel was covered with a low evaporation vessel cover. Samples (5 mL) were removed at 0.25, 0.5, 1, 2, 4, 6, 8, and 12 hours. Each sample was filtered through a 0.45 μm filter and analyzed by HPLC using standard procedures.

The results of these dissolution studies are summarized in Table 21 and FIGS. 13 and 14.

TABLE 21
Mean acetaminophen and hydrocodone dissolution data.

| | Fast (%) (RSD) | | Medium (%) (RSD) | | Slow (%) (RSD) | |
|---|---|---|---|---|---|---|
| Time (hr) | APAP | Hydrocodone | APAP | Hydrocodone | APAP | Hydrocodone |
| 0.08 | 52.6(2.2) | 26.3(4.1) | 52.4(2.7) | 28.4(4.3) | 52.2(2.1) | 28.8(4.0) |
| 0.25 | 55.8(2.0) | 34.3(2.1) | 54.4(2.7) | 33.4(2.9) | 54.2(1.7) | 33.8(2.8) |
| 0.5 | 58.9(1.9) | 41.2(1.4) | 56.9(2.5) | 38.6(2.4) | 56.2(1.5) | 38.2(3.3) |
| 1.0 | 64.0(1.8) | 51.1(1.4) | 60.9(2.3) | 46.8(1.9) | 59.5(1.5) | 45.2(3.5) |
| 2.0 | 72.8(1.6) | 65.9(2.0) | 67.9(2.0) | 59.7(2.1) | 65.0(1.4) | 56.3(3.5) |
| 4.0 | 87.0(1.8) | 86.7(2.6) | 79.8(1.6) | 79.6(2.1) | 74.6(1.4) | 74.0(2.6) |
| 8.0 | 98.5(1.1) | 100.7(1.2) | 93.9(1.0) | 99.7(1.9) | 88.2(1.4) | 94.9(2.4) |
| 12.0 | 97.9(1.2) | 100.5(1.5) | 96.9(0.8) | 102.9(1.6) | 95.6(1.7) | 103.0(2.4) |
| 18.0 | 96.8(1.2) | 100.3(1.6) | 96.1(0.8) | 102.8(1.7) | 97.0(1.5) | 104.6(2.1) |

### Example 7

### Varying Polyox Grades Comprising 25% by Weight of the Extended Release Portion of Bilayer Formulations Containing Hydrocodone

Bilayer formulations described herein were prepared, each containing a total of 15 mg of hydrocodone bitartrate and a total of 500 mg of acetaminophen. 50% of the acetaminophen was contained in the immediate release portion, and the other 50% was contained in the ER layer. 25% of the hydrocodone bitartrate was contained in the immediate release portion of the formulation, and the other 75% was contained in the ER layer. In a first formulation, POLYOX® 205 was employed as the extended release component in an amount of 25% by weight of the ER portion. In a second formulation, POLYOX® 1105 was employed as the extended release component in an amount of 25% by weight of the ER portion. In a third formulation, POLYOX® N-12K was employed as the extended release component in an amount of 25% by weight of the ER portion. In a fourth formulation, POLYOX® N-60K was employed as the extended release component in an amount of 25% by weight of the ER portion. In a fifth formulation, POLYOX® 301 was employed as the extended release component in an amount of 25% by weight of the ER portion. The other excipients in the extended release portion were microcrystalline cellulose, spress B825, citric acid anhydrous, EDTA, hydroxypropyl cellulose, silicon dioxide, and magnesium stearate.

Dissolution profiles for the five above-described compositions were determined in a USP Type II apparatus. Five tablets of each composition were weighed, placed in a sinker, and dropped into an equilibrated dissolution bath vessel containing 900 mL of (helium sparged) 0.1 N HCl heated to 37° C.±0.5° C. The mixture was stirred at 150±6 rpm, and the temperature was maintained at 37° C.±0.5° C. for 12 hr. The bath vessel was covered with a low evaporation vessel cover. Samples (5 mL) were removed at 0.25 hr, 0.5 hr, 1 hr, 2 hr, 4 hr, 6, hr, 8 hr, 12 hr, and 18 hr. Each sample was filtered through a 0.45 μm filter and analyzed by HPLC using standard procedures.

The cumulative release profiles of acetaminophen and hydrocodone from these compositions are shown in FIGS. 15 and 16, respectively. These figures demonstrate that as the average molecular weight of the POLYOX® extended release component increases, the rate of dissolution at each time point decreases. For example, the formulations containing POLYOX® 205, 1105, N-12K, N-60K, and 301 had released about 59%, about 58%, about 56%, about 55%, and about 52% acetaminophen after 15 minutes, respectively; about 62%, about 61%, about 58%, about 57%, and about 56% acetaminophen after 30 minutes, respectively; about 68%, about 66%, about 63%, about 61%, and about 60% acetaminophen after 1 hr, respectively; about 78%, about 76%, about 71%, about 67%, and about 65% acetaminophen after 2 hr, respectively; about 92%, about 90%, about 83%, about 76%, and about 73% acetaminophen after 4 hr, respectively; about 98%, about 97%, about 92%, about 84%, and about 79% acetaminophen after 6 hr, respectively; about 99%, about 98%, about 96%, about 90%, and about 85% acetaminophen after 8 hr, respectively; about 98%, about 97%, about 96%, about 97%, and about 92% acetaminophen after 12 hr, respectively; and about 98%, about 97%, about 96%, about 97%, and about 97% acetaminophen after 18 hr, respectively.

A decreased release rate with a higher molecular weight POLYOX® grade was observed for the hydrocodone bitratrate. For example, the formulations containing POLYOX® 205, 1105, N-12K, N-60K, and 301 had released about 38%, about 39%, about 39%, about 34%, and about 32% hydrocodone bitratrate after 15 minutes, respectively; about 48%, about 47%, about 46%, about 41%, and about 39% hydrocodone bitratrate after 30 minutes, respectively; about 60%, about 57%, about 55%, about 49%, and about 47% hydrocodone bitratrate after 1 hr, respectively; about 76%, about 72%, about 68%, about 60%, and about 58% hydrocodone bitratrate after 2 hr, respectively; about 96%, about 93%, about 87%, about 77%, and about 73% hydrocodone bitratrate after 4 hr, respectively; about 105%, about 102%, about 99%, about 89%, and about 83% hydrocodone bitratrate after 6 hr, respectively; about 105%, about 102%, about 103%, about 97%, and about 91% hydrocodone bitratrate after 8 hr, respectively; about 105%, about 102%, 103%, about 104%, and about 100% hydrocodone bitratrate after 12 hr, respectively; and about 106%, about 103%, about 104%, about 104%, and about 104% hydrocodone bitratrate after 18 hr, respectively.

### Example 8

### Varying Polyox Grades Comprising 45% by Weight of the Extended Release Portion of Bilayer Formulations Containing Hydrocodone

The release rate studies described in Example 7 were repeated, except that the five bilayer formulations were prepared such that they included POLYOX® 205, 1105, N-12K, N-60K, and 301 in an amount of 45% by weight of the ER portion.

The cumulative release profiles of acetaminophen and hydrocodone from these compositions are shown in FIGS. 17 and 18, respectively. Consistent with the results of Example 7, the rate of dissolution at each time point generally decreases as the average molecular weight of POLYOX® increases. For example, the formulations containing POLYOX® 205, 1105, N-12K, N-60K, and 301 had released about 55%, about 53%, about 55%, about 54%, and about 54% acetaminophen after 15 minutes, respectively; about 57%, about 56%, about 57%, about 55%, and about 56% acetaminophen after 30 minutes, respectively; about 62%, about 60%, about 60%, about 58%, and about 59% acetaminophen after 1 hr, respectively; about 70%, about 68%, about 67%, about 64%, and about 63% acetaminophen after 2 hr, respectively; about 84%, about 81%, about 78%, about 72%, and about 70% acetaminophen after 4 hr, respectively; about 95%, about 91%, about 87%, about 80%, and about 77% acetaminophen after 6 hr, respectively; about 99%, about 96%, about 93%, about 86%, and about 82% acetaminophen after 8 hr, respectively; about 99%, about 98%, about 99%, about 95%, and about 90% acetaminophen after 12 hr, respectively; and about 98%, about 97%, about 98%, about 99%, and about 96% acetaminophen after 18 hr, respectively.

A decreased release rate with a higher molecular weight POLYOX® grade was observed for the hydrocodone bitratrate. For example, the formulations containing POLYOX® 205, 1105, N-12K, N-60K, and 301 had released about 33%, about 33%, about 32%, about 31%, and about 32% hydrocodone bitratrate after 15 minutes, respectively; about 39%, about 39%, about 38%, about 36%, and about 37% hydrocodone bitratrate after 30 minutes, respectively; about 48%, about 48%, about 46%, about 43%, and about 43% hydrocodone bitratrate after 1 hr, respectively; about 63%, about 63%, about 59%, about 54%, and about 53% hydrocodone bitratrate after 2 hr, respectively; about 85%, about 84%, about 79%, about 71%, and about 68% hydrocodone bitratrate after 4 hr, respectively; about 99%, about 97%, about 92%, about 84%, and about 79% hydrocodone bitratrate after 6 hr, respectively; about 102%, about 103%, about 100%, about 93%, and about 88% hydrocodone bitratrate after 8 hr, respectively; about 103%, about 104%, about 104%, about 102%, and about 98% hydrocodone bitratrate after 12 hr, respectively; and about 104%, about 103%, about 104%, about 105%, and about 103% hydrocodone bitratrate after 18 hr, respectively.

### Example 9

### Varying the Concentrations of a Specific Polyox Grade in the Extended Release Portion of Bilayer Formulations Containing Hydrocodone

The data from Examples 7 and 8 indicate that an increase in the amount of POLYOX® in the pharmaceutical composition also retards release of the actives from the pharmaceutical composition. To confirm this observation, bilayer formulations described herein were prepared, each containing a total of 15 mg of hydrocodone bitartrate and a total of 500 mg of acetaminophen. 50% of the acetaminophen was contained in the immediate release portion, and the other 50% was contained in the ER layer. 25% of the hydrocodone bitartrate was contained in the immediate release portion of the formulation, and the other 75% was contained in the ER layer. In a first formulation, POLYOX® 1105 was employed as the extended release component in an amount of 25% by weight of the ER portion. In a second formulation, POLYOX™ 1105 was employed as the extended release component in an amount of 35% by weight of the ER portion. In a third formulation, POLYOX™ 1105 was employed as the extended release component in an amount of 45% by weight of the ER portion.

The cumulative release profiles of acetaminophen and hydrocodone bitartrate from these compositions are shown in FIGS. 19 and 20, respectively. These profiles confirm that as the amount of POLYOX® 1105 used in the pharmaceutical formulations increase, the release rate of the actives generally decreases. For example, the formulations containing 25%, 35%, and 45% POLYOX® 1105 had released about 58%, about 54%, and about 53% acetaminophen after 15 minutes, respectively; about 61%, about 56%, and about 56% acetaminophen after 30 minutes, respectively; about 66%, about 61%, and about 60% acetaminophen after 1 hr, respectively; about 76%, about 70%, and about 68% acetaminophen after 2 hr, respectively; about 90%, about 85%, and about 81% acetaminophen after 4 hr, respectively; about 97%, about 94%, and about 91% acetaminophen after 6 hr, respectively; about 98%, about 97%, and about 96% acetaminophen after 8 hr, respectively; about 97%, about 97%, and about 98% acetaminophen after 12 hr, respectively; and about 97%, about 96%, and about 97% acetaminophen after 18 hr, respectively.

Similar trends were observed for the cumulative release of hydrocodone bitartrate. For example, the formulations containing 25%, 35%, and 45% POLYOX® 1105 had released about 39%, about 34%, and about 33% hydrocodone bitartrate after 15 minutes, respectively; about 47%, about 39%, and about 39% hydrocodone bitartrate after 30 minutes, respectively; about 57%, about 49%, and about 48% hydrocodone bitartrate after 1 hr, respectively; about 72%, about 65%, and about 63% hydrocodone bitartrate after 2 hr, respectively; about 93%, about 88%, and about 84% hydrocodone bitartrate after 4 hr, respectively; about 102%, about 100%, about 97% hydrocodone bitartrate after 6 hr, respectively; about 102%, about 103%, and about 103% hydrocodone bitartrate after 8 hr, respectively; about 102%, about 104%, and about 104% hydrocodone bitartrate after 12 hr, respectively; and about 103%, about 103%, and about 103% hydrocodone bitartrate after 18 hr, respectively.

### Example 10

### Varying the Concentrations of a Specific Polyox Grade in the Extended Release Portion of Bilayer Formulations Containing Hydrocodone

The release rate studies described in Example 9 were repeated, except that the three bilayer formulations were prepared such that they included POLYOX® N-60K instead of 1105.

The cumulative release profiles of acetaminophen and hydrocodone bitartrate from these compositions are shown in FIGS. 21 and 22, respectively. These profiles confirm that as the amount of POLYOX® N-60K used in the pharmaceutical formulations increase, the release rate of the actives generally decreases. For example, the formulations containing 25%, 35%, and 45% POLYOX® N-60K had released about 55%, about 54%, and about 54% acetaminophen after 15 minutes, respectively; about 57%, about 56%, and about 55% acetaminophen after 30 minutes, respectively; about 61%, about 60%, and about 58% acetaminophen after 1 hr, respectively; about 67%, about 65%, and about 64% acetaminophen after 2 hr, respectively; about 76%, about 74%, and about 72% acetaminophen after 4 hr, respectively; about 84%, about 82%, and about 80% acetaminophen after 6 hr, respectively; about 90%, about 88%, and about 86% acetaminophen after 8 hr, respectively; about 97%, about 96%, and about 95% acetaminophen after 12 hr, respectively; and about 97%, about 98%, and about 99% acetaminophen after 18 hr, respectively.

Similar trends were observed for the cumulative release of hydrocodone bitartrate. For example, the formulations containing 25%, 35%, and 45% POLYOX® N-60K had released about 34%, about 32%, and about 31% hydrocodone bitartrate after 15 minutes, respectively; about 41%, about 37%, and about 36% hydrocodone bitartrate after 30 minutes, respectively; about 49%, about 44%, and about 43% hydrocodone bitartrate after 1 hr, respectively; about 60%, about 55%, and about 54% hydrocodone bitartrate after 2 hr, respectively; about 77%, about 72%, and about 71% hydrocodone bitartrate after 4 hr, respectively; about 89%, about 85%, about 84% hydrocodone bitartrate after 6 hr, respectively; about 97%, about 93%, and about 93% hydrocodone bitartrate after 8 hr, respectively; about 104%, about 100%, and about 102% hydrocodone bitartrate after 12 hr, respectively; and about 104%, about 102%, and about 105% hydrocodone bitartrate after 18 hr, respectively.

While the cumulative release profiles of the formulations generally decrease as the amount of the extended release component is increased, this trend is more pronounced for POLYOX® 1105 than for POLYOX® N-60K.

### Example 11

### In Vitro Dissolution of Controlled-Release Bilayer Tablets Containing 15 Mg Hydrocodone and 650 Mg Acetaminophen Performed at a 150 rpm Paddle Speed

Bilayer formulations described herein were prepared, each containing a total of 15 mg of hydrocodone bitartrate and a total of 650 mg of acetaminophen. 50% of the acetaminophen was contained in the immediate release portion, and the other 50% was contained in the ER layer. 25% of the hydrocodone bitartrate was contained in the immediate release portion of the formulation, and the other 75% was contained in the ER layer. POLYOX® N60k was employed as the extended release component in an amount of 45% by weight of the ER portion.

Dissolution profiles for the formulations were determined in a USP Type II apparatus. Six tablets were weighed, placed in a sinker, and dropped into an equilibrated dissolution bath vessel containing 900 mL of (helium sparged) 0.1N HCl heated to 37° C.±0.5° C. The mixture was stirred at 150±6 rpm, and the temperature was maintained at 37° C.±0.5° C. for 12 hr. The bath vessel was covered with a low evaporation vessel cover. Samples (5 mL) were removed at 0.25 hr, 0.5 hr, 1 hr, 2 hr, 4 hr, 6, hr, 8 hr, 12 hr, and 18 hr. Each sample was filtered through a 0.45 μm filter and analyzed by HPLC using standard procedures.

The cumulative percent release of acetaminophen and hydrocodone bitartrate from each batch are described in Table 22.

TABLE 22
Release rate data of bilayer tablets (15 mg hydrocodone bitartrate;
325 mg acetaminophen) using a 150 rpm dissolution method.

| Time | Hydrocodone Bitartrate | | Acetaminophen | |
|---|---|---|---|---|
| (hr) | Mean (%) | RSD (%) | Mean (%) | RSD (%) |
| 0.25 | 32.6 | 1.5 | 53.4 | 0.9 |
| 0.50 | 37.1 | 2.1 | 55.3 | 1.0 |
| 1 | 44.2 | 2.2 | 58.4 | 0.9 |
| 2 | 55.0 | 1.3 | 63.4 | 1.0 |
| 4 | 71.8 | 0.8 | 72.3 | 1.2 |
| 6 | 83.9 | 1.3 | 79.6 | 1.1 |
| 8 | 92.2 | 0.6 | 85.7 | 1.1 |
| 12 | 99.5 | 0.7 | 93.7 | 1.1 |
| 18 | 101.0 | 0.7 | 97.2 | 1.0 |

### Example 12

### Clinical Pharmacokinetic Analysis of an Extended Release Formulation of 7.5 mg Hydrocodone/325 mg Acetaminophen—Single Dose

An open-label, randomized, four-period crossover study to evaluate the single and multiple dose pharmacokinetics, bioavailability, and safety of an bilayer, immediate release/extended release tablet formulation comprising 7.5 mg hydrocodone and 325 mg acetaminophen (see selected example from Chart No. 1) with a commercially-available tablet containing 37.5 mg tramadol and 325 mg acetaminophen and a commercially-available tablet containing 7.5 mg hydrocodone and 200 mg ibuprofen in normal, healthy subjects under fasted conditions was conducted.

Subjects received the following treatments:
Treatment A: Two tablets of a bilayer tablet formulation disclosed herein containing 7.5 mg hydrocodone and 325 mg acetaminophen administered orally as 1 dose under fasted conditions on Day 1 followed by 2 tablets given Q12h (beginning on Day 3 for a total of 9 doses);
Treatment B: One tablet of a commercially-available tablet containing 7.5 mg hydrocodone and 200 mg ibuprofen administered orally Q6h for 2 doses under fasted conditions on Day 1 followed by 1 tablet given Q6h (beginning on Day 3 for a total of 18 doses);
Treatment C: One tablet of a commercially-available tablet containing 37.5 mg tramadol and 325 mg acetaminophen administered orally Q6h for 2 doses under fasted conditions on Day 1 followed by 1 tablet given Q6h (beginning on Day 3 for a total of 18 doses); and
Treatment D: Three tablets of a bilayer tablet formulation disclosed herein containing 7.5 mg hydrocodone and 325 mg acetaminophen administered orally as 1 dose under fasted conditions on Day 1 followed by 3 tablets administered as a single dose at Hour 48. Subjects then received 2 tablets given Q12h (beginning at Hour 60 for a total of 8 doses).

Subjects were randomly assigned to receive each study treatment in 1 of 4 treatment sequences (Treatment Periods 1 to 4) according to the randomization schedule. There was at least a 14-day washout period between each of the Treatment Periods (1 to 4).

The pharmacokinetic parameters for hydrocodone following a single dose of Treatments A and B to a subject are presented in Table 23, and the corresponding hydrocodone plasma concentration versus time profiles are presented in FIG. 23. Further, the pharmacokinetic parameters for acetaminophen following a single dose of Treatments A and B to a subject are presented in Table 24, and the acetaminophen plasma concentration versus time profiles are presented in FIG. 24.

TABLE 23
Mean Pharmacokinetic Parameters for Hydrocodone—Single Dose

| | Treatment A | | |
|---|---|---|---|
| | Two Tablets | Three Tablets | Treatment B |
| Cmax (ng/mL) | 17.68 | 26.34 | 25.46 |
| | (3.40) | (5.50) | (5.73) |
| AUCt (ng.hr/mL) | 249.83 | 373.80 | 268.53 |
| | (59.32) | (90.64) | (60.08) |
| AUCinf | 256.17 | 378.83 | 270.30 |
| (ng.hr/mL) | (68.93) | (92.89) | (60.79) |
| tlag (hr) | 0.1 | 0.042 | 0.042 |
| | (0.141) | (0.095) | (0.095) |
| Tmax* (hr) | 3 | 3 | 8 |
| | (0.5-6) | (0.75-8) | (0.5-10) |
| T1/2 (hr) | 7.48 | 6.92 | 5.75 |
| | (3.31) | (1.20) | (0.73) |

TABLE 24
Mean Pharmacokinetic Parameters for Acetaminophen—Single Dose

| | Treatment A | | |
|---|---|---|---|
| | Two Tablets | Three Tablets | Treatment C |
| Cmax | 5719 | 8989 | 5144.67 |
| (ng/mL) | (1811.44) | (2290.88) | (1354.16) |
| AUCt | 32708.63 | 51388.32 | 33186.7 |
| (ng.hr/mL) | (9979.09) | (15182.50) | (9816.33) |
| AUCinf | 35649.65 | 54438.7 | 34615.85 |
| (ng.hr/mL) | (9731.33) | (14776.62) | (10132.85) |
| tlag | 0.033 | 0 | 0.017 |
| (hr) | (0.109) | (0) | (0.063) |
| Tmax* | 0.63 | 0.5 | 0.5 |
| (hr) | (0.25-3) | (0.25-2) | (0.25-8) |
| T1/2 | 9.12 | 9.04 | 5.00 |
| (hr) | (4.18) | (4.14) | (1.76) |

The total exposure (measured as AUC0-inf and AUC0-t) and peak exposure (measured as Cmax) were dose proportional between Treatment A (two tablets of an extended release tablet formulation disclosed herein containing 7.5 mg hydrocodone and 325 mg acetaminophen) and Treatment D (three tablets of an extended release tablet formulation disclosed herein containing 7.5 mg hydrocodone and 325 mg acetaminophen) for both hydrocodone and acetaminophen following single dose administration as well as steady state. Further, no difference was observed in dose-normalized total exposure (measured as AUC0-inf and AUC0-t) of hydrocodone and acetaminophen between Treatment A and Treatment D compared to Treatment B and Treatment C.

The dose-normalized Cmax of acetaminophen following administration of Treatment A and Treatment D was equivalent to the Cmax following 2 doses of Treatment C administered as 1 tablet Q6h. Moreover, the dose normalized Cmax of hydrocodone was approximately 30% lower for Treatment A and Treatment D compared to 2 doses of Treatment B administered as 1 tablet Q6h. The reduced Cmax relative to Treatment B, which is an immediate release formulation, was expected because Treatments A and D are bilayer extended release formulations that contain only 25% of the hydrocodone in the immediate layer.

No significant difference in Tmax was observed for both hydrocodone and acetaminophen between Treatment A and Treatment D. However, for hydrocodone, Tmax was significantly shorter with Treatment A and Treatment D compared to Treatment B, which may be due to the dosage regimen differences (i.e., Q6h for Treatment B versus Q12h for Treatments A and D). As for acetaminophen, Tmax was not significantly different between Treatment C and the Treatment D, but there was a significant difference (p=0.035) between Treatment A and Treatment C.

Median tlag for plasma acetaminophen levels was 0 hrs for all of the treatments, and there was no significant difference across treatments. The tlag for hydrocodone levels for all of the treatments was also 0 hrs. No difference was observed in tlag between Treatment B versus Treatment A and Treatment D. However, the median tlag between Treatment A and Treatment D was statistically different, but not clinically significant.

The mean steady state pharmacokinetic parameters for hydrocodone following a loading dose and subsequent dose of Treatments A and B to a subject are presented in Table 25. The corresponding hydrocodone plasma concentration versus time profiles are presented in FIG. 25. Further, the pharmacokinetic parameters for acetaminophen following a loading dose and subsequent dose of Treatments A and B to a subject are presented in Table 26. The acetaminophen plasma concentration versus time profiles are presented in FIG. 26.

TABLE 25
Mean Steady State Pharmacokinetic Parameters for Hydrocodone

| | Treatment A | | |
|---|---|---|---|
| | Two Tablets | Three Tablets | Treatment B |
| Cmax (ss) | 30.57 | 30.52 | 35.95 |
| (ng/mL) | (6.37) | (7.11) | (7.74) |
| Cmin (ss) | 12.53 | 12.29 | 15.89 |
| (ng/mL) | (4.19) | (3.37) | (5.29) |
| Cavg (ss) | 22.37 | 22.03 | 24.92 |
| (ng/mL) | (5.20) | (5.19) | (6.40) |
| AUC0-12 (ss) | 268.40 | 264.30 | 299.05 |
| (ng.hr/mL) | (62.37) | (62.32) | (76.82) |
| Tmax (SS) | 146 | 147 | 152 |
| (hr) | (144.5-150) | (144.75-150) | (144.5-154) |
| Swing | 1.57 | 1.56 | 1.43 |
| | (0.57) | (0.50) | (0.81) |
| Fluctuation | 82.57 | 83.66 | 84.14 |
| (%) | (19.78) | (18.34) | (28.0) |
| Accumulation | 1.33 | 1.36 | 1.34 |
| Index | (0.15) | (0.16) | (0.40) |

TABLE 26
Mean Steady State Pharmacokinetic Parameters for Acetaminophen

| | Treatment A | | |
|---|---|---|---|
| | Two Tablets | Three Tablets | Treatment C |
| Cmax (ss) | 6563.79 | 6485.86 | 5878.97 |
| (ng/mL) | (2011.38) | (1601.53) | (1772.00) |
| Cmin (ss) | 976.52 | 1005.03 | 1158.86 |
| (ng/mL) | (350.92) | (294.79) | (360.80) |
| Cavg (ss) | 2674.43 | 2685.48 | 2756.11 |
| (ng/mL) | (773.93) | (678.01) | (676.15) |
| AUC0-12 (ss) | 32093.17 | 32225.76 | 33073.27 |
| (ng.hr/mL) | (9287.12) | (8136.08) | (8113.76) |
| Tmax (SS) | 144.5 | 144.75 | 144.75 |
| (hr) | (144.25-148) | (144.25-147) | (144.25-152) |
| Swing | 6.38 | 5.75 | 4.39 |
| | (3.13) | (1.82) | (1.90) |
| Fluctuation | 215.58 | 207.03 | 172.11 |
| (%) | (69.84) | (44.94) | (44.47) |
| Accumulation | 1.19 | 1.22 | 1.20 |
| Index | (0.08) | (0.18) | (0.48) |

For both hydrocodone and acetaminophen, the steady-state

PK parameters (AUC0-12 hrss, Claxss, Cminss, Cavgss, and DFL) following multiple doses, with and without the loading dose of 3 tablets of the extended release tablet formulation disclosed herein containing 7.5 mg hydrocodone and 325 mg acetaminophen (i.e., Treatment A versus Treatment D) were comparable. In addition, Swing values for acetaminophen were also comparable with and without the loading dose; however, the hydrocodone Swing values with the loading dose was 12% higher compared to Swing values without the loading dose.

For hydrocodone, steady-state was attained by 48 hours for all of the treatments. For acetaminophen, steady-state was attained by 24 hours for Treatment A, and by 48 hours for Treatment C, and Treatment D.

At steady-state, no difference was observed in dose-normalized AUC0-12 hrss, Cmaxss, and Cavgss of hydrocodone between Treatment A, Treatment D, and Treatment B. Similarly, no difference was observed in dose-normalized AUC₀₋₁₂ hrss, Cmaxss, Cminss, and Cavss of APAP between Treatment A, Treatment D, and Treatment C.

No significant difference in Tmaxss was observed for hydrocodone and acetaminophen between Treatment A and Treatment D. However, for both hydrocodone and acetaminophen, Tmaxss was significantly shorter with both Treatment A and Treatment D compared to Treatments B and C, primarily due to the dosage regimen differences (i.e., Q6h for Treatments B and C versus Q12h for Treatments A and D).

There was no difference in the degree of fluctuation (“DFL”) of plasma hydrocodone concentrations for either Treatment A or Treatment D compared with Treatment B or between each other. Also, the swing value of Treatment A was not different from Treatment C for hydrocodone. However, the swing value for hydrocodone was greater by 9% and 12% for Treatment D compared to Treatment C and Treatment A.

As for acetaminophen, the DFL and swing values for Treatment A and Treatment D were greater by approximately 21% (for DFL) and 32% (for swing) as compared to Treatment C. Moreover, the steady-state trough plasma concentrations of acetaminophen from Treatment A and Treatment D were lower than those from Treatment C, suggesting that multiple days of dosing with the extended release tablet formulation disclosed herein containing 7.5 mg hydrocodone and 325 mg acetaminophen (as used in Treatments A and D) is less likely to be associated with drug induced liver injury.

### Example 13

### Single- and Multiple-Dose Pharmacokinetics of Extended-Release Hydrocodone Bitartrate/Acetaminophen Tablets with a Loading Dose of 3 Tablets Followed by 2 Tablets Administered Every 12 Hours Compared with Immediate-Release Hydrocodone Bitartrate/Acetaminophen Tablets

An open-label, randomized, single- and multiple-dose, two-period, crossover Phase I study was conducted to evaluate the single- and multi-dose pharmacokinetics and bioavailability of a multi-layer extended release tablet formulation as disclosed herein comprising 7.5 mg hydrocodone and 325 mg APAP compared to an immediate release tablet formulation comprising 7.5 mg hydrocodone and 325 mg APAP. Doses were administered to healthy volunteers under fasted conditions. In the single-dose portion of the study, subjects were administered one 3-tablet dose of the multi-layer extended release formulation or three 1-tablet doses of immediate release hydrocodone/APAP every 4 hours. In the multiple-dose portion of the study, subjects were randomly assigned one of two sequences: AB or BA. In Treatment A, subjects were administered three tablets of the multi-layer extended release tablet formulation described herein at Hour 0 followed by 8 doses of 2 tablets every 12 hours (4.5 days) starting at Hour 12. In Treatment B, subjects were administered 1 tablet of an immediate release hydrocodone/APAP (7.5/325) every 4 hours starting at Hour 0 for three doses followed by 16 doses of 1 tablet administered every 6 hours (4.5 days) starting at Hour 12.

The most commonly reported adverse events (AEs) were nausea, and vomiting. No serious AEs were reported. Plasma hydrocodone and APAP concentrations were rapidly obtained after administration of the multi-layer extended release hydrocodone/APAP formulation. Hydrocodone concentrations were sustained throughout the 12-hour dosing interval, and APAP levels were low after about 10-12 hours from dosing. Steady-state conditions were observed in 3 days for hydrocodone and 2 days for APAP. Total exposures (dose-normalized AUC₀₋₁₂) to hydrocodone and APAP after administration of the multi-layer extended release hydrocodone/APAP formulation as a single-dose or at steady state were comparable. At steady state, total exposure (dose-normalized AUC) to hydrocodone and APAP and peak exposure (dose-normalized Cmax) to hydrocodone with the loading dose were equivalent to those for immediate release hydrocodone/APAP. Although peak exposure (dose-normalized Cmax) at steady state for APAP was higher after administration of the multi-layer extended release formulation, average plasma concentrations were equivalent between the multi-layer extended release and immediate release formulations. Consistent with earlier studies, trough APAP plasma concentrations after administration of the multi-layer extended release formulation with a loading dose were lower compared to the immediate release formulation. These findings support a 12-hour dosing interval for the multi-layer extended release hydrocodone/APAP tablet formulation.

### Example 14

### Randomized, Double-Blind, Placebo-Controlled, Phase III Study of the Safety and Analgesic Efficacy of Extended-Release Hydrocodone Bitartrate/Acetaminophen Tablets (ER HB/APAP) in an Acute Pain Model

A randomized, double-blind, placebo-controlled Phase III clinical study was conducted to evaluate the safety and efficacy of a multi-layer extended release tablet formulation as disclosed herein comprising 7.5 mg hydrocodone and 325 mg APAP in patients with moderate to moderately severe acute pain. The safety and efficacy was evaluated versus placebo over the first 48 hours in patients with acute moderate to severe pain following a unilateral bunionectomy. Patients were administered 3 tablets of the multi-layer extended release hydrocodone/APAP formulation at Hour 0 followed by 2 tablets every 12 hours for a total daily dose on Day 1 of 37.5 mg hydrocodone/1625 mg APAP and 30 mg hydrocodone/1300 mg APAP per day thereafter. The multi-layer extended release formulation demonstrated greater efficacy over the placebo across a variety of validated pain measures. The primary endpoint, summed pain intensity difference at 48 hours (SPID₄₈) was statistically significantly greater in patients administered the multi-layer extended release formulation versus placebo (P<0.001). Cumulative SPID demonstrated that the multi-layer extended release formulation provided superior pain relief throughout the 48-hour double-blind dosing period, and SPID dosing interval analyses demonstrated consistent, superior pain relief for each dosing interval. Mean total pain relief (TOTPAR) scores also demonstrated greater pain relief with the multi-layer extended release formulation versus placebo over the 48-hour double-blind dosing period and within each interval. Mean pain intensity difference (PID) was statistically significantly greater in patients receiving the multi-layer extended release formulation versus placebo beginning 30 minutes after the first dose, and this greater PID was maintained throughout the dosing interval. Time to onset of perceptible pain relief, meaningful pain relief, and confirmed perceptible pain relief were statistically significantly shorter, with maximum PID statistically significantly greater in patients receiving the multi-layer extended release formulation versus placebo throughout the 48-hour double-blind dosing period. The multi-layer extended release formulation provided rapid, significant, and consistent analgesic efficacy over the 12-hour dosing interval and was well tolerated

### Example 15

### Open-Label Safety of an Extended-Release Hydrocodone Bitartrate/Acetaminophen Tablets (ER HB/APAP), in Patients with Osteoarthritis or Chronic Low Back Pain

An open-label safety study was conducted to evaluate effective pain management in patients with osteoarthritis (n=73) or chronic lower back pain (n=80) using a multi-layer extended release tablet formulation comprising 7.5 mg hydrocodone and 325 mg APAP as described herein. Subjects were administered a loading dose of 3 tablets of the multi-layer extended release tablet formulation followed by two tablets every 12 hours for up to 35 days. Several measures of pain control and relief were demonstrated in patients with either osteoarthritis or chronic lower back pain. Mean scores on the modified Brief Pain Inventory-Short Form (mBPI-SF, scale of 0 to 10) for worst pain in the last 24 hours, least pain in the last 24 hours, average pain in the last 24 hours, and current pain all decreased over the course of the study. Mean score changes from baseline to the end of treatment were −3.3, −3.1, −3.4, and −3.7, respectively. The greatest improvement occurred at the final visit (Day 36). Percent pain relief increased steadily from baseline through Day 36 with a mean improvement of 48.8%. Pain-related quality of life, as measured by the mBPI-SF pain interference score, continually improved at each visit. Mean Western Ontario and McMaster Universities Arthritis Index (WOMAC®) pain scores in patients with osteoarthritis decreased from baseline through Day 36 by 4.66 points on a 20-point scale, with the mean total WOMAC pain scores decreasing 20.53 points (total pain score ranged from 0 to 96 points). Mean Roland-Morris Disability Questionnaire (RMDQ) lower back pain and disability scores for patients with chronic lower back pain decreased from 10.6 points at baseline to 7.7 points at Day 36. Lower back pain and disability scores ranged from 0 to 24 points, with higher scores representing greater disability. The safety profile of the multi-layer extended release formulation was similar to that of low-dose opioid/APAP combination products.

### Example 16

### Relationship Between Hydrocodone Pharmacokinetics and Subjective Drug Effects Following Oral Administration of an Immediate-Release Combination of Hydrocodone Bitartrate and Acetaminophen and an Extended-Release Hydrocodone Bitartrate/Acetaminophen (ER HB/APAP) Tablets

A randomized, double-blind, double-dummy, active- and placebo-controlled, seven-way crossover study to evaluate the abuse resistance of a multi-layer extended release tablet formulation for as disclosed herein comprising 7.5 mg hydrocodone and 325 mg APAP was conducted. This study evaluated the correlation of certain subjective effects associated with drug abuse in recreational opioid users, such as drug liking, drug high, and good drug effects, with the pharmacokinetic (PK) characteristics of intact multi-layer extended release tablet formulation, crushed multi-layer extended release tablet formulation, and a similar immediate release hydrocodone/APAP formulation (Norco®). Subjects received a single dose of one of 7 study treatments: (A) low-dose, intact multi-layer extended release tablet formulation (22.5 mg hydrocodone/975 mg APAP, 3 tablets); (B) high-dose, intact multi-layer extended release tablet formulation (45 mg hydrocodone/1950 mg APAP, 6 tablets); (C) low-dose, intact immediate release hydrocodone/APAP (22.5 mg HB/975 mg APAP, 3 tablets); (D) high-dose, intact immediate release hydrocodone/APAP (45 mg HB/1950 mg APAP, 6 tablets); (E) high-dose, crushed (encapsulated) multi-layer extended release tablet formulation (45 mg HB/1950 mg APAP, 6 capsules); (F) high-dose, crushed (encapsulated) immediate release hydrocodone/APAP (45 mg HB/1950 mg APAP, 6 capsules); and (G) placebo. Intact high- and low-doses of the multi-layer extended release hydrocodone/APAP tablet formulation produced lower maximum plasma concentration (Cmax) and longer time to maximum plasma concentration (Tmax) for hydrocodone than comparable doses of immediate release hydrocodone/APAP.

FIG. 27 presents the mean plasma concentration (ng/mL) of hydrocodone over 24 hours by treatment. The PK parameters are summarized in Table 27. Statistical analyses of dose-normalized PK parameters of hydrocodone in various treatments are provided in Table 28.

TABLE 27
PK Parameters for Hydrocodone in Abuse Resistance Studies

| | Treatment | | | | | |
|---|---|---|---|---|---|---|
| Parameter | A | B | C | D | E | F |
| AUC_{0-1 h} | 8.55 | 19.5 | 21.3 | 44.2 | 2.6 | 31.5 |
| (ng · hr/mL) | | | | | | |
| AUC_{0-2 h} | 30.5 | 64.5 | 73.6 | 143.7 | 25.5 | 121.9 |
| (ng · hr/mL) | | | | | | |
| AUC_{0-4 h} | 77.1 | 158.9 | 157.1 | 297.5 | 118.2 | 267.1 |
| (ng · hr/mL) | | | | | | |
| AUC_{0-8 h} | 159.0 | 325.4 | 256.4 | 510.7 | 319.0 | 464.8 |
| (ng · hr/mL) | | | | | | |
| AUC_{0-12 h} | 213.9 | 441.5 | 302.7 | 618.5 | 442.5 | 564.3 |
| (ng · hr/mL) | | | | | | |
| AUC_{0-inf} | 345.9 | 708.2 | 354.8 | 742.0 | 624.0 | 680.2 |
| (ng · hr/mL) | | | | | | |
| AUC₀₋ₜ | 296.8 | 619.1 | 344.8 | 719.6 | 584.6 | 658.4 |
| (ng · hr/mL) | | | | | | |
| Cmax (ng/mL) | 25.97 | 53.51 | 60.5 | 114.2 | 58.5 | 101.1 |
| Tmax (hr) | 2.58 | 2.61 | 1.1 | 1.1 | 4.1 | 1.1 |

TABLE 28
Statistical Analyses of Dose-Normalized PK Parameters
for Hydrocodone

| | Treat- | Treat- | Treat- | Treat- | Treat- |
|---|---|---|---|---|---|
| | ment | ment | ment | ment | ment |
| PK Parameter | B/D | A/C | E/D | E/F | B/C |
| AUC₀₋ₜ/dose | 85.559 | 85.185 | 81.492 | 89.756 | 88.675 |
| (ng · hr/mL/mg) | | | | | |
| AUC_{0-inf}/dose | 96.502 | 95.878 | 84.958 | 93.344 | 100.154 |
| (ng · hr/mL/mg) | | | | | |
| Cₘₐₓ/dose | 46.42 | 42.495 | 51.021 | 58.063 | 43.858 |
| (ng · hr/mL/mg) | | | | | |

FIG. 28 presents the mean drug liking score over a period of 12 hours by treatment. FIG. 29 presents the mean high scores over a period of 12 hours by treatment. FIG. 30 presents the mean good drug effects score over a period of 12 hours by treatment. Table 29 provides a statistical analysis for the LS median difference in Emax for drug liking, high, and good drug effects of various treatment comparisons. A negative value indicates that the first-listed treatment exhibits a lower drug liking, high, and good drug effects. A positive value indicates that the first-listed treatment exhibits a higher drug liking, high, and good drug effects. The data demonstrates that in comparison to the high-dose, intact immediate release formulation (Treatment D), both high-dose, intact and crushed multi-layer extended release formulation (Treatments B and E) as described herein exhibit substantially less drug liking, high, and good drug effects scores. Moreover, the data also surprisingly demonstrates that high-dose, crushed multi-layer extended release tablet formulation (Treatment E) has a lower drug liking, high, and good drug effects than high-dose, intact multi-layer extended release tablet formulation (Treatment B). This discovery was unexpected given that opioid abusers typically crush other tablet formulations to increase the drug liking, high, and good drug effects, which is the opposite of what is observed for the multi-layer extended release tablet formulation described herein. Study validity was confirmed by high scores for drug liking, drug high, and good drug effects for low-dose, high-dose, and crushed immediate release hydrocodone/APAP compared with placebo.

TABLE 29
ANOVA Analysis for the LS Median Difference in Emax for
Drug Liking, High, and Good Drug Effects

| | Treatment | Treatment | Treatment | Treatment |
|---|---|---|---|---|
| PD Parameter | B vs. D | E vs. D | D vs. G | B vs. E |
| Drug Liking | −8.5 | −14.5 | 31 | 6 |
| High | −22 | −37 | 61 | 9 |
| Good Drug | −16 | −31.5 | 70 | 8 |
| Effects | | | | |

The PK characteristics presented in FIG. 27 and Table 27 positively correlated with the pharmacodynamic outcomes of lesser drug liking, drug high, and good drug effects associated with the multi-layer extended release hydrocodone/APAP tablet formulation. Crushing the multi-layer extended release hydrocodone/APAP tablet formulation further slowed the rate of hydrocodone release and produced corresponding decreases in drug liking, drug high, and good drug effects relative to comparable doses of the intact form and intact and crushed immediate release hydrocodone/APAP. Subjective drug effects resulting from administration of the multi-layer extended release hydrocodone/APAP tablet formulation were strongly correlated with the PK profile of hydrocodone. The multi-layer extended release hydrocodone/APAP tablet formulation, which produced decreased hydrocodone Cmax and relatively lower early hydrocodone exposure, was surprisingly associated with lesser degrees of drug liking, drug high, and good drug effects compared to immediate release hydrocodone/APAP. This data suggests that the multi-layer extended release hydrocodone/APAP tablet formulation as described herein surprisingly may be associated with a lower potential for abuse.

### Example 17

### Half-Value Duration Analysis for Acetaminophen after Multiple Doses of Oral Controlled-Release Hydrocodone/Acetaminophen (CR HC/APAP) Tablets

Post hoc analysis of PK data from the clinical study described in Example 13 was performed to evaluate the half-value duration (HVD) for APAP after multiple doses (administered q12h) of the CR HC/APAP formulation compared to the commercially-available IR HC/APAP under the described dosing regimens.

Blood samples for bioanalysis of APAP were collected up to 36 hours after dosing in the single-dose study and up to 132 hours after the hour-0 dose in the multiple-dose study. HVD was calculated for APAP. Descriptive statistics were used to report demographics and baseline characteristics. Analyses were performed for both the initial dose period (day 1, 0-12 h) and at steady state (day 5, 0- to 12-h dosing interval; i.e., 96-108 h). PK analyses included subjects who completed each study. Mean concentration-time profiles were presented on a linear scale. Individual plasma concentration versus actual time data were used to estimate the PK parameters of APAP. HVD of APAP after CR HC/APAP (single or multiple dose) was compared with that after IR HC/APAP using paired 2-tailed t tests. Descriptive statistics and paired t tests were calculated to determine whether HVD of plasma concentrations for hydrocodone and APAP are statistically different for the CR HC/APAP and the commercial IR HC/APAP formulations. This data is provided in Table 30. Table 31 provides half-value Cmax data for the CR HC/APAP and the commercial IR HC/APAP formulations. FIGS. 31 and 32 present the plasma concentration over time and HVD for hydrocodone and APAP, respectively, on Day 1. FIGS. 33 and 34 present the plasma concentration over time and HVD for hydrocodone and APAP, respectively, on Day 5. The data demonstrates that the HVD of plasma concentrations achieved under Treatment A is statistically different from that achieved under Treatment A for APAP on both Day 1 and Day 5 and is statistically different for hydrocodone on Day 1 (P<0.05).

TABLE 30
Summary of mean Half-Value Data (HVD) of plasma
concentrations for Treatments A and B of Example 13

| | | Half Value Duration (hr) | |
|---|---|---|---|
| API | Treatment | Day 1 | Day 5 |
| APAP | A | 2.79 (1.24) | 2.09 (0.84) |
| | B | 3.95 (1.64) | 3.68 (1.18) |
| Hydrocodone | A | 9.99 (1.43) | 9.13 (1.35) |
| | B | 8.58 (1.9) | 8.97 (1.35) |

TABLE 31
Summary of Half-Value Cmax for Treatments
A and B of Example 13

| | | Day 1 | | | Day 5 | |
|---|---|---|---|---|---|---|
| | | Half | Half | Half | Half | Half |
| API | Treatment | Cmax1 | Cmax2 | Cmax3 | Cmax1 | Cmax2 |
| APAP | A | 3918.16 | — | — | 3448.68 | — |
| | B | 2747.63 | 2062.89 | 2647.63 | 2717.63 | 2047.37 |
| HC | A | 13.71 | — | — | 16.36 | — |
| | B | 11.51 | 13.22 | 15.31 | 17.17 | 15.64 |

All references cited herein are hereby incorporated by reference. The foregoing is offered primarily for purposes of illustration. It will be readily apparent to those skilled in the art that further drugs can be included, and that the shapes, components, additives, proportions, methods of formulation, and other parameters described herein can be modified further or substituted in various ways without departing from the spirit and scope of the invention.

What is claimed:

1. A solid oral dosage form comprising:
(a) at least one immediate release portion comprising acetaminophen and hydrocodone or a pharmaceutically acceptable salt thereof; and
(b) at least one extended release portion comprising acetaminophen, hydrocodone or a pharmaceutically acceptable salt thereof, and an extended release component;
wherein the total amount of acetaminophen in the dosage form is about 325 mg to about 650 mg, and the total amount of hydrocodone or its pharmaceutically acceptable salt in the dosage form is about 5 mg to about 15 mg; and
wherein upon oral administration of the dosage form to a subject, the dosage form provides a higher AUC for hydrocodone when the dosage form is administered to the subject in an intact state versus when the dosage form is administered to the subject in a crushed or ground state.

2. The solid oral dosage form of claim 1, wherein the extended release component is an extended release polymer.

3. The solid oral dosage form of claim 2, wherein the extended release portion comprises, by weight of the extended release portion, from about 30% to about 50% of the extended release polymer.

4. The solid oral dosage form of claim 2, wherein the extended release polymer is polyethylene oxide.

5. The solid oral dosage form of claim 4, wherein the polyethylene oxide has a molecular weight of about 900,000 Daltons to about 7,000,000 Daltons.

6. The solid oral dosage form of claim 1, wherein upon oral administration of the dosage form to a subject, the dosage form provides a longer Tₘₐₓ for hydrocodone when the dosage form is administered to the subject in a crushed or ground state versus when the dosage form is administered to the subject in an intact state.

7. The solid oral dosage form of claim 6, wherein administration of the dosage form to a subject produces a mean Tₘₐₓ for hydrocodone that is at least about 30 minutes greater when the dosage form is administered in a crushed or ground state versus when the dosage form is administered in an intact state.

8. The solid oral dosage form of claim 6, wherein administration of the dosage form to a subject produces a mean Tₘₐₓ for hydrocodone that is at least about 45 minutes greater when the dosage form is administered in a crushed or ground state versus when the dosage form is administered in an intact state.

9. The solid oral dosage form of claim 6, wherein administration of the dosage form to a subject produces a mean Tₘₐₓ for hydrocodone that is at least about 60 minutes greater when the dosage form is administered in a crushed or ground state versus when the dosage form is administered in an intact state.

10. The solid oral dosage form of claim 6, wherein administration of the dosage form to a subject produces a mean Tₘₐₓ for hydrocodone that is at least about 75 minutes greater when the dosage form is administered in a crushed or ground state versus when the dosage form is administered in an intact state.

11. The solid oral dosage form of claim 6, wherein administration of the dosage form to a subject produces a mean Tₘₐₓ for hydrocodone that is at least about 90 minutes greater when the dosage form is administered in a crushed or ground state versus when the dosage form is administered in an intact state.

12. The solid oral dosage form of claim 6, wherein administration of the dosage form to a subject produces a mean Tₘₐₓ for hydrocodone that is at least about 105 minutes greater when the dosage form is administered in a crushed or ground state versus when the dosage form is administered in an intact state.

13. The solid oral dosage form of claim 6, wherein administration of the dosage form to a subject produces a mean Tₘₐₓ for hydrocodone that is at least about 120 minutes greater when the dosage form is administered in a crushed or ground state versus when the dosage form is administered in an intact state.

14. The solid oral dosage form of claim 1, wherein upon oral administration of the dosage form to a subject, the dosage form provides a AUC(0-1 hr) for hydrocodone that is about 50% to about 1000% higher when the dosage form is administered to the subject in an intact state versus when the dosage form is administered to the subject in a crushed or ground state.

15. The solid oral dosage form of claim 14, wherein upon oral administration of the dosage form to a subject, the dosage form provides a AUC(0-1 hr) for hydrocodone that is about 100% to about 900% higher when the dosage form is administered to the subject in an intact state versus when the dosage form is administered to the subject in a crushed or ground state.

16. The solid oral dosage form of claim 14, wherein upon oral administration of the dosage form to a subject, the dosage form provides a AUC(0-1 hr) for hydrocodone that is about 200% to about 800% higher when the dosage form is administered to the subject in an intact state versus when the dosage form is administered to the subject in a crushed or ground state.

17. The solid oral dosage form of claim 14, wherein upon oral administration of the dosage form to a subject, the dosage form provides a AUC(0-1 hr) for hydrocodone that is about 300% to about 700% higher when the dosage form is administered to the subject in an intact state versus when the dosage form is administered to the subject in a crushed or ground state.

18. The solid oral dosage form of claim 1, wherein upon oral administration of the dosage form to a subject, the dosage form provides a AUC(0-2 hr) for hydrocodone that is about 50% to about 500% higher when the dosage form is administered to the subject in an intact state versus when the dosage form is administered to the subject in a crushed or ground state.

19. The solid oral dosage form of claim 18, wherein upon oral administration of the dosage form to a subject, the dosage form provides a AUC(0-2 hr) for hydrocodone that is about 100% to about 400% higher when the dosage form is administered to the subject in an intact state versus when the dosage form is administered to the subject in a crushed or ground state.

20. The solid oral dosage form of claim 18, wherein upon oral administration of the dosage form to a subject, the dosage form provides a AUC(0-2 hr) for hydrocodone that is about 150% to about 300% higher when the dosage form is administered to the subject in an intact state versus when the dosage form is administered to the subject in a crushed or ground state.

21. The solid oral dosage form of claim 18, wherein upon oral administration of the dosage form to a subject, the dosage form provides a AUC(0-2 hr) for hydrocodone that is about 50% to about 250% higher when the dosage form is administered to the subject in an intact state versus when the dosage form is administered to the subject in a crushed or ground state.

22. The solid dosage form of claim 1, wherein upon oral administration of the dosage form to a subject, the dosage form provides a Tmax for hydrocodone that decreases by about 5% to about 70% when the dosage form is administered in an intact state versus when the dosage form is administered in a crushed or ground state.

23. The solid dosage form of claim 22, wherein the Tmax for hydrocodone is decreased by about 5% to about 50% when the dosage form is administered in an intact state versus when the dosage form is administered in a crushed or ground state.

24. The solid dosage form of claim 22, wherein the Tmax for hydrocodone is decreased by about 5% to about 40% when the dosage form is administered in an intact state versus when the dosage form is administered in a crushed or ground state.

25. The solid dosage form of claim 22, wherein the Tmax for hydrocodone is decreased by about 5% to about 30% when the dosage form is administered in an intact state versus when the dosage form is administered in a crushed or ground state.

26. The solid dosage form of claim 22, wherein the Tmax for hydrocodone is decreased by about 5% to about 20% when the dosage form is administered in an intact state versus when the dosage form is administered in a crushed or ground state.

27. The solid dosage form of claim 22, wherein the Tmax for hydrocodone is decreased by about 10% to about 40% when the dosage form is administered in an intact state versus when the dosage form is administered in a crushed or ground state.

28. The solid dosage form of claim 22, wherein the Tmax for hydrocodone is decreased by about 20% to about 60% when the dosage form is administered in an intact state versus when the dosage form is administered in a crushed or ground state.

29. The solid dosage form of claim 6, wherein the solid dosage form contains a total of about 325 mg of acetaminophen and about 5 mg of hydrocodone or its pharmaceutically acceptable salt.

30. The solid dosage form of claim 6, wherein the solid dosage form contains a total of about 325 mg of acetaminophen and about 7.5 mg of hydrocodone or its pharmaceutically acceptable salt.

31. The solid dosage form of claim 6, wherein the solid dosage form contains a total of about 325 mg of acetaminophen and about 10 mg of hydrocodone or its pharmaceutically acceptable salt.

32. The solid dosage form of claim 6, wherein the solid dosage form contains a total of about 325 mg of acetaminophen and about 15 mg of hydrocodone or its pharmaceutically acceptable salt.

33. The solid dosage form of claim 14, wherein the solid dosage form contains a total of about 325 mg of acetaminophen and about 5 mg of hydrocodone or its pharmaceutically acceptable salt.

34. The solid dosage form of claim 14, wherein the solid dosage form contains a total of about 325 mg of acetaminophen and about 7.5 mg of hydrocodone or its pharmaceutically acceptable salt.

35. The solid dosage form of claim 14, wherein the solid dosage form contains a total of about 325 mg of acetaminophen and about 10 mg of hydrocodone or its pharmaceutically acceptable salt.

36. The solid dosage form of claim 14, wherein the solid dosage form contains a total of about 325 mg of acetaminophen and about 15 mg of hydrocodone or its pharmaceutically acceptable salt.

37. The solid dosage form of claim 18, wherein the solid dosage form contains a total of about 325 mg of acetaminophen and about 5 mg of hydrocodone or its pharmaceutically acceptable salt.

38. The solid dosage form of claim 18, wherein the solid dosage form contains a total of about 325 mg of acetaminophen and about 7.5 mg of hydrocodone or its pharmaceutically acceptable salt.

39. The solid dosage form of claim 18, wherein the solid dosage form contains a total of about 325 mg of acetaminophen and about 10 mg of hydrocodone or its pharmaceutically acceptable salt.

40. The solid dosage form of claim 18, wherein the solid dosage form contains a total of about 325 mg of acetaminophen and about 15 mg of hydrocodone or its pharmaceutically acceptable salt.

41. The solid oral dosage form of claim 1, wherein upon oral administration of the dosage form to a subject, the dosage form provides a longer Tₘₐₓ for acetaminophen when the dosage form is administered to the subject in a crushed or ground state versus when the dosage form is administered to the subject in an intact state.

42. The solid oral dosage form of claim 41, wherein administration of the dosage form to a subject produces a mean Tₘₐₓ for acetaminophen that is at least about one hour greater when the dosage form is administered in a crushed or ground state as compared to an intact state.

43. The solid oral dosage form of claim 1, wherein upon oral administration of the dosage form to a subject, the dosage form provides a higher Cₘₐₓ for acetaminophen when the dosage form is administered to the subject in an intact state versus when the dosage form is administered to the subject in a crushed or ground state.

44. The solid oral dosage form of claim 1, wherein the total amount of acetaminophen in the composition is about 325 mg and the total amount of hydrocodone or its pharmaceutically acceptable salt in the dosage form is about 7.5 mg.

45. The solid oral dosage form of claim 1, wherein the total amount of acetaminophen in the dosage form is about 325 mg and the total amount of hydrocodone or its pharmaceutically acceptable salt in the dosage form is about 5 mg.

46. The solid oral dosage form of claim 1, wherein the total amount of acetaminophen in the dosage form is about 325 mg and the total amount of hydrocodone or its pharmaceutically acceptable salt in the dosage form is about 10 mg.

47. The solid oral dosage form of claim 1, wherein the total amount of acetaminophen in the dosage form is about 325 mg and the total amount of hydrocodone or its pharmaceutically acceptable salt in the dosage form is about 15 mg.

48. A solid oral dosage form comprising:
(a) at least one immediate release portion comprising acetaminophen and hydrocodone or a pharmaceutically acceptable salt thereof; and
(b) at least one extended release portion comprising acetaminophen, hydrocodone or a pharmaceutically acceptable salt thereof, and an extended release component;
wherein the total amount of acetaminophen in the dosage form is about 325 mg to about 650 mg, and the total amount of hydrocodone or its pharmaceutically acceptable salt in the dosage form is about 5 mg to about 15 mg; and
wherein upon oral administration of the dosage form to a subject, the dosage form provides an abuse quotient for hydrocodone that is higher when the dosage form is administered to the subject in an intact state versus when the dosage form is administered to the subject in a crushed or ground state.

49. The solid oral dosage form of claim 48, wherein the abuse quotient for hydrocodone is decreased by about 5% to about 90% when the dosage form is administered in a crushed or ground state versus when the dosage form is administered in an intact state.

50. The solid oral dosage form of claim 49, wherein the abuse quotient for hydrocodone is decreased by about 10% to about 80% when the dosage form is administered in a crushed or ground state versus when the dosage form is administered in an intact state.

51. The solid oral dosage form of claim 49, wherein the abuse quotient for hydrocodone is decreased by about 15% to about 70% when the dosage form is administered in a crushed or ground state versus when the dosage form is administered in an intact state.

52. The solid oral dosage form of claim 49, wherein the abuse quotient for hydrocodone is decreased by about 20% to about 60% when the dosage form is administered in a crushed or ground state versus when the dosage form is administered in an intact state.

53. The solid oral dosage form of claim 48, wherein the solid dosage form contains a total of about 325 mg of acetaminophen and about 5 mg of hydrocodone or its pharmaceutically acceptable salt.

54. The solid oral dosage form of claim 48, wherein the solid dosage form contains a total of about 325 mg of acetaminophen and about 7.5 mg of hydrocodone or its pharmaceutically acceptable salt.

55. The solid oral dosage form of claim 48, wherein the solid dosage form contains a total of about 325 mg of acetaminophen and about 10 mg of hydrocodone or its pharmaceutically acceptable salt.

56. The solid oral dosage form of claim 48, wherein the solid dosage form contains a total of about 325 mg of acetaminophen and about 15 mg of hydrocodone or its pharmaceutically acceptable salt.

57. The solid oral dosage form of claim 48, wherein the extended release component is an extended release polymer.

58. The solid oral dosage form of claim 57, wherein the extended release portion comprises, by weight of the extended release portion, from about 30% to about 50% of the extended release polymer.

59. The solid oral dosage form of claim 57, wherein the extended release polymer is polyethylene oxide.

60. The solid oral dosage form of claim 59, wherein the polyethylene oxide has a molecular weight of about 900,000 Daltons to about 7,000,000 Daltons.

61. The solid oral dosage form of claim 48, wherein administration of the dosage form to a subject produces a mean Tₘₐₓ for hydrocodone that is at least about 30 minutes greater when the dosage form is administered in a crushed or ground state versus when the dosage form is administered in an intact state.

62. The solid oral dosage form of claim 48, wherein administration of the dosage form to a subject produces a mean Tₘₐₓ for hydrocodone that is at least about 45 minutes greater when the dosage form is administered in a crushed or ground state versus when the dosage form is administered in an intact state.

63. The solid oral dosage form of claim 48, wherein administration of the dosage form to a subject produces a mean Tₘₐₓ for hydrocodone that is at least about 60 minutes greater when the dosage form is administered in a crushed or ground state versus when the dosage form is administered in an intact state.

64. The solid oral dosage form of claim 48, wherein administration of the dosage form to a subject produces a mean Tₘₐₓ for hydrocodone that is at least about 75 minutes greater when the dosage form is administered in a crushed or ground state versus when the dosage form is administered in an intact state.

65. The solid oral dosage form of claim 48, wherein administration of the dosage form to a subject produces a mean Tₘₐₓ for hydrocodone that is at least about 90 minutes greater when the dosage form is administered in a crushed or ground state versus when the dosage form is administered in an intact state.

66. A solid oral dosage form comprising:
(a) at least one immediate release portion comprising acetaminophen and hydrocodone or a pharmaceutically acceptable salt thereof; and
(b) at least one extended release portion comprising acetaminophen, hydrocodone or a pharmaceutically acceptable salt thereof, and an extended release component;
wherein the total amount of acetaminophen in the dosage form is about 325 mg to about 650 mg, and the total amount of hydrocodone or its pharmaceutically acceptable salt in the dosage form is about 5 mg to about 15 mg; and
wherein administration of the dosage form to a subject produces a mean Tₘₐₓ for hydrocodone that is at least about 30 minutes greater when the dosage form is administered in a crushed or ground state versus when the dosage form is administered in an intact state.

67. The solid oral dosage form of claim 66, wherein administration of the dosage form to a subject produces a mean Tₘₐₓ for hydrocodone that is at least about 60 minutes greater when the dosage form is administered in a crushed or ground state versus when the dosage form is administered in an intact state.

68. The solid oral dosage form of claim 66, wherein administration of the dosage form to a subject produces a mean Tₘₐₓ for hydrocodone that is at least about 75 minutes greater when the dosage form is administered in a crushed or ground state versus when the dosage form is administered in an intact state.

69. The solid oral dosage form of claim 66, wherein administration of the dosage form to a subject produces a mean Tₘₐₓ for hydrocodone that is at least about 90 minutes greater when the dosage form is administered in a crushed or ground state versus when the dosage form is administered in an intact state.

70. The solid oral dosage form of claim 66, wherein the extended release portion comprises, by weight of the extended release portion, from about 30% to about 50% of an extended release polymer comprising polyethylene oxide having a molecular weight of about 900,000 Daltons to about 7,000,000 Daltons.

71. The solid oral dosage form of claim 66, wherein the solid dosage form contains a total of about 325 mg of acetaminophen and about 5 mg of hydrocodone or its pharmaceutically acceptable salt.

72. The solid oral dosage form of claim 66, wherein the solid dosage form contains a total of about 325 mg of acetaminophen and about 7.5 mg of hydrocodone or its pharmaceutically acceptable salt.

73. The solid oral dosage form of claim 66, wherein the solid dosage form contains a total of about 325 mg of acetaminophen and about 10 mg of hydrocodone or its pharmaceutically acceptable salt.

74. The solid oral dosage form of claim 66, wherein the solid dosage form contains a total of about 325 mg of acetaminophen and about 15 mg of hydrocodone or its pharmaceutically acceptable salt.

75. The solid oral dosage form of claim 70, wherein upon oral administration of the dosage form to a subject, the dosage form provides a AUC(0-1 hr) for hydrocodone that is about 50% to about 1000% higher when the dosage form is administered to the subject in an intact state versus when the dosage form is administered to the subject in a crushed or ground state.
